(19) United States
(12) Patent Application Publication
(10) Pub. No.: US 2026/0078119 A1
(43) Pub. Date: Mar. 19, 2026

(54) DEGRADER COMPOUNDS AND USES THEREOF

(71) Applicant: Bristol-Myers Squibb Company, Princeton, NJ (US)

(72) Inventors: Daniel K. CASHION, San Diego, CA (US); Jesus MORENO, Lemon Grove, CA (US); David S. PETERS, Encinitas, CA (US); Thomas J. CUMMINS, San Diego, CA (US); Vera PRYTKOVA, San Diego, CA (US); Jennifer R. RIGGS, Cardiff, CA (US); Sophie M. PERRIN, La Jolla, CA (US)

(21) Appl. No.: 19/086,425

(22) Filed: Mar. 21, 2025

Related U.S. Application Data
(60) Provisional application No. 63/568,728, filed on Mar. 22, 2024.

(51) Int. Cl. C07D 471/04; A61K 31/4545; A61K 31/496; A61K 31/501; A61K 31/506; A61K 31/519; A61K 31/5377; A61K 45/06; A61P 35/00; C07D 401/14; C07D 487/04; C07D 519/00

(52) U.S. Cl. CPC C07D 471/04; A61K 31/4545; A61K 31/496; A61K 31/501; A61K 31/506; A61K 31/519; A61K 31/5377; A61K 45/06; A61P 35/00; C07D 401/14; C07D 487/04; C07D 519/00

(57) ABSTRACT

Provided herein are compounds and compositions thereof that reduce FAK protein levels. In some embodiments, the compounds have structures of Formula I:

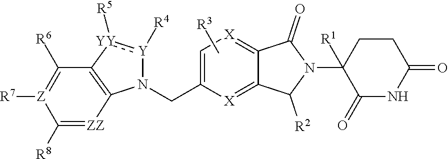

In some embodiments, the compounds and compositions are provided for treatment of FAK associated diseases such as cancer.

### CROSS REFERENCE TO RELATED APPLICATIONS

This application claims the benefit under 35 U.S.C. § 119(e) of U.S. Provisional Application Ser. No. 63/568,728, filed Mar. 22, 2024; the disclosure of which is incorporated herein by reference.

### FIELD OF INVENTION

This disclosure relates to novel Focal Adhesion Kinase (FAK) degraders and methods comprising administering a FAK degrader that are useful in the treatment of abnormal cell growth, such as cancer, in mammals, especially humans.

### BACKGROUND OF INVENTION

Convincing evidence suggests that focal adhesion kinase (FAK), i.e., PTK2, a cytoplasmic, non-receptor tyrosine kinase, plays an essential role in cell-matrix signal transduction pathways (Clark and Brugge 1995, Science 268:233-239) and its aberrant activation is associated with an increase in the metastatic potential of tumors (Owens et al. 1995, Cancer Research 55:2752-2755). FAK is encoded by the PTK2 gene in humans, and was originally identified as a 125 kDa protein highly tyrosine-phosphorylated in cells transformed by v-Src. FAK was subsequently found to be a tyrosine kinase that localizes to focal adhesions, which are macromolecular assemblies that form contact points between the cytoskeleton of cells and the extracellular matrix (ECM), and which serve as both mechanical sensors and a signal transducing hub (Geiger et al. 2009, Nat Rev Mol Cell Biol. 10:21-33). FAK is phosphorylated and, thus, activated in response to extracellular matrix-binding to integrins. Recently, studies have demonstrated that an increase in FAK mRNA levels accompanied invasive transformation of tumors and attenuation of the expression of FAK (through the use of antisense oligonucleotides) induces apoptosis in tumor cells (Xu et al 1996, Cell Growth and Diff 7: 413-418). In addition to being expressed in most tissue types, FAK is found at elevated levels in most human cancers, for example in highly invasive metastases. For example, U.S. Pat. No. 8,247,411 relates to a broad class of novel pyrimidine derivatives that are kinase inhibitors, and more specifically, inhibitors of FAK. Compounds such as these may be useful in the treatment of abnormal cell growth.

Cancers can be recognized by the immune system, and regulate and even eliminate tumors. Immune checkpoints refer to a plethora of inhibitory pathways that help maintain self-tolerance and modulate the duration and amplitude of physiological immune responses in peripheral tissues in order to minimize collateral tissue damage. Tumors co-opt certain immune-checkpoint pathways as a mechanism of immune resistance, particularly against T-cells that are specific for tumor antigens. The development of checkpoint blocking antibodies, e.g., inhibitory receptors, that target or are directed against, e.g., cytotoxic T-lymphocyte antigen 4 (CTLA-4) and programmed death 1 receptor (PD-1), can facilitate the treatment of a disease or disorder described herein (e.g., abnormal cell growth, e.g., cancer (e.g., a cancer described herein)) Substantial efforts are ongoing to identify agents that can augment T-cell mediated killing of tumor cells and potentiate the effects of checkpoint inhibitors. Focal Adhesion Kinase (FAK) and the closely related family member PYK2 are potentially valuable targets in this regard due to the roles of these enzymes in regulating key cellular populations in the tumor microenvironment. FAK inhibitors may increase cytotoxic T-cells (CD8+ expressing cytotoxic T-cells) in tumors, and decrease the immune cell populations that suppress the host anti-tumor immune response (T-regs, M2 tumor associated macrophages, myeloid-derived suppressor cells)

The compounds described herein, e.g., FAK inhibitors may be used to prevent and treat a disease or disorder described herein, e.g., abnormal cell growth (e.g., a cancer described herein).

### SUMMARY OF THE INVENTION

This specification relates to compounds having the Formula (I),

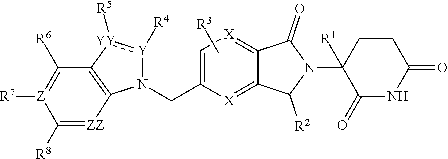

or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
[symbol] may be absent or a double bond;
X is independently selected from C and N;
Y is independently selected from C and N;
YY is independently selected from C and N;
Z is independently selected from C and N;
ZZ is independently selected from C and N;
R¹ is independently selected from hydrogen, halogen, and —C₁-C₆ alkyl;
R² is independently selected from hydrogen, halogen, —(═O), —C₁-C₆ alkyl, a 3 to 6 membered cycloalkyl, wherein the alkyl or cycloalkyl may be optionally substituted with —R⁹, —N(R⁹R¹⁰), and —OR⁹;
R³ is independently absent or selected from hydrogen and halogen;
R⁴ is independently absent or selected from hydrogen and —C₁-C₆ alkyl;
R⁵ is independently absent or selected from hydrogen, halogen and —C₁-C₆ alkyl;
R⁶ is independently selected from a 5 to 12 membered aryl and a 5 to 12 membered heteroaryl ring, wherein the aryl and heteroaryl may be optionally substituted with 1, 2, or 3-R⁹, —N(R⁹R¹⁰), and —OR⁹;
R⁷ is hydrogen or halogen;
R⁸ is independently selected from —N(R⁹R¹⁰), and a 4 to 12 membered heterocyclic ring, wherein the heterocyclic ring may be optionally substituted with 1, 2 or 3-R⁹, —N(R⁹R¹⁰), and —OR⁹;
R⁹ is independently selected from hydrogen, halogen, —OR¹⁰, —N(R¹⁰R¹⁰), —C₁-C₆ alkyl, —O—C₁-C₆ alkyl, —CN, a 3 to 12 membered cycloalkyl, and a 4 to 12 membered heterocyclic ring;
wherein the alkyl, cycloalkyl, or heterocyclic ring in R⁹ are each independently unsubstituted or substituted with 1, 2, or 3 R¹⁰ substituents;
R¹⁰ in each instance is independently selected from hydrogen, —OH, —C₁-C₆ alkyl, —C₂-C₆ alkenyl, halogen, —O—(C₁-C₆ alkyl)-, —N(R¹¹R¹¹), a 3 to 12 membered cycloalkyl, a 4 to 12 membered heterocyclic, a 5 to 12 membered aryl and a 5 to 12 membered heteroaryl ring; wherein the alkyl, alkenyl, cycloalkyl, heterocyclic, aryl or heteroaryl ring in R¹⁰ are each independently unsubstituted or substituted with 1, 2, or 3 R¹¹ substituents; and
R¹¹ is independently selected from hydrogen, halogen, —OH, and —C₁-C₆ alkyl, and further wherein the alkyl, heterocyclic and heteroaryl ring in each R⁶, R⁸, R⁹, and R¹⁰ may include 1, 2 or 3 heteroatoms independently selected from O, N or S.

### DETAILED DESCRIPTION OF THE INVENTION

Compounds disclosed herein can be used for use as a medicament and for use in a method of treating cancer, comprising administering to a mammal having cancer a therapeutically effective amount of the compound.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean ±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include-isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, cycloalkylalkyloxy, arylalkyloxy, heterocyclylalkyloxy, heteroarylalkyloxy; oxo (═O); amino, alkylamino, cycloalkylamino, arylamino, heterocyclylamino, heteroarylamino, cycloalkylalkylamino, arylalkylamino, heterocyclylalkylamino, heteroarylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or —O(alkyl)aminocarbonyl.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2 (1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1 (2H)-onyl), tetrahydroquinolinyl, quinoxalinyl, and quinazolinyl groups. A heteroaryl group can be substituted or unsubstituted.

A “halogen” or “halo” is fluorine, chlorine, bromine or iodine.

An “alkoxy” group is-O-(alkyl), wherein alkyl is defined above.

An “oxo” group is a “═O” group bonded to a carbon.

An “amino” group is —NH₂, wherein one or both of the hydrogen atoms may be substituted with alkyl, cycloalkyl, heterocyclyl, aryl, or heteroaryl.

An “amido” group is an amide group with the formula —NHC(O)—, wherein the hydrogen atom may be substituted with alkyl, cycloalkyl, heterocyclyl, aryl, or heteroaryl.

A “heteroaryl-oxy” group is-O-(heteroaryl), wherein the heteroaryl is defined above. A “heterocyclyl-oxy” group is-O-(heterocyclyl), wherein the heterocyclyl is defined above. A “cycloalkyl-oxy” group is-O-(cycloalkyl), wherein the cycloalkyl is defined above

When the groups described herein, with the exception of alkyl group, amino group, and amido group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxo (═O); B(OH)₂, —O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl); cycloalkyloxy, aryloxy, heterocyclyloxy, heteroaryloxy, cycloalkylalkyloxy, arylalkyloxy, heterocyclylalkyloxy, and heteroarylalkyloxy.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of Formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KgaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

“Tautomers” refers to isomeric forms of a compound that are in equilibrium based on proton transfers. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

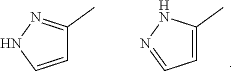

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an FAK mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

1. In a first aspect, the invention provides a group of compounds having a Formula (I),

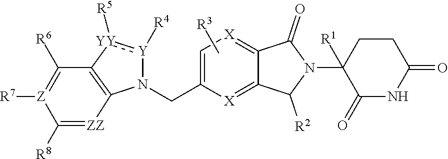

or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
[symbol] may be absent or a double bond;
X is independently selected from C and N;
Y is independently selected from C and N;
YY is independently selected from C and N;
Z is independently selected from C and N;
ZZ is independently selected from C and N;
R¹ is independently selected from hydrogen, halogen, and —C₁-C₆ alkyl;
R² is independently selected from hydrogen, halogen, —(═O), —C₁-C₆ alkyl, a 3 to 6 membered cycloalkyl, wherein the alkyl or cycloalkyl may be optionally substituted with —R⁹, —N(R⁹R¹⁰), and —OR⁹;
R³ is independently absent or selected from hydrogen and halogen;
R⁴ is independently absent or selected from hydrogen and —C₁-C₆ alkyl;
R⁵ is independently absent or selected from hydrogen, halogen and —C₁-C₆ alkyl;
R⁶ is independently selected from a 5 to 12 membered aryl and a 5 to 12 membered heteroaryl ring, wherein the aryl and heteroaryl may be optionally substituted with 1, 2, or 3-R⁹, —N(R⁹R¹⁰), and —OR⁹;
R⁷ is hydrogen or halogen;
R⁸ is independently selected from —N(R⁹R¹⁰), and a 4 to 12 membered heterocyclic ring, wherein the heterocyclic ring may be optionally substituted with 1, 2 or 3-R⁹, —N(R⁹R¹⁰), and —OR⁹;
R⁹ is independently selected from hydrogen, halogen, —OR¹⁰, —N(R¹⁰R¹⁰), —C₁-C₆ alkyl, —O—C₁-C₆ alkyl, —CN, a 3 to 12 membered cycloalkyl, and a 4 to 12 membered heterocyclic ring;
wherein the alkyl, cycloalkyl, or heterocyclic ring in R⁹ are each independently unsubstituted or substituted with 1, 2, or 3 R¹⁰ substituents;
R¹⁰ in each instance is independently selected from hydrogen, —OH, —C₁-C₆ alkyl, —C₂-C₆ alkenyl, halogen, —O—(C₁-C₆ alkyl)-, —N(R¹¹R¹¹), a 3 to 12 membered cycloalkyl, a 4 to 12 membered heterocyclic, a 5 to 12 membered aryl and a 5 to 12 membered heteroaryl ring; wherein the alkyl, alkenyl, cycloalkyl, heterocyclic, aryl or heteroaryl ring in R¹⁰ are each independently unsubstituted or substituted with 1, 2, or 3 R¹¹ substituents; and
R¹¹ is independently selected from hydrogen, halogen, —OH, and —C₁-C₆ alkyl, and further wherein the alkyl, heterocyclic and heteroaryl ring in each R⁶, R⁸, R⁹, and R¹⁰ may include 1, 2 or 3 heteroatoms independently selected from O, N or S.
2. In some embodiments, the compound of embodiment 1, wherein [symbol] is a double bond.
3. In some embodiments, the compound of embodiment 1, wherein [symbol] is absent.
4. In some embodiments, the compound of any one of embodiments 1, 2 or 3, wherein X is C.
5. In some embodiments, the compound of any one of embodiments 1-4, wherein Y is N.
6. In some embodiments, the compound of any one of embodiments 1-4, wherein Y is C.
7. In some embodiments, the compound of any one of embodiments 1-6, wherein YY is N.
8. In some embodiments, the compound of any one of embodiments 1-6, wherein YY is C.
9. In some embodiments, the compound of any one of embodiments 1-8, wherein Z is N.
10. In some embodiments, the compound of any one of embodiments 1-8, wherein Z is C.
11. In some embodiments, the compound of any one of embodiments 1-10, wherein ZZ is N.
12. In some embodiments, the compound of any one of embodiments 1-10, wherein ZZ is C.
13. In some embodiments, the compound of any one of embodiments 1-12, wherein R¹ is hydrogen.
14. In some embodiments, the compound of any one of embodiments 1-12, wherein R² is hydrogen.
15. In some embodiments, the compound of any one of embodiments 1-14, wherein R³ is hydrogen.
16. In some embodiments, the compound of any one of embodiments 1-15, wherein R⁴ is absent.
17. In some embodiments, the compound of any one of embodiments 1-15, herein R⁴ is hydrogen.
18. In some embodiments, the compound of any one of embodiments 1-15, wherein R⁴ is —C₁-C₆ alkyl.
19. In some embodiments, the compound of embodiment 18, wherein R⁴ is —CH₃.
20. In some embodiments, the compound of embodiment 18, wherein R⁴ is —CH₂CH₃.
21. In some embodiments, the compound of any one of embodiments 1-20, wherein R⁵ is hydrogen.
22. In some embodiments, the compound of any one of embodiments 1-20, wherein R⁵ is —C₁-C₆ alkyl.
23. In some embodiments, the compound of embodiment 22, wherein R⁵ is —CH₃.
24. In some embodiments, the compound of any one of embodiments 1-20, wherein R⁵ is halogen.
25. In some embodiments, the compound according to any one of embodiments 1-2, 4-5, 8, 10-11, 13-15, and 21-24, having a Formula (Ia),

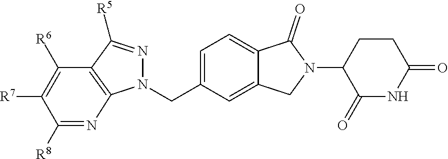

or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
R⁵ is independently absent or selected from hydrogen, halogen or —C₁-C₆ alkyl;
R⁶ is independently selected from a 5 to 12 membered aryl or a 5 to 12 membered heteroaryl ring, wherein the aryl, and heteroaryl may be optionally substituted with 1, 2, or 3-R⁹, —N(R⁹R¹⁰), or —OR⁹;
R⁷ is hydrogen or halogen;
R⁸ is independently selected from —N(R⁹R¹⁰), a 4 to 12 membered heterocyclic, ring, wherein the heterocyclic ring may be optionally substituted with 1, 2 or 3-R⁹, —N(R⁹R¹⁰), or —OR⁹;
R⁹ is independently selected from hydrogen, halogen, —C₁-C₆ alkyl, —OR¹⁰, —N(R¹⁰R¹⁰), —CN, a 3 to 12 membered cycloalkyl, or a 4 to 12 membered heterocyclic ring;
wherein the alkyl, cycloalkyl, or heterocyclic ring in R⁹ are each independently unsubstituted or substituted with 1, 2, or 3 R¹⁰ substituents;
R¹⁰ in each instance is independently selected from hydrogen, halogen, —OH, —C₁-C₆ alkyl, 3 to 12 membered cycloalkyl, and 4 to 12 membered heterocyclic ring; wherein the alkyl, cycloalkyl, and heterocyclic ring in R¹⁰ are each independently unsubstituted or substituted with 1, 2, or 3 R¹¹ substituents;
R¹¹ is independently hydrogen, halogen, —OH, and —C₁-C₆ alkyl, further
wherein the cycloalkyl, heterocyclic and heteroaryl cyclic ring in each R⁶, R⁸, R⁹, and R¹⁰ may include 1, 2 or 3 heteroatoms independently selected from O, N or S.
26. In some embodiments, the compound of embodiment 25, wherein R⁵ is hydrogen.
27. In some embodiments, the compound of embodiment 25, wherein R⁵ is halogen.
28. In some embodiments, the compound of embodiment 27, wherein R⁵ is Cl or F.
29. In some embodiments, the compound of embodiment 25, wherein R⁵ is —C₁-C₆ alkyl.
30. In some embodiments, the compound of embodiment 29, wherein R⁵ is —CH₃.
31. In some embodiments, the compound of any one of embodiments 25-30, wherein R⁶ is selected from a 5 to 12 membered aryl ring, optionally substituted with 1, 2, or 3-R⁹, —N(R⁹R¹⁰), or —OR⁹.
32. In some embodiments, the compound of any one of embodiments 25-30, wherein R⁶ is a 5 to 12 membered heteroaryl ring, optionally substituted with 1, 2, or 3-R⁹, —N(R⁹R¹⁰), or —OR⁹.
33. In some embodiments, the compound of any one of embodiments 25-32, wherein R⁷ is hydrogen.
34. In some embodiments, the compound of any one of embodiments 25-32, wherein R⁷ is halogen.
35. In some embodiments, the compound of embodiment 34, wherein the halogen is F or C₁.
36. In some embodiments, the compound of any one of embodiments 25-34, wherein R⁸ is independently selected from —N(R⁹R¹⁰), and a 4 to 12 membered heterocyclic ring.
37. In some embodiments, the compound of embodiment 36, wherein R⁸ is —N(R⁹R¹⁰).
38. In some embodiments, the compound of embodiment 36, wherein R⁸ is a 4 to 12 membered heterocyclic ring.
39. In some embodiments, the compound of any one of embodiments 25-40, wherein R⁹ is hydrogen.
40. In some embodiments, the compound of any one of embodiments 25-40, wherein R⁹ is halogen.
41. In some embodiments, the compound of embodiment 42, wherein the halogen is selected from Cl and F.
42. In some embodiments, the compound of embodiment 43, wherein the halogen is selected from Cl.
43. In some embodiments, the compound of embodiment 43, wherein the halogen is selected from F.
44. In some embodiments, the compound of any one of embodiments 25-38, having the Formula (Ia), wherein R⁹ is —C₁-C₆ alkyl.
45. In some embodiments, the compound of embodiment 41, wherein the —C₁-C₆ alkyl is selected from —CH₃, —CH₂CH₃, —CH(CH₃)₂, and —C(CH₃) 3.
46. In some embodiments, the compound of any one of embodiments 25-38, wherein R¹⁰ is —OR¹⁰, —N(R¹⁰R¹⁰), —CN, a 3 to 12 membered cycloalkyl, or a 4 to 12 membered heterocyclic ring;
wherein the alkyl, cycloalkyl, or heterocyclic ring in R⁹ are each independently unsubstituted or substituted with 1, 2, or 3 R¹⁰ substituents.
47. In some embodiments, the compound of any one of embodiments 25-46, wherein R¹⁰ in each instance is independently selected from hydrogen, halogen, —OH, —C₁-C₆ alkyl, —N(R¹¹R¹¹), a 3 to 12 membered cycloalkyl, a 4 to 12 membered heterocyclic, ring; wherein the alkyl, cycloalkyl, heterocyclic ring in R¹⁰ are each independently unsubstituted or substituted with 1, 2, or 3 R¹¹ substituents.
48. In some embodiments, the compound of any one of embodiments 25-47, wherein R¹¹ is independently hydrogen, halogen, —OH, or —C₁-C₆ alkyl.
49. In some embodiments, the compound of any one of embodiments 1, and 25-48, wherein R⁶ is independently selected from:

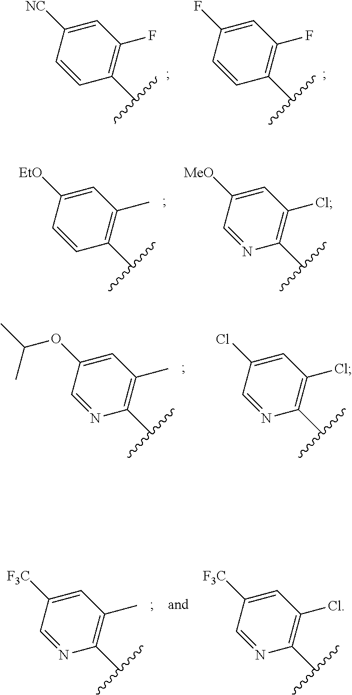

50. In some embodiments, the compound of any one of embodiments 1, and 25-49, wherein R⁸ is independently selected from:

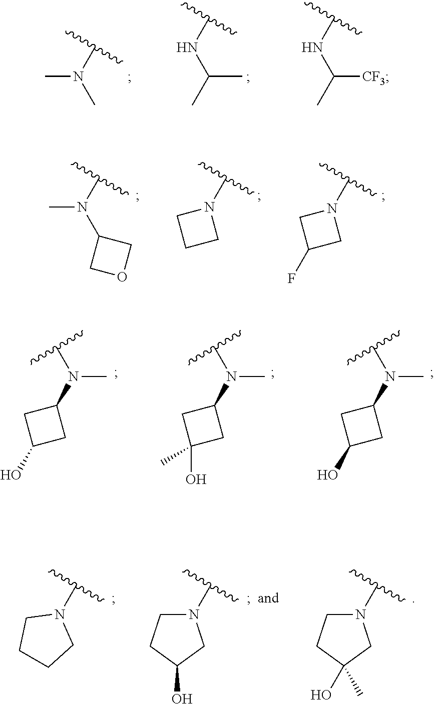

51. In some embodiments, the compound of embodiment 1, having a Formula (Ib),

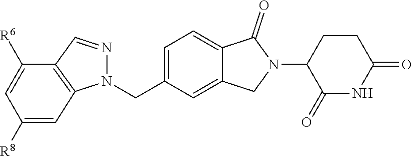

or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof, wherein:
R⁶ is

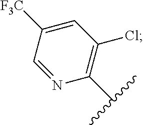 [SMILES: *c1ncc(C(F)(F)F)cc1Cl]

and
R⁸ is

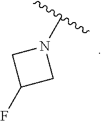 [SMILES: *N1CC(F)C1]

52. In some embodiments, the compound of embodiment 1, having a Formula (Ic),

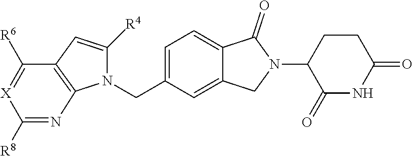

or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof, wherein:
X is C or N;
R⁴ is independently selected from hydrogen or —C₁-C₆ alkyl;
R⁶ is independently selected from

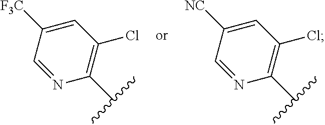 [SMILES: *c1ncc(C(F)(F)F)cc1Cl ; *c1ncc(C#N)cc1Cl]

and
R⁸ is independently selected from

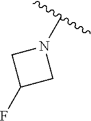 [SMILES: *N1CC(F)C1]

or —N(CH₃CH₃).
53. In some embodiments, the compound of embodiment 52, wherein R⁴ is hydrogen.
54. In some embodiments, the compound of embodiment 52, wherein R⁴ is —C₁-C₆ alkyl.
55. In some embodiments, the compound of embodiment 54, wherein-C₁-C₆ alkyl is-CH₃.
56. In some embodiments, the compound of any one of embodiments 52-55, wherein R⁶ is

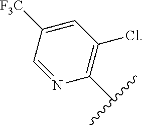 [SMILES: *c1ncc(C(F)(F)F)cc1Cl]

57. In some embodiments, the compound of any one of embodiments 52-55, wherein R⁶ is

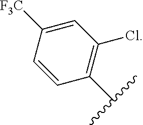 [SMILES: *c1ccc(C(F)(F)F)cc1Cl]

58. In some embodiments, the compound of any one of embodiments 52-57, wherein R⁸ is —N(CH₃CH₃).
59. In some embodiments, the compound of any one of embodiments 52-57, wherein R⁸ is

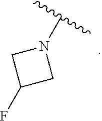 [SMILES: *N1CC(F)C1]

60. In some embodiments, the compound of any one of embodiments 52-59, wherein X is C.

61. In some embodiments, the compound of any one of embodiments 52-59, wherein X is N.

62. In another embodiment, the compound of embodiment 1, having a Formula (Id),

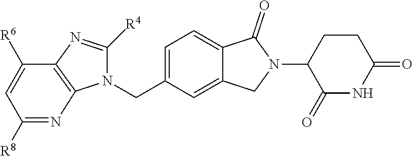

or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
R⁴ is independently selected from hydrogen or —C₁-C₆ alkyl;
R⁶ is independently selected from

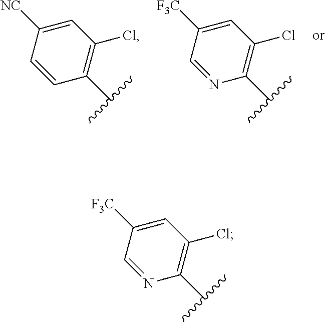 [SMILES: *c1ccc(C#N)cc1Cl ; *c1ncc(C(F)(F)F)cc1Cl ; *c1ncc(C(F)(F)F)cc1Cl]

and
R⁸ is independently selected from

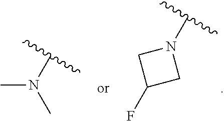

63. In another embodiment, the compound of embodiment 1, having a Formula (Ie),

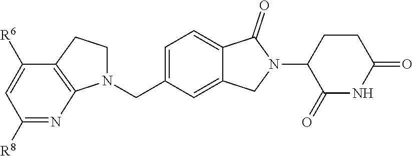

or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
R⁶ is

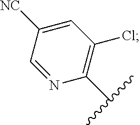 [SMILES: *c1ncc(C#N)cc1Cl]

and
R⁸ is —N(CH₃CH₃).
64. In some embodiments, the compound, selected from

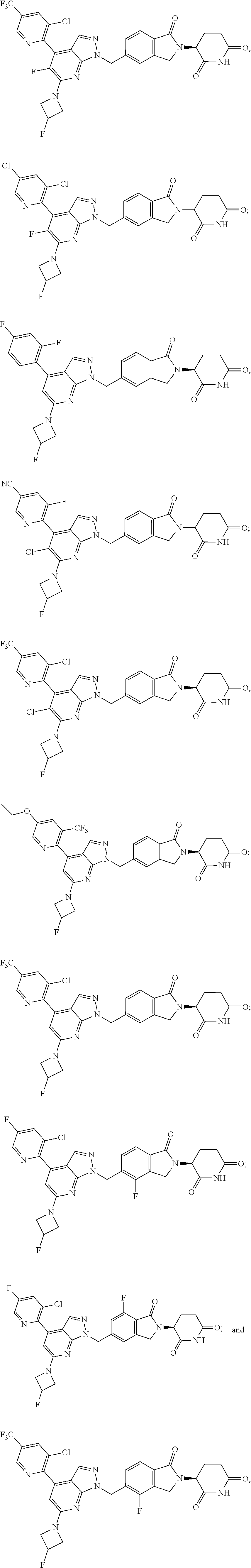

or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof.
65. In some embodiments, the method for reducing FAK protein levels, the method comprising contacting a cell with an effective amount of a compound of any one of embodiments 1-64 or a pharmaceutically acceptable salt, tautomer, isotopologue, or stereoisomer thereof.
66. In some embodiments, the method of embodiment 65, wherein the cell is in a subject.
67. In some embodiments, the method of preventing or treating cancer in a subject comprising administering to a subject in need thereof an effective amount of a compound of any one of embodiments 1-66 or a pharmaceutically acceptable salt, tautomer, isotopologue, or stereoisomer thereof.
68. In some embodiments, the method according to embodiment 67, where the cancer is selected from gastric, lung, pancreatic, ovarian, breast, skin, colon, neuroblastoma, osteosarcoma, uterine, rectal, and kidney cancer.
69. In some embodiments, the method according to embodiment 68, wherein the cancer is selected from pancreatic ductal adenocarcinoma (PDAC), small cell lung cancer, non-small cell lung cancer (NSCLC), high grade serous ovarian cancer, triple negative breast cancer, uterine serous carcinoma, Ewing's sarcoma, melanoma, colon, and clear cell renal cell carcinoma (ccRCC).
70. In some embodiments, the compound of any one of embodiments 1-64 for use as a medicament.
71. In some embodiments, the compound of any one of embodiments 1-64 and 70 for use in a method of treating cancer, comprising administering to a mammal having cancer a therapeutically effective amount of the compound.
72. In some embodiments, the use of a compound of any one of embodiments 1-64 and 70-71 or a pharmaceutically acceptable salt, tautomer, isotopologue, or stereoisomer thereof, in the manufacture of a medicament for reducing FAK protein levels.
73. In some embodiments, the use of a compound of any one of embodiments 1-64 and 70-71 or a pharmaceutically acceptable salt, tautomer, isotopologue, or stereoisomer thereof, in the manufacture of a medicament for the prevention or treatment of cancer.
74. In some embodiments, the compound for use according to embodiment 72, wherein the cancer is selected from gastric, lung, pancreatic, ovarian, breast, skin, colon, neuroblastoma, osteosarcoma, uterine, rectal, and kidney cancer.
75. In some embodiments, the compound for use according to embodiment 72, wherein the cancer is selected from pancreatic ductal adenocarcinoma (PDAC), small cell lung cancer, non-small cell lung cancer (NSCLC), high grade serous ovarian cancer, triple negative breast cancer, uterine serous carcinoma, Ewing's sarcoma, melanoma, colon, and clear cell renal cell carcinoma (ccRCC).
76. In some embodiments, the compound for use according to any one of embodiments 70-75, wherein the use further comprises administering a therapeutically effective amount of another second active agent or a support care therapy, wherein the other second active agent is a therapeutic antibody that specifically binds to a cancer antigen, hematopoietic growth factor, cytokine, anti-cancer agent, antibiotic, cox-2 inhibitor, immunomodulatory agent, immunosuppressive agent, corticosteroid or a pharmacologically active mutant or derivative thereof.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to R¹ of Formula (I) may be combined with every description, variation, embodiment, or aspect of, R², R³, R⁴, R⁵, R⁶, R⁷, R⁸, X, Y, YY, Z and ZZ the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (Ia), (Ib), (Ic), (Id), and (Ie), and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure are herein described.

### Methods of Use

Embodiments of the present disclosure provide a method for degrading FAK, a method for reducing FAK proteins levels, and a method of preventing or treating diseases such as cancer in a subject in need thereof.

In one aspect, provided herein is a method for degrading FAK in a subject in need thereof, the method comprising contacting a cell with an effective amount of a compound of Formula (I). Degradation of FAK can be assessed and demonstrated by a wide variety of methods known in the art. Kits and commercially available assays, including cell-based assays, can be utilized for determining whether and to what degree FAK has been degraded. In some embodiments, the compound of Formula (I) partially degrades FAK. In some embodiments, the compound of Formula (I) fully degrades FAK.

In some embodiments, a compound of Formula (I) degrades FAK by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) degrades FAK by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In some embodiments, provided herein is a method for reducing FAK kinase protein levels, the method comprising contacting a cell with an effective amount of a compound of Formula (I). Reduction of FAK kinase protein levels can be assessed and demonstrated by a wide variety of methods known in the art. Kits and commercially available assays, including cell-based assays, can be utilized for determining whether and to what degree kinase protein levels have been reduced.

In some embodiments, a compound of Formula (I) reduces FAK kinase protein levels by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) reduces FAK kinase protein levels by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In some embodiments, a compound of Formula (I) has an EC₅₀ value as measured in a FAK degradation assay of from about 0.0003 μM to about 1 μM or from about 0.0003 μM to about 0.2 μM or from about 0.0003 μM to about 0.05 μM. In some embodiments, a compound of Formula (I) has an EC₅₀ of from about 0.05 μM to about 0.2 μM. In some embodiments, a compound of Formula (I) has an EC₅₀ of from about 0.2 μM to about 1 μM. In some embodiments, a compound of Formula (I) has an EC50 of less than about 1 μM. In some embodiments, a compound of Formula (I) has an EC50 value of less than 0.2 μM, less than 0.05 μM, less than 0.001 μM, or less than about 0.0003 μM.

In another aspect, provided herein is a method for treating cancer in a subject in need thereof, comprising administering to the subject an effective amount of a compound of Formula (I). In some embodiments, provided herein is a method for preventing cancer in a subject in need thereof, comprising administering to the subject an effective amount of a compound of Formula (I). In some embodiments, the cancer is selected from a brain cancer, a cervicocerebral cancer, an esophageal cancer, a thyroid cancer, small cell cancer, a non-small cell cancer, a breast cancer, a lung cancer, a stomach cancer, gallbladder/bile duct cancer, a liver cancer, a pancreatic cancer, a colon cancer, a rectal cancer, an ovarian cancer, a choriocarcinoma, an uterus body cancer, an uterocervical cancer, a renal pelvis/ureter cancer, a bladder cancer, a prostate cancer, a penis cancer, a testicular cancer, a fetal cancer, Wilms' cancer, a skin cancer, malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

In some embodiments, the cancer is selected from gastric, lung, pancreatic, ovarian, breast, skin, colon, neuroblastoma, osteosarcoma, uterine, rectal, and kidney cancer. In some embodiments, the cancer is selected from pancreatic ductal adenocarcinoma (PDAC), small cell lung cancer, non-small cell lung cancer (NSCLC), high grade serous ovarian cancer, triple negative breast cancer, uterine serous carcinoma, Ewing's sarcoma, melanoma, colon, and clear cell renal cell carcinoma (ccRCC).

In some embodiments, administering a compound of Formula (I) to a subject that is predisposed to cancer prevents the subject from developing any symptoms of the cancer (such as tumor growth or metastasis). In some embodiments, administering a compound of Formula (I) to a subject that does not yet display symptoms of cancer prevents the subject from developing any symptoms of the cancer. In some embodiments, administering a compound of Formula (I) to a subject in need thereof diminishes the extent of the cancer in the subject. In some embodiments, administering a compound of Formula (I) to a subject in need thereof stabilizes the cancer (prevents or delays the worsening of the cancer). In some embodiments, administering a compound of Formula (I) to a subject in need thereof delays the occurrence or recurrence of the cancer. In some embodiments, administering a compound of Formula (I) to a subject in need thereof slows the progression of the cancer. In some embodiments, administering a compound of Formula (I) to a subject in need thereof provides a partial remission of the cancer. In some embodiments, administering a compound of Formula (I) to a subject in need thereof provides a total remission of the cancer. In some embodiments, administering a compound of Formula (I) to a subject in need thereof decreases the dose of one or more other medications required to treat the cancer. In some embodiments, administering a compound of Formula (I) to a subject in need thereof enhances the effect of another medication used to treat the cancer. In some embodiments, administering a compound of Formula (I) to a subject in need thereof delays the progression of the cancer. In some embodiments, administering a compound of Formula (I) to a subject in need thereof increases the quality of life of the subject having cancer. In some embodiments, administering a compound of Formula (I) to a subject in need thereof prolongs survival of a subject having cancer.

In one aspect, provided herein is method of preventing a subject that is predisposed to cancer from developing cancer, the method comprising administering a compound of Formula (I) to the subject.

In some aspects, provided herein is a method of diminishing the extent of cancer in a subject, the method comprising administering a compound of Formula (I) to the subject. In some embodiments, provided herein is a method of stabilizing cancer in a subject, the method comprising administering a compound of Formula (I) to the subject. In some embodiments, the method prevents the worsening of the cancer.

In another aspect, provided herein is a method of delaying the occurrence or recurrence of cancer in a subject, the method comprising administering a compound of Formula (I) to the subject.

In some embodiments, provided herein is a method of slowing the progression of cancer in a subject, the method comprising administering a compound of Formula (I) to the subject. In some embodiments, the method provides a partial remission of the cancer. In some embodiments, the method provides a total remission of the cancer.

In further aspects, provided herein is a method of decreasing the dose of one or more other medications required to treat cancer in a subject, the method comprising administering a compound of Formula (I) to the subject. In some embodiments, provided herein is a method of enhancing the effect of another medication used to treat cancer in a subject, the method comprising administering a compound of Formula (I) to the subject.

Also provided here is a method of delaying the progression of cancer in a subject, the method comprising administering a compound of Formula (I) to the subject. In some embodiments, the method increases the quality of life of the subject having cancer. In some embodiments, the method prolongs survival of the subject having cancer.

In some embodiments, compounds of Formula (I) are useful in the manufacture of a medicament for reducing FAK kinase protein levels. In some embodiments, compounds of Formula (I) are useful in the manufacture of a medicament for the prevention or treatment of a disease associates with FAK.

The methods and uses of the present disclosure may include a compound of Formula (I) used alone or in combination with one or more additional therapies (e.g., non-drug treatments or therapeutic agents).

In some embodiments, the compound for use according to any one of embodiments 70-75, wherein the use further comprises administering a therapeutically effective amount of another second active agent or a support care therapy, wherein the other second active agent is a therapeutic antibody that specifically binds to a cancer antigen, hematopoietic growth factor, cytokine, anti-cancer agent, antibiotic, cox-2 inhibitor, immunomodulatory agent, immunosuppressive agent, corticosteroid or a pharmacologically active mutant or derivative thereof.

A compound of Formula (I) may be administered before, after, or concurrently with one or more of such additional therapies. When combined, dosages of the compound of Formula (I) and dosages of the one or more additional therapies (e.g., non-drug treatment or therapeutic agent) may provide a therapeutic effect (e.g., synergistic or additive therapeutic effect). A compound of Formula (I) and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment). For example, in some embodiments, the compounds of Formula (I) can be used in combination with a therapeutic agent that treats nausea. Examples of agents that can be used to treat nausea include, but are not limited to, dronabinol, granisetron, metoclopramide, ondansetron, prochlorperazine, and pharmaceutically acceptable salts thereof.

In some embodiments, one or more additional therapies includes a non-drug treatment (e.g., surgery or radiation therapy). In some embodiments, one or more additional therapies includes a therapeutic agent (e.g., a compound or biologic that is an antiproliferative agent). In some embodiments, one or more additional therapies includes a non-drug treatment and a therapeutic agent. In other embodiments, one or more additional therapies includes two therapeutic agents. In still other embodiments, one or more additional therapies includes three therapeutic agents. In some embodiments, one or more additional therapies includes four or more therapeutic agents.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of Formula (I) administered will depend on such factors as the solubility of the active component, the formulation used, and the route of administration.

In some embodiments, a compound of Formula (I) is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of Formula (I).

In a particular embodiment, provided herein are unit dosage formulations comprising about 0.1 mg or 100 mg of a compound of Formula (I).

In another embodiment, provided herein are unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg, or 1400 mg of a compound of Formula (I).

A compound of Formula (I) can be administered once, twice, three, four or more times daily. As a nonlimiting example, doses of 100 mg or less are administered as a once daily dose and doses of more than 100 mg are administered twice daily in an amount equal to one half of the total daily dose.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories, spray dried dispersions, and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants, and disintegrators as well as the compound. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose, and waxes can also serve as binders.

A lubricant might be necessary in a tablet formulation to prevent the tablet and punches from sticking in the dye. The lubricant can be selected from such slippery solids as talc, magnesium and calcium stearate, stearic acid, and hydrogenated vegetable oils. Tablet disintegrators are substances that swell when wetted to break up the tablet and release the compound. They include starches, clays, celluloses, algins, and gums. More particularly, corn and potato starches, methylcellulose, agar, bentonite, wood cellulose, powdered natural sponge, cation-exchange resins, alginic acid, guar gum, citrus pulp, and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### EXAMPLES

The following Examples are presented by way of illustration, not limitation. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

All reactions are at room temperature unless noted otherwise.

The following abbreviations may be relevant for the application.

### Abbreviations

| | |
|---|---|
| Boc | tert-butyloxycarbonyl |
| AIBN | azobisisobutyronitrile |
| ACN | acetonitrile |
| BSA | bis(trimethylsilyl)acetamide |
| DCE | 1,2-dichloroethane |
| DCM | dichloromethane |
| DIEA | N,N-diisopropylethylamine |
| DIPEA | N,N-diisopropylethylamine |
| DME | dimethyl ether |
| DMF | dimethyl formamide |
| DMSO | dimethyl sulfoxide |
| DTBAD | di-tert-butyl azodicarboxylate |
| Dtbbpy | 4,4′-di-tert-butyl-2,2′-bipyridine |
| equiv | equivalent |
| ESI | electrospray ionization |
| EtOH | ethanol |
| FA | formic acid |
| h | hour |
| HATU | O-(7-azabenzotriazol-1-yl)-N,N,N′,N′-tetramethyluronium hexafluorophosphate |
| HCl | hydrochloric acid |
| HOAc | Acetic acid |
| HPLC | high performance liquid chromatography |
| LAH | lithium aluminum hydride |
| LED | light-emitting diode |
| LiOtBu | lithium tert-butoxide |
| MeCN | acetonitrile |
| MeOH | methanol |
| min | minute |
| MS | mass spectrometry |
| NBS | N-bromo succinimide |
| NMO | 4-methylmorpholine 4-oxide |
| NMP | N-methyl-2-pyrrolidone |
| NMR | nuclear magnetic resonance |
| PivOH | pivalic acid |
| PMBCl | 4-methoxybenzyl chloride |
| Py | pyridine |
| rt | room temperature |
| RT | retention time |
| sat. | saturated |
| t-BuOH | tert-butyl alcohol |
| t-BuOLi | lithium tert-butoxide |
| TEA | triethylamine |
| TFA | trifluoroacetic acid |
| TfOH | triflic acid |
| THF | tetrahydrofuran |
| TLC | thin layer chromatography |
| TMP | 2,2,6,6-tetramethylpiperidine |

### SYNTHETIC EXAMPLES

### Methods of Synthesis

The compounds described herein can be made using conventional organic syntheses and commercially available starting materials, or the methods provided herein. By way of example and not limitation, compounds of Formula (I) can be prepared as outlined in Scheme 1 as well as in the examples set forth herein. It should be noted that one skilled in the art would know how to modify the procedures set forth in the illustrative schemes and examples to arrive at the desired products, including, for example, selecting starting materials having different stereochemistry (or racemic starting materials) to arrive at desired products having different stereochemistry.

In general, the compounds of Formula (I) can be obtained as shown below in Scheme 1. An aryl stannane (2a) or boronate (2b) may be reacted with aryl iodide (1) to obtain biaryl Intermediate 3. The fluoride of Intermediate 3 may be displaced by amine (4) to obtain Intermediate 5. The amide and ester of Intermediate 5 can be reacted under various conditions, including acid treatment, to form a glutarimide ring. In some embodiments, the iodide of Intermediate 1 may be replaced with a stannane or boronate and Intermediate 2 may be an aryl halide. In some embodiments, Intermediate 1 may be composed of different heterocycles and differently substituted from what is shown in Scheme 1. In some embodiments, the order of the reactions steps may rearranged. All reactions in the schemes below are at room temperature unless noted otherwise.

The practice of this invention can be further understood by reference to the following examples, which are provided by way of illustration and not of limitation.

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). In the formulaE, sometimes the N2 regioisomers are not shown for convenience, but it is to be understood that they are present in the initial product mixture and separated at a later time, for example by preparative HPLC.

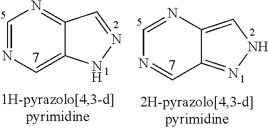

The mixture of regioisomers can be separated at an early stage of the synthesis and the remaining synthetic steps carried out with the 1H regioisomer or, alternatively, the synthesis can be progressed carrying the mixture of regioisomers and separation effected at a later stage, as desired.

The foregoing detailed description includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various formulae and descriptions herein relate to specific embodiments of the invention, it is to be understood that where a specific feature is disclosed in the context of a particular formula or embodiment, such feature can also be used, to the extent appropriate, in the context of another formula or embodiment, in combination with another feature, or in the invention in general.

All starting materials were made using the procedures below or were purchased by commercial suppliers, such as Sigma-Aldrich, Combi-Blocks, Enamine, and eMolecules. All reagents were purchased through commercial suppliers, such as Sigma-Aldrich.

Further, while the present invention has been particularly described in terms of certain preferred embodiments, the invention is not limited to such preferred embodiments. Rather, the scope of the invention is defined by the appended claims.

Scheme I illustrates the most common route used to synthesize the Examples in the present specification. Variations of this route are shown in the examples below.

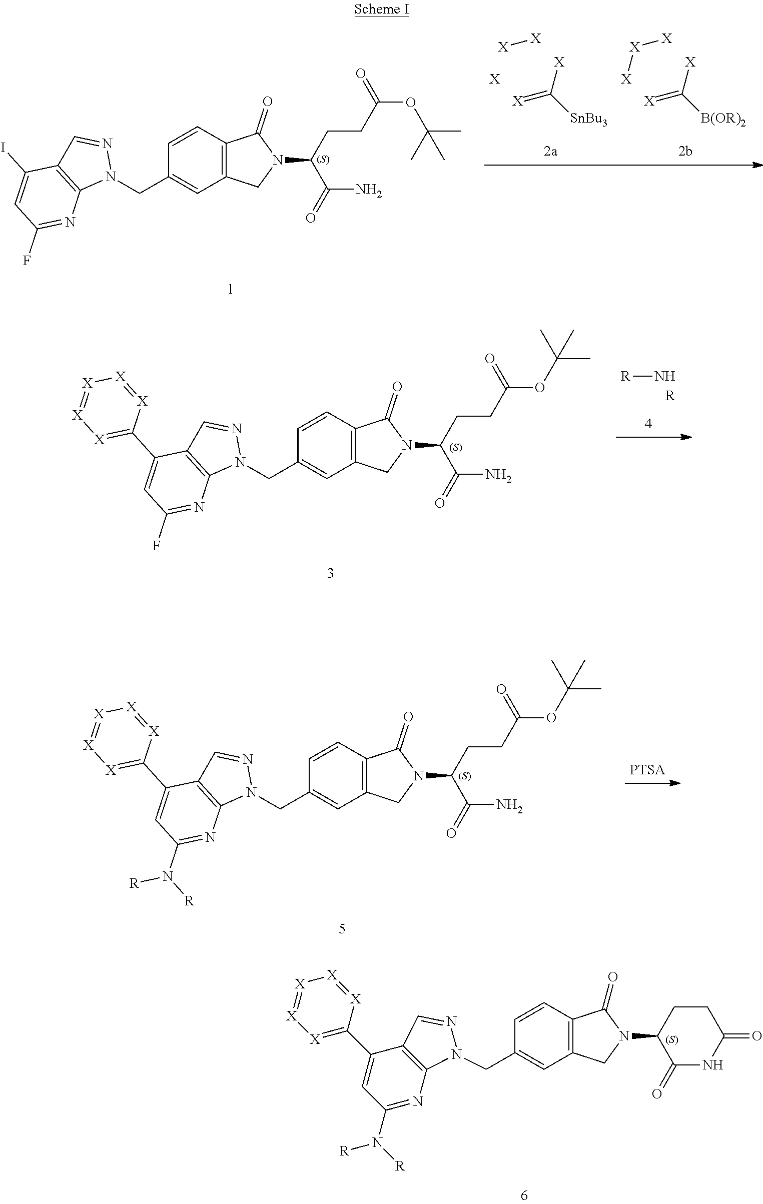

As will be apparent to one skilled in the art, the compounds disclosed below and in Table 1 can exist in various stereochemical forms. Where stereochemistry is shown between the piperidine-dione to the isoindolinone in the compounds in the Examples and Table 1, the stereochemistry is absolute. Other stereocenters in which stereochemistry is shown and indicated as R or S, may be relative stereochemistry. Racemates and single enantiomers were prepared following the experimental procedure below. In certain instances, enantiomers were separated from racemates by chiral SFC method to obtain the individual enantiomers.

The synthesis of all requisite cores and precursor intermediates involved in the aforementioned key steps are described in the following experimentals herein.

### Intermediate Syntheses

### Intermediate A: 6-fluoro-4-iodo-1H-pyrazolo[3,4-b]pyridine

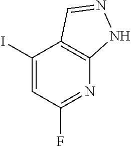 [SMILES: Fc1cc(I)c2cn[nH]c2n1]

### Step A1: 2,6-difluoro-4-iodo-pyridine-3-carbaldehyde

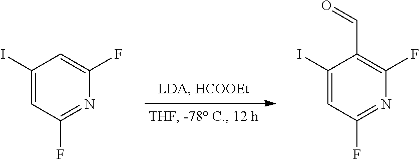 [SMILES: Fc1cc(I)cc(F)n1 ; O=Cc1c(I)cc(F)nc1F]

To a solution of 2,6-difluoro-4-iodo-pyridine (10.000 g, 41.5 mmol) in tetrahydrofuran (100 mL) was added Lithium diisopropylamide (24.9 mL, 49.8 mmol) dropwise at −78° C. under nitrogen. The mixture was stirred at 78° C. under nitrogen for 1 h. Then the mixture was added ethyl formate (15.37 g, 207.49 mmol). The mixture was stirred at −78° C. under nitrogen for 3 hours. The mixture was poured into saturated ammonium chloride aqueous solution (100 mL) and extracted with ethyl acetate (100 mL×3). The combined organic layers were washed with brine (100 mL×1), dried with anhydrous sodium sulfate, filtered, and concentrated under vacuum. The residue was purified by silica gel chromatography (0˜30% ethyl acetate in petroleum ether) to afford 2,6-difluoro-4-iodo-pyridine-3-carbaldehyde (3.200 g, 11.90 mmol, 29% yield) as a yellow solid. ¹H NMR (400 MHz, CDCl₃) δ 10.12 (s, 1H), 7.55 (d, J=4.0, 1H).

### Step A2:6-fluoro-4-iodo-1H-pyrazolo[3,4-b]pyridine

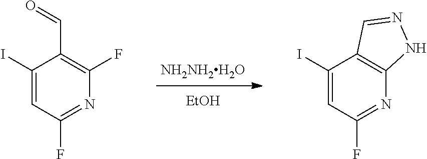 [SMILES: "EtOH" ; O=Cc1c(I)cc(F)nc1F ; "NH2NH2·H2O" ; Fc1cc(I)c2cn[nH]c2n1]

To a solution of 2,6-difluoro-4-iodo-pyridine-3-carbaldehyde (4.700 g, 17.47 mmol) in ethanol (50 mL) was added hydrazine hydrate (1.340 g, 22.71 mmol). The mixture was stirred at 80° C. for 12 hours. The mixture was filtered and concentrated under vacuum. The residue was purified by silica gel chromatography (30˜60% ethyl acetate in petroleum ether) to afford 6-fluoro-4-iodo-1H-pyrazolo[3,4-b]pyridine (3.000 g, 11.41 mmol, 66% yield) as a yellow solid. ¹1H NMR (400 MHz, DMSO-d₆) δ 14.0 (s, 1H), 8.00 (s, 1H), 7.51 (s, 1H), 3.53 (q, J=6.0 Hz, 2H), 3.00-2.99 (m, 6H), 2.52 (t, J=6.0 Hz, 2H), 1.46 (s, 9H). MS (ESI) m/z: 263.9 [M+1]⁺.

### Intermediate B: 3-chloro-6-fluoro-4-iodo-1H-pyrazolo[3,4-b]pyridine

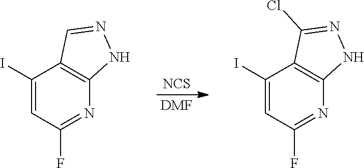

A mixture of 6-fluoro-4-iodo-1H-pyrazolo[3,4-b]pyridine (Intermediate A)(0.400 g, 1.52 mmol) and N-chlorosuccinimide (0.305 g, 2.28 mmol) in DMF (10 mL) was stirred at 25° C. for 12 h under nitrogen. The reaction mixture was diluted with water (100 mL) and extracted with ethyl acetate (30 mL×2). The combined organic layers were dried over anhydrous sodium sulfate solid, filtered, and concentrated under reduced pressure. The residue was purified by prep-TLC (10% ethyl acetate in petroleum ether) to give 3-chloro-6-fluoro-4-iodo-1H-pyrazolo[3,4-b]pyridine (0.280 g, 0.94 mmol, 62% yield) as a light yellow solid. ¹H NMR (400 MHz, DMSO-d₆) δ 14.34-14.20 (m, 1H), 7.67 (d, J=1.2 Hz, 1H). MS (ESI) m/z: 297.8 [M+1]⁺.

### Intermediate C: tert-butyl(S)-5-amino-4-(5-(bromomethyl)-1-oxoisoindolin-2-yl)-5-oxopentanoate

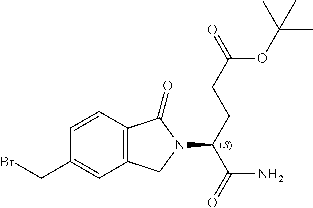

### Step C1: methyl 4-bromo-2-methylbenzoate

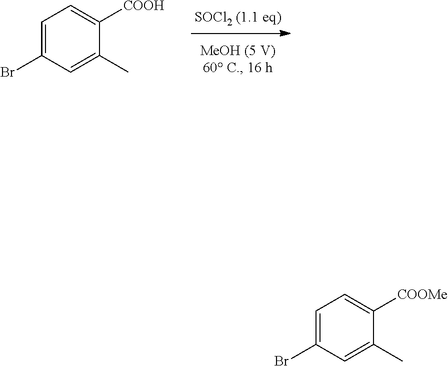

To a solution of compound 4-bromo-2-methylbenzoic acid (600 g, 2.79 mol, 1.00 eq) in methanol (3.00 L) was added thionyl chloride (365 g, 3.07 mol, 1.10 eq) drop-wise at 15° C. and the mixture was stirred at 60° C. for 16 hrs. TLC (Petroleum ether:ethyl acetate=1:1, R_{f}=0.9) showed the reaction was complete. The reaction mixture was concentrated under vacuum. The residue was mixed with ethyl acetate (15.0 L) and water (5.00 L), and adjusted to pH=8 with saturated sodium bicarbonate solution. The organic layer was washed with brine (5.00 L), dried over sodium sulfate and concentrated under vacuum to give compound methyl 4-bromo-2-methylbenzoate (3.15 kg, 99% yield) as yellow oil. ¹H NMR (400 MHz CDCl₃) δ 7.78 (d, J=8.4 Hz, 1H), 7.42 (s, 1H), 7.38 (dd, J=8.4, 2.0 Hz, 1H), 3.88 (s, 3H), 2.58 (s, 3H).

### Step C2:4-bromo-2-(bromomethyl)benzoate

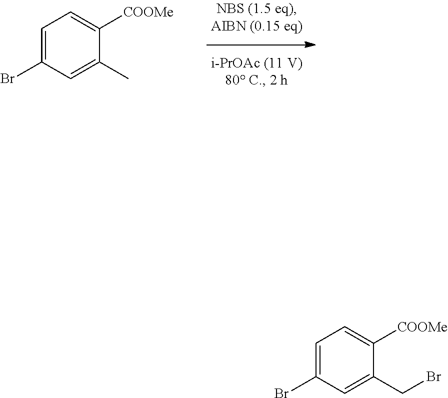

A mixture of methyl 4-bromo-2-methylbenzoate (275 g, 1.20 mol, 1.00 eq), NBS (321 g, 1.80 mol, 1.50 eq) and AIBN (29.6 g, 180 mmol, 0.15 eq) in isopropyl acetate (3.00 L) was stirred at 80° C. for 2 hrs and irradiated with a 1000 Watt lamp. TLC (Petroleum ether:ethyl acetate=10:1, R_{f}=0.45) showed that the reaction was mostly complete. The reaction mixture was cooled to room temperature and diluted with methyl tert-butyl ether (3.00 L). The mixture was filtered. The filtrate was washed with water (20.0 L×2), 5% sodium bicarbonate aqueous solution (10.0 L), brine (10.0 L), dried over anhydrous sodium sulfate and concentrated under reduced pressure to give compound methyl 4-bromo-2-(bromomethyl)benzoate (2.60 kg, crude) as light yellow solid. ¹H NMR (400 MHz CDCl₃) δ 7.84 (d, J=8.4 Hz, 1H), 7.63 (d, J=2.0 Hz, 1H), 7.51 (dd, J=8.4, 2.0 Hz, 1H), 4.90 (s, 2H) 3.94 (s, 3H).

### Step C3: tert-butyl(S)-5-amino-4-(5-bromo-1-oxoisoindolin-2-yl)-5-oxopentanoate

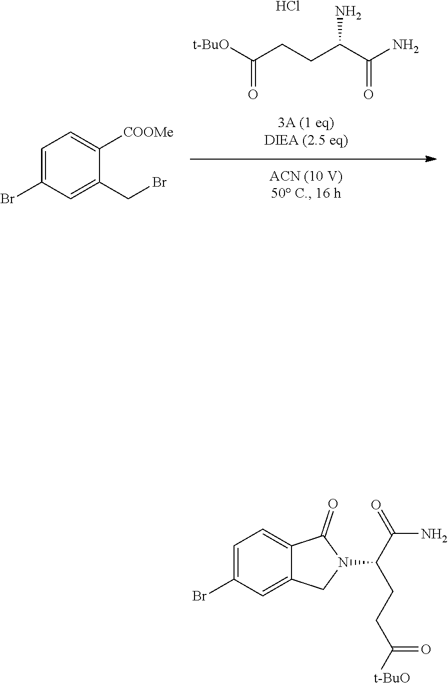 [SMILES: Cl ; CC(C)(C)OC(=O)CC[C@H](N)C(N)=O ; COC(=O)c1ccc(Br)cc1CBr ; CC(C)(C)OC(=O)CC[C@@H](C(N)=O)N1Cc2cc(Br)ccc2C1=O]

To a solution of compound methyl 4-bromo-2-(bromomethyl)benzoate (200 g, 649 mmol, 1.00 eq) and compound tert-butyl(S)-4,5-diamino-5-oxopentanoate hydrochloride (155 g, 649 mmol, 1.00 eq) in acetonitrile (2.00 L) was added DIEA (210 g, 1.62 mol, 2.50 eq) at 20° C. The reaction mixture was stirred at 50° C. for 16 hrs. TLC (Petroleum ether:ethyl acetate=10:1, R_{f}=0.02) showed the reaction was complete. The reaction mixture was concentrated under vacuum. The combined residue was mixed with ethyl acetate:THF (4:1, 30.0 L) and water (20.0 L). The aqueous phase was extracted with ethyl acetate (7.50 L×2). The combined organic layer was washed with brine (10.0 L), dried over anhydrous sodium sulfate and concentrated under reduced pressure. The residue was triturated with methyl tert-butyl ether (8.00 L) for 0.5 hr and filtered. The filter cake was washed with methyl tert-butyl ether (1.00 L) and dried under reduced pressure to give tert-butyl(S)-5-amino-4-(5-bromo-1-oxoisoindolin-2-yl)-5-oxopentanoate (1.60 kg, 99.3% purity, 48% yield for 2 steps) as white solid. ¹H NMR (400 MHz DMSO-d₆) δ 7.88 (s, 1H), 7.67-7.69 (m, 1H), 7.62-7.64 (m, 1H), 7.59 (br.s, 1H), 7.21 (br.s, 1H), 4.71-4.74 (m, 1H), 4.44-4.63 (m, 2H), 2.13-2.18 (m, 3H), 1.89-2.00 (m, 1H), 1.32 (s, 9H). MS (ESI) m/z: 397.1 (M+1)⁺.

### Step C4: (tributylstannyl)methanol

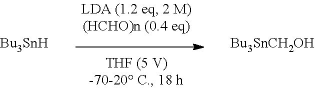

To a solution of LDA (2.00 M, 1.03 L, 1.20 eq) in dry THF (2.50 L) was added compound tributyltin hydride (500 g, 1.72 mol, 455 mL, 1.00 eq) at −70° C. After stirring at 0° C. for 2 hrs, to the reaction was added (HCHO)ₙ (108 g, 0.40 eq) at −70° C. After addition, the reaction was allowed to stir at 20° C. for 16 hrs. TLC (petroleum ether/ethyl acetate=10:1, R_{f}=0.84) showed starting material was consumed. The reaction was quenched with sat. aqueous ammonium chloride (10.0 L) at 0° C. and ethyl acetate (4.00 L) was added. The organic layer was separated, washed by brine (4.00 L×2) and dried over anhydrous sodium sulfate. The mixture was filtered, and the filtrate was concentrated under vacuum. The residue was purified by silica gel chromatography with petroleum ether/ethyl acetate (1:0˜10:1) to obtain (tributylstannyl) methanol (2.0 kg, 6.23 mol, 72.27% yield) as yellow oil.

### Step C5: tert-butyl(S)-5-amino-4-(5-(hydroxymethyl)-1-oxoisoindolin-2-yl)-5-oxopentanoate

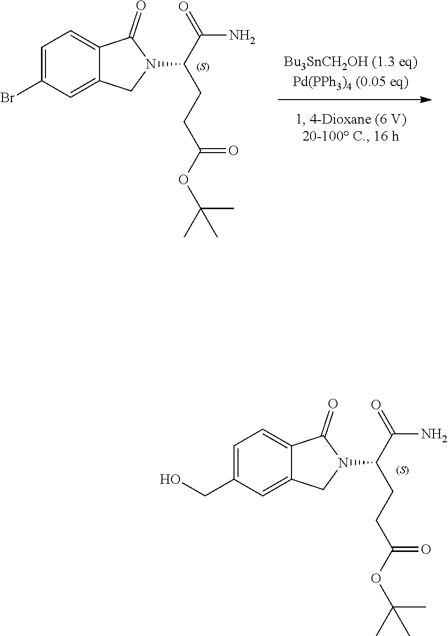

To a degassed solution of compound tert-butyl(S)-5-amino-4-(5-bromo-1-oxoisoindolin-2-yl)-5-oxopentanoate (500 g, 1.26 mol, 1.00 eq) in dry 1,4-dioxane (3.0 L) was added (tributylstannyl) methanol (525 g, 1.64 mol, 1.30 eq) and tetrakis(triphenylphosphine)-palladium (0)(72.7 g, 62.9 mmol, 0.05 eq) at 20° C. After addition, the reaction mixture was stirred at 100° C. for 16 hrs. The reaction mixture was concentrated under vacuum. The crude product was triturated with methyl tert-butyl ether/petroleum ether (5/1, 6 L) at 25° C. for 4 h. The suspension was filtered, and the filter cake was washed with petroleum ether (5.0 L) and dried under vacuum to give tert-butyl(S)-5-amino-4-(5-(hydroxymethyl)-1-oxoisoindolin-2-yl)-5-oxopentanoate (900 g, 2.30 mol, 61% yield, 88.9% purity) as an off-white solid. ¹H NMR 400 MHz CDCl₃ δ 7.57 (d, J=7.6 Hz, 1H), 7.37 (s, 1H), 7.27-7.30 (m, 1H), 7.00 (s, 1H), 4.85-4.88 (m, 1H), 4.29-4.44 (m, 2H), 2.16-2.28 (m, 4H), 1.37 (s, 9H).

### Step C6: tert-butyl(S)-5-amino-4-(5-formyl-1-oxoisoindolin-2-yl)-5-oxopentanoate

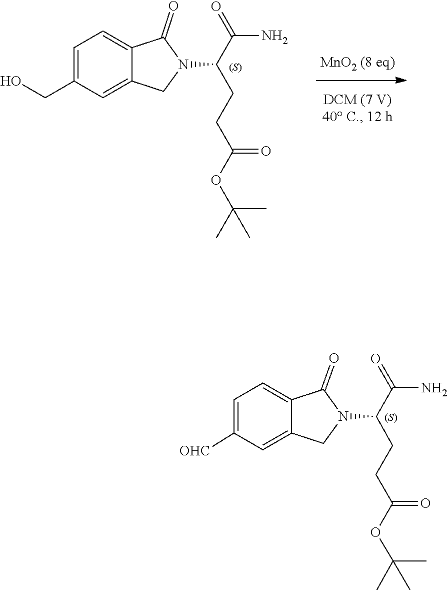

A mixture of tert-butyl(S)-5-amino-4-(5-(hydroxymethyl)-1-oxoisoindolin-2-yl)-5-oxopentanoate (100 g, 287 mmol, 1.00 eq) and manganese dioxide (200 g, 2.30 mol, 8.00 eq) in DCM (700 mL) was degassed and purged with nitrogen 3 times. The mixture was stirred at 40° C. for 12 hrs under nitrogen atmosphere. The reaction mixture was filtered, and the filtrate was concentrated under vacuum. The crude product was triturated with ethyl acetate/DCM (9/1, 4 L) at 25° C. for 60 min. The suspension was filtered, and the filter cake was washed with ethyl acetate (1 L) and dried under vacuum to give tert-butyl(S)-5-amino-4-(5-formyl-1-oxoisoindolin-2-yl)-5-oxopentanoate (510 g, 1.37 mol, 60% yield, 93.3% purity) as an off-white solid. ¹H NMR 400 MHz CDCl₃ δ 10.14 (s, 1H), 8.14 (s, 1H), 8.03 (d, J=7.6 Hz, 1H), 7.89-7.91 (m, 1H), 7.61 (s, 1H), 7.22 (s, 1H), 4.72-4.77 (m, 1H), 4.68 (s, 1H), 4.56-4.60 (m, 1H), 1.99-2.20 (m, 4H), 1.32 (s, 9H).

### Step C7: (S)-tert-butyl 5-amino-4-(5-(hydroxymethyl)-1-oxoisoindolin-2-yl)-5-oxopentanoate

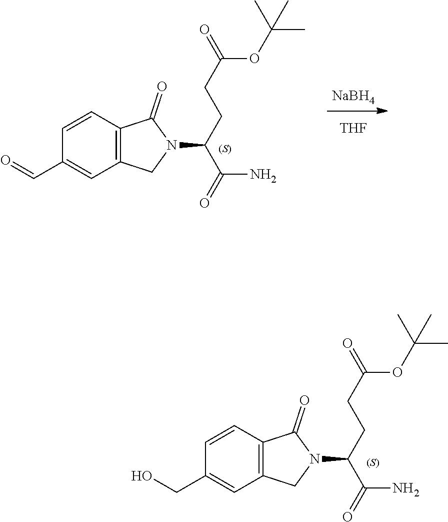

To a solution of(S)-tert-butyl 5-amino-4-(5-formyl-1-oxoisoindolin-2-yl)-5-oxopentanoate (10.000 g, 28.87 mmol) in tetrahydrofuran (200 mL) was added sodium tetrahydroborate (2.270 g, 60.05 mmol) at 0° C. Then the mixture was stirred at 0° C. for 1 h under nitrogen. The reaction mixture was quenched by 1 M phosphoric acid and extracted with ethyl acetate (100 mL×2). The combined organic layers were dried over anhydrous sodium sulfate, filtered, and concentrated under vacuum to give the product(S)-tert-butyl 5-amino-4-(5-(hydroxymethyl)-1-oxoisoindolin-2-yl)-5-oxopentanoate (10.000 g, 28.70 mmol, 99% yield) as a light brown solid. ¹H NMR (400 MHz, DMSO-d₆) δ 7.64 (d, J=7.6 Hz, 1H), 7.54 (s, 2H), 7.42 (d, J=7.6 Hz, 1H), 7.16 (s, 1H), 5.37 (t, J=5.6 Hz, 1H), 4.73 (dd, J=4.4, 10.4 Hz, 1H), 4.63-4.54 (m, 3H), 4.50-4.39 (m, 1H), 2.20-2.10 (m, 3H), 1.98-1.92 (m, 1H), 1.33 (s, 9H). MS (ESI) m/z: 349.2 [M+1]⁺.

### Step C8: (S)-tert-butyl 5-amino-4-(5-(bromomethyl)-1-oxoisoindolin-2-yl)-5-oxopentanoate

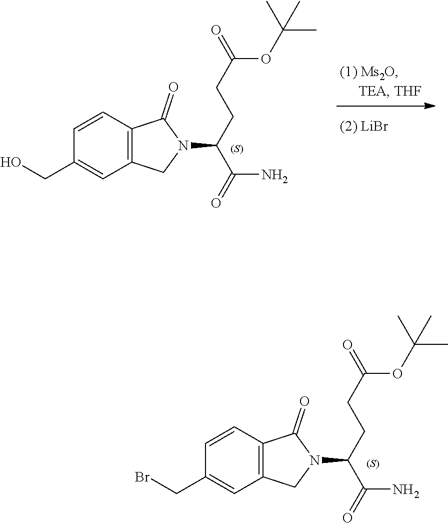

To a solution of(S)-tert-butyl 5-amino-4-(5-(hydroxymethyl)-1-oxoisoindolin-2-yl)-5-oxopentanoate (9.000 g, 25.83 mmol) and triethylamine (7.842 g, 77.5 mmol) in THF (200 mL) was added methylsulfonyl methanesulfonate (9.000 g, 51.67 mmol) at 0° C. Then the mixture was warmed to 25° C. and stirred for 4 h. Then bromolithium (22.436 g, 258.33 mmol) was added. The mixture was stirred for 6 h at 25° C. The reaction mixture was diluted with water (200 mL) and extracted with ethyl acetate (100 mL×2). The combined organic layers were dried over anhydrous sodium sulfate, filtered and concentrated under vacuum. The residue was purified by silica column chromatography (0-75% ethyl acetate in petroleum ether) to give the product(S)-tert-butyl 5-amino-4-(5-(bromomethyl)-1-oxoisoindolin-2-yl)-5-oxopentanoate (5.600 g, 13.62 mmol, 53% yield) as a light yellow solid. ¹H NMR (400 MHz, DMSO-d₆) δ 7.73-7.65 (m, 2H), 7.60-7.52 (m, 2H), 7.20 (s, 1H), 4.82 (s, 2H), 4.77-4.69 (m, 1H), 4.63-4.55 (m, 1H), 4.51-4.43 (m, 1H), 2.21-2.11 (m, 3H), 1.98-1.93 (m, 1H), 1.32 (s, 9H). MS (ESI) m/z: 355.2 [M−55]⁺

### Intermediate D: tert-butyl 5-amino-4-[5-[(6-fluoro-4-iodo-pyrazolo[3,4-b]pyridin-1-yl)methyl]-1-oxo-isoindolin-2-yl]-5-oxo-pentanoate

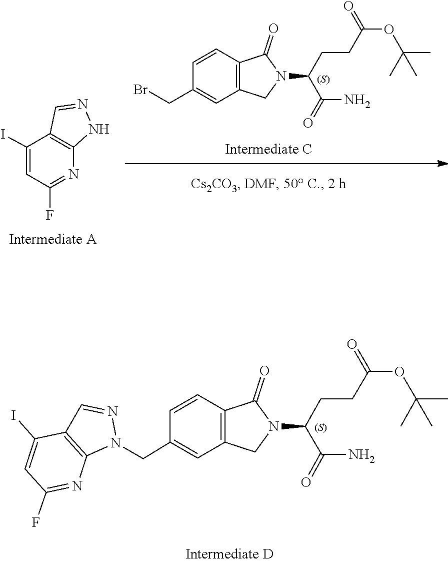

To a solution of 6-fluoro-4-iodo-1H-pyrazolo[3,4-b]pyridine (Intermediate A) (0.844 g, 3.21 mmol) in DMF (8.00 mL) was added tert-butyl 5-amino-4-[5-(bromomethyl)-1-oxo-isoindolin-2-yl]-5-oxo-pentanoate (Intermediate C)(1.200 g, 2.92 mmol) and cesium carbonate (2.850 g, 8.75 mmol). The mixture was stirred at 50° C. for 2 hours. The reaction mixture was diluted with water (15 mL) and extracted with ethyl acetate (20 mL×3). The combined organic layers were washed with saturated brine (20 mL×2) and dried over anhydrous sodium sulfate. The organic layer was filtered, and the filtrate was concentrated under vacuum. The residue was purified by preparative HPLC (42-72% acetonitrile+0.225% formic acid in water, over 13 min). The desired fractions were combined and concentrated under vacuum. The aqueous solution was extracted with ethyl acetate (20 mL×3). The combined organic layers were dried over anhydrous sodium sulfate. The organic layer was filtered, and the filtrate was concentrated under vacuum to afford tert-butyl 5-amino-4-[5-[(6-fluoro-4-iodo-pyrazolo[3,4-b]pyridin-1-yl)methyl]-1-oxo-isoindolin-2-yl]-5-oxo-pentanoate (0.360 g, 0.61 mmol, 21% yield) as a yellow solid. ¹1H NMR (400 MHz, CDCl₃) δ 7.90 (s, 1H), 7.80 (d, J=8.0 Hz, 1H), 7.47 (d, J=8.4 Hz, 1H), 7.42 (s, 1H), 7.27 (s, 1H), 6.36 (s, 1H), 5.67 (s, 2H), 5.57 (s, 1H), 4.89 (dd, J=6.0, 8.8 Hz, 1H), 4.52-4.48 (m, 1H), 4.42-4.38 (m, 1H), 2.33 (d, J=3.6 Hz, 1H), 2.30-2.27 (m, 1H), 2.23 (s, 1H), 2.17-2.11 (m, 1H), 1.41 (s, 9H). MS (ESI) m/z: 537.9 [M+1]⁺.

### Intermediate E: tert-butyl(S)-5-amino-4-(5-((6-fluoro-4-(tributylstannyl)-1H-pyrazolo[3,4-b]pyridin-1-yl)methyl)-1-oxoisoindolin-2-yl)-5-oxopentanoate

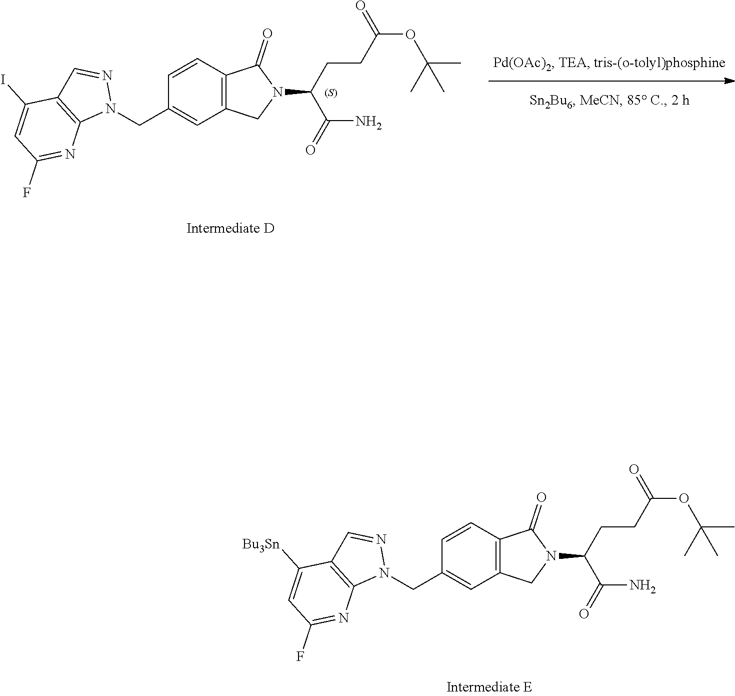

A mixture of tert-butyl(S)-5-amino-4-(5-((6-fluoro-4-iodo-1H-pyrazolo[3,4-b]pyridin-1-yl)methyl)-1-oxoisoindolin-2-yl)-5-oxopentanoate (2.000 g, 3.37 mmol), tributyl(tributylstannyl) stannane (2.542 g, 4.38 mmol), tris-o-tolylphosphane (0.205 g, 0.67 mmol), triethylamine (1.5 mL, 8.43 mmol) and palladium acetate (0.076 g, 0.34 mmol) in acetonitrile (30 mL) was stirred at 85° C. for 2 h under nitrogen. The reaction mixture was concentrated under reduced pressure. The residue was purified by alumina column chromatography (0-100% ethyl acetate in petroleum ether) to give the crude product tert-butyl (S)-5-amino-4-(5-((6-fluoro-4-(tributylstannyl)-1H-pyrazolo[3,4-b]pyridin-1-yl)methyl)-1-oxoisoindolin-2-yl)-5-oxopentanoate (2.000 g, 2.64 mmol, 78% yield) as a brown oil. MS (ESI) m/z: 758.4 [M+1]⁺.

### Intermediate F: tert-butyl(S)-5-amino-4-(5-((6-(3-fluoroazetidin-1-yl)-4-iodo-1H-pyrazolo[3,4-b]pyridin-1-yl)methyl)-1-oxoisoindolin-2-yl)-5-oxopentanoate

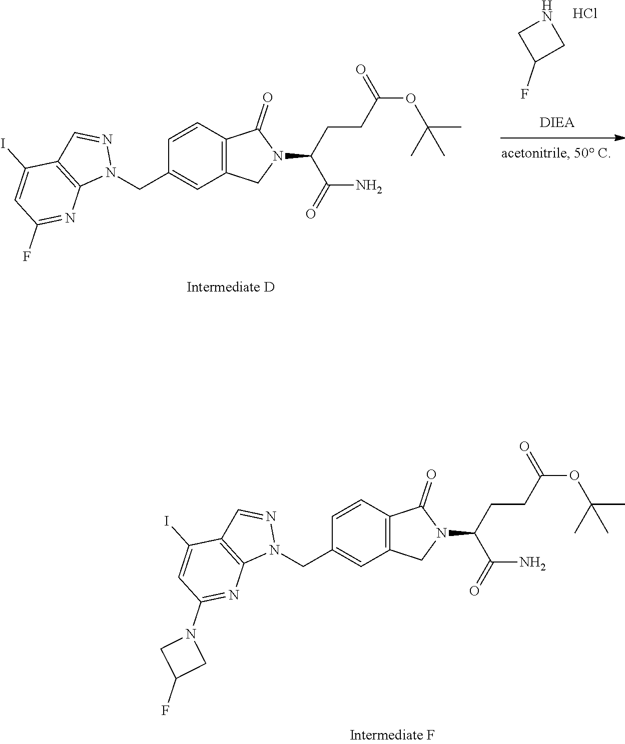 [SMILES: Cl ; CC(C)(C)OC(=O)CC[C@@H](C(N)=O)N1Cc2cc(Cn3ncc4c(I)cc(F)nc43)ccc2C1=O ; FC1CNC1 ; CC(C)(C)OC(=O)CC[C@@H](C(N)=O)N1Cc2cc(Cn3ncc4c(I)cc(N5CC(F)C5)nc43)ccc2C1=O]

tert-butyl(S)-5-amino-4-(5-((6-(3-fluoroazetidin-1-yl)-4-iodo-1H-pyrazolo[3,4-b]pyridin-1-yl)methyl)-1-oxoisoindolin-2-yl)-5-oxopentanoate. To a solution of tert-butyl(S)-5-amino-4-(5-((6-fluoro-4-iodo-1H-pyrazolo[3,4-b]pyridin-1-yl)methyl)-1-oxoisoindolin-2-yl)-5-oxopentanoate (Intermediate D)(3.000 g, 5.06 mmol) in acetonitrile (20 mL) was added 3-fluoroazetidine hydrochloride (0.677 g, 6.07 mmol) and N-ethyl-N-isopropylpropan-2-amine (4 mL, 20.22 mmol). The mixture was stirred at 50° C. for 2 h. The mixture was diluted with water (60 mL) and extracted with ethyl acetate (30 mL×3). The combined organic layer was dried over anhydrous sodium sulfate, filtered, and concentrated under vacuum to afford tert-butyl(S)-5-amino-4-(5-((6-(3-fluoroazetidin-1-yl)-4-iodo-1H-pyrazolo[3,4-b]pyridin-1-yl)methyl)-1-oxoisoindolin-2-yl)-5-oxopentanoate (2.700 g, 4.16 mmol, 82% yield) as a yellow oil. ¹H NMR (400 MHz, CDCl₃) δ 7.78 (d, J=8.0 Hz, 1H), 7.65 (s, 1H), 7.45 (d, J=7.6 Hz, 1H), 7.35 (s, 1H), 6.64 (s, 1H), 6.25 (s, 1H), 5.57-5.39 (m, 3H), 5.29 (s, 1H), 4.87 (dd, J=6.6, 8.8 Hz, 1H), 4.48-4.35 (m, 4H), 4.27-4.18 (m, 2H), 2.34-2.13 (m, 4H), 1.41 (s, 9H).

### Intermediate G: tert-butyl(S)-5-amino-4-(5-((6-(3-fluoroazetidin-1-yl)-4-(4,4,5,5-tetramethyl-1,3,2-dioxaborolan-2-yl)-1H-pyrazolo[3,4-b]pyridin-1-yl)methyl)-1-oxoisoindolin-2-yl)-5-oxopentanoate

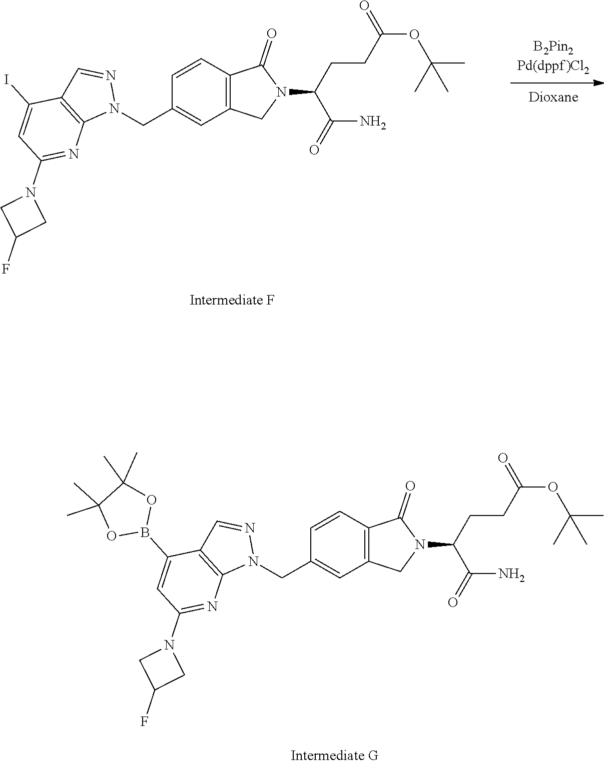

In a 40 mL Screw cap vial, tert-butyl(S)-5-amino-4-(5-((6-(3-fluoroazetidin-1-yl)-4-iodo-1H-pyrazolo[3,4-b]pyridin-1-yl)methyl)-1-oxoisoindolin-2-yl)-5-oxopentanoate (Intermediate F)(1.0 g, 1.542 mmol) was dissolved in 1,4-dioxane (20 mL). To this solution was added bis(pinacolato)diboron (0.783 g, 3.08 mmol), potassium acetate (0.454 g, 4.63 mmol), and PdCl₂(dppf)·DC adduct (0.126 g, 0.154 mmol). The mixture was purged with nitrogen for 5 min and stirred at 80° C. for 2 h. The mixture was filtered through celite and washed with ethyl acetate (100 mL). The filtrate was concentrated under reduced pressure to afford tert-butyl(S)-5-amino-4-(5-((6-(3-fluoroazetidin-1-yl)-4-(4,4,5,5-tetramethyl-1,3,2-dioxaborolan-2-yl)-1H-pyrazolo[3,4-b]pyridin-1-yl)methyl)-1-oxoisoindolin-2-yl)-5-oxopentanoate (1.2 g, 0.722 mmol, 47% yield) as a brown gummy solid. MS (ESI, +ve ion) m/z: 649.2 (M+1)⁺.

### Intermediate H: tert-butyl(S)-5-amino-4-(5-((5-fluoro-6-(3-fluoroazetidin-1-yl)-4-iodo-1H-pyrazolo[3,4-b]pyridin-1-yl)methyl)-1-oxoisoindolin-2-yl)-5-oxopentanoate

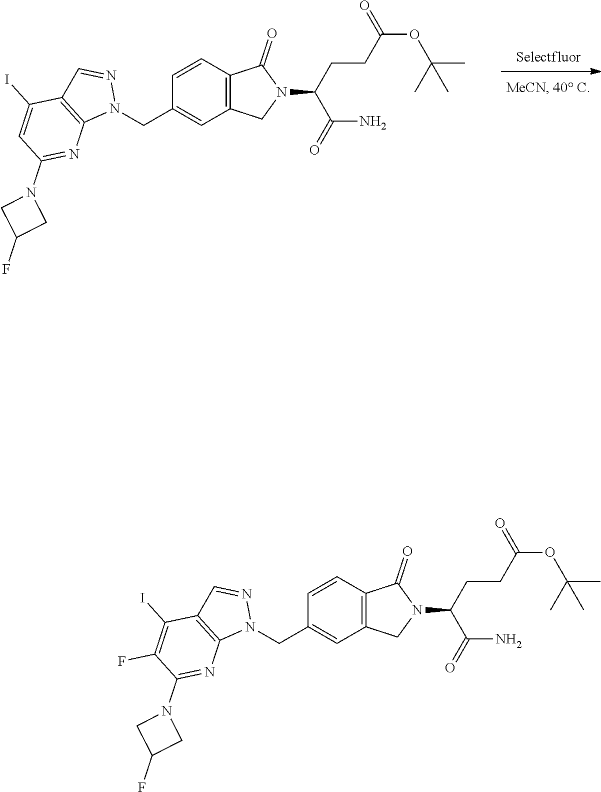 [SMILES: CC(C)(C)OC(=O)CC[C@@H](C(N)=O)N1Cc2cc(Cn3ncc4c(I)cc(N5CC(F)C5)nc43)ccc2C1=O ; CC(C)(C)OC(=O)CC[C@@H](C(N)=O)N1Cc2cc(Cn3ncc4c(I)c(F)c(N5CC(F)C5)nc43)ccc2C1=O]

To a solution of tert-butyl(S)-5-amino-4-(5-((6-(3-fluoroazetidin-1-yl)-4-iodo-1H-pyrazolo[3,4-b]pyridin-1-yl)methyl)-1-oxoisoindolin-2-yl)-5-oxopentanoate (Intermediate F) (2.700 g, 4.16 mmol) in acetonitrile (1 mL) was added 1-(chloromethyl)-4-fluoro-1,4-diazoniabicyclo[2.2.2]octane; ditetrafluoroborate (1.480 g, 4.16 mmol). The mixture was stirred at 40° C. for 12 h. The mixture was filtered and purified by semi-preparative reverse phase HPLC (52-82% acetonitrile in water+0.225% formic acid, over 10 min). The desired fractions were concentrated to remove most of the acetonitrile, then the aqueous solution was extracted with ethyl acetate (30 mL×3). The combined organic layer was dried over anhydrous sodium sulfate, filtered, and concentrated under vacuum to afford the product tert-butyl(S)-5-amino-4-(5-((5-fluoro-6-(3-fluoroazetidin-1-yl)-4-iodo-1H-pyrazolo[3,4-b]pyridin-1-yl)methyl)-1-oxoisoindolin-2-yl)-5-oxopentanoate (0.520 g, 0.78 mmol, 19% yield) as a yellow solid. 1H NMR (400 MHz, CDCl₃) δ 7.79 (d, J=7.2 Hz, 1H), 7.64 (s, 1H), 7.44 (d, J=8.0 Hz, 1H), 7.35 (s, 1H), 6.29 (d, J=2.0 Hz, 1H), 5.58-5.41 (m, 4H), 4.90-4.86 (m, 1H), 4.58-4.33 (m, 6H), 2.36-2.11 (m, 4H), 1.41 (s, 9H). MS (ESI) m/z: 667.1 [M+1]⁺.

### Intermediate I: tert-butyl 5-amino-4-(2-(bromomethyl)-5-oxo-5,7-dihydro-6H-pyrrolo[3,4-b]pyridin-6-yl)-5-oxopentanoate

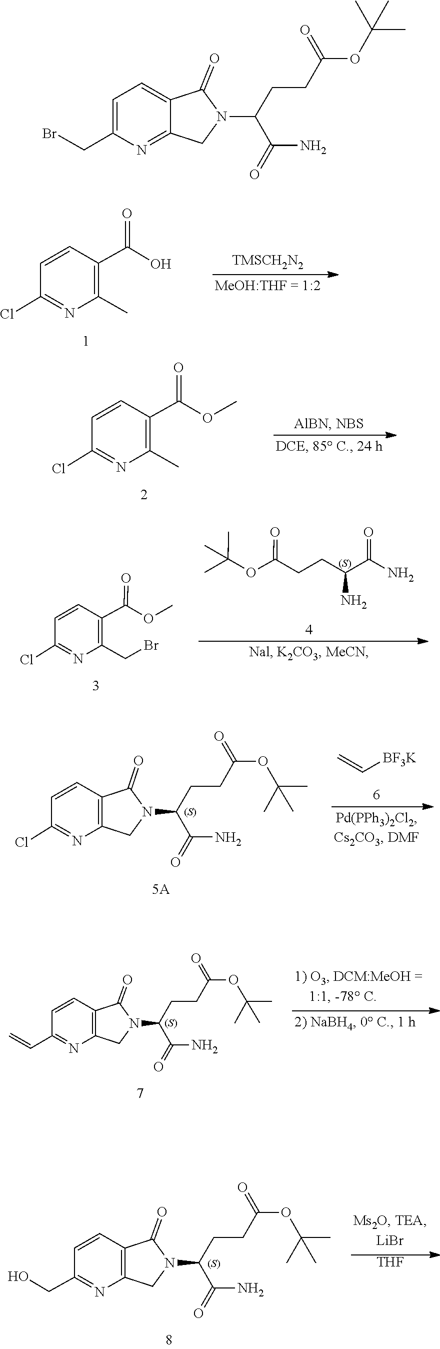

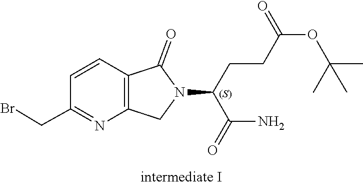

Step I1. methyl 6-chloro-2-methylnicotinate. To a solution of 6-chloro-2-methylnicotinic acid (25.00 g, 115.72 mmol) in methanol (50 mL) and tetrahydrofuran (100 mL) was added diazomethyl(trimethyl) silane (289.3 mL, 578.62 mmol) at 0° C. The mixture was stirred at 25° C. for 12 h. The reaction mixture was quenched by addition of acetic acid 5 mL at 0° C., diluted with water (100 mL), and extracted with ethyl acetate (100 mL×2). The combined organic layers were washed with brine (100 mL), dried over anhydrous sodium sulfate, filtered, and concentrated under reduced pressure. The residue was purified by silica gel chromatography (0-5% ethyl acetate in petroleum ether) to afford methyl 6-chloro-2-methylnicotinate (25.0 g, 108.67 mmol, 94% yield) as a white solid. ¹H NMR (400 MHz, CDCl₃) δ 8.15 (d, J=8.4 Hz, 1H), 7.23 (d, J=8.4 Hz, 1H), 3.91 (s, 3H), 2.81 (s, 3H).

Step I2. methyl 2-(bromomethyl)-6-chloronicotinate. A mixture of methyl 6-chloro-2-methylnicotinate (25.000 g, 134.69 mmol), N-bromosuccinimide (22.670 g, 202.04 mmol) and AIBN (17.694 g, 107.75 mmol) in DCM (150 mL) was stirred for 24 hours at 85° C. The mixture was diluted with water (100 mL) and extracted with ethyl acetate (100 mL×3). Then organic layers were dried over anhydrous sodium sulfate, filtered, and concentrated under reduced pressure. The residue was purified by semi-preparative reverse phase-HPLC (30-60% acetonitrile in water+0.225% formic acid, over 18 min). The desired fractions were extracted with ethyl acetate (100 mL×2). The combined organic layers were dried over anhydrous sodium sulfate, filtered, and concentrated under reduced pressure to give methyl 2-(bromomethyl)-6-chloronicotinate (12.300 g, 46.50 mmol, 34% yield) as pink solid. ¹H NMR (400 MHz, CDCl₃) δ 8.25 (d, J=8.4 Hz, 1H), 7.39 (d, J=8.4 Hz, 1H), 5.05 (s, 2H), 3.97 (s, 3H).

Step 13. tert-butyl(S)-5-amino-4-(2-chloro-5-oxo-5,7-dihydro-6H-pyrrolo[3,4-b]pyridin-6-yl)-5-oxopentanoate. A mixture of methyl 2-(bromomethyl)-6-chloronicotinate (8.300 g, 31.38 mmol), tert-butyl(S)-4,5-diamino-5-oxopentanoate (7.616 g, 37.66 mmol), potassium carbonate (10.826 g, 78.45 mmol) and potassium iodide (5.209 g, 31.38 mmol) in acetonitrile (100 mL) was stirred for 12 hours at 80° C. The mixture was diluted with water (100 mL) and extracted with ethyl acetate (80 mL×3). Then organic layers were dried over anhydrous sodium sulfate, filtered, and concentrated under reduced pressure. The residue was purified by silica gel chromatography (0-60% ethyl acetate in petroleum ether) to give tert-butyl (S)-5-amino-4-(2-chloro-5-oxo-5,7-dihydro-6H-pyrrolo[3,4-b]pyridin-6-yl)-5-oxopentanoate (9.100 g, 25.72 mmol, 82% yield) as yellow solid. ¹H NMR (400 MHz, CDCl₃) δ 8.00 (d, J=8.0 Hz, 1H), 7.41 (d, J=8.0 Hz, 1H), 6.64 (s, 1H), 6.02 (s, 1H), 4.96 (dd, J=6.0, 8.8 Hz, 1H), 4.71-4.43 (m, 2H), 2.35-2.24 (m, 3H), 2.15-2.08 (m, 1H), 1.39 (s, 9H). MS (ESI) m/z: 386.0 [M+1]⁺.

Step 14. tert-butyl(S)-5-amino-5-oxo-4-(5-oxo-2-vinyl-5,7-dihydro-6H-pyrrolo[3,4-b]pyridin-6-yl) pentanoate. A mixture of tert-butyl(S)-5-amino-4-(2-chloro-5-oxo-5,7-dihydro-6H-pyrrolo[3,4-b]pyridin-6-yl)-5-oxopentanoate (7.000 g, 19.79 mmol), trifluoro (vinyl)-14-borane, potassium salt (3.975 g, 29.68 mmol), bis(triphenylphosphine) palladium chloride (2.286 g, 1.98 mmol) and cesium carbonate (16.075 g, 49.46 mmol) in water (5 mL) and DMF (50 mL) was stirred for 12 hours at 90° C. The mixture was diluted with water (100 mL) and extracted with ethyl acetate (80 mL×3). The combined organic layers were washed with saturated brine (80 mL×2), dried over anhydrous sodium sulfate, filtered, and concentrated under reduced pressure. The residue was purified by silica column chromatography (0-80% ethyl acetate in petroleum ether). The desired fractions were combined and concentrated under reduced pressure. The residue was further purified by semi-preparative reverse phase-HPLC (20-50% acetonitrile in water+0.225% formic acid, over 20 min). The desired fractions were combined and extracted with ethyl acetate (100 mL×3). The combined organic extracts were dried over anhydrous sodium sulfate, filtered, and concentrated to afford the product tert-butyl(S)-5-amino-5-oxo-4-(5-oxo-2-vinyl-5,7-dihydro-6H-pyrrolo[3,4-b]pyridin-6-yl) pentanoate (2.800 g, 8.107 mmol, 41% yield) as a yellow solid. ¹H NMR (400 MHz, CDCl₃) δ 8.03 (d, J=8.0 Hz, 1H), 7.40 (d, J=8.0 Hz, 1H), 6.88 (dd, J=10.8, 17.6 Hz, 1H), 6.44 (s, 1H), 6.38 (d, J=17.2 Hz, 1H), 5.71 (s, 1H), 5.64 (d, J=10.8 Hz, 1H), 4.97 (dd, J=6.4, 8.8 Hz, 1H), 4.68-4.44 (m, 2H), 2.44-2.23 (m, 3H), 2.21-2.10 (m, 1H), 1.42 (s, 9H). MS (ESI) m/z: 289.2 [M+1]⁺.

Step 15. tert-butyl(S)-5-amino-4-(2-(hydroxymethyl)-5-oxo-5,7-dihydro-6H-pyrrolo[3,4-b]pyridin-6-yl)-5-oxopentanoate. To a solution of tert-butyl(S)-5-amino-5-oxo-4-(5-oxo-2-vinyl-5,7-dihydro-6H-pyrrolo[3,4-b]pyridin-6-yl) pentanoate (1.000 g, 2.90 mmol) in methanol (10 mL) and DCM (10 mL) was stirred for 0.5 hours at −78° C. under ozone. Then sodium borohydride (0.340. g, 8.99 mmol) was added. The mixture was stirred for 1 hours at 0° C. The reaction mixture was quenched with water and extracted with ethyl acetate (50 mL×3). The combined organic layers were concentrated under vacuum to give tert-butyl(S)-5-amino-4-(2-(hydroxymethyl)-5-oxo-5,7-dihydro-6H-pyrrolo[3,4-b]pyridin-6-yl)-5-oxopentanoate (0.800 g, 2.29 mmol, 79% yield) as a grey solid.

Step 16. tert-butyl(S)-5-amino-4-(2-(bromomethyl)-5-oxo-5,7-dihydro-6H-pyrrolo[3,4-b]pyridin-6-yl)-5-oxopentanoate. To a solution of tert-butyl(S)-5-amino-4-(2-(hydroxymethyl)-5-oxo-5,7-dihydro-6H-pyrrolo[3,4-b]pyridin-6-yl)-5-oxopentanoate (0.800 g, 2.29 mmol) and triethylamine (1.0 mL, 6.87 mmol) in THF (30 mL), was added methylsulfonyl methanesulfonate (0.997 g, 5.72 mmol). The reaction mixture was stirred for 10 minutes at 0° C. Then bromolithium (1.989 g, 22.9 mmol) was added. The resulting mixture was stirred at 0° C. for 0.5 h. The mixture was diluted with water (50 mL) and extracted with ethyl acetate (50 mL×3). The collected organic layers were dried over anhydrous sodium sulfate, filtered, and concentrated under reduced pressure. It was purified by silica gel chromatography (0˜90% ethyl acetate in petroleum ether) to afford the product tert-butyl(S)-5-amino-4-(2-(bromomethyl)-5-oxo-5,7-dihydro-6H-pyrrolo[3,4-b]pyridin-6-yl)-5-oxopentanoate (0.740 g, 1.79 mmol, 78% yield) as yellow solid. MS (ESI) m/z: 412.1 [M+1]⁺.

### Intermediate J: tert-butyl(S)-5-amino-4-(3-(bromomethyl)-7-oxo-5,7-dihydro-6H-pyrrolo[3,4-b]pyridin-6-yl)-5-oxopentanoate

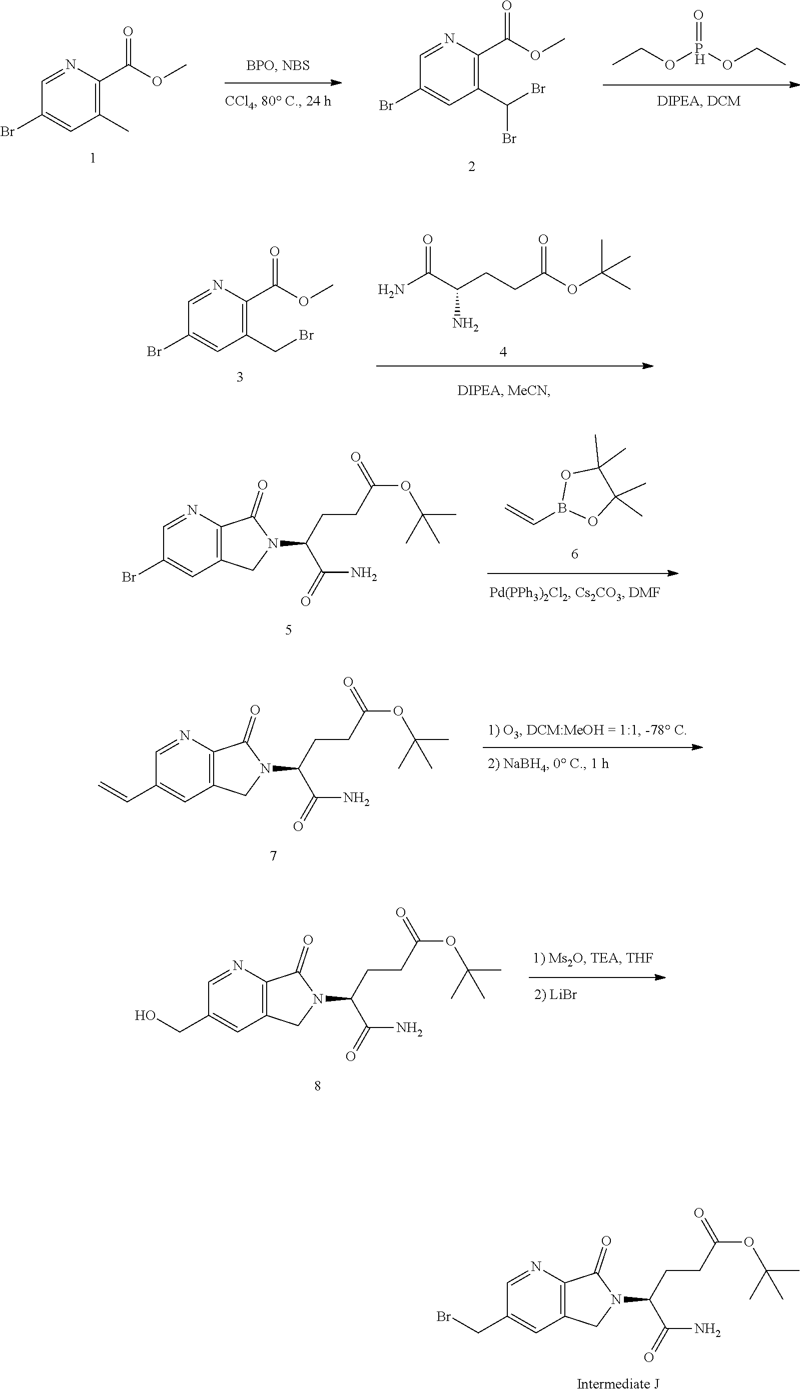

### Step J1: methyl 5-bromo-3-(dibromomethyl)picolinate

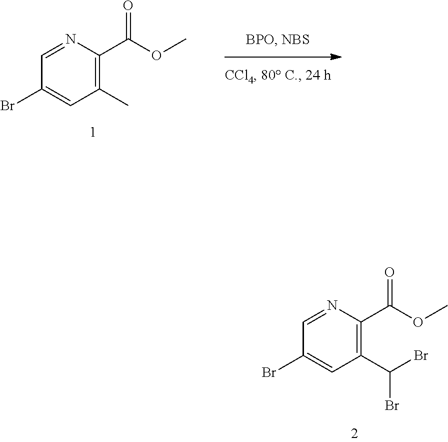

A mixture of methyl 5-bromo-3-methyl-pyridine-2-carboxylate (8.120 g, 35.30 mmol), NBS (12.564 g, 70.59 mmol) and benzoyl peroxide (0.427 g, 1.76 mmol) in carbon tetrachloride (80 mL) was stirred at 80° C. for 24 h under nitrogen. The mixture was concentrated under reduced pressure. The residue was purified by silica gel chromatography (0-25% ethyl acetate in petroleum ether) to give methyl 5-bromo-3-(dibromomethyl)picolinate (13.689 g, 35.30 mmol, 100.0% yield) as a yellow oil. MS (ESI) m/z: 387.8 [M+1]⁺.

### Step J2: methyl 5-bromo-3-(bromomethyl)picolinate

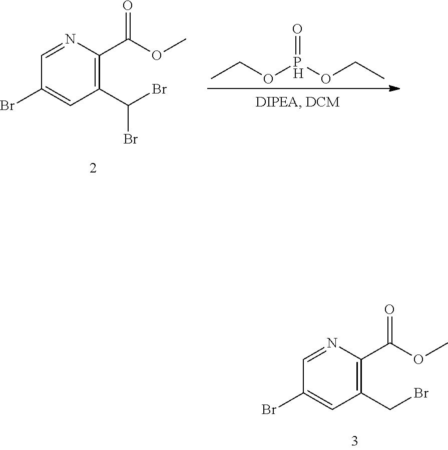

A mixture of methyl 5-bromo-3-(dibromomethyl)picolinate (13.689 g, 35.29 mmol), N-ethyl-N-isopropylpropan-2-amine (5.018 g, 38.82 mmol) and 1-ethoxyphosphonoyloxyethane (5.362 g, 38.82 mmol) in THF (150 mL) was stirred at 25° C. for 12 h. The reaction mixture was diluted with water (200 mL) and extracted with ethyl acetate (50 mL×2). The combined organic layers were concentrated under reduced pressure. The residue was purified by silica gel chromatography (0-25% ethyl acetate in petroleum ether) to give methyl 5-bromo-3-(bromomethyl)picolinate (10.670 g, 34.54 mmol, 98% yield) as a yellow solid. ¹H NMR (400 MHz, CDCl₃) δ 8.69 (d, J=2.4 Hz, 1H), 8.06 (d, J=2.4 Hz, 1H), 4.89 (s, 2H), 4.02 (s, 3H). MS (ESI) m/z: 309.9 [M+1]⁺.

### Step J3: tert-butyl(S)-5-amino-4-(3-bromo-7-oxo-5,7-dihydro-6H-pyrrolo[3,4-b]pyridin-6-yl)-5-oxopentanoate

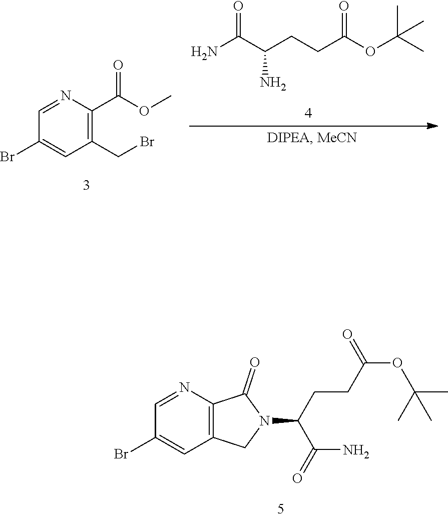 [SMILES: COC(=O)c1ncc(Br)cc1CBr ; CC(C)(C)OC(=O)CC[C@H](N)C(N)=O ; CC(C)(C)OC(=O)CC[C@@H](C(N)=O)N1Cc2cc(Br)cnc2C1=O]

A mixture of methyl 5-bromo-3-(bromomethyl)picolinate (10.670 g, 34.54 mmol), tert-butyl(S)-4,5-diamino-5-oxopentanoate (9.080 g, 44.90 mmol) and N-ethyl-N-isopropylpropan-2-amine (12.0 mL, 69.07 mmol) in acetonitrile (150 mL) was stirred at 100° C. for 12 h under nitrogen. The reaction mixture was concentrated under reduced pressure. The residue was purified by silica gel chromatography (0-80% ethyl acetate in petroleum ether) to give tert-butyl(S)-5-amino-4-(3-bromo-7-oxo-5,7-dihydro-6H-pyrrolo[3,4-b]pyridin-6-yl)-5-oxopentanoate (13.750 g, 34.53 mmol, 100% yield) as a light yellow solid. ¹H NMR (400 MHz, DMSO-d₆) δ 8.84 (d, J=2.0 Hz, 1H), 8.38 (d, J=2.0 Hz, 1H), 7.62 (s, 1H), 7.24 (s, 1H), 4.82-4.71 (m, 1H), 4.64-4.56 (m, 1H), 4.53-4.43 (m, 1H), 2.23-2.13 (m, 3H), 1.95 (s, 1H), 1.32 (s, 9H). MS (ESI) m/z: 400.0 [M+1]⁺.

### Step J4: tert-butyl(S)-5-amino-5-oxo-4-(7-oxo-3-vinyl-5,7-dihydro-6H-pyrrolo[3,4-b]pyridin-6-yl) pentanoate

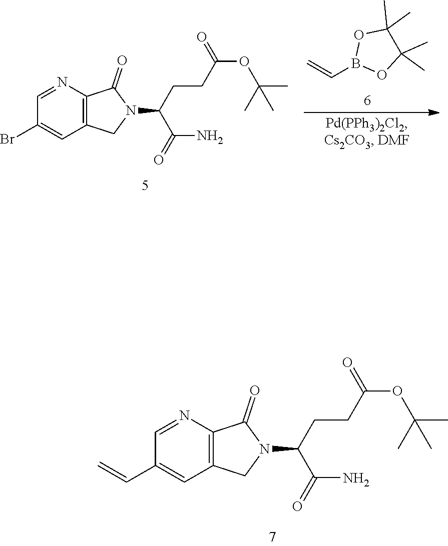

A mixture of tert-butyl(S)-5-amino-4-(3-bromo-7-oxo-5,7-dihydro-6H-pyrrolo[3,4-b]pyridin-6-yl)-5-oxopentanoate (2.000 g, 5.02 mmol), 4,4,5,5-tetramethyl-2-vinyl-1,3,2-dioxaborolane (1.160 g, 7.53 mmol), 2 M potassium carbonate (5.0 mL, 10.04 mmol) and bis-triphenylphosphine-palladium (II) chloride (0.353 g, 0.50 mmol) in DMF (20 mL) was stirred at 110° C. for 1 h under nitrogen. The reaction mixture was diluted with water (200 mL) and extracted with ethyl acetate (50 mL×2). The combined organic layers were concentrated under reduced pressure. The residue was purified by silica gel chromatography (0-81% ethyl acetate in petroleum ether) to give tert-butyl(S)-5-amino-5-oxo-4-(7-oxo-3-vinyl-5,7-dihydro-6H-pyrrolo[3,4-b]pyridin-6-yl) pentanoate (1.700 g, 4.92 mmol, 98% yield) as a yellow oil. ¹H NMR (400 MHz, DMSO-d₆) δ 8.80 (d, J=1.6 Hz, 1H), 8.19 (d, J=1.6 Hz, 1H), 7.61 (s, 1H), 7.22 (s, 1H), 6.91 (dd, J=11.2, 17.6 Hz, 1H), 6.12 (d, J=17.6 Hz, 1H), 5.53 (d, J=11.2 Hz, 1H), 4.77 (dd, J=4.4, 10.4 Hz, 1H), 4.64-4.55 (m, 1H), 4.53-4.43 (m, 1H), 2.23-2.13 (m, 3H), 2.07-1.99 (m, 1H), 1.35-1.30 (m, 9H). MS (ESI) m/z: 346.1 [M+1]⁺.

### Step J5: tert-butyl(S)-5-amino-4-(3-(hydroxymethyl)-7-oxo-5,7-dihydro-6H-pyrrolo[3,4-b]pyridin-6-yl)-5-oxopentanoate

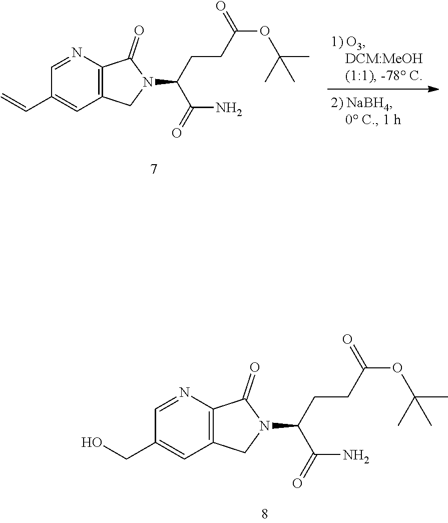 [SMILES: C=Cc1cnc2c(c1)CN([C@@H](CCC(=O)OC(C)(C)C)C(N)=O)C2=O ; CC(C)(C)OC(=O)CC[C@@H](C(N)=O)N1Cc2cc(CO)cnc2C1=O]

tert-butyl(S)-5-amino-5-oxo-4-(7-oxo-3-vinyl-5,7-dihydro-6H-pyrrolo[3,4-b]pyridin-6-yl) pentanoate (1.700 g, 4.92 mmol) was dissolved in dichloromethane (10 mL) and methanol (10 mL), then cooled to −78° C. This solution was saturated with ozone and a stream ozone was bubbled through for 30 minutes while stirring. Then nitrogen is then passed in with stirring for 10 min. The solution was warmed to 0° C. and sodium borohydride (0.344 g, 9.10 mmol) was added. The mixture was stirred at 0° C. for 1 h. The reaction mixture was quenched by water (2 mL), dried over anhydrous sodium sulfate solid, filtered, and concentrated under reduced pressure to give crude product tert-butyl(S)-5-amino-4-(3-(hydroxymethyl)-7-oxo-5,7-dihydro-6H-pyrrolo[3,4-b]pyridin-6-yl)-5-oxopentanoate (1.700 g, 4.87 mmol, 98.9% yield) as a brown solid. MS (ESI) m/z: 350.3 [M+1]⁺.

### Step J6: tert-butyl(S)-5-amino-4-(3-(bromomethyl)-7-oxo-5,7-dihydro-6H-pyrrolo[3,4-b]pyridin-6-yl)-5-oxopentanoate

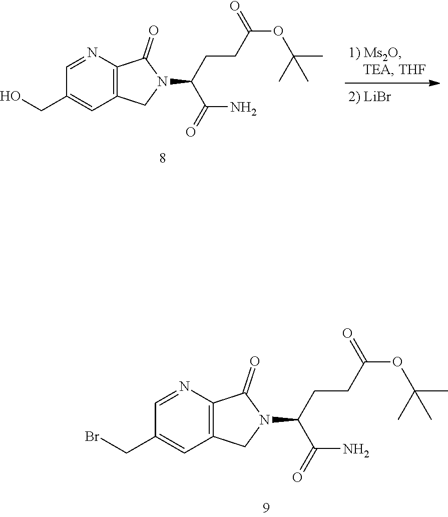 [SMILES: CC(C)(C)OC(=O)CC[C@@H](C(N)=O)N1Cc2cc(CO)cnc2C1=O ; CC(C)(C)OC(=O)CC[C@@H](C(N)=O)N1Cc2cc(CBr)cnc2C1=O]

To a solution of tert-butyl(S)-5-amino-4-(3-(hydroxymethyl)-7-oxo-5,7-dihydro-6H-pyrrolo[3,4-b]pyridin-6-yl)-5-oxopentanoate (1.700 g, 4.87 mmol) and triethylamine (2 mL, 14.6 mmol) in tetrahydrofuran (20 mL) was added methanesulfonic anhydride (1.695 g, 9.73 mmol) at 0° C. Then the mixture was warmed to 25° C. and stirred for 2 h. Lithium bromide (2.113 g, 24.33 mmol) was added and the reaction mixture was stirred at 0° C. for 1 h. The reaction mixture was diluted with water (100 mL) and extracted with ethyl acetate (20 mL×2). The combined organic layers were dried over anhydrous sodium sulfate solid, filtered, and concentrated under reduced pressure to give tert-butyl(S)-5-amino-4-(3-(bromomethyl)-7-oxo-5,7-dihydro-6H-pyrrolo[3,4-b]pyridin-6-yl)-5-oxopentanoate (1.600 g, 3.88 mmol, 80% yield) as a yellow oil. MS (ESI) m/z: 411.8 [M+1]⁺.

### Intermediate K: tert-butyl(S)-5-amino-4-(4-fluoro-5-(((methylsulfonyl)oxy)methyl)-1-oxoisoindolin-2-yl)-5-oxopentanoate

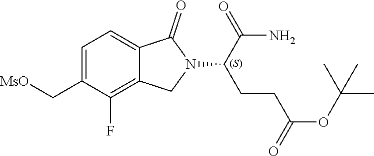

### Step K1: tert-butyl(S)-5-amino-4-(4-fluoro-5-(hydroxymethyl)-1-oxoisoindolin-2-yl)-5-oxopentanoate

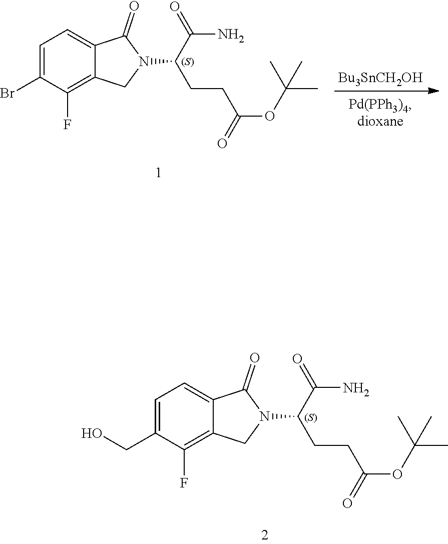

A mixture of tert-butyl(S)-5-amino-4-(5-bromo-4-fluoro-1-oxoisoindolin-2-yl)-5-oxopentanoate (prepared as described within WO2022216644)(5.000 g, 12.04 mmol), tributylstannylmethanol (4.639 g, 14.45 mmol) and tetrakis(triphenylphosphine) palladium (0) (1.391 g, 1.20 mmol) in dioxane (50 mL) was stirred at 100° C. for 48 h under nitrogen. The reaction mixture was quenched by potassium fluoride solution and filtered. The filtrate was extracted with ethyl acetate (50 mL×2). The combined organic layers were concentrated under reduced pressure. The residue was purified by silica gel chromatography (0-100% ethyl acetate in petroleum ether) to give tert-butyl(S)-5-amino-4-(4-fluoro-5-(hydroxymethyl)-1-oxoisoindolin-2-yl)-5-oxopentanoate (2.600 g, 7.10 mmol, 59% yield) as a white solid. MS (ESI) m/z: 372.2 [M+1]⁺.

### Step K2: tert-butyl(S)-5-amino-4-(4-fluoro-5-(((methylsulfonyl)oxy)methyl)-1-oxoisoindolin-2-yl)-5-oxopentanoate

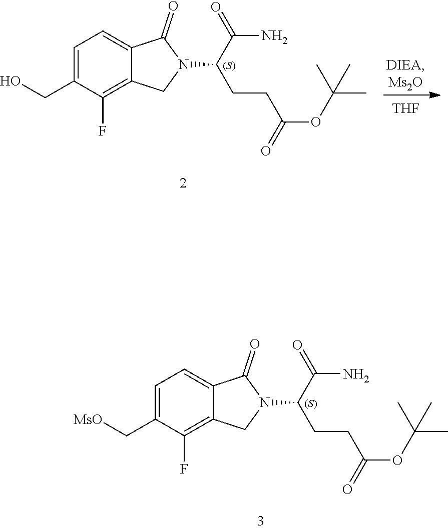

To a solution of tert-butyl(S)-5-amino-4-(4-fluoro-5-(hydroxymethyl)-1-oxoisoindolin-2-yl)-5-oxopentanoate (2.600 g, 7.10 mmol) and N-ethyl-N-isopropylpropan-2-amine (6.2 mL, 35.48 mmol) in tetrahydrofuran (100 mL) was added methylsulfonyl methanesulfonate (3.708 g, 21.29 mmol) at 0° C. Then the mixture was stirred at 25° C. for 30 min. The reaction mixture was diluted with water (100 mL) and extracted with ethyl acetate (50 mL×2). The combined organic layers were dried over anhydrous sodium sulfate solid, filtered, and concentrated under reduced pressure to give the crude product tert-butyl(S)-5-amino-4-(4-fluoro-5-(((methylsulfonyl)oxy)methyl)-1-oxoisoindolin-2-yl)-5-oxopentanoate (2.550 g, 5.74 mmol, 81% yield) as a yellow oil. MS (ESI) m/z: 445.2 [M+1]⁺.

### Intermediate L: tert-butyl(S)-5-amino-4-(7-fluoro-5-(((methylsulfonyl)oxy)methyl)-1-oxoisoindolin-2-yl)-5-oxopentanoate

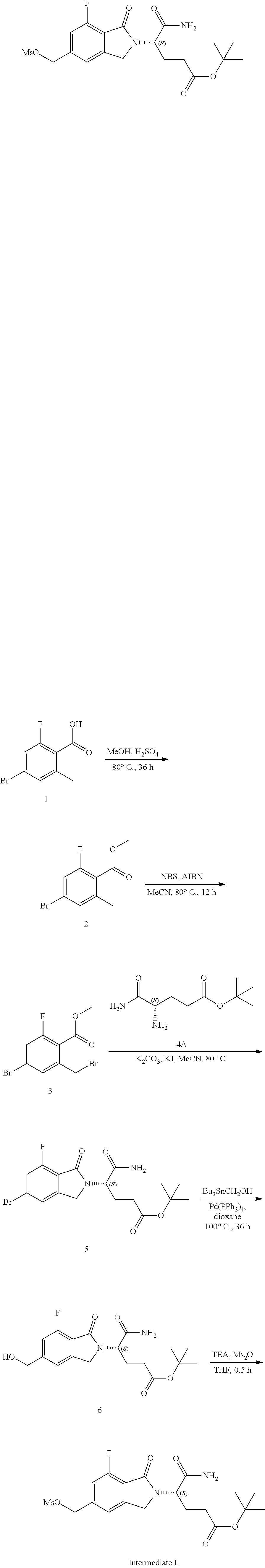

### Step L1: methyl 4-bromo-2-fluoro-6-methylbenzoate

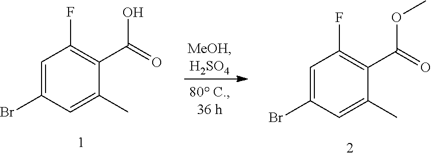

To a solution of 4-bromo-2-fluoro-6-methylbenzoic acid (35.000 g, 150.20 mmol) in methanol (250 mL) was added sulfuric acid (12 mL, 225.31 mmol). The mixture was stirred at 80° C. for 36 h. The reaction mixture was concentrated under reduced pressure. The residue was diluted with water 200 mL and extracted with ethyl acetate (200 mL×2). The combined organic layers were washed with brine 200 mL, dried over anhydrous sodium sulfate, filtered and concentrated under reduced pressure. The residue was purified by silica gel chromatography (0˜30% ethyl acetate in petroleum ether) to afford the product methyl 4-bromo-2-fluoro-6-methylbenzoate (35.000 g, 141.70 mmol, 94% yield) as a colorless oil. NMR (400 MHz, chloroform) δ 7.13 (s, 1H), 7.08 (d, J=9.2 Hz, 1H), 3.87 (s, 3H), 2.32 (s, 3H).

### Step L2: methyl 4-bromo-2-(bromomethyl)-6-fluorobenzoate

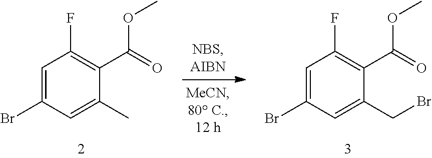

To a solution of methyl 4-bromo-2-fluoro-6-methylbenzoate (5.000 g, 20.24 mmol) in acetonitrile (250 mL) was added N-bromosuccinimide (9.010 g, 50.59 mmol) and (E)-2,2′-(diazene-1,2-diyl)bis(2-methylpropanenitrile)(3.320 g, 20.24 mmol). The mixture was stirred at 80° C. for 12 h. The reaction mixture was concentrated under reduced pressure. The residue was diluted with water (200 mL) and extracted with ethyl acetate (100 mL×2). The combined organic layers were washed with brine 200 mL, dried over anhydrous sodium sulfate, filtered and concentrated under reduced pressure. The residue was purified by silica gel chromatography (0˜5% ethyl acetate in petroleum ether) to afford the crude product methyl 4-bromo-2-(bromomethyl)-6-fluorobenzoate (4.500 g, 13.81 mmol, 68.2% yield) as a yellow oil. ¹H NMR (400 MHz, chloroform) § 7.39 (s, 1H), 7.30-7.27 (m, 1H), 4.61 (s, 2H), 3.98 (s, 3H).

### Step L3: tert-butyl(S)-5-amino-4-(5-bromo-7-fluoro-1-oxoisoindolin-2-yl)-5-oxopentanoate

To a solution of methyl 4-bromo-2-(bromomethyl)-6-fluorobenzoate (3.500 g, 10.74 mmol) and tert-butyl(S)-4,5-diamino-5-oxopentanoate (3.260 g, 16.11 mmol) and potassium carbonate (4.450 g, 32.21 mmol) in acetonitrile (50 mL) was added potassium iodide (1.78 g, 10.74 mmol). The reaction was stirred for 12 hours at 80° C. The mixture was diluted with water (100 mL) extracted with ethyl acetate (150 mL×3). The collected organic layers were washed with saturated brine (80 mL×3). Then organic layers were dried over anhydrous sodium sulfate, filtered, and concentrated under reduced pressure. The residue was purified by silica gel chromatography (0-60% ethyl acetate in petroleum ether) and concentrated to give tert-butyl(S)-5-amino-4-(5-bromo-7-fluoro-1-oxoisoindolin-2-yl)-5-oxopentanoate (4.000 g, 9.63 mmol, 90% yield) as yellow solid. ¹H NMR (400 MHz, chloroform) δ 7.43 (d, J=0.8 Hz, 1H), 7.32-7.27 (m, 1H), 6.51 (s, 1H), 5.41 (s, 1H), 4.90 (dd, J=6.4, 8.8 Hz, 1H), 4.66-4.40 (m, 2H), 2.44-2.44 (m, 1H), 2.38-2.20 (m, 3H), 2.17-2.07 (m, 1H), 1.42 (s, 9H). MS (ESI) m/z: 359.0 [M−55]⁺.

### Step L4: tert-butyl(S)-5-amino-4-(7-fluoro-5-(hydroxymethyl)-1-oxoisoindolin-2-yl)-5-oxopentanoate

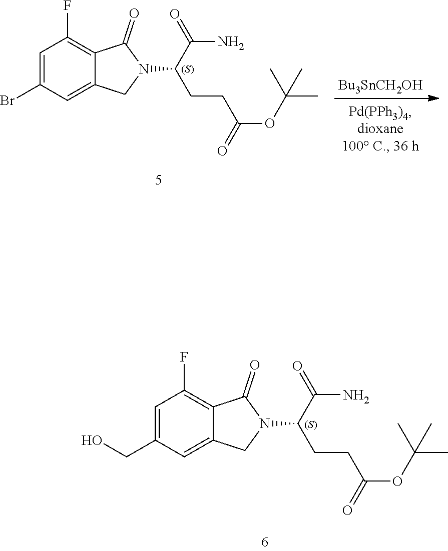

To a solution of tert-butyl(S)-5-amino-4-(5-bromo-7-fluoro-1-oxoisoindolin-2-yl)-5-oxopentanoate (0.500 g, 1.20 mmol) and tetrakis(triphenylphosphine) palladium (0.278 g, 0.24 mmol) in 1,4-dioxane (20 mL) was added (tributylstannyl) methanol (0.464 g, 1.44 mmol). The reaction was stirred for 36 hours at 100° C. The combined mixture was quenched by potassium fluoride solution and extracted with ethyl acetate (80 mL×2). The combined organic layers were concentrated under reduced pressure. The residue was purified by silica gel chromatography (0-95% ethyl acetate in petroleum ether) and concentrated to give tert-butyl(S)-5-amino-4-(7-fluoro-5-(hydroxymethyl)-1-oxoisoindolin-2-yl)-5-oxopentanoate (0.450 g, 1.23 mmol, 102% yield) as yellow solid. ¹H NMR (400 MHz, chloroform) δ 7.19 (s, 1H), 7.05 (s, 1H), 6.98 (d, J=10.0 Hz, 1H), 5.67 (s, 1H), 4.92 (dd, J=6.0, 8.4 Hz, 1H), 4.74 (s, 2H), 4.58-4.33 (m, 2H), 3.45-2.70 (m, 1H), 2.37-2.18 (m, 3H), 2.15-2.06 (m, 1H), 1.41 (s, 9H). MS (ESI) m/z: 311.0 [M−55]⁺.

### Step L5: tert-butyl(S)-5-amino-4-(7-fluoro-5-(((methylsulfonyl)oxy)methyl)-1-oxoisoindolin-2-yl)-5-oxopentanoate

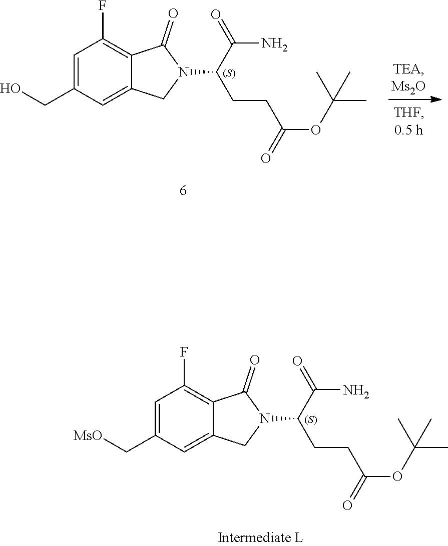

To a solution of tert-butyl(S)-5-amino-4-(7-fluoro-5-(hydroxymethyl)-1-oxoisoindolin-2-yl)-5-oxopentanoate (0.450 g, 1.23 mmol) and triethylamine (0.5 mL, 3.00 mmol) in THF (20 mL) was added methanesulfonic anhydride (0.387 g, 1.47 mmol) at 0° C. The reaction was stirred for 0.5 hours at 25° C. The reaction mixture was diluted with water (100 mL) and extracted with ethyl acetate (50 mL×3). The combined organic layers were dried over anhydrous sodium sulfate solid, filtered, and concentrated under reduced pressure to give tert-butyl(S)-5-amino-4-(7-fluoro-5-(((methylsulfonyl)oxy)methyl)-1-oxoisoindolin-2-yl)-5-oxopentanoate (0.480 g, 1.08 mmol, 88% yield) as yellow solid. MS (ESI) m/z: 445.1 [M+1]⁺.

### Intermediate M: 3-chloro-2-(tributylstannyl)-5-(trifluoromethyl)pyridine

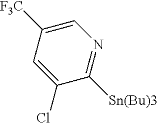

To a solution of 2-bromo-3-chloro-5-(trifluoromethyl)pyridine (5.000 g, 19.20 mmol) in toluene (80 mL) was added 2.5 M n-butyllithium (9.2 mL, 23.04 mmol) at −78° C. The mixture was stirred at 78° C. for 1 h under nitrogen. Then tributylchlorostannane (7.8 mL, 28.85 mmol) was added and the mixture was stirred at 78° C. for 2 h under nitrogen. The reaction mixture was quenched by saturated aqueous solution of ammonium chloride (200 mL) and extracted with ethyl acetate (100 mL×3). The organic layers were combined, dried over anhydrous sodium sulfate, filtered, and concentrated under vacuum. The residue was purified by neutral aluminium oxide chromatography, eluting with 100% petroleum ether to afford 3-chloro-2-(tributylstannyl)-5-(trifluoromethyl)pyridine (7.000 g, 14.88 mmol, 78% yield) as a colorless oil. ¹H NMR (400 MHz, CDCl₃) δ 8.87 (s, 1H), 7.73 (s, 1H), 1.60-1.56 (m, 6H), 1.37-1.29 (m, 7H), 1.26-1.21 (m, 5H), 0.89 (t, J=7.2 Hz, 9H).

### Intermediate N: 2-(2-chloro-4-(2,2,2-trifluoroethoxy)phenyl)-4,4,5,5-tetramethyl-1,3,2-dioxaborolane

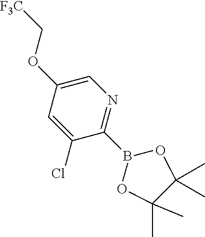 [SMILES: CC1(C)OB(c2ncc(OCC(F)(F)F)cc2Cl)OC1(C)C]

Step N1:1-bromo-2-chloro-4-(2,2,2-trifluoroethoxy)benzene. To a solution of 4-bromo-3-chlorophenol (1.000 g, 4.82 mmol) in dimethylformamide (10 mL) was added potassium carbonate (1.330 g, 9.64 mmol). The mixture was stirred at 25° C. for 0.5 h. Then 2,2,2-trifluoroethyl trifluoromethanesulfonate (1.170 g, 5.06 mmol) dissolved into dimethylformamide (10 mL) was added and the mixture was stirred at 100° C. for 12 h. The mixture was diluted with water (20 mL) and extracted with ethyl acetate (20 mL×3). The combined organic layers were washed with saturated sodium chloride (5 mL), dried over anhydrous sodium sulfate, filtered, and concentrated under reduced pressure to afford 1-bromo-2-chloro-4-(2,2,2-trifluoroethoxy)benzene (1.300 g, 4.49 mmol, 93% yield) as a yellow oil. 1H NMR (400 MHz, CDCl₃) δ 7.53 (d, J=8.8 Hz, 1H), 7.07 (d, J=2.8 Hz, 1H), 6.75 (dd, J=2.8, 8.8 Hz, 1H), 4.33 (q, J=8.0 Hz, 2H).

Step N2:2-(2-chloro-4-(2,2,2-trifluoroethoxy)phenyl)-4,4,5,5-tetramethyl-1,3,2-dioxaborolane. To a solution of 1-bromo-2-chloro-4-(2,2,2-trifluoroethoxy)benzene (0.200 g, 0.69 mmol) and 4,4,4′,4′,5,5,5′,5′-octamethyl-2,2′-bi (1,3,2-dioxaborolane)(0.211 g, 0.83 mmol) in 1,4-dioxane (2 mL) was added potassium acetate (0.203 g, 2.07 mmol) and (1,1′-bis(diphenylphosphino) ferrocene) palladium (II) dichloride (0.056 g, 0.07 mmol). The mixture was stirred at 100° C. for 12 h under nitrogen. The mixture was filtered and concentrated under reduced pressure. The residue was purified by prep-TLC (9% ethyl acetate in petroleum ether) to give 2-(2-chloro-4-(2,2,2-trifluoroethoxy)phenyl)-4,4,5,5-tetramethyl-1,3,2-dioxaborolane (0.050 g, 0.15 mmol, 22% yield) as a yellow oil. ¹H NMR (400 MHz, CDCl₃) δ 7.69 (d, J=8.4 Hz, 1H), 6.96 (d, J=2.4 Hz, 1H), 6.83 (dd, J=2.4, 8.4 Hz, 1H), 4.40-4.33 (m, 2H).

### Intermediate O: 2-[3-chloro-4-(4,4,5,5-tetramethyl-1,3,2-dioxaborolan-2-yl)phenyl]propan-2-ol

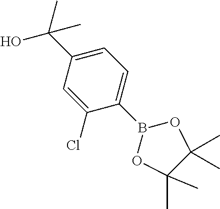 [SMILES: CC(C)(O)c1ccc(B2OC(C)(C)C(C)(C)O2)c(Cl)c1]

Step 01:2-(4-bromo-3-chloro-phenyl) propan-2-ol. To a solution of methyl 4-bromo-3-chloro-benzoate (2.000 g, 8.02 mmol) in tetrahydrofuran (20 mL) was added methyl magnesium bromide (5.34 mL, 16.03 mmol) at −78° C. under nitrogen. The mixture was stirred at 25° C. under nitrogen for 2 hours. The mixture was poured into saturated ammonium chloride (50 mL) and extracted with ethyl acetate (50 mL×3). The combined organic layers were washed with brine (50 mL×1), dried with anhydrous sodium sulfate, filtered, and concentrated under reduced pressure. The residue was purified by silica gel chromatography (10˜30% ethyl acetate in petroleum ether) to afford the product 2-(4-bromo-3-chloro-phenyl) propan-2-ol (1.500 g, 6.01 mmol, 75% yield) as a colorless oil. ¹H NMR (400 MHz, CDCl₃) δ 7.60 (d, J=2.4, 1H), 7.60 (d, J=8.4, 1H), 7.24-7.22 (m, 1H), 1.57 (s, 6H).

Step O2:2-[3-chloro-4-(4,4,5,5-tetramethyl-1,3,2-dioxaborolan-2-yl)phenyl]propan-2-ol. To a solution of 2-(4-bromo-3-chloro-phenyl) propan-2-ol (1.500 g, 6.01 mmol) in 1,4-dioxane (15 mL) was added bis(pinacolato)diboron (3.050 g, 12.02 mmol), potassium acetate (1.770 g, 18.03 mmol) and (1,1′-bis(diphenylphosphino) ferrocene) palladium (II) dichloride (0.491 g, 0.60 mmol). The mixture was stirred at 85° C. under nitrogen for 12 hours. The mixture was filtered and concentrated under reduced pressure. The residue was purified by silica gel chromatography (10˜30% ethyl acetate in petroleum ether) to afford 2-[3-chloro-4-(4,4,5,5-tetramethyl-1,3,2-dioxaborolan-2-yl)phenyl]propan-2-ol (1.500 g, 5.06 mmol, 84% yield) as a colorless oil. ¹H NMR (400 MHz, CDCl₃) δ 7.67 (d, J=7.6 Hz, 1H), 7.34 (d, J=1.6 Hz, 1H), 1.57 (s, 6H), 1.37 (s, 12H).

### Intermediate P: 2-[4-chloro-3-(4,4,5,5-tetramethyl-1,3,2-dioxaborolan-2-yl)phenyl]propan-2-ol

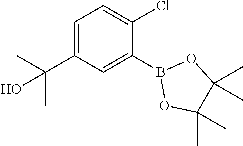 [SMILES: CC(C)(O)c1ccc(Cl)c(B2OC(C)(C)C(C)(C)O2)c1]

Step P1:2-(3-bromo-4-chloro-phenyl) propan-2-ol. To a solution of methyl 3-bromo-4-chloro-benzoate (1.000 g, 4.01 mmol) in tetrahydrofuran (10 mL) was added methyl magnesium bromide (4.01 mL, 12.02 mmol) at 0° C. The mixture was stirred at 25° C. for 12 hour. The reaction mixture was diluted with water (10 mL) and extracted with ethyl acetate (15 mL×3). The combined organic layer was washed with saturated brine (10 mL×2) and dried over anhydrous sodium sulfate. The organic layer was filtered and concentrated under reduced pressure. The residue was purified by silica column chromatography (9%-33% of ethyl acetate in petroleum ether) to afford 2-(3-bromo-4-chloro-phenyl) propan-2-ol (0.870 g, 3.49 mmol, 87% yield) as a white oil. ¹H NMR (400 MHz, CDCl₃) δ 7.77 (s, 1H), 7.42-7.40 (m, 1H), 7.37-7.35 (m, 1H), 1.57 (s, 6H).

Step P2:2-[4-chloro-3-(4,4,5,5-tetramethyl-1,3,2-dioxaborolan-2-yl)phenyl]propan-2-ol. To a solution of 2-(3-bromo-4-chloro-phenyl) propan-2-ol (1.300 g, 5.21 mmol) and bis(pinacolato)diboron (1.455 g, 5.73 mmol) in 1,4-dioxane (15 mL) was added (1, l′-bis(diphenylphosphino) ferrocene) palladium (II) dichloride (0.425 g, 0.52 mmol) and potassium acetate (1.531 g, 15.63 mmol). The mixture was stirred at 85° C. for 12 hours under nitrogen. The mixture was concentrated under reduced pressure and purified by silica gel chromatography (0%-25% of ethyl acetate in petroleum ether) to afford 2-[4-chloro-3-(4,4,5,5-tetramethyl-1,3,2-dioxaborolan-2-yl)phenyl]propan-2-ol (1.100 g, 3.71 mmol, 71% yield) as a colorless oil. ¹H NMR (400 MHz, CDCl₃) δ 7.78 (d, J=2.4 Hz, 1H), 7.42 (s, 1H), 7.30 (d, J=2.0 Hz, 1H), 1.57 (s, 6H), 1.38 (s, 12H).

### EXAMPLE SYNTHESES

### Example 2: (3S)-3-[5-({5-chloro-4-[3-chloro-5-(trifluoromethyl)pyridin-2-yl]-6-(3-fluoroazetidin-1-yl)-1H-pyrazolo[3,4-b]pyridin-1-yl}methyl)-1-oxo-2,3-dihydro-1H-isoindol-2-yl]piperidine-2,6-dione

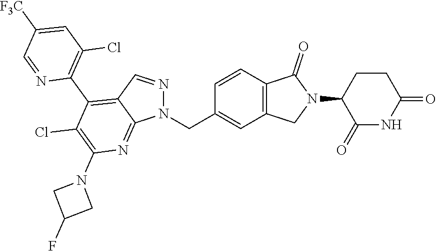 [SMILES: O=C1CC[C@H](N2Cc3cc(Cn4ncc5c(-c6ncc(C(F)(F)F)cc6Cl)c(Cl)c(N6CC(F)C6)nc54)ccc3C2=O)C(=O)N1]

A. (3S)-3-(5-((5-chloro-4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione. (S)-3-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione (165 mg, 0.263 mmol)(synthesized according to Example 6) was dissolved in DMF (3 mL) and treated with N-chlorosuccinimide (42.1 mg, 0.315 mmol). The reaction mixture was sealed and heated at 100° C. for 30 min in a microwave reactor. The reaction mixture was purified by preparative HPLC purification using the following method. Column: X-bridge C18 (150×19) mm, 5 micron, Diluent: THF:Water:ACN (50:20:30), Mobile phase A: 5 mM Ammonium formate in water, Mobile phase B: Acetonitrile to yield (3S)-3-(5-((5-chloro-4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione (54 mg, 0.079 mmol, 30.2% yield) as an off-white solid. ¹H NMR (400 MHz, DMSO-d₆) δ 10.98 (br s, 1H), 9.16 (d, J=1.0 Hz, 1H), 8.79 (d, J=1.5 Hz, 1H), 7.72 (d, J=8.0 Hz, 1H), 7.67 (s, 1H), 7.55 (s, 1H), 7.48 (d, J=7.5 Hz, 1H), 5.73-5.61 (m, 2H), 5.59-5.38 (m, 1H), 5.10 (dd, J=5.3, 13.3 Hz, 1H), 4.77-4.61 (m, 2H), 4.50-4.26 (m, 4H), 2.98-2.85 (m, 1H), 2.65-2.56 (m, 1H), 2.43-2.34 (m, 1H), 2.06-1.94 (m, 1H); MS (ESI, +ve) m/z: 662.1 (M+1)⁺.

### Example 3:3-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl) piperidine-2,6-dione

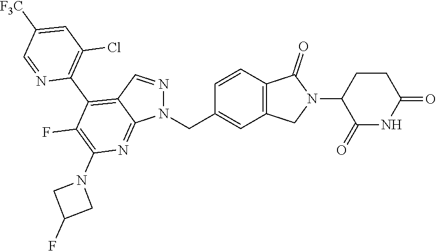 [SMILES: O=C1CCC(N2Cc3cc(Cn4ncc5c(-c6ncc(C(F)(F)F)cc6Cl)c(F)c(N6CC(F)C6)nc54)ccc3C2=O)C(=O)N1]

A. tert-butyl (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoate. To a solution of tert-butyl(S)-5-amino-4-(5-((5-fluoro-6-(3-fluoroazetidin-1-yl)-4-iodo-1H-pyrazolo[3,4-b]pyridin-1-yl)methyl)-1-oxoisoindolin-2-yl)-5-oxopentanoate (Intermediate H)(0.120 g, 0.18 mmol) in 1,4-dioxane (3 mL) was added 3-chloro-2-(tributylstannyl)-5-(trifluoromethyl)pyridine (Intermediate M)(0.085 g, 0.18 mmol), tetrakis(triphenylphosphine) palladium (0)(0.016 g, 0.02 mmol) and copper (I) iodide (0.003 g, 0.02 mmol) under nitrogen. The mixture was stirred at 85° C. for 12 h. The reaction mixture was quenched by addition of aqueous potassium fluoride solution 50 mL, and then diluted with water (50 mL) and extracted with ethyl acetate (50 mL×2). The combined organic layers were washed with brine (50 mL), dried over anhydrous sodium sulfate, filtered and concentrated under reduced pressure. The residue was purified by preparative TLC. The material was further purified by semi-preparative reverse phase-HPLC (55%-85% acetonitrile in water+0.225% formic acid, 5 min). The desired fractions were concentrated under vacuum and then extracted with ethyl acetate (100 mL×3). The combined organic layers were dried over anhydrous sodium sulfate, filtered and concentrated under reduced pressure to give a tert-butyl (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoate (0.060 g, 0.08 mmol, 46% yield) as a yellow solid. MS (ESI) m/z: 720.2 [M+1]⁺.

B. (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoic acid. To a solution of tert-butyl (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoate (0.060 g, 0.08 mmol) in dichloromethane (2 mL) was added TFA (0.5 mL). The reaction mixture was quenched by addition N-ethyl-N-isopropylpropan-2-amine (5 mL) and then diluted with water (20 mL) and extracted with ethyl acetate (20 mL×2). The combined organic layers were concentrated under reduced pressure to give a crude (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoic acid (0.050 g, 0.08 mmol, 90.4% yield). The material was used into next step without purification. MS (ESI) m/z: 664.1 [M+1]⁺.

C. 3-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl) piperidine-2,6-dione. To a solution of (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoic acid (0.050 g, 0.08 mmol) in acetonitrile (3 mL) was added N-ethyl-N-isopropylpropan-2-amine (0.02 mL, 0.23 mmol), di(1H-imidazol-1-yl) methanone (0.012 g, 0.08 mmol), N,N-dimethylpyridin-2-amine (0.010 g, 0.08 mmol). The mixture was stirred at 80° C. for 12 h. The reaction was purified by semi-preparative reverse phase-HPLC (47%-77% acetonitrile in water+0.225% formic acid over 5 min). The desired fractions were concentrated under vacuum and then lyophilized to afford 3-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl) piperidine-2,6-dione (0.025 g, 0.04 mmol, 51% yield, 100% purity) as a yellow solid. ¹H NMR (400 MHz, DMSO) δ 11.17-10.76 (m, 1H), 9.16 (d, J=1.2 Hz, 1H), 8.78 (d, J=1.6 Hz, 1H), 7.74-7.68 (m, 2H), 7.53-7.49 (m, 1H), 7.43 (d, J=7.6 Hz, 1H), 5.64 (s, 2H), 5.62-5.43 (m, 1H), 5.10 (dd, J=5.2, 12.8 Hz, 1H), 4.65-4.53 (m, 2H), 4.47-4.41 (m, 1H), 4.37-4.24 (m, 3H), 2.96-2.84 (m, 1H), 2.58-2.56 (m, 1H), 2.43-2.36 (m, 1H), 2.03-1.94 (m, 1H). MS (ESI) m/z: 646.2 [M+1]⁺.

### Example 6: (3S)-3-[5-({4-[3-chloro-5-(trifluoromethyl)pyridin-2-yl]-6-(3-fluoroazetidin-1-yl)-1H-pyrazolo[3,4-b]pyridin-1-yl}methyl)-1-oxo-2,3-dihydro-1H-isoindol-2-yl]piperidine-2,6-dione

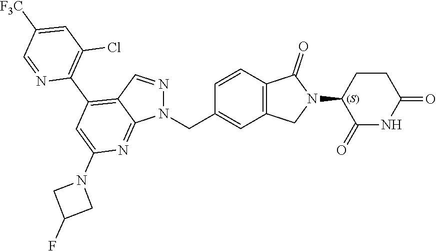

A. tert-butyl(S)-5-amino-4-(5-((4-(3-chloro-5-(trifluoromethyl)pyridin-2-yl)-6-fluoro-1H-pyrazolo[3,4-b]pyridin-1-yl)methyl)-1-oxoisoindolin-2-yl)-5-oxopentanoate. To a solution of 3-chloro-2-(tributylstannyl)-5-(trifluoromethyl)pyridine (Intermediate M)(119 mg, 0.253 mmol) in 1,4-Dioxane (5 mL) was added tert-butyl(S)-5-amino-4-(5-((6-fluoro-4-iodo-1H-pyrazolo[3,4-b]pyridin-1-yl)methyl)-1-oxoisoindolin-2-yl)-5-oxopentanoate (Intermediate C)(100 mg, 0.169 mmol), copper (I) iodide (48.1 mg, 0.253 mmol), and tetrakis(triphenylphosphine) palladium (0)(29.2 mg, 0.025 mmol). The mixture was stirred at 100° C. for 12 h under nitrogen. The reaction mixture was filtered through celite, washed with ethyl acetate, and concentrated under vacuum. The residue was purified by reverse phase HPLC using a gradient of acetonitrile in 0.1% formic acid to obtain t-butyl(S)-5-amino-4-(5-((4-(3-chloro-5-(trifluoromethyl)pyridin-2-yl)-6-fluoro-1H-pyrazolo[3,4-b]pyridin-1-yl)methyl)-1-oxoisoindolin-2-yl)-5-oxopentanoate (70 mg, 0.108 mmol, 64.1% yield) as an off white solid. MS (ESI) m/z: 647.0 [M]⁺.

B. tert-butyl(S)-5-amino-4-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate. To a stirred solution of tert-butyl(S)-5-amino-4-(5-((4-(3-chloro-5-(trifluoromethyl)pyridin-2-yl)-6-fluoro-1H-pyrazolo[3,4-b]pyridin-1-yl)methyl)-1-oxoisoindolin-2-yl)-5-oxopentanoate (100 mg, 0.147 mmol) in acetonitrile (3 mL) was added 3-fluoroazetidine HCl (35 mg, 0.314 mmol) and the mixture was stirred at 80° C. for 4 h. The mixture was evaporated under vacuum and the residue was diluted in DCM (5 mL), washed with water (5 mL), the organic layer was concentrated to afford tert-butyl(S)-5-amino-4-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate 3 (100 mg, 0.122 mmol, 83% yield) as a yellow solid. MS (ESI, +ve) m/z: 702.3 (M+1)⁺.

(S)-3-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione. To a stirred solution of tert-butyl(S)-5-amino-4-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5 oxopentanoate (100 mg, 0.122 mmol) in acetonitrile (3 mL) was added p-toluenesulfonic acid (106 mg, 0.558 mmol) and the mixture was stirred at 60° C. for overnight. The reaction mixture was evaporated, and the crude was purified by preparative HPLC using the following conditions: (Column: Xselect CSH C18 (250×19) mm 10.0 μm, Mobile phase A: 0.1% formic acid in water, Mobile phase B: acetonitrile, Flow: 14 mL/min, 15-90% B over 20 min.). The product fraction was evaporated, and the residue was lyophilized by dissolving in ACN/water (1:1 mixture, 5 mL) for 24 h, to afford(S)-3-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione (39 mg, 0.062 mmol, 50.7% yield) as an off-white solid. ¹H NMR (400 MHz, DMSO-d₆) δ:10.98 (s, 1H), 9.13 (dd, J=1.9, 0.8 Hz, 1H), 8.70-8.74 (m, 1H), 7.80 (s, 1H), 7.70 (d, J=7.9 Hz, 1H), 7.50 (s, 1H), 7.42 (d, J=8.8 Hz, 1H), 6.63 (s, 1H), 5.59-5.70 (m, 3H), 5.48 (dq, J=5.8, 3.0 Hz, 1H), 5.10 (dd, J=13.3, 5.1 Hz, 1H), 4.38-4.52 (m, 2H), 4.12-4.34 (m, 3H), 2.85-2.96 (m, 1H), 2.28-2.49 (m, 2H), 1.92-2.04 (m, 1H). MS (ESI, +ve) m/z: 628.2 (M+1)⁺.

### Example 9: (3S)-3-(5-((4-(3-chloro-5-fluoropyridin-2-yl)-5-fluoro-6-(isopropylamino)-1H-pyrazolo[3,4-b]pyridin-1-yl)methyl)-1-oxoisoindolin-2-yl) piperidine-2,6-dione

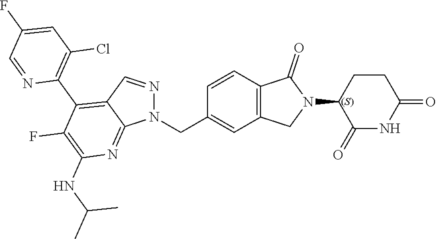

A. tert-butyl(S)-5-amino-4-(5-((4-iodo-6-(isopropylamino)-1H-pyrazolo[3,4-b]pyridin-1-yl)methyl)-1-oxoisoindolin-2-yl)-5-oxopentanoate. To a solution of tert-butyl(S)-5-amino-4-(5-((6-fluoro-4-iodo-1H-pyrazolo[3,4-b]pyridin-1-yl)methyl)-1-oxoisoindolin-2-yl)-5-oxopentanoate (Intermediate C)(0.350 g, 0.59 mmol) in acetonitrile (4 mL) was added propan-2-amine (0.042 g, 0.71 mmol) and N-ethyl-N-isopropylpropan-2-amine (0.3 mL, 1.77 mmol). The mixture was stirred at 50° C. for 12 h. The mixture was diluted with water (20 mL), extracted with ethyl acetate (20 mL×3). The combined organic layer was dried over anhydrous sodium sulfate, filtered and concentrated under reduced pressure to afford tert-butyl(S)-5-amino-4-(5-((4-iodo-6-(isopropylamino)-1H-pyrazolo[3,4-b]pyridin-1-yl)methyl)-1-oxoisoindolin-2-yl)-5-oxopentanoate (0.350 g, 0.55 mmol, 93.8% yield) as a yellow oil. MS (ESI) m/z: 633.3 [M+1]⁺.

B. tert-butyl(S)-5-amino-4-(5-((5-fluoro-4-iodo-6-(isopropylamino)-1H-pyrazolo[3,4-b]pyridin-1-yl)methyl)-1-oxoisoindolin-2-yl)-5-oxopentanoate. To a solution of tert-butyl(S)-5-amino-4-(5-((4-iodo-6-(isopropylamino)-1H-pyrazolo[3,4-b]pyridin-1-yl)methyl)-1-oxoisoindolin-2-yl)-5-oxopentanoate (0.350 g, 0.55 mmol) in acetonitrile (4 mL) was added 1-chloromethyl-4-fluoro-1,4-diazoniabicyclo[2.2.2]octane fluoride (tetrafluoroborate) (0.235 g, 0.66 mmol). The mixture was stirred at 25° C. for 3 h. The mixture was diluted with water (10 mL), extracted with ethyl acetate (10 mL×3). The combined organic layer was dried over anhydrous sodium sulfate, filtered and concentrated under reduced pressure. The residue was purified by semi-preparative reverse phase HPLC (53-83% acetonitrile in water+0.225% formic acid, over 7 min). Then the collected fraction was concentrated to remove most of the acetonitrile, then lyophilized to afford the product tert-butyl(S)-5-amino-4-(5-((5-fluoro-4-iodo-6-(isopropylamino)-1H-pyrazolo[3,4-b]pyridin-1-yl)methyl)-1-oxoisoindolin-2-yl)-5-oxopentanoate (0.120 g, 0.18 mmol, 33.3% yield) as a yellow oil. ¹H NMR (400 MHz, CDCl₃) δ 7.78 (d, J=7.6 Hz, 1H), 7.60 (s, 1H), 7.48 (d, J=8.0 Hz, 1H), 7.40 (s, 1H), 6.39 (s, 1H), 5.67 (s, 1H), 5.58 (s, 2H), 4.91-4.85 (m, 2H), 4.50-4.36 (m, 2H), 4.33-4.28 (m, 1H), 2.36-2.10 (m, 4H), 1.41 (s, 9H), 1.31 (d, J=6.4 Hz, 6H). MS (ESI) m/z: 651.1 [M+1]⁺.

C. tert-butyl (4S)-5-amino-4-(5-((4-(3-chloro-5-fluoropyridin-2-yl)-5-fluoro-6-(isopropylamino)-1H-pyrazolo[3,4-b]pyridin-1-yl)methyl)-1-oxoisoindolin-2-yl)-5-oxopentanoate. To a solution of tert-butyl(S)-5-amino-4-(5-((5-fluoro-4-iodo-6-(isopropylamino)-1H-pyrazolo[3,4-b]pyridin-1-yl)methyl)-1-oxoisoindolin-2-yl)-5-oxopentanoate (0.100 g, 0.15 mmol) in dioxane (1 mL) was added 3-chloro-5-fluoro-2-(tributylstannyl)pyridine (Intermediate G)(0.071 g, 0.17 mmol), tetrakis(triphenylphosphine) palladium (0)(0.014 g, 0.02 mmol) and cuprous iodide (0.029 g, 0.15 mmol). The mixture was stirred at 85° C. for 12 h under nitrogen. The mixture was filtered and concentrated. Then the mixture was purified by preparative TLC (80% ethyl acetate in petroleum ether) to afford the product tert-butyl (4S)-5-amino-4-(5-((4-(3-chloro-5-fluoropyridin-2-yl)-5-fluoro-6-(isopropylamino)-1H-pyrazolo[3,4-b]pyridin-1-yl)methyl)-1-oxoisoindolin-2-yl)-5-oxopentanoate (0.050 g, 0.08 mmol, 49.7% yield) as a yellow oil. MS (ESI) m/z: 654.2 [M+1]⁺.

D. (3S)-3-(5-((4-(3-chloro-5-fluoropyridin-2-yl)-5-fluoro-6-(isopropylamino)-1H-pyrazolo[3,4-b]pyridin-1-yl)methyl)-1-oxoisoindolin-2-yl) piperidine-2,6-dione. To a solution of tert-butyl (45)-5-amino-4-(5-((4-(3-chloro-5-fluoropyridin-2-yl)-5-fluoro-6-(isopropylamino)-1H-pyrazolo[3,4-b]pyridin-1-yl)methyl)-1-oxoisoindolin-2-yl)-5-oxopentanoate (0.040 g, 0.06 mmol) in acetonitrile (1 mL) was added p-toluenesulfonic acid (0.021 g, 0.12 mmol). The mixture was stirred at 60° C. for 12 h and then stirred at 80° C. for 3 h. The mixture was treated with sodium bicarbonate aqueous solution (8 mL), diluted with water (2 mL), and extracted with ethyl acetate (10 mL×3). The combined organic layers were dried over anhydrous sodium sulfate, filtered and concentrated under reduced pressure. The residue was purified by semi-preparative reverse phase HPLC (43-73% acetonitrile in water+0.225% formic acid, over 7 min). The desired fractions were concentrated under vacuum and then lyophilized to afford the product. The product was separated by SFC separation (Column: DAICEL CHIRALPAK IC (250 mm*30 mm, 10 μm), Mobile phase: Phase A for CO₂, and Phase B for IPA (0.1% NH₃H₂O); Gradient elution: IPA (0.1% NH₃H₂O) in CO₂ from 60% to 60%, Flow rate: 80 mL/min; 7 min, 50 min) to afford two fractions. The earlier eluting fraction was concentrated in vacuum to afford the residue. The residue was diluted with water (10 mL) and extracted with ethyl acetate (10 mL×3). The combined organic layer was dried over anhydrous sodium sulfate, filtered and concentrated under reduced pressure. The residue was re-dissolved in water (10 mL) and acetonitrile (10 mL), then lyophilized to afford the product (3S)-3-(5-((4-(3-chloro-5-fluoropyridin-2-yl)-5-fluoro-6-(isopropylamino)-1H-pyrazolo[3,4-b]pyridin-1-yl)methyl)-1-oxoisoindolin-2-yl) piperidine-2,6-dione (0.013 g, 0.02 mmol, 37.5% yield, 99.2% purity, 100% ee) as an off-white solid. ¹H NMR (400 MHz, DMSO-d₆) δ 10.97 (d, J=2.0 Hz, 1H), 8.81 (d, J=2.4 Hz, 1H), 8.37 (dd, J=2.8, 8.0 Hz, 1H), 7.74-7.68 (m, 1H), 7.52 (s, 2H), 7.44 (d, J=8.0 Hz, 1H), 7.18 (d, J=7.6 Hz, 1H), 5.60 (s, 2H), 5.09 (dd, J=4.8, 13.2 Hz, 1H), 4.45-4.27 (m, 3H), 2.88 (d, J=13.6 Hz, 1H), 2.59 (s, 1H), 2.40-2.37 (m, 1H), 1.99-1.95 (m, 1H), 1.25 (dd, J=2.8, 6.8 Hz, 6H). MS (ESI) m/z: 580.2 [M+1]⁺.

### Example 13:5-chloro-6-(6-(dimethylamino)-1-((2-(2,6-dioxopiperidin-3-yl)-1-oxoisoindolin-5-yl)methyl)-1H-pyrrolo[2,3-b]pyridin-4-yl)nicotinonitrile

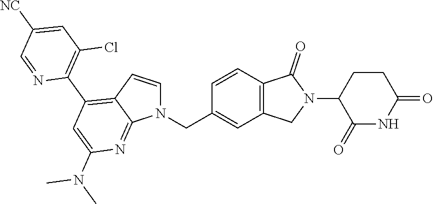 [SMILES: CN(C)c1cc(-c2ncc(C#N)cc2Cl)c2ccn(Cc3ccc4c(c3)CN(C3CCC(=O)NC3=O)C4=O)c2n1]

A. tert-butyl 5-amino-4-[5-[(6-amino-4-bromo-pyrrolo[2,3-b]pyridin-1-yl)methyl]-1-oxo-isoindolin-2-yl]-5-oxo-pentanoate. To a solution of 4-bromo-1H-pyrrolo[2,3-b]pyridin-6-amine (0.420 g, 1.98 mmol) in DMF (4 mL) was added tert-butyl 5-amino-4-[5-(bromomethyl)-1-oxo-isoindolin-2-yl]-5-oxo-pentanoate (Intermediate C)(0.815 g, 1.98 mmol) and cesium carbonate (1.931 g, 5.94 mmol). The mixture was stirred at 40° C. for 2 h. The mixture was purified by semi-preparative reverse phase-HPLC (Phenomenex Synergi Max-RP 250×50 mm×10 μm, 25-55% acetonitrile in water+0.1% TFA, over 22 min). The desired fractions were concentrated to remove most of the acetonitrile, and then lyophilized to afford the product tert-butyl 5-amino-4-[5-[(6-amino-4-bromo-pyrrolo[2,3-b]pyridin-1-yl)methyl]-1-oxo-isoindolin-2-yl]-5-oxo-pentanoate (0.450 g, 0.83 mmol, 41.9% yield) as a white solid. ¹H NMR (400 MHz, DMSO-d₆) δ 7.62 (d, J=7.6 Hz, 1H), 7.51 (s, 1H), 7.35 (s, 1H), 7.25 (d, J=7.6 Hz, 1H), 7.19-7.12 (m, 2H), 6.56 (s, 1H), 6.22 (d, J=3.6 Hz, 1H), 5.39 (s, 2H), 4.70-4.65 (m, 1H), 4.54-4.50 (m, 1H), 4.42-4.37 (s, 1H), 2.17-2.05 (m, 3H), 1.98-1.87 (m, 1H), 1.30 (s, 9H). MS (ESI) m/z: 542.0 [M+1]⁺.

B. tert-butyl 5-amino-4-[5-[[4-bromo-6-(dimethylamino) pyrrolo[2,3-b]pyridin-1-yl]methyl]-1-oxo-isoindolin-2-yl]-5-oxo-pentanoate. To a solution of tert-butyl 5-amino-4-[5-[(6-amino-4-bromo-pyrrolo[2,3-b]pyridin-1-yl)methyl]-1-oxo-isoindolin-2-yl]-5-oxo-pentanoate (0.400 g, 0.74 mmol) in methanol (4 mL) was added formaldehyde (0.12 mL, 2.21 mmol) and acetic acid (0.1 mL). The mixture was stirred at 25° C. for 10 min. Then the mixture was added borane; 2-methylpyridine (0.237 g, 2.21 mmol). The mixture was stirred at 25° C. for 2 h. The mixture was purified by semi-preparative reverse phase-HPLC (Waters Xbridge C18 150×50 mm×10 μm, 46-76% acetonitrile in water+10 mM ammonium bicarbonate, over 11 min). Then the collected fraction was concentrated to remove most of the acetonitrile, and then lyophilized to afford the product tert-butyl 5-amino-4-[5-[[4-bromo-6-(dimethylamino) pyrrolo[2,3-b]pyridin-1-yl]methyl]-1-oxo-isoindolin-2-yl]-5-oxo-pentanoate (0.070 g, 0.12 mmol, 16.6% yield) as a white solid. ¹H NMR (400 MHz, DMSO-d₆) δ 7.63 (d, J=7.6 Hz, 1H), 7.50 (s, 2H), 7.39 (d, J=7.6 Hz, 1H), 7.29 (d, J=3.6 Hz, 1H), 7.14 (s, 1H), 6.71 (s, 1H), 6.23 (d, J=3.6 Hz, 1H), 5.41 (s, 2H), 4.72-4.65 (m, 1H), 4.56-4.49 (m, 1H), 4.44-4.37 (m, 1H), 3.05 (s, 6H), 2.12 (s, 3H), 1.97-1.88 (m, 1H), 1.30 (s, 9H). MS (ESI) m/z: 570.1 [M+1]⁺.

C. tert-butyl 5-amino-4-[5-[[4-(2-chloro-4-cyano-phenyl)-6-(dimethylamino) pyrrolo[2,3-b]pyridin-1-yl]methyl]-1-oxo-isoindolin-2-yl]-5-oxo-pentanoate. To a solution of tert-butyl 5-amino-4-[5-[[4-bromo-6-(dimethylamino) pyrrolo[2,3-b]pyridin-1-yl]methyl]-1-oxo-isoindolin-2-yl]-5-oxo-pentanoate (0.080 g, 0.14 mmol) in 1,4-dioxane (2 mL) was added 3-chloro-4-(4,4,5,5-tetramethyl-1,3,2-dioxaborolan-2-yl)benzonitrile (0.055 g, 0.21 mmol), sodium carbonate (0.21 mL, 0.42 mmol) and tetrakis[triphenylphosphine]palladium (0)(0.012 g, 0.01 mmol). The mixture was stirred at 100° C. under nitrogen for 12 h. The mixture was purified by semi-preparative reverse phase-HPLC (Phenomenex Synergi C18 150×25 mm×10 μm, 60-90% acetonitrile in water+0.1% TFA, over 10 min). Then the collected fraction was concentrated to remove most of the acetonitrile, and then lyophilized to afford the product tert-butyl 5-amino-4-[5-[[4-(2-chloro-4-cyano-phenyl)-6-(dimethylamino) pyrrolo[2,3-b]pyridin-1-yl]methyl]-1-oxo-isoindolin-2-yl]-5-oxo-pentanoate (0.050 g, 0.08 mmol, 56.8% yield) as a yellow solid. MS (ESI) m/z: 627.3 [M+1]⁺.

D. 3-chloro-4-[6-(dimethylamino)-1-[[2-(2,6-dioxo-3-piperidyl)-1-oxo-isoindolin-5-yl]methyl]pyrrolo[2,3-b]pyridin-4-yl]benzonitrile. To a solution of tert-butyl 5-amino-4-[5-[[4-(2-chloro-4-cyano-phenyl)-6-(dimethylamino) pyrrolo[2,3-b]pyridin-1-yl]methyl]-1-oxo-isoindolin-2-yl]-5-oxo-pentanoate (0.050 g, 0.08 mmol) in acetonitrile (2 mL) was added methane sulfonic acid (0.023 g, 0.24 mmol). The mixture was stirred at 80° C. for 12 h. The mixture was purified by semi-preparative reverse phase-HPLC (Phenomenex Luna C18 75×30 mm×3 μm, 48-78% acetonitrile in water+0.1% TFA, over 7 min). Then the collected fraction was concentrated to remove most of the acetonitrile, and then lyophilized to afford the product 3-chloro-4-[6-(dimethylamino)-1-[[2-(2,6-dioxo-3-piperidyl)-1-oxo-isoindolin-5-yl]methyl]pyrrolo[2,3-b]pyridin-4-yl]benzonitrile (24.06 mg, 0.0428 mmol, 54% yield) as a yellow solid. ¹H NMR (400 MHz, DMSO-d₆) δ 10.98 (s, 1H), 8.22 (d, J=1.6 Hz, 1H), 7.96-7.91 (m, 1H), 7.71-7.64 (m, 2H), 7.58 (s, 1H), 7.49 (d, J=8.0 Hz, 1H), 7.27 (d, J=3.6 Hz, 1H), 6.46 (s, 1H), 5.99 (d, J=3.6 Hz, 1H), 5.46 (s, 2H), 5.13-5.05 (m, 1H), 4.45-4.41 (m, 1H), 4.32-4.25 (m, 1H), 3.09 (s, 6H), 2.95-2.85 (m, 1H), 2.61-2.55 (m, 1H), 2.42-2.31 (m, 1H), 2.03-1.90 (m, 1H). MS (ESI) m/z: 553.2 [M+1]⁺.

### Example 14:5-chloro-6-(6-(dimethylamino)-1-((2-(2,6-dioxopiperidin-3-yl)-1-oxoisoindolin-5-yl)methyl)-2,3-dihydro-1H-pyrrolo[2,3-b]pyridin-4-yl)nicotinonitrile

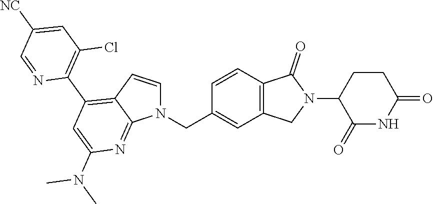 [SMILES: CN(C)c1cc(-c2ncc(C#N)cc2Cl)c2ccn(Cc3ccc4c(c3)CN(C3CCC(=O)NC3=O)C4=O)c2n1]

A. tert-butyl 5-amino-4-[5-[(6-amino-4-bromo-pyrrolo[2,3-b]pyridin-1-yl)methyl]-1-oxo-isoindolin-2-yl]-5-oxo-pentanoate. To a solution of 4-bromo-1H-pyrrolo[2,3-b]pyridin-6-amine (0.240 g, 1.13 mmol) in DMF (4 mL) was added tert-butyl 5-amino-4-[5-(chloromethyl)-1-oxo-isoindolin-2-yl]-5-oxo-pentanoate (prepared analogously to Intermediate C)(0.415 g, 1.13 mmol) and cesium carbonate (1.103 g, 3.40 mmol). The mixture was stirred at 40° C. for 2 h. The mixture was purified by semi-preparative reverse phase-HPLC (Phenomenex luna C18 150×40 mm×15 μm, 31-61% acetonitrile in water+0.1% TFA, over 11 min). The desired fractions were combined, concentrated under vacuum, and then lyophilized to afford the product tert-butyl 5-amino-4-[5-[(6-amino-4-bromo-pyrrolo[2,3-b]pyridin-1-yl)methyl]-1-oxo-isoindolin-2-yl]-5-oxo-pentanoate (0.240 g, 0.44 mmol, 39.1% yield) as a brown solid. MS (ESI) m/z: 542.0 [M+1]⁺.

B. tert-butyl 5-amino-4-[5-[[4-bromo-6-(dimethylamino) pyrrolo[2,3-b]pyridin-1-yl]methyl]-1-oxo-isoindolin-2-yl]-5-oxo-pentanoate. To a solution of tert-butyl 5-amino-4-[5-[(6-amino-4-bromo-pyrrolo[2,3-b]pyridin-1-yl)methyl]-1-oxo-isoindolin-2-yl]-5-oxo-pentanoate (0.230 g, 0.42 mmol) in methanol (3 mL) was added aldehyde (0.07 mL, 1.27 mmol) and acetic acid (0.1 mL). The mixture was stirred at 25° C. for 10 min. Then the mixture was added sodium cyanoborohydride (0.081 g, 1.27 mmol). The mixture was stirred at 25° C. for 2 h. The mixture was purified by semi-preparative reverse phase-HPLC (Waters Xbridge 150×25 mm×5 μm, 50-80% acetonitrile in water+10 mM ammonium bicarbonate, over 9 min). The desired fractions were concentrated under vacuum and lyophilized to afford the product tert-butyl 5-amino-4-[5-[[4-bromo-6-(dimethylamino) pyrrolo[2,3-b]pyridin-1-yl]methyl]-1-oxo-isoindolin-2-yl]-5-oxo-pentanoate (0.040 g, 0.07 mmol, 16.5% yield) as a white solid. MS (ESI) m/z: 570.1 [M+1]⁺.

C. tert-butyl 5-amino-4-[5-[[4-bromo-6-(dimethylamino)-2,3-dihydropyrrolo[2,3-b]pyridin-1-yl]methyl]-1-oxo-isoindolin-2-yl]-5-oxo-pentanoate. To a solution of tert-butyl 5-amino-4-[5-[[4-bromo-6-(dimethylamino) pyrrolo[2,3-b]pyridin-1-yl]methyl]-1-oxo-isoindolin-2-yl]-5-oxo-pentanoate (0.040 g, 0.07 mmol) in acetic acid (1 mL) was added sodium cyanoborohydride (0.022 g, 0.35 mmol). The mixture was stirred at 25° C. for 36 h. The mixture was purified by semi-preparative reverse phase-HPLC (Phenomenex Luna C18 75×30 mm×3 μm, 38-68% acetonitrile in water+0.1% TFA, over 7 min). The desired fractions were concentrated under vacuum and lyophilized to afford tert-butyl 5-amino-4-[5-[[4-bromo-6-(dimethylamino)-2,3-dihydropyrrolo[2,3-b]pyridin-1-yl]methyl]-1-oxo-isoindolin-2-yl]-5-oxo-pentanoate (0.020 g, 0.03 mmol, 49.8% yield) as a yellow solid. MS (ESI) m/z: 572.0 [M+1]⁺.

D. tert-butyl 5-amino-4-[5-[[4-(2-chloro-4-cyano-phenyl)-6-(dimethylamino)-2,3-dihydropyrrolo[2,3-b]pyridin-1-yl]methyl]-1-oxo-isoindolin-2-yl]-5-oxo-pentanoate. To a solution of tert-butyl 5-amino-4-[5-[[4-bromo-6-(dimethylamino)-2,3-dihydropyrrolo[2,3-b]pyridin-1-yl]methyl]-1-oxo-isoindolin-2-yl]-5-oxo-pentanoate (0.020 g, 0.03 mmol) in 1,4-dioxane (1 mL) was added 3-chloro-4-(4,4,5,5-tetramethyl-1,3,2-dioxaborolan-2-yl)benzonitrile (0.014 g, 0.05 mmol), sodium carbonate (0.05 mL, 0.10 mmol) and tetrakis[triphenylphosphine]palladium (0)(0.010 g, 0.01 mmol). The mixture was stirred at 100° C. under nitrogen for 12 h. The mixture was purified by preparative TLC (9.1% methanol in dichloromethane) to afford tert-butyl 5-amino-4-[5-[[4-(2-chloro-4-cyano-phenyl)-6-(dimethylamino)-2,3-dihydropyrrolo[2,3-b]pyridin-1-yl]methyl]-1-oxo-isoindolin-2-yl]-5-oxo-pentanoate (0.020 g, 0.03 mmol, 91.0% yield) as a yellow solid. MS (ESI) m/z: 629.2 [M+1]⁺.

E. 3-chloro-4-[6-(dimethylamino)-1-[[2-(2,6-dioxo-3-piperidyl)-1-oxo-isoindolin-5-yl]methyl]-2,3-dihydropyrrolo[2,3-b]pyridin-4-yl]benzonitrile. To a solution of tert-butyl 5-amino-4-[5-[[4-(2-chloro-4-cyano-phenyl)-6-(dimethylamino)-2,3-dihydropyrrolo[2,3-b]pyridin-1-yl]methyl]-1-oxo-isoindolin-2-yl]-5-oxo-pentanoate (0.020 g, 0.03 mmol) in acetonitrile (1 mL) was added methane sulfonic acid (0.009 g, 0.10 mmol). The mixture was stirred at 80° C. for 12 h. The mixture was purified by semi-preparative reverse phase-HPLC (Phenomenex Synergi C18 150×25 mm×10 μm, 31-61% acetonitrile in water+0.1% TFA, over 10 min). The desired fractions were concentrated under vacuum and then lyophilized to afford the 3-chloro-4-[6-(dimethylamino)-1-[[2-(2,6-dioxo-3-piperidyl)-1-oxo-isoindolin-5-yl]methyl]-2,3-dihydropyrrolo[2,3-b]pyridin-4-yl]benzonitrile (7.7 mg, 0.013 mmol, 41% yield) as a yellow solid. ¹H NMR (400 MHz, DMSO-d₆) δ 10.98 (s, 1H), 8.16 (d, J=1.6 Hz, 1H), 7.90-7.85 (m, 1H), 7.70 (d, J=8.0 Hz, 1H), 7.61-7.54 (m, 2H), 7.49 (d, J=8.0 Hz, 1H), 5.57 (s, 1H), 5.14-5.07 (m, 1H), 4.62 (s, 2H), 4.43 (s, 1H), 4.33-4.29 (m, 1H), 3.37 (t, J=8.0 Hz, 2H), 2.97 (s, 6H), 2.92-2.87 (m, 1H), 2.65-2.61 (m, 3H), 2.44-2.34 (m, 1H), 2.04-1.95 (m, 1H). MS (ESI) m/z: 555.1 [M+1]⁺.

### Example 15:3-(5-((4-(3-chloro-5-(trifluoromethyl)pyridin-2-yl)-6-(3-fluoroazetidin-1-yl)-1H-pyrrolo[2,3-b]pyridin-1-yl)methyl)-1-oxoisoindolin-2-yl) piperidine-2,6-dione

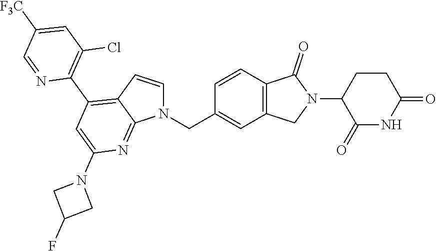 [SMILES: O=C1CCC(N2Cc3cc(Cn4ccc5c(-c6ncc(C(F)(F)F)cc6Cl)cc(N6CC(F)C6)nc54)ccc3C2=O)C(=O)N1]

A. 4-chloro-1H-pyrrolo[2,3-b]pyridine 7-oxide 3-chlorobenzoate. 4-chloro-1H-pyrrolo[2,3-b]pyridine (2.00 g, 13.11 mmol) suspended in a mixture of ethyl acetate (32.8 ml) and hexanes (32.8 ml), then cooled to 0° C. To this suspension was added mCPBA (3.92 g, 17.04 mmol) in portions over about 4 minutes. After complete addition, the reaction was allowed to warm to room temperature and stir for 2 hours. The resulting slurry was filtered through a Buchner funnel and the filter cake rinsed with a 1:1 mixture of ethyl acetate/Hexanes, then rinsed with pure hexanes. The resulting pink solid was collected and dried under vacuum to provide 4-chloro-1H-pyrrolo[2,3-b]pyridine 7-oxide 3-chlorobenzoate (3.32 g, 10.22 mmol, 78% yield) as a pink solid which was not purified further. ¹H NMR (400 MHz, DMSO-d₆) δ 13.29 (br s, 1H), 12.85 (br s, 1H), 8.14 (d, J=6.6 Hz, 1H), 7.95-7.84 (m, 2H), 7.70 (ddd, J=8.1, 2.1, 1.2 Hz, 1H), 7.61-7.50 (m, 2H), 7.21 (d, J=6.6 Hz, 1H), 6.59 (d, J=3.2 Hz, 1H); MS (ESI) m/z: 169.0 [M+1]⁺.

B. 4-chloro-6-(3-fluoroazetidin-1-yl)-1H-pyrrolo[2,3-b]pyridine, 4-chloro-1H-pyrrolo[2,3-b]pyridine 7-oxide 3-chlorobenzoate (1.25 g, 3.84 mmol) was placed in a round bottom flask and placed under nitrogen. Acetonitrile (15.38 ml) was added followed by dimethyl sulfate (0.441 ml, 4.61 mmol). The reaction was sealed and stirred at 60° C. overnight. Upon completion, the reaction was cooled to room temperature and 3-fluoroazetidine hydrochloride (0.858 g, 7.69 mmol) and DIPEA (2.69 ml, 15.38 mmol) were added to the reaction vessel. The reaction was sealed again and heated to 60° C. for 4 hours. The reaction was then cooled to room temperature and the solvent was removed under reduced pressure. The crude material was purified by silica gel chromatography using ethyl acetate and hexanes to provide 4-chloro-6-(3-fluoroazetidin-1-yl)-1H-pyrrolo[2,3-b]pyridine (400 mg, 1.77 mmol, 46% yield). MS (ESI) m/z: 226.0 [M+1]⁺.

C. 4-(3-chloro-5-(trifluoromethyl)pyridin-2-yl)-6-(3-fluoroazetidin-1-yl)-1H-pyrrolo[2,3-b]pyridine, 4-chloro-6-(3-fluoroazetidin-1-yl)-1H-pyrrolo[2,3-b]pyridine (80 mg, 0.355 mmol), potassium acetate (104 mg, 1.064 mmol), bis(pinacolato)diboron (108 mg, 0.425 mmol), XPhos-Pd-G2 (20.89 mg, 0.027 mmol) and XPhos (12.68 mg, 0.027 mmol) were all placed in a vial and placed under nitrogen. 1,4-Dioxane (1.18 mL) was added and the resulting suspension was heated to 80° C. and stirred overnight. After reacting overnight, the reaction was cooled to room temperature and PdCl₂(dppf)(25.9 mg, 0.035 mmol) and 2-bromo-3-chloro-5-(trifluoromethyl)pyridine (102 mg, 0.390 mmol) were added to the reaction and the vial was quickly recapped. potassium carbonate (2.0 M aqueous soln., 0.36 ml, 0.709 mmol) and 1,4-dioxane (0.5 mL) were added via syringe. The reaction was degassed for 5 minutes with nitrogen. The reaction was then heated to 80° C. and stirred for 7 hours. Upon completion, the reaction was cooled to room temperature and diluted with DCM. The mixture was filtered through a pad of celite and the filtrate was concentrated under reduced pressure. The resulting crude residue was purified by silica gel chromatography (SiO₂; ethyl acetate/hexanes gradient) to provide 4-(3-chloro-5-(trifluoromethyl)pyridin-2-yl)-6-(3-fluoroazetidin-1-yl)-1H-pyrrolo[2,3-b]pyridine (120 mg, 0.320 mmol, 90% yield) as an orange solid. MS (ESI) m/z: 371.0 [M+1]⁺.

D. 3-(5-((4-(3-chloro-5-(trifluoromethyl)pyridin-2-yl)-6-(3-fluoroazetidin-1-yl)-1H-pyrrolo[2,3-b]pyridin-1-yl)methyl)-1-oxoisoindolin-2-yl) piperidine-2,6-dione. 4-(3-chloro-5-(trifluoromethyl)pyridin-2-yl)-6-(3-fluoroazetidin-1-yl)-1H-pyrrolo[2,3-b]pyridine (55 mg, 0.148 mmol), tert-butyl(S)-5-amino-4-(5-(bromomethyl)-1-oxoisoindolin-2-yl)-5-oxopentanoate (Intermediate C)(85 mg, 0.208 mmol) and cesium carbonate (193 mg, 0.593 mmol) were placed in an oven-dried vial and placed under nitrogen. DMF (742 μl) was added via syringe and the reaction was heated to 75° C. overnight while stirring. Upon completion, the reaction was cooled to room temperature and diluted with MeCN. The resulting suspension was filtered through a pad of celite and rinsed with MeCN. The filtrate was concentrated under reduced pressure then resuspended in DMF and DMSO and filtered through a micron syringe filter. The resulting filtrate was purified by reverse phase prep HPLC (Column: Xselect CSH C18, 30 mm×100 mm, 5 μm particles; mobile phase B: acetonitrile, mobile phase A: 10 mM aqueous ammonium acetate; Flow Rate: 40.00 mL/min; Column Temperature: 25° C.). The pure fractions were combined and lyophilized to provide 3-(5-((4-(3-chloro-5-(trifluoromethyl)pyridin-2-yl)-6-(3-fluoroazetidin-1-yl)-1H-pyrrolo[2,3-b]pyridin-1-yl)methyl)-1-oxoisoindolin-2-yl) piperidine-2,6-dione (12.8 mg, 0.020 mmol, 14% yield) as a yellow solid. ¹H NMR (400 MHz, DMSO-d₆) δ 10.98 (s, 1H), 9.11-9.07 (m, 1H), 8.66 (d, J=1.3 Hz, 1H), 7.71 (d, J=7.8 Hz, 1H), 7.56 (s, 1H), 7.49 (s, 1H), 7.35 (d, J=3.5 Hz, 1H), 6.44 (s, 1H), 6.16 (d, J=3.4 Hz, 1H), 5.64-5.42 (m, 3H), 5.10 (dd, J=13.2, 5.0 Hz, 1H), 4.49-4.40 (m, 1H), 4.40-4.25 (m, 3H), 4.15-3.98 (m, 2H), 2.97-2.85 (m, 1H), 2.62-2.57 (m, 1H), 2.40-2.33 (m, 1H), 2.03-1.93 (m, 1H). MS (ESI) m/z: 627.0 [M+1]⁺.

### Example 16:3-(5-((4-(3-chloro-5-(trifluoromethyl)pyridin-2-yl)-2-ethyl-6-(3-fluoroazetidin-1-yl)-1H-pyrrolo[2,3-b]pyridin-1-yl)methyl)-1-oxoisoindolin-2-yl) piperidine-2,6-dione

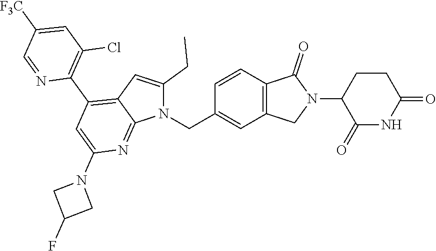 [SMILES: CCc1cc2c(-c3ncc(C(F)(F)F)cc3Cl)cc(N3CC(F)C3)nc2n1Cc1ccc2c(c1)CN(C1CCC(=O)NC1=O)C2=O]

A. 4-bromo-1-(phenylsulfonyl)-1H-pyrrolo[2,3-b]pyridine. To a solution of 4-bromo-1H-pyrrolo[2,3-b]pyridine (5.000 g, 25.38 mmol) in tetrahydrofuran (50 mL) was added sodium hydride (1.218 g, 30.45 mmol, 60% purity) at 0° C. Then the mixture was stirred for 0.5 h. Benzenesulfonyl chloride (5.378 g, 30.45 mmol) was added and the reaction mixture was stirred for 1 h at 25° C. The reaction mixture was quenched by water (50 mL) and extracted with ethyl acetate (20 mL×3). The combined organic layers were concentrated under vacuum. The residue was further purified by silica gel chromatography (0-17% ethyl acetate in petroleum ether) to give the product 4-bromo-1-(phenylsulfonyl)-1H-pyrrolo[2,3-b]pyridine (8.000 g, 23.73 mmol, 93.5% yield) as a white solid. ¹H NMR (400 MHz, DMSO-d₆) δ 8.24 (d, J=5.2 Hz, 1H), 8.15-8.09 (m, 2H), 8.05 (d, J=4.0 Hz, 1H), 7.76-7.70 (m, 1H), 7.67-7.57 (m, 3H), 6.79 (d, J=4.0 Hz, 1H).

B. 4-bromo-2-ethyl-1-(phenylsulfonyl)-1H-pyrrolo[2,3-b]pyridine. To a solution of 4-bromo-1-(phenylsulfonyl)-1H-pyrrolo[2,3-b]pyridine (5.000 g, 14.83 mmol) in tetrahydrofuran (100 mL) was added 2 M (diisopropylamino) lithium (8.9 mL, 17.79 mmol) at −40° C. under nitrogen. Then the mixture was stirred at −40° C. for 1 h. iodoethane (11.564 g, 74.14 mmol) was added and the mixture was stirred at 25° C. for 3 h under nitrogen. The reaction mixture was quenched by water (100 mL) and extracted with ethyl acetate (50 mL×2). The combined organic layers were concentrated under reduced pressure. The residue was purified by semi-preparative reverse phase-HPLC (50-80% acetonitrile in water+0.2251% formic acid, over 10 min). The desired fractions were extracted with ethyl acetate (50 mL×2). The combined organic layers were dried over anhydrous sodium sulfate solid, filtered and concentrated under reduced pressure to give the product 4-bromo-2-ethyl-1-(phenylsulfonyl)-1H-pyrrolo[2,3-b]pyridine (1.880 g, 5.15 mmol, 35% yield) as a light yellow solid. ¹H NMR (400 MHz, DMSO-d₆) δ 8.15 (d, J=5.2 Hz, 1H), 8.11-8.07 (m, 2H), 7.73-7.68 (m, 1H), 7.63-7.57 (m, 2H), 7.53 (d, J=5.2 Hz, 1H), 6.53 (s, 1H), 3.22-3.14 (m, 2H), 1.36 (t, J=7.2 Hz, 3H). MS (ESI) m/z: 367.0 [M+1]⁺.

C. 4-bromo-2-ethyl-1H-pyrrolo[2,3-b]pyridine. A mixture of 4-bromo-2-ethyl-1-(phenylsulfonyl)-1H-pyrrolo[2,3-b]pyridine (0.500 g, 1.37 mmol) and 1 M tetrabutyl ammonium fluoride (2.7 mL, 2.74 mmol) in tetrahydrofuran (20 mL) was stirred at 80° C. for 4 h under nitrogen. The reaction mixture was diluted with water (30 mL) and filtered. The filter cake was concentrated under reduced pressure to give the crude product 4-bromo-2-ethyl-1H-pyrrolo[2,3-b]pyridine (0.320 g, 1.42 mmol) as a yellow solid, which was used in the next step directly. MS (ESI) m/z: 224.9 [M+1]⁺.

D. Bromo-2-ethyl-1H-pyrrolo[2,3-b]pyridine 7-oxide; 3-chlorobenzoic acid. A mixture of 4-bromo-2-ethyl-1H-pyrrolo[2,3-b]pyridine (0.320 g, 1.42 mmol) and 3-chlorobenzeneboronic acid (0.577 g, 2.84 mmol, 85% purity) in ethyl acetate (10 mL) was stirred at 0° C. for 4 h. The reaction mixture was filtered and the filter cake was concentrated under reduced pressure to give the crude product 4-Bromo-2-ethyl-1H-pyrrolo[2,3-b]pyridine 7-oxide; 3-chlorobenzoic acid (0.500 g, 1.26 mmol, 88% yield) as a yellow solid, which was used in the next step directly. MS (ESI) m/z: 241.1 [M+1]⁺.

E. 4-bromo-2-ethyl-6-(3-fluoroazetidin-1-yl)-1H-pyrrolo[2,3-b]pyridine. To a solution of 4-bromo-2-ethyl-1H-pyrrolo[2,3-b]pyridine 7-oxide; 3-chlorobenzoic acid (0.500 g, 1.26 mmol) in acetonitrile (10 mL) was added dimethyl sulfate (0.2 mL, 1.89 mmol) under nitrogen. Then the mixture was stirred at 60° C. for 12 h. The reaction mixture was cooled to 25° C. N,N-diisopropylpropan-2-amine (1.0 mL, 5.03 mmol) and 3-fluoroazetidine; hydrochloride (0.281 g, 2.51 mmol) was added. The mixture was warmed to 60° C. and stirred for another 4 h. The reaction mixture was concentrated under vacuum. The residue was purified by silica gel chromatography (0-30% ethyl acetate in petroleum ether) to give the product 4-bromo-2-ethyl-6-(3-fluoroazetidin-1-yl)-1H-pyrrolo[2,3-b]pyridine (0.070 g, 0.23 mmol, 19% yield) as a light yellow oil. ¹H NMR (400 MHz, DMSO-d₆) δ 11.45 (s, 1H), 6.41 (d, J=1.6 Hz, 1H), 5.92 (s, 1H), 5.62-5.38 (m, 1H), 4.34-4.16 (m, 2H), 4.06-3.88 (m, 2H), 2.66-2.60 (m, 2H), 1.26-1.19 (m, 3H). MS (ESI) m/z: 300.0 [M+1]⁺.

F. 4-(3-chloro-5-(trifluoromethyl)pyridin-2-yl)-2-ethyl-6-(3-fluoroazetidin-1-yl)-1H-pyrrolo[2,3-b]pyridine. A mixture of 4-bromo-2-ethyl-6-(3-fluoroazetidin-1-yl)-1H-pyrrolo[2,3-b]pyridine (0.200 g, 0.67 mmol), 3-chloro-2-(tributylstannyl)-5-(trifluoromethyl)pyridine (Intermediate M)(0.379 g, 0.80 mmol), tetrakis(triphenylphosphine) palladium (0)(0.078 g, 0.07 mmol) and copper iodide (0.025 g, 0.13 mmol) in dioxane (10 mL) was stirred at 100° C. for 12 h under nitrogen. The reaction mixture was quenched by potassium fluoride solution and extracted with ethyl acetate (10 mL×2). The combined organic layers were concentrated under vacuum. The residue was purified by preparative TLC (30% ethyl acetate in petroleum ether) to give 4-(3-chloro-5-(trifluoromethyl)pyridin-2-yl)-2-ethyl-6-(3-fluoroazetidin-1-yl)-1H-pyrrolo[2,3-b]pyridine (0.060 g, 0.15 mmol, 22% yield) as a yellow solid. MS (ESI) m/z: 399.1 [M+1]⁺.

G. tert-butyl(S)-5-amino-4-(5-((4-(3-chloro-5-(trifluoromethyl)pyridin-2-yl)-2-ethyl-6-(3-fluoroazetidin-1-yl)-1H-pyrrolo[2,3-b]pyridin-1-yl)methyl)-1-oxoisoindolin-2-yl)-5-oxopentanoate. A mixture of 4-(3-chloro-5-(trifluoromethyl)pyridin-2-yl)-2-ethyl-6-(3-fluoroazetidin-1-yl)-1H-pyrrolo[2,3-b]pyridine (0.060 g, 0.15 mmol), tert-butyl(S)-5-amino-4-(5-(bromomethyl)-1-oxoisoindolin-2-yl)-5-oxopentanoate (Intermediate C)(0.075 g, 0.18 mmol, and N-ethyl-N-isopropylpropan-2-amine (0.019 g, 0.15 mmol) in acetonitrile (3 mL) was stirred at 80° C. for 6 h. The reaction mixture was concentrated under reduced pressure. The residue was purified by semi-preparative reverse phase-HPLC (25-55% acetonitrile in water+0.225% formic acid, over 7 min). The desired fractions were lyophilized to afford the crude product tert-butyl(S)-5-amino-4-(5-((4-(3-chloro-5-(trifluoromethyl)pyridin-2-yl)-2-ethyl-6-(3-fluoroazetidin-1-yl)-1H-pyrrolo[2,3-b]pyridin-1-yl)methyl)-1-oxoisoindolin-2-yl)-5-oxopentanoate (0.020 g, 0.03 mmol, 18% yield) as a green solid. MS (ESI) m/z: 729.5 [M+1]⁺.

H. 3-(5-((4-(3-chloro-5-(trifluoromethyl)pyridin-2-yl)-2-ethyl-6-(3-fluoroazetidin-1-yl)-1H-pyrrolo[2,3-b]pyridin-1-yl)methyl)-1-oxoisoindolin-2-yl) piperidine-2,6-dione formate. A mixture of tert-butyl(S)-5-amino-4-(5-((4-(3-chloro-5-(trifluoromethyl)pyridin-2-yl)-2-ethyl-6-(3-fluoroazetidin-1-yl)-1H-pyrrolo[2,3-b]pyridin-1-yl)methyl)-1-oxoisoindolin-2-yl)-5-oxopentanoate (0.015 g, 0.02 mmol) and 4-methylbenzenesulfonic acid (0.018 g, 0.10 mmol) in acetonitrile (1.5 mL) was stirred at 80° C. for 2 h. The reaction was diluted with water (20 mL) and adjusted to pH 7 with sodium carbonate solid. The mixture was extracted with ethyl acetate (5 mL×2). The combined organic layers were concentrated under vacuum. The residue was purified by semi-preparative reverse phase-HPLC (22-52% acetonitrile in water+0.225% formic acid, over 7 min). The desired fractions were lyophilized to afford the product 3-(5-((4-(3-chloro-5-(trifluoromethyl)pyridin-2-yl)-2-ethyl-6-(3-fluoroazetidin-1-yl)-1H-pyrrolo[2,3-b]pyridin-1-yl)methyl)-1-oxoisoindolin-2-yl) piperidine-2,6-dione formate (0.002 g, 0.01 mmol, 17% yield, 95.5% purity) as a yellow solid. ¹H NMR (400 MHz, DMSO-d₆) δ 10.97 (s, 1H), 9.04 (s, 1H), 8.58 (s, 1H), 8.22 (s, 1H), 7.71 (d, J=8.0 Hz, 1H), 7.55 (s, 1H), 7.47 (d, J=7.6 Hz, 1H), 6.43-6.29 (m, 1H), 5.89-5.80 (m, 1H), 5.80-5.65 (m, 1H), 5.36-5.22 (m, 1H), 5.16-5.06 (m, 1H), 5.05-4.95 (m, 1H), 4.93-4.84 (m, 1H), 4.58-4.45 (m, 1H), 4.41 (d, J=17.6 Hz, 1H), 4.34-4.23 (m, 1H), 4.12-3.77 (m, 2H), 2.98-2.82 (m, 1H), 2.70-2.64 (m, 2H), 2.62-2.55 (m, 1H), 2.40-2.34 (m, 1H), 2.04-1.94 (m, 1H), 1.19 (t, J=7.6 Hz, 3H). MS (ESI) m/z: 655.1 [M+1]⁺.

### Example 17:3-(5-((4-(3-chloro-5-fluoropyridin-2-yl)-6-(3-fluoroazetidin-1-yl)-1H-pyrrolo[2,3-b]pyridin-1-yl)methyl)-1-oxoisoindolin-2-yl) piperidine-2,6-dione

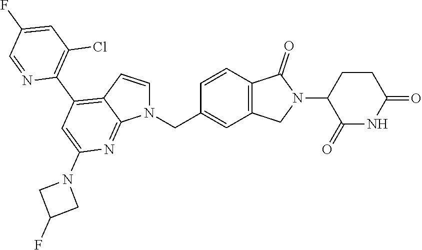 [SMILES: O=C1CCC(N2Cc3cc(Cn4ccc5c(-c6ncc(F)cc6Cl)cc(N6CC(F)C6)nc54)ccc3C2=O)C(=O)N1]

A. 4-(3-chloro-5-fluoropyridin-2-yl)-6-(3-fluoroazetidin-1-yl)-1H-pyrrolo[2,3-b]pyridine, 4-chloro-6-(3-fluoroazetidin-1-yl)-1H-pyrrolo[2,3-b]pyridine (250 mg, 1.108 mmol), potassium acetate (326 mg, 3.32 mmol), B2pin2 (338 mg, 1.329 mmol), XPhos-Pd-G2 (65.3 mg, 0.083 mmol) and XPhos (39.6 mg, 0.083 mmol) were all placed in a vial and placed under nitrogen. 1,4-Dioxane (3.69 mL) was added and the resulting suspension was heated to 80° C. and stirred overnight. After reacting overnight, the reaction was cooled to room temperature and PdCl₂(dppf)(81 mg, 0.111 mmol) and 2-bromo-3-chloro-5-fluoropyridine (256 mg, 1.219 mmol) were added to the reaction and the vial was quickly recapped. potassium carbonate (2.0 M aqueous soln., 0.55 mL, 1.108 mmol) and 1,4-dioxane (0.5 mL) were added via syringe. The reaction was degassed for 5 minutes with nitrogen. The reaction was then heated to 80° C. and stirred for 7 hours. Upon completion the reaction was cooled to room temperature and diluted with DCM. The mixture was filtered through a pad of celite and the filtrate was concentrated under reduced pressure. The resulting crude residue was purified by silica gel chromatography (SiO₂; ethyl acetate/hexanes gradient) to provide 4-(3-chloro-5-fluoropyridin-2-yl)-6-(3-fluoroazetidin-1-yl)-1H-pyrrolo[2,3-b]pyridine (223 mg, 0.696 mmol, 63% yield) as an orange solid. MS (ESI) m/z: 321.0 [M+1]⁺.

B. 3-(5-((4-(3-chloro-5-fluoropyridin-2-yl)-6-(3-fluoroazetidin-1-yl)-1H-pyrrolo[2,3-b]pyridin-1-yl)methyl)-1-oxoisoindolin-2-yl) piperidine-2,6-dione. 4-(3-chloro-5-fluoropyridin-2-yl)-6-(3-fluoroazetidin-1-yl)-1H-pyrrolo[2,3-b]pyridine (68 mg, 0.212 mmol), tert-butyl(S)-5-amino-4-(5-(bromomethyl)-1-oxoisoindolin-2-yl)-5-oxopentanoate (Intermediate C)(122 mg, 0.297 mmol) and cesium carbonate (276 mg, 0.848 mmol) were placed in an oven-dried vial and placed under nitrogen. DMF (1.06 mL) was added via syringe and the reaction was heated to 75° C. overnight while stirring. Upon completion, the reaction was cooled to room temperature and diluted with MeCN. The resulting suspension was filtered through a pad of celite and rinsed with MeCN. The filtrate was concentrated under reduced pressure then resuspended in DMF and DMSO and filtered through a micron syringe filter. The resulting filtrate was purified by prep reverse phase HPLC (Column: Xselect CSH C18, 30 mm×100 mm, 5 μm particles; Eluent: water/MeCN w/0.1% TFA; Flow Rate: 40.00 mL/min; Column Temperature: 25° C.). The pure fractions were combined and lyophilized to provide 3-(5-((4-(3-chloro-5-fluoropyridin-2-yl)-6-(3-fluoroazetidin-1-yl)-1H-pyrrolo[2,3-b]pyridin-1-yl)methyl)-1-oxoisoindolin-2-yl) piperidine-2,6-dione (12.8 mg, 0.020 mmol, 14% yield) as a yellow solid. 1H NMR (500 MHz, DMSO-d₆) δ ppm 1.91-2.03 (m, 1H) 2.32-2.41 (m, 2H) 2.57-2.62 (m, 1H) 2.83-2.96 (m, 1H) 4.00-4.12 (m, 2H) 4.25-4.47 (m, 4H) 5.04-5.14 (m, 1H) 5.42-5.61 (m, 3H) 6.12 (d, J=3.46 Hz, 1H) 6.38 (s, 1H) 7.29-7.35 (m, 1H) 7.47 (d, J=7.87 Hz, 1H) 7.55 (s, 1H) 7.67-7.73 (m, 1H) 8.26-8.32 (m, 1H) 8.73-8.76 (m, 1H) 10.97 (s, 1H). MS (ESI) m/z: 577.7 [M+1]⁺.

### Example 19:3-(5-((7-(2-chloro-4-(trifluoromethyl)phenyl)-5-(3-fluoroazetidin-1-yl)-3H-imidazo[4,5-b]pyridin-3-yl)methyl)-1-oxoisoindolin-2-yl) piperidine-2,6-dione

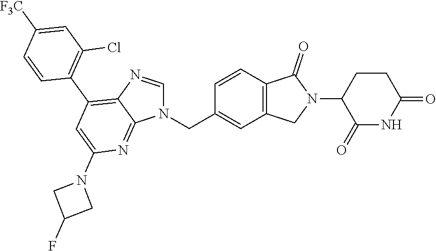 [SMILES: O=C1CCC(N2Cc3cc(Cn4cnc5c(-c6ccc(C(F)(F)F)cc6Cl)cc(N6CC(F)C6)nc54)ccc3C2=O)C(=O)N1]

A. 2,6-difluoro-4-iodo-3-nitropyridine. To a solution of 2,6-difluoro-4-iodo-pyridine (10.000 g, 41.50 mmol) in sulfuric acid (30.0 mL, 562.81 mmol) was added nitric acid (11.4 mL, 268.21 mmol) at 0° C. Then the mixture was stirred at 25° C. for 12 h under nitrogen. The reaction mixture was added to ice water (500 mL) slowly. Then the mixture was adjusted to pH 7 with sodium carbonate solid and extracted with ethyl acetate (200 mL×2). The combined organic layers were concentrated under vacuum. The residue was purified by silica gel chromatography (0-1% ethyl acetate in petroleum ether) to give the product 2,6-difluoro-4-iodo-3-nitropyridine (8.444 g, 29.53 mmol, 71% yield) as a brown solid. ¹H NMR (400 MHz, CDCl₃) δ 7.45 (d, J=3.2 Hz, 1H).

B. 6-fluoro-4-iodo-3-nitropyridin-2-amine. To a solution of 2,6-difluoro-4-iodo-3-nitropyridine (8.444 g, 29.53 mmol) in tetrahydrofuran (100 mL) was added ammonium hydroxide (5.5 mL, 35.43 mmol) slowly at 0° C. Then the mixture was stirred at 0° C. for 2 h. The reaction mixture was adjusted pH to 5 with 1 M phosphoric acid and extracted with ethyl acetate (30 mL×2). The combined organic layers were concentrated under vacuum. The residue was purified by silica gel chromatography (0-30% ethyl acetate in petroleum ether) to give the product 6-fluoro-4-iodo-3-nitropyridin-2-amine (7.330 g, 25.90 mmol, 88% yield) as a yellow solid. ¹H NMR (400 MHz, DMSO-d₆) δ 7.56 (s, 2H), 6.95 (dd, J=1.2, 2.8 Hz, 1H).

C. 6-fluoro-4-iodopyridine-2,3-diamine. The mixture of 6-fluoro-4-iodo-3-nitropyridin-2-amine (7.330 g, 25.90 mmol), iron (7.232 g, 129.51 mmol) and ammonium chloride (6.927 g, 129.51 mmol) in ethanol (100 mL) and water (20 mL) was stirred at 50° C. for 1 h. The reaction mixture was filtered and the filtrate was concentrated under vacuum. The residue was purified by silica gel chromatography (0-35% ethyl acetate in petroleum ether) to give the product 6-fluoro-4-iodopyridine-2,3-diamine (4.100 g, 16.20 mmol, 63% yield) as a brown solid. ¹H NMR (400 MHz, DMSO-d₆) δ 13.07-12.44 (m, 1H), 8.07 (s, 1H), 6.79 (s, 1H), 5.61-5.33 (m, 1H), 4.36-4.22 (m, 2H), 4.08-3.95 (m, 2H).

D. 5-fluoro-7-iodo-3H-imidazo[4,5-b]pyridine. The mixture of 6-fluoro-4-iodopyridine-2,3-diamine (4.100 g, 16.20 mmol) in formic acid (12.0 mL, 318.05 mmol) was stirred at 80° C. for 12 h. The reaction mixture was diluted with water (50 mL) and adjusted to 7 with sodium carbonate solid. Then the mixture was filtered and the filter cake was dried under vacuum to give the product 5-fluoro-7-iodo-3H-imidazo[4,5-b]pyridine (3.700 g, 14.068 mmol, 86.816% yield) as an off-white solid. The filtrate was extracted with ethyl acetate (30 mL×2). The combined organic layers were dried over anhydrous sodium sulfate solid, filtered and concentrated under reduced pressure to give additional desired product 5-fluoro-7-iodo-3H-imidazo[4,5-b]pyridine (0.900 g, 3.42 mmol, 21% yield) as a brown solid. ¹H NMR (400 MHz, DMSO-d₆) δ 13.99-12.63 (m, 1H), 8.48 (s, 1H), 7.52 (d, J=1.2 Hz, 1H). MS (ESI) m/z: 263.9 [M+1]⁺.

E. 5-(3-fluoroazetidin-1-yl)-7-iodo-3H-imidazo[4,5-b]pyridine. The mixture of 5-fluoro-7-iodo-3H-imidazo[4,5-b]pyridine (1.500 g, 5.70 mmol), 3-fluoroazetidine hydrochloride (0.763 g, 6.84 mmol) and N-ethyl-N-isopropylpropan-2-amine (2.0 mL, 11.41 mmol) in acetonitrile (15 mL) was stirred at 100° C. for 3 d. The reaction was divided into three batches. The reaction mixture was concentrated under reduced pressure. The residue was purified by semi-preparative reverse phase-HPLC (5-35% acetonitrile in water+0.225% formic acid, over 15 min). Then the collection fraction was extracted with ethyl acetate (50 mL×2). The combined organic layers were dried over anhydrous sodium sulfate solid, filtered and concentrated under reduced pressure to give the crude product. The crude product was further purified by semi-preparative reverse phase-HPLC (16-46% acetonitrile in water+10 mM ammonium bicarbonate, over 9 min). The desired fractions were lyophilized to afford the product 5-(3-fluoroazetidin-1-yl)-7-iodo-3H-imidazo[4,5-b]pyridine (0.070 g, 0.22 mmol, 4% yield) as a white solid. ¹H NMR (400 MHz, DMSO-d₆) δ 13.07-12.44 (m, 1H), 8.07 (s, 1H), 6.79 (s, 1H), 5.61-5.33 (m, 1H), 4.36-4.22 (m, 2H), 4.08-3.95 (m, 2H). MS (ESI) m/z: 319.0 [M+1]⁺.

F. tert-butyl(S)-5-amino-4-(5-((5-(3-fluoroazetidin-1-yl)-7-iodo-3H-imidazo[4,5-b]pyridin-3-yl)methyl)-1-oxoisoindolin-2-yl)-5-oxopentanoate. A mixture of 5-(3-fluoroazetidin-1-yl)-7-iodo-3H-imidazo[4,5-b]pyridine (0.070 g, 0.22 mmol), tert-butyl(S)-5-amino-4-(5-(chloromethyl)-1-oxoisoindolin-2-yl)-5-oxopentanoate (prepared analogously to Intermediate C)(0.097 g, 0.26 mmol) and cesium carbonate (0.143 g, 0.44 mmol) in DMF (2 mL) was stirred at 50° C. for 2 h. The reaction mixture was diluted with water (20 mL) and extracted with ethyl acetate (10 mL×2). The combined organic layers were concentrated under reduced pressure. The residue was purified by preparative-TLC (80% ethyl acetate in petroleum ether) to give the product tert-butyl(S)-5-amino-4-(5-((5-(3-fluoroazetidin-1-yl)-7-iodo-3H-imidazo[4,5-b]pyridin-3-yl)methyl)-1-oxoisoindolin-2-yl)-5-oxopentanoate (0.054 g, 0.08 mmol, 38% yield) as a white solid. ¹H NMR (400 MHz, CDCl₃) δ 7.85-7.79 (m, 2H), 7.46-7.40 (m, 1H), 7.37-7.30 (m, 1H), 6.75 (s, 1H), 6.37-6.21 (m, 1H), 5.56-5.35 (m, 2H), 5.31 (s, 1H), 4.89 (dd, J=6.4, 8.8 Hz, 1H), 4.55-4.47 (m, 1H), 4.40 (d, J=17.6 Hz, 1H), 4.37-4.28 (m, 2H), 4.22-4.10 (m, 2H), 2.43-2.07 (m, 4H), 1.41 (s, 9H). MS (ESI) m/z: 649.2 [M+1]⁺.

G. tert-butyl(S)-5-amino-4-(5-((7-(2-chloro-4-(trifluoromethyl)phenyl)-5-(3-fluoroazetidin-1-yl)-3H-imidazo[4,5-b]pyridin-3-yl)methyl)-1-oxoisoindolin-2-yl)-5-oxopentanoate. The mixture of tert-butyl(S)-5-amino-4-(5-((5-(3-fluoroazetidin-1-yl)-7-iodo-3H-imidazo[4,5-b]pyridin-3-yl)methyl)-1-oxoisoindolin-2-yl)-5-oxopentanoate (0.050 g, 0.08 mmol), (2-chloro-4-(trifluoromethyl)phenyl) boronic acid (0.021 g, 0.09 mmol), tetrakis(triphenylphosphine) palladium (0)(0.009 g, 0.01 mmol) and 2 M potassium carbonate (0.1 mL, 0.15 mmol) in dioxane (3 mL) was stirred at 100° C. for 12 h under nitrogen. The reaction mixture was diluted with water (20 mL) and extracted with ethyl acetate (20 mL×2). The combined organic layers were concentrated under reduced pressure. The residue was purified by preparative-TLC (60% ethyl acetate in petroleum ether) to give the product tert-butyl(S)-5-amino-4-(5-((7-(2-chloro-4-(trifluoromethyl)phenyl)-5-(3-fluoroazetidin-1-yl)-3H-imidazo[4,5-b]pyridin-3-yl)methyl)-1-oxoisoindolin-2-yl)-5-oxopentanoate (0.020 g, 0.03 mmol, 37% yield) as a white solid. MS (ESI) m/z: 701.1 [M+1]⁺.

H. 3-(5-((7-(2-chloro-4-(trifluoromethyl)phenyl)-5-(3-fluoroazetidin-1-yl)-3H-imidazo[4,5-b]pyridin-3-yl)methyl)-1-oxoisoindolin-2-yl) piperidine-2,6-dione. To a solution of tert-butyl(S)-5-amino-4-(5-((7-(2-chloro-4-(trifluoromethyl)phenyl)-5-(3-fluoroazetidin-1-yl)-3H-imidazo[4,5-b]pyridin-3-yl)methyl)-1-oxoisoindolin-2-yl)-5-oxopentanoate (0.020 g, 0.03 mmol) in dichloromethane (1 mL) was added TFA (0.3 mL, 4.04 mmol) at 25° C. Then the mixture was stirred at 25° C. for 1 h. The reaction mixture was concentrated under reduced pressure to give the product(S)-5-amino-4-(5-((7-(2-chloro-4-(trifluoromethyl)phenyl)-5-(3-fluoroazetidin-1-yl)-3H-imidazo[4,5-b]pyridin-3-yl)methyl)-1-oxoisoindolin-2-yl)-5-oxopentanoic acid (0.018 g, 0.03 mmol, 97.8% yield) as a yellow oil, which was used in the next step directly. A mixture of(S)-5-amino-4-(5-((7-(2-chloro-4-(trifluoromethyl)phenyl)-5-(3-fluoroazetidin-1-yl)-3H-imidazo[4,5-b]pyridin-3-yl)methyl)-1-oxoisoindolin-2-yl)-5-oxopentanoic acid (0.018 g, 0.03 mmol), 4-dimethylaminopyridine (0.003 g, 0.03 mmol), di(1H-imidazol-1-yl) methanone (0.009 g, 0.06 mmol) and N-ethyl-N-isopropylpropan-2-amine (0.1 mL, 0.08 mmol) in acetonitrile (1 mL) was stirred at 80° C. for 2 h. The reaction mixture was purified by semi-preparative reverse phase-HPLC (42-72% acetonitrile in water+0.225% formic acid, over 7 min). The desired fractions were lyophilized to afford the product 3-(5-((7-(2-chloro-4-(trifluoromethyl)phenyl)-5-(3-fluoroazetidin-1-yl)-3H-imidazo[4,5-b]pyridin-3-yl)methyl)-1-oxoisoindolin-2-yl) piperidine-2,6-dione (0.004 g, 0.01 mmol, 23% yield, 98% purity) as a yellow solid. ¹H NMR (400 MHz, DMSO-d₆) δ 10.98 (s, 1H), 8.27 (s, 1H), 8.03 (s, 1H), 7.89-7.81 (m, 1H), 7.79-7.69 (m, 2H), 7.68-7.64 (m, 1H), 7.62-7.54 (m, 1H), 6.42 (s, 1H), 5.64-5.42 (m, 3H), 5.13-5.06 (m, 1H), 4.49-4.42 (m, 1H), 4.39-4.29 (m, 3H), 4.15-4.03 (m, 3H), 2.89 (dd, J=2.4, 5.6 Hz, 1H), 2.58 (s, 1H), 2.40 (d, J=1.6 Hz, 1H), 2.03-1.93 (m, 1H). MS (ESI) m/z: 627.2 [M+1]⁺.

### Example 21:3-(5-((7-(3-chloro-5-(trifluoromethyl)pyridin-2-yl)-5-(3-fluoroazetidin-1-yl)-2-methyl-3H-imidazo[4,5-b]pyridin-3-yl)methyl)-1-oxoisoindolin-2-yl) piperidine-2,6-dione

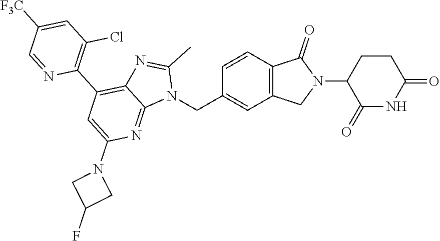 [SMILES: Cc1nc2c(-c3ncc(C(F)(F)F)cc3Cl)cc(N3CC(F)C3)nc2n1Cc1ccc2c(c1)CN(C1CCC(=O)NC1=O)C2=O]

A. 2,6-difluoro-4-iodo-3-nitropyridine. To a solution of 2,6-difluoro-4-iodopyridine (20.000 g, 82.99 mmol) in sulfuric acid (60. mL, 1126.5 mmol) was added nitric acid (20 mL, 479.37 mmol) at 0° C. The mixture was stirred at 25° C. for 12 h. The reaction mixture was added to water (500 mL) slowly. The mixture was extracted with ethyl acetate (100 mL×2). The combined organic layers were dried over anhydrous sodium sulfate solid, filtered and concentrated under vacuum to give 2,6-difluoro-4-iodo-3-nitropyridine (17.000 g, 59.45 mmol, 72% yield) as a yellow solid. ¹H NMR (400 MHz, CDCl₃) δ 7.45 (d, J=2.8 Hz, 1H).

B. 6-fluoro-4-iodo-3-nitropyridin-2-amine. To a solution of 2,6-difluoro-4-iodo-3-nitropyridine (12.000 g, 41.96 mmol) in tetrahydrofuran (70 mL) was added ammonium hydroxide (9.70 mL, 62.94 mmol) at 0° C. The mixture was stirred at 0° C. for 2 h. The reaction mixture was adjusted to pH 7 with 1 M phosphoric acid and extracted with ethyl acetate (100 mL×2). The combined organic layers were dried over anhydrous sodium sulfate solid, filtered and concentrated under reduced pressure to give the crude product 6-fluoro-4-iodo-3-nitropyridin-2-amine (9.000 g, 31.80 mmol, 76% yield) as a yellow solid, which was used into next step directly. MS (ESI) m/z: 283.9 [M+1]⁺.

C. 6-(3-fluoroazetidin-1-yl)-4-iodo-3-nitropyridin-2-amine. To a solution of 6-fluoro-4-iodo-3-nitropyridin-2-amine (12.000 g, 42.40 mmol) in acetonitrile (200 mL) was added 3-fluoroazetidine hydrochloride (5.680 g, 50.88 mmol) and N-ethyl-N-isopropylpropan-2-amine (14.74 mL, 84.81 mmol). The mixture was stirred at 50° C. for 1 h. The mixture was diluted with water (20 mL), extracted with ethyl acetate (20 mL×3). The combined organic layer was dried over anhydrous sodium sulfate, filtered and concentrated under reduced pressure. The residue was triturated with ethyl acetate (20 mL). The resultant precipitate solid was collected by filtration and dried in vacuo to afford 6-(3-fluoroazetidin-1-yl)-4-iodo-3-nitropyridin-2-amine (11.000 g, 32.54 mmol, 77% yield) as a yellow solid. ¹H NMR (400 MHz, DMSO-d₆) δ 6.73 (s, 2H), 5.54 (s, 1H), 4.64-4.46 (m, 1H), 3.51-3.42 (m, 2H), 3.24-3.15 (m, 2H). MS (ESI) m/z: 339.1 [M+1]⁺.

D. 6-(3-fluoroazetidin-1-yl)-4-iodopyridine-2,3-diamine. To a solution of 6-(3-fluoroazetidin-1-yl)-4-iodo-3-nitropyridin-2-amine (11.000 g, 32.54 mmol) in ethanol (15 mL) and water (5 mL) was added ferrum (14.540 g, 260.29 mmol) and ammonium chloride (17.240 g, 325.37 mmol). The mixture was stirred at 50° C. for 1 h. The mixture was filtered and concentrated. It was purified by silica gel chromatography (0˜80% of ethyl acetate in petroleum ether) to afford 6-(3-fluoroazetidin-1-yl)-4-iodopyridine-2,3-diamine (7.200 g, 23.37 mmol, 72% yield) as a brown solid. ¹H NMR (400 MHz, DMSO-d₆) δ 5.94 (s, 1H), 5.65 (s, 2H), 5.48-5.29 (m, 1H), 4.07-3.98 (m, 4H), 3.79-3.70 (m, 2H).

E. 5-(3-fluoroazetidin-1-yl)-7-iodo-2-methyl-3H-imidazo[4,5-b]pyridine. The mixture of 6-(3-fluoroazetidin-1-yl)-4-iodopyridine-2,3-diamine (4.000 g, 12.98 mmol), ethyl acetamidate hydrochloride (4.810 g, 38.95 mmol) and 3A MS (1.000 g, 25.97 mmol) in ethanol (160 mL) was stirred at 100° C. for 12 h under nitrogen. The reaction mixture was filtered and the filtrate was concentrated under vacuum. The residue was purified by silica gel chromatography (0-96% ethyl acetate in petroleum ether) to give 5-(3-fluoroazetidin-1-yl)-7-iodo-2-methyl-3H-imidazo[4,5-b]pyridine (3.300 g, 9.94 mmol, 77% yield) as a yellow solid. 1H NMR (400 MHz, CDCl₃) δ 10.29-9.88 (m, 1H), 6.64 (s, 1H), 5.53-5.35 (m, 1H), 4.36-4.27 (m, 2H), 4.18-4.08 (m, 2H), 2.60 (s, 3H). MS (ESI) m/z: 333.0 [M+1]⁺.

F. tert-butyl(S)-5-amino-4-(5-((5-(3-fluoroazetidin-1-yl)-7-iodo-2-methyl-3H-imidazo[4,5-b]pyridin-3-yl)methyl)-1-oxoisoindolin-2-yl)-5-oxopentanoate. To a solution of 5-(3-fluoroazetidin-1-yl)-7-iodo-2-methyl-3H-imidazo[4,5-b]pyridine (1.000 g, 3.01 mmol) in acetonitrile (20 mL) was added tert-butyl(S)-5-amino-4-(5-(bromomethyl)-1-oxoisoindolin-2-yl)-5-oxopentanoate (Intermediate C)(1.490 g, 3.61 mmol) and N-ethyl-N-isopropylpropan-2-amine (1.05 mL, 6.02 mmol). The mixture was stirred at 70° C. for 12 h. The mixture was divided into two batches. The mixture was concentrated under vacuum and the residue was purified by semi-preparative reverse phase HPLC (15-45% acetonitrile in water+0.225% formic acid, over 15 min). The desired fractions were concentrated under vacuum and lyophilized to afford the product tert-butyl(S)-5-amino-4-(5-((5-(3-fluoroazetidin-1-yl)-7-iodo-2-methyl-3H-imidazo[4,5-b]pyridin-3-yl)methyl)-1-oxoisoindolin-2-yl)-5-oxopentanoate (0.500 g, 0.75 mmol, 25% yield) as a yellow solid. ¹H NMR (400 MHz, DMSO-d₆) δ 7.67-7.64 (m, 1H), 7.54 (s, 1H), 7.41 (s, 1H), 7.33 (d, J=8.4 Hz, 1H), 7.18 (s, 1H), 6.77 (s, 1H), 5.55-5.42 (m, 3H), 4.71-4.69 (m, 1H), 4.56-4.52 (m, 1H), 4.43-4.24 (m, 3H), 4.07-4.00 (m, 2H), 2.43 (s, 3H), 2.13 (s, 3H), 1.94 (d, J=10.4 Hz, 1H), 1.29 (s, 9H). MS (ESI) m/z: 663.2 [M+1]⁺.

G. tert-butyl(S)-5-amino-4-(5-((7-(3-chloro-5-(trifluoromethyl)pyridin-2-yl)-5-(3-fluoroazetidin-1-yl)-2-methyl-3H-imidazo[4,5-b]pyridin-3-yl)methyl)-1-oxoisoindolin-2-yl)-5-oxopentanoate. To a solution of tert-butyl(S)-5-amino-4-(5-((5-(3-fluoroazetidin-1-yl)-7-iodo-2-methyl-3H-imidazo[4,5-b]pyridin-3-yl)methyl)-1-oxoisoindolin-2-yl)-5-oxopentanoate (0.110 g, 0.17 mmol) in N,N-dimethylacetamide (0.8 mL) was added 3-chloro-2-(tributylstannyl)-5-(trifluoromethyl)pyridine (Intermediate M)(0.391 g, 0.83 mmol) and methanesulfonato (2-dicyclohexylphosphino-3,6-dimethoxy-2′,4′,6′-tri-i-propyl-1, l′-biphenyl)(2′-methylamino-1,1′-biphenyl-2-yl) palladium (II)(0.013 g, 0.02 mmol). The mixture was stirred at 90° C. for 12 h under nitrogen. The mixture was divided into eleven batches. The mixture was quenched with saturated potassium fluoride aqueous solution (10 mL) and diluted with water (5 mL), extracted with ethyl acetate (10 mL×3). The combined organic layers were dried over anhydrous sodium sulfate, filtered and concentrated under vacuum. The residue was purified by semi-preparative reverse phase HPLC (28-58% acetonitrile in water+0.225% formic acid, over 10 min). The desired fractions were lyophilized to afford the product. The residue was re-purified by semi-preparative reverse phase HPLC (42-72% acetonitrile in water+0.225% formic acid, over 9 min). The desired fractions were concentrated under vacuum and lyophilized to afford the product tert-butyl(S)-5-amino-4-(5-((7-(3-chloro-5-(trifluoromethyl)pyridin-2-yl)-5-(3-fluoroazetidin-1-yl)-2-methyl-3H-imidazo[4,5-b]pyridin-3-yl)methyl)-1-oxoisoindolin-2-yl)-5-oxopentanoate (0.015 g, 0.02 mmol, 13% yield) as a yellow oil. MS (ESI) m/z: 716.1 [M+1]⁺.

H. (S)-5-amino-4-(5-((7-(3-chloro-5-(trifluoromethyl)pyridin-2-yl)-5-(3-fluoroazetidin-1-yl)-2-methyl-3H-imidazo[4,5-b]pyridin-3-yl)methyl)-1-oxoisoindolin-2-yl)-5-oxopentanoic acid. To a solution of tert-butyl(S)-5-amino-4-(5-((7-(3-chloro-5-(trifluoromethyl)pyridin-2-yl)-5-(3-fluoroazetidin-1-yl)-2-methyl-3H-imidazo[4,5-b]pyridin-3-yl)methyl)-1-oxoisoindolin-2-yl)-5-oxopentanoate (0.015 g, 0.02 mmol) in dichloromethane (0.60 mL) was added TFA (0.3 mL, 0 mmol). The mixture was stirred at 25° C. for 1 h. The mixture was concentrated under vacuum to afford(S)-5-amino-4-(5-((7-(3-chloro-5-(trifluoromethyl)pyridin-2-yl)-5-(3-fluoroazetidin-1-yl)-2-methyl-3H-imidazo[4,5-b]pyridin-3-yl)methyl)-1-oxoisoindolin-2-yl)-5-oxopentanoic acid (0.013 g, 0.02 mmol, 94% yield) as a yellow oil. MS (ESI) m/z: 660.3 [M+1]⁺.

I. 3-(5-((7-(3-chloro-5-(trifluoromethyl)pyridin-2-yl)-5-(3-fluoroazetidin-1-yl)-2-methyl-3H-imidazo[4,5-b]pyridin-3-yl)methyl)-1-oxoisoindolin-2-yl) piperidine-2,6-dione. To a solution of(S)-5-amino-4-(5-((7-(3-chloro-5-(trifluoromethyl)pyridin-2-yl)-5-(3-fluoroazetidin-1-yl)-2-methyl-3H-imidazo[4,5-b]pyridin-3-yl)methyl)-1-oxoisoindolin-2-yl)-5-oxopentanoic acid (0.013 g, 0.02 mmol) in acetonitrile (1 mL) was added di(1H-imidazol-1-yl) methanone (0.006 g, 0.04 mmol) and dimethylaminopyridine (0.005 g, 0.04 mmol). The mixture was stirred at 80° C. for 3 h under nitrogen. The mixture was concentrated under vacuum and the residue was purified by semi-preparative reverse phase HPLC (18-48% acetonitrile in water+0.225% formic acid, over 10 min). The desired fractions were concentrated under vacuum and lyophilized to afford 3-(5-((7-(3-chloro-5-(trifluoromethyl)pyridin-2-yl)-5-(3-fluoroazetidin-1-yl)-2-methyl-3H-imidazo[4,5-b]pyridin-3-yl)methyl)-1-oxoisoindolin-2-yl) piperidine-2,6-dione (0.005 g, 0.008 mmol, 41% yield) as a yellow solid. ¹H NMR (400 MHz, DMSO-d₆) δ 10.97 (s, 1H), 9.07 (d, J=0.8 Hz, 1H), 8.64 (d, J=1.2 Hz, 1H), 7.73 (d, J=7.6 Hz, 1H), 7.48 (s, 1H), 7.43 (d, J=8.0 Hz, 1H), 6.42 (s, 1H), 5.59-5.44 (m, 3H), 5.09 (dd, J=5.2, 13.2 Hz, 1H), 4.47-4.29 (m, 4H), 4.11-4.03 (m, 2H), 2.94-2.85 (m, 1H), 2.61 (s, 1H), 2.41 (s, 3H), 2.35 (dd, J=4.8, 13.6 Hz, 1H), 2.02-1.94 (m, 1H). MS (ESI) m/z: 642.3 [M+1]⁺.

### Example 26:3-(5-((4-(3-chloro-5-(trifluoromethyl)pyridin-2-yl)-6-(3-fluoroazetidin-1-yl)-1H-indazol-1-yl)methyl)-1-oxoisoindolin-2-yl) piperidine-2,6-dione

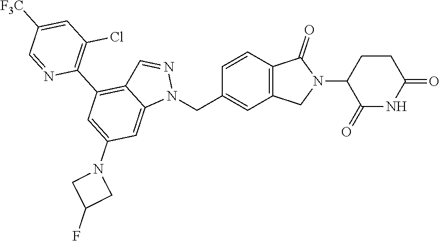 [SMILES: O=C1CCC(N2Cc3cc(Cn4ncc5c(-c6ncc(C(F)(F)F)cc6Cl)cc(N6CC(F)C6)cc54)ccc3C2=O)C(=O)N1]

A. 6-bromo-4-chloro-1-((2-(trimethylsilyl) ethoxy)methyl)-1H-indazole. 6-bromo-4-chloro-1H-indazole (1.852 g, 8 mmol), and tetrabutylammonium bromide (0.258 g, 0.800 mmol) were placed in a flask and DCM (48.0 mL) was added. The starting materials were dissolved and the resulting solution was cooled to 0° C. Next, 50% potassium hydroxide (aqueous, 32.0 mL) was added slowly. The resulting biphasic mixture was stirred for a few more minutes after the addition to the mixture was at 0° C. Then, 2-(Trimethylsilyl) ethoxymethyl chloride (2.128 mL, 9.60 mmol) was added dropwise. The resulting solution was stirred overnight. Upon completion, the mixture was transferred to a separatory funnel. The organic layer was removed and the aqueous layer was extracted two more times with DCM. The combined organic layers were washed with water and then brine. The organic layer was dried over anhydrous magnesium sulfate and filtered. The solvent was removed under reduced pressure. The crude material was purified by silica gel chromatography (SiO₂; dry load; ethyl acetate/hexane gradient) to obtain 6-bromo-4-chloro-1-((2-(trimethylsilyl) ethoxy)methyl)-1H-indazole (2.01 g, 1.478 mmol, 70% yield). MS (ESI) m/z: 361.0 [M+1]⁺.

B. 3-(5-(((4-(2-chloro-4-(trifluoromethyl)phenyl)-6-cyclobutoxypyrimidin-2-yl)(methyl)amino)methyl)-1-oxoisoindolin-2-yl) piperidine-2,6-dione. 6-bromo-4-chloro-1-((2-(trimethylsilyl) ethoxy)methyl)-1H-indazole (1.25 g, 3.46 mmol), 3-fluoroazetidine hydrochloride (0.424 g, 3.80 mmol) PdCl₂(dppf)(0.126 g, 0.173 mmol), and tribasic potassium phosphate (2.200 g, 10.37 mmol) were placed in a vial. The vial was evacuated and backfilled with nitrogen three times. 1,4-Dioxane (6.91 ml) was added via syringe and the mixture was degassed with nitrogen for 5 minutes. The reaction was then heated to 85° C. and stirred at that temperature for 2.5 days. Upon completion, the reaction mixture was cooled to room temperature, then diluted with DCM and filtered. The solvent was removed under reduced pressure. The resulting residue was purified by silica gel chromatography (SiO₂; ethyl acetate/hexanes gradient) to provide 3-(5-(((4-(2-chloro-4-(trifluoromethyl)phenyl)-6-cyclobutoxypyrimidin-2-yl)(methyl)amino)methyl)-1-oxoisoindolin-2-yl) piperidine-2,6-dione (916 mg, 2.57 mmol, 75% yield). MS (ESI) m/z: 356.0 [M+1]⁺.

C. 6-(3-fluoroazetidin-1-yl)-4-(4,4,5,5-tetramethyl-1,3,2-dioxaborolan-2-yl)-1-((2-(trimethylsilyl) ethoxy)methyl)-1H-indazole. 4-chloro-6-(3-fluoroazetidin-1-yl)-1-((2-(trimethylsilyl) ethoxy)methyl)-1H-indazole (470 mg, 1.321 mmol), potassium acetate (389 mg, 3.96 mmol), B2pin2 (419 mg, 1.651 mmol), Xphos (31.5 mg, 0.066 mmol), and Xphos-Pd-G2 (51.9 mg, 0.066 mmol) were all placed in a vial and placed under nitrogen. 1,4-Dioxane (4.402 mL) was added and the resulting suspension was degassed with nitrogen for 5 minutes while stirring. The solution was then heated to 50° C. and stirred overnight. Upon completion, the reaction was cooled to room temperature and diluted with DCM. The mixture was filtered through a pad of celite and the solvent was removed under reduced pressure. The crude residue was purified by silica gel chromatography (SiO₂; ethyl acetate/hexanes gradient) to provide 6-(3-fluoroazetidin-1-yl)-4-(4,4,5,5-tetramethyl-1,3,2-dioxaborolan-2-yl)-1-((2-(trimethylsilyl) ethoxy)methyl)-1H-indazole (377.5 mg, 0.844 mmol, 64% yield). MS (ESI) m/z: 448.2 [M+1]⁺.

D. 4-(3-chloro-5-(trifluoromethyl)pyridin-2-yl)-6-(3-fluoroazetidin-1-yl)-1-((2-(trimethylsilyl) ethoxy)methyl)-1H-indazole. PdCl₂(dppf)(61.7 mg, 0.084 mmol), K₂CO₃ (1266 μl, 2.53 mmol), 2-bromo-3-chloro-5-(trifluoromethyl)pyridine (264 mg, 1.012 mmol), and 6-(3-fluoroazetidin-1-yl)-4-(4,4,5,5-tetramethyl-1,3,2-dioxaborolan-2-yl)-1-((2-(trimethylsilyl) ethoxy)methyl)-1H-indazole (377.5 mg, 0.844 mmol) were all placed in a vial and placed under nitrogen. 1,4-Dioxane (3.8 mL) and water (0.42 mL) were added via syringe and the resulting solution was degassed for 5 minutes. the reaction was sealed and heated to 80° C. for 5 hours. Upon completion, the reaction was cooled to room temperature and a biphasic mixture formed. The aqueous layer was removed via pipette. The reaction was diluted with DCM and filtered through a pad of celite. The filtrate was concentrated under reduced pressure and the resulting residue was purified by silica gel chromatography (SiO₂; ethyl acetate/hexanes gradient) to provide 4-(3-chloro-5-(trifluoromethyl)pyridin-2-yl)-6-(3-fluoroazetidin-1-yl)-1-((2-(trimethylsilyl) ethoxy)methyl)-1H-indazole (291 mg, 0.580 mmol, 69% yield) as a yellow semi-solid. MS (ESI) m/z: 501.2 [M+1]⁺.

E. 4-(3-chloro-5-(trifluoromethyl)pyridin-2-yl)-6-(3-fluoroazetidin-1-yl)-1H-indazole. 4-(3-chloro-5-(trifluoromethyl)pyridin-2-yl)-6-(3-fluoroazetidin-1-yl)-1-((2-(trimethylsilyl) ethoxy)methyl)-1H-indazole (158 mg, 0.315 mmol) was placed in a vial and placed under nitrogen. Tetrahydrofuran (1577 μl) was added and the starting material was dissolved. Tetrabutylammonium fluoride (3154 μl, 3.15 mmol) was added (solution in THF) and the reaction was heated to 50° C. overnight. Upon completion, the reaction was cooled to room temperature then quenched with 0.5 M potassium phosphate buffer (pH=7). The solution was extracted three times with ethyl acetate. The combined organic layers were rinsed with brine, dried over anhydrous magnesium sulfate and filtered. The crude material was purified by silica gel chromatography (SiO₂, ethyl acetate/hexanes gradient) to provide 4-(3-chloro-5-(trifluoromethyl)pyridin-2-yl)-6-(3-fluoroazetidin-1-yl)-1H-indazole as a yellow solid (101 mg, 0.271 mmol, 86% yield). MS (ESI) m/z: 371.0 [M+1]⁺.

F. tert-butyl(S)-5-amino-4-(5-((4-(3-chloro-5-(trifluoromethyl)pyridin-2-yl)-6-(3-fluoroazetidin-1-yl)-1H-indazol-1-yl)methyl)-1-oxoisoindolin-2-yl)-5-oxopentanoate. 4-(3-chloro-5-(trifluoromethyl)pyridin-2-yl)-6-(3-fluoroazetidin-1-yl)-1H-indazole (0.060 g, 0.162 mmol), tert-butyl(S)-5-amino-4-(5-(bromomethyl)-1-oxoisoindolin-2-yl)-5-oxopentanoate (Intermediate C)(0.080 g, 0.194 mmol), and cesium carbonate (0.127 g, 0.388 mmol) were placed in a vial and placed under nitrogen. DMF (1.62 ml) was added and the reaction was stirred at 30° C. for 4 hours. Upon completion, the reaction was diluted with DCM, then filtered through a pad of celite. The solvent was removed under reduced pressure. The crude material was purified by silica gel chromatography (SiO₂, methanol/dichloromethane gradient) to provide tert-butyl(S)-5-amino-4-(5-((4-(3-chloro-5-(trifluoromethyl)pyridin-2-yl)-6-(3-fluoroazetidin-1-yl)-1H-indazol-1-yl)methyl)-1-oxoisoindolin-2-yl)-5-oxopentanoate (104.0 mg, 82% purity, 0.122 mmol, 75% yield). Isolated product contains 18% of the regioisomer tert-butyl(S)-5-amino-4-(5-((4-(3-chloro-5-(trifluoromethyl)pyridin-2-yl)-6-(3-fluoroazetidin-1-yl)-2H-indazol-2-yl)methyl)-1-oxoisoindolin-2-yl)-5-oxopentanoate which was carried forward. MS (ESI) m/z: 701.2 [M+1]⁺.

G. 3-(5-((4-(3-chloro-5-(trifluoromethyl)pyridin-2-yl)-6-(3-fluoroazetidin-1-yl)-1H-indazol-1-yl)methyl)-1-oxoisoindolin-2-yl) piperidine-2,6-dione. In a vial, tert-butyl(S)-5-amino-4-(5-((4-(3-chloro-5-(trifluoromethyl)pyridin-2-yl)-6-(3-fluoroazetidin-1-yl)-1H-indazol-1-yl)methyl)-1-oxoisoindolin-2-yl)-5-oxopentanoate (30 mg, 0.043 mmol) was dissolved in 0.5 mL of DCM. TFA (0.25 mL) was then added dropwise and the resulting solution was stirred at room temperature for 2 hours. The volatiles were removed under reduced pressure. The crude material was resuspended in toluene (0.5 mL). and the solvent removed again under reduced pressure. The crude material was placed under high vacuum for 1 hour. Next, the crude material was dissolved in MeCN (0.86 mL) To this was added 1,1′-carbonyldiimidazole (10.41 mg, 0.064 mmol) and 4-dimethylaminopyridine (1.046 mg, 8.56 μmol). After complete dissolution of the solids, DIPEA (0.037 mL, 0.214 mmol) was added and the vial was sealed. The reaction was then heated overnight at 40° C. while stirring. Upon completion, the reaction was cooled and diluted with DMSO. The crude material was filtered through a micron syringe filter and purified by prep HPLC (Xselect CSH C18, 30 mm×150 mm, 5 μm particles; Water/MeCN w/0.1% formic acid; Flow Rate: 40 mL/min; Column Temperature: 25° C.). The pure fractions were combined and lyophilized to provide 3-(5-((4-(3-chloro-5-(trifluoromethyl)pyridin-2-yl)-6-(3-fluoroazetidin-1-yl)-1H-indazol-1-yl)methyl)-1-oxoisoindolin-2-yl) piperidine-2,6-dione (10.2 mg, 0.016 mmol, 38% yield) as a pale yellow solid. ¹H NMR (400 MHz, DMSO-d₆) δ 10.90 (s, 1H), 9.01 (s, 1H), 8.58 (d, J=1.8 Hz, 1H), 7.72 (s, 1H), 7.63 (d, J=7.8 Hz, 1H), 7.37 (s, 1H), 7.31 (d, J=7.7 Hz, 1H), 6.73 (s, 1H), 6.62 (d, J=1.5 Hz, 1H), 5.66 (s, 2H), 5.57-5.34 (m, 1H), 5.07-4.96 (m, 1H), 4.41-4.31 (m, 1H), 4.25-4.13 (m, 3H), 3.98-3.86 (m, 2H), 2.81 (br dd, J=13.1, 4.8 Hz, 1H), 2.53 (br d, J=2.0 Hz, 1H), 2.35-2.26 (m, 1H), 1.96-1.86 (m, 1H). MS (ESI) m/z: 627.2 [M+1]⁺.

### Example 27:5-chloro-6-(2-(dimethylamino)-7-((2-(2,6-dioxopiperidin-3-yl)-1-oxoisoindolin-5-yl)methyl)-7H-pyrrolo[2,3-d]pyrimidin-4-yl)nicotinonitrile

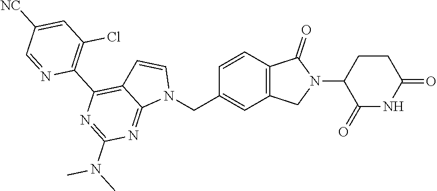 [SMILES: CN(C)c1nc(-c2ncc(C#N)cc2Cl)c2ccn(Cc3ccc4c(c3)CN(C3CCC(=O)NC3=O)C4=O)c2n1]

### A. 4-(2-amino-7H-pyrrolo[2,3-d]pyrimidin-4-yl)-3-chlorobenzonitrile

To a solution of 4-chloro-7H-pyrrolo[2,3-d]pyrimidin-2-amine (0.200 g, 1.19 mmol) in dioxane (3 mL) was added (2-chloro-4-cyanophenyl) boronic acid (0.538 g, 2.97 mmol), potassium carbonate (0.491 g, 3.56 mmol, 2 M) and Xphos-Pd-G2 (0.093 g, 0.12 mmol). The mixture was stirred at 100° C. for 12 hours. The mixture was purified by semi-preparative reverse phase-HPLC (14%-44% acetonitrile in water+0.1% TFA, over 10 min). The solution was lyophilized to give product 4-(2-amino-7H-pyrrolo[2,3-d]pyrimidin-4-yl)-3-chlorobenzonitrile (0.060 g, 0.22 mmol, 19% yield) as a yellow solid. MS (ESI) m/z: 269.8 [M+1]⁺.

### B. 4-(2-amino-7-((2-(2,6-dioxopiperidin-3-yl)-1-oxoisoindolin-5-yl)methyl)-7H-pyrrolo[2,3-d]pyrimidin-4-yl)-3-chlorobenzonitrile

To a solution of 4-(2-amino-7H-pyrrolo[2,3-d]pyrimidin-4-yl)-3-chlorobenzonitrile (0.040 g, 0.15 mmol) in tetrahydrofuran (10 mL) was added 3-(5-(bromomethyl)-1-oxoisoindolin-2-yl) piperidine-2,6-dione (synthesis described in WO2023015283)(0.100 g, 0.3 mmol) and cesium carbonate (0.145 g, 0.44 mmol). The mixture was stirred at 90 degree for 24 hours. The mixture was filtered and the filtrate was concentrated under reduced pressure. The residue was purified by semi-preparative reverse phase-HPLC (20%-50% acetonitrile in water+0.1% TFA, over 10 min). The desired fractions were combined and lyophilized to give product 4-(2-amino-7-((2-(2,6-dioxopiperidin-3-yl)-1-oxoisoindolin-5-yl)methyl)-7H-pyrrolo[2,3-d]pyrimidin-4-yl)-3-chlorobenzonitrile (0.020 g, 0.04 mmol, 26% yield) as a yellow solid. MS (ESI) m/z: 526.2 [M+1]⁺.

### C. 3-chloro-4-(2-(dimethylamino)-7-((2-(2,6-dioxopiperidin-3-yl)-1-oxoisoindolin-5-yl)methyl)-7H-pyrrolo[2,3-d]pyrimidin-4-yl)benzonitrile

To a solution of 4-(2-amino-7-((2-(2,6-dioxopiperidin-3-yl)-1-oxoisoindolin-5-yl)methyl)-7H-pyrrolo[2,3-d]pyrimidin-4-yl)-3-chlorobenzonitrile (0.005. g, 0.01 mmol) in methanol (1.0 mL) and acetic acid (0.1 mL) was added methyl aldehyde (0.1 mL, 2.40 mmol, 37% purity) and borane; 2-methylpyridine (0.005 g, 0.05 mmol). The mixture was stirred at 20 degree for 12 hours. The mixture was purified by semi-preparative reverse phase-HPLC (38%-68% acetonitrile in water+0.1% TFA, over 10 min). The solution was lyophilized to give 3-chloro-4-(2-(dimethylamino)-7-((2-(2,6-dioxopiperidin-3-yl)-1-oxoisoindolin-5-yl)methyl)-7H-pyrrolo[2,3-d]pyrimidin-4-yl)benzonitrile (0.003 g, 0.01 mmol, 52% yield, purity 100%). 1H NMR (400 MHz, DMSO-d₆) δ 10.98 (s, 1H), 8.23 (s, 1H), 8.55 (s, 1H), 7.96 (d, 1H), 7.77 (dd, J=8.0, 16.0, Hz, 2H), 7.60 (s, 1H), 7.52 (d, J=7.6, Hz, 1H), 7.33 (d, J=3.2, Hz, 1H), 6.16 (d, J=3.6 Hz, 1H), 5.45 (s, 2H), 5.12-5.08 (m, 1H), 4.47-4.29 (m, 2H), 3.18 (s, 3H), 2.94-2.90 (m, 1H), 2.61-2.59 (m, 1H), 2.36-2.34 (m, 1H), 2.00-1.97 (m, 1H). MS (ESI) m/z: 554.2 [M+1]⁺.

### Example 30: (3S)-3-(5-{[4-(2,4-difluorophenyl)-6-(3-fluoroazetidin-1-yl)-1H-pyrazolo[3,4-b]pyridin-1-yl]methyl}-1-oxo-2,3-dihydro-1H-isoindol-2-yl) piperidine-2,6-dione

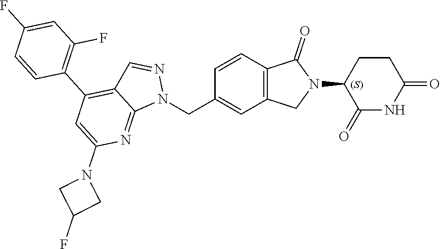

A. (S)-5-amino-4-(5-((4-(2,4-difluorophenyl)-6-(3-fluoroazetidin-1-yl)-1H-pyrazolo[3,4-b]pyridin-1-yl)methyl)-1-oxoisoindolin-2-yl)-5-oxopentanoate. To a 8 mL vial was added 2-(2,4-difluorophenyl)-4,4,5,5-tetramethyl-1,3,2-dioxaborolane (2)(55.5 mg, 0.231 mmol), tert-butyl(S)-5-amino-4-(5-((6-(3-fluoroazetidin-1-yl)-4-iodo-1H-pyrazolo[3,4-b]pyridin-1-yl)methyl)-1-oxoisoindolin-2-yl)-5-oxopentanoate (Intermediate F), 1,4-dioxane (0.9 mL), water (0.100 mL) and sodium carbonate (49.0 mg, 0.463 mmol). The reaction mixture was purged with nitrogen gas for 10 minutes. To the reaction mixture, Tetrakis(triphenylphosphine) palladium (0)(17.82 mg, 0.015 mmol) was added at rt, and the mixture was stirred at 100° C. for 16 h. The reaction mixture was diluted with ethyl acetate (10 mL) dried with anhydrous sodium sulphate and concentrated under reduced pressure. The crude material was purified via preparative HPLC (FA method-Column: Sunfire C₁₈ (250×19) mm 5.0 μm, Mobile phase A: 0.1% formic acid in water, Mobile phase B: acetonitrile, Flow: 15 mL/min, Time: 0-25 min, Gradient: 10-75% B). The desired fractions were concentrated to afford tert-butyl(S)-5-amino-4-(5-((4-(2,4-difluorophenyl)-6-(3-fluoroazetidin-1-yl)-1H-pyrazolo[3,4-b]pyridin-1-yl)methyl)-1-oxoisoindolin-2-yl)-5-oxopentanoate 3 (60 mg, 0.093 mmol, 60% yield). MS (ESI, +ve) m/z: 635.2 (M+1)⁺.

B. (S)-3-(5-((4-(2,4-difluorophenyl)-6-(3-fluoroazetidin-1-yl)-1H-pyrazolo[3,4-b]pyridin-1-yl)methyl)-1-oxoisoindolin-2-yl) piperidine-2,6-dione. In a 8 mL vial, tert-butyl (S)-5-amino-4-(5-((4-(2,4-difluorophenyl)-6-(3-fluoroazetidin-1-yl)-1H-pyrazolo[3,4-b]pyridin-1-yl)methyl)-1-oxoisoindolin-2-yl)-5-oxopentanoate (60 mg, 0.095 mmol) was dissolved in acetonitrile (1 mL) and p-Toluenesulfonic acid (71.9 mg, 0.378 mmol) was added to the mixture at rt, then reaction mixture was stirred at 60° C. for 16 h. The mixture was concentrated under reduced pressure. The residue was purified by Prep-HPLC (FA method-Column: Sunfire C18 (250×19) mm 5.0 μm, Mobile phase A: 0.1% formic acid in water, Mobile phase B: acetonitrile, Flow: 15 mL/min, Time: 0-20 min, Gradient: 10-65% B). The desired fractions were concentrated and lyophilized to afford(S)-3-(5-((4-(2,4-difluorophenyl)-6-(3-fluoroazetidin-1-yl)-1H-pyrazolo[3,4-b]pyridin-1-yl)methyl)-1-oxoisoindolin-2-yl) piperidine-2,6-dione (30 mg, 0.053 mmol, 56% yield). ¹H NMR (400 MHz, DMSO-d6, 2 rotamers) δ:10.98 (s, 1H), 7.73-7.82 (m, 2H), 7.70 (d, J=7.8 Hz, 1H), 7.45-7.54 (m, 2H), 7.42 (d, J=7.9 Hz, 1H), 7.29 (t, J=8.3 Hz, 1H), 6.45 (s, 1H), 5.44-5.68 (m, 3H), 5.10 (dd, J=13.3, 5.1 Hz, 1H), 4.39-4.50 (m, 3H), 4.12-4.33 (m, 3H), 2.85-2.95 (m, 1H), 2.55-2.63 (m, 1H), 2.32-2.39 (m, 1H), 1.93-2.03 (m, 1H). MS (ESI, +ve) m/z: 561.0 (M+1)⁺.

### Example 32: (3S)-3-[5-({4-[5-ethoxy-3-(trifluoromethyl)pyridin-2-yl]-6-(3-fluoroazetidin-1-yl)-1H-pyrazolo[3,4-b]pyridin-1-yl}methyl)-1-oxo-2,3-dihydro-1H-isoindol-2-yl]piperidine-2,6-dione

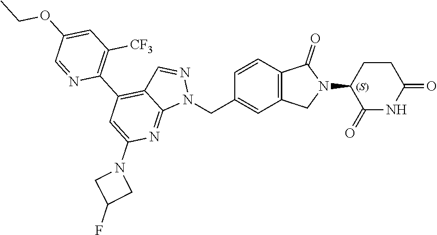

A. 2-chloro-5-ethoxy-3-(trifluoromethyl)pyridine. 6-chloro-5-(trifluoromethyl)pyridin-3-ol (2.0 g, 10.12 mmol) was dissolved in DMF (20 mL). To this mixture was added potassium carbonate (2.80 g, 20.25 mmol) and bromoethane (0.907 mL, 12.15 mmol). The resulted reaction mixture was stirred at 25° C. for 2 h. The reaction mixture was treated with ice cold water and extracted with ethyl acetate (2×75 mL). The combined organic layers were washed with brine. The resulting organic layer was dried over anhydrous sodium sulfate and concentrated under reduced pressure to afford 2-chloro-5-ethoxy-3-(trifluoromethyl)pyridine (1.7 g, 7.08 mmol, 70% yield) as a brown color liquid. MS (ESI, +ve ion) m/z: 226.0 (M+1)⁺.

B. tert-butyl(S)-5-amino-4-(5-((4-(5-ethoxy-3-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate. Tert-butyl(S)-5-amino-4-(5-((6-(3-fluoroazetidin-1-yl)-4-(4,4,5,5-tetramethyl-1,3,2-dioxaborolan-2-yl)-1H-pyrazolo[3,4-b]pyridin-1-yl)methyl)-1-oxoisoindolin-2-yl)-5-oxopentanoate (Intermediate G)(1009 mg, 1.556 mmol) was dissolved in 1,4-Dioxane (15 mL) and water (1.667 mL) in a 40 mL Screw cap vial. To this mixture was added 2-chloro-5-ethoxy-3-(trifluoromethyl)pyridine (270 mg, 1.197 mmol), potassium carbonate (331 mg, 2.394 mmol) and PdCl₂(dppf) DCM adduct (98 mg, 0.120 mmol). The resulting mixture was purged with nitrogen 10 min., then, stirred for 2 h at 130° C. After completion the reaction mixture was cooled to room temperature and extracted with ethyl acetate (2×50 mL), washed with water and brine. The combine organic layers were dried over anhydrous sodium sulfate, filtered, concentrated under reduced pressure. The residue was purified by RP column purification to afford tert-butyl(S)-5-amino-4-(5-((4-(5-ethoxy-3-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.260 g, 0.281 mmol, 24% yield) as a pale brown gummy solid. MS (ESI, +ve ion) m z 712.0 (M+1)⁺.

C. (S)-3-(5-((4-(5-ethoxy-3-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione. Tert-butyl(S)-5-amino-4-(5-((4-(5-ethoxy-3-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.255 g, 0.358 mmol) was weighed in a 15 mL Screw cap vial. To this Acetonitrile (5 mL) was added followed by p-toluenesulfonic acid (0.136 g, 0.717 mmol). The reaction mixture was stirred at 80° C. for 4 h. After completion, the reaction mixture was purified by reverse phase preparative HPLC purification using the following conditions. Column: YMC C18 Phenyl (250×21) mm, 5 micron, Diluent: THF:Water:ACN (50:20:30), Mobile phase A: 5 mM Ammonium formate in water, Mobile phase B: Acetonitrile. (S)-3-(5-((4-(5-ethoxy-3-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione (7.5 mg, 0.011 mmol, 3% yield) was obtained as an off white solid. ¹H NMR (400 MHz, DMSO-d₆) δ 10.97 (br s, 1H), 8.68 (d, J=3.0 Hz, 1H), 7.87 (d, J=3.0 Hz, 1H), 7.70 (d, J=7.5 Hz, 1H), 7.59 (s, 1H), 7.49 (s, 1H), 7.45-7.38 (m, 1H), 6.32 (s, 1H), 5.66-5.43 (m, 3H), 5.09 (dd, J=5.0, 13.0 Hz, 1H), 4.49-4.36 (m, 3H), 4.35-4.27 (m, 3H), 4.23-4.09 (m, 2H), 2.97-2.85 (m, 1H), 2.63-2.60 (m, 1H), 2.44-2.36 (m, 1H), 2.03-1.94 (m, 1H), 1.41 (t, J=7.0 Hz, 3H); MS (ESI, +ve ion) m/z: 638.2 (M+1)⁺.

### Example 41:4-(1-{[2-(2,6-dioxopiperidin-3-yl)-1-oxo-2,3-dihydro-1H-isoindol-5-yl]methyl}-5-fluoro-6-(3-fluoroazetidin-1-yl)-1H-pyrazolo[3,4-b]pyridin-4-yl)-3-fluorobenzonitrile

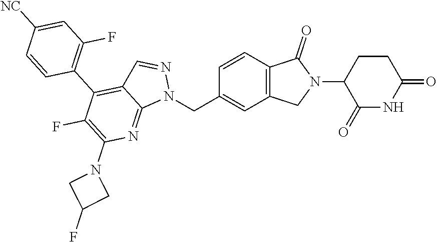 [SMILES: N#Cc1ccc(-c2c(F)c(N3CC(F)C3)nc3c2cnn3Cc2ccc3c(c2)CN(C2CCC(=O)NC2=O)C3=O)c(F)c1]

A. tert-butyl (4S)-5-amino-4-(5-((4-(4-cyano-2-fluorophenyl)-5-fluoro-6-(3-fluoroazetidin-1-yl)-1H-pyrazolo[3,4-b]pyridin-1-yl)methyl)-1-oxoisoindolin-2-yl)-5-oxopentanoate. To a solution of tert-butyl(S)-5-amino-4-(5-((5-fluoro-6-(3-fluoroazetidin-1-yl)-4-iodo-1H-pyrazolo[3,4-b]pyridin-1-yl)methyl)-1-oxoisoindolin-2-yl)-5-oxopentanoate (Intermediate H)(0.080 g, 0.12 mmol and 3-fluoro-4-(4,4,5,5-tetramethyl-1,3,2-dioxaborolan-2-yl)benzonitrile (0.044 g, 0.18 mmol) in 1,4-Dioxane (2 mL) was added 1.5 M potassium phosphate (0.2 mL, 0.36 mmol) and XPhos-Pd-G4 (0.010 g, 0.01 mmol). The mixture was stirred at 55° C. for 1 h under nitrogen. The mixture was diluted with water (10 mL), extracted with ethyl acetate (10 mL×3). The combined organic layer was dried over anhydrous sodium sulfate, filtered and concentrated under reduced pressure. Then the mixture was purified by preparative TLC (80% ethyl acetate in petroleum ether) to afford the product tert-butyl (4S)-5-amino-4-(5-((4-(4-cyano-2-fluorophenyl)-5-fluoro-6-(3-fluoroazetidin-1-yl)-1H-pyrazolo[3,4-b]pyridin-1-yl)methyl)-1-oxoisoindolin-2-yl)-5-oxopentanoate (0.070 g, 0.11 mmol, 88% yield) as a yellow oil. MS (ESI) m/z: 660.3 [M+1]⁺.

B. (4S)-5-amino-4-(5-((4-(4-cyano-2-fluorophenyl)-5-fluoro-6-(3-fluoroazetidin-1-yl)-1H-pyrazolo[3,4-b]pyridin-1-yl)methyl)-1-oxoisoindolin-2-yl)-5-oxopentanoic acid. To a solution of tert-butyl (4S)-5-amino-4-(5-((4-(4-cyano-2-fluorophenyl)-5-fluoro-6-(3-fluoroazetidin-1-yl)-1H-pyrazolo[3,4-b]pyridin-1-yl)methyl)-1-oxoisoindolin-2-yl)-5-oxopentanoate (0.070 g, 0.11 mmol) in dichloromethane (3 mL) was added TFA (0.6 mL, 7.84 mmol). The mixture was stirred at 25° C. for 1 h. The mixture was diluted with water (8 mL), extracted with ethyl acetate (8 mL×3). The combined organic layer was dried over anhydrous sodium sulfate, filtered and concentrated under reduced pressure to afford (4S)-5-amino-4-(5-((4-(4-cyano-2-fluorophenyl)-5-fluoro-6-(3-fluoroazetidin-1-yl)-1H-pyrazolo[3,4-b]pyridin-1-yl)methyl)-1-oxoisoindolin-2-yl)-5-oxopentanoic acid (0.060 g, 0.10 mmol, 94% yield) as a yellow oil. MS (ESI) m/z: 604.2 [M+1]⁺.

C. 4-(1-((2-(2,6-dioxopiperidin-3-yl)-1-oxoisoindolin-5-yl)methyl)-5-fluoro-6-(3-fluoroazetidin-1-yl)-1H-pyrazolo[3,4-b]pyridin-4-yl)-3-fluorobenzonitrile. To a solution of (4S)-5-amino-4-(5-((4-(4-cyano-2-fluorophenyl)-5-fluoro-6-(3-fluoroazetidin-1-yl)-1H-pyrazolo[3,4-b]pyridin-1-yl)methyl)-1-oxoisoindolin-2-yl)-5-oxopentanoic acid (0.060 g, 0.10 mmol) in acetonitrile (2 mL) was added N-ethyl-N-isopropylpropan-2-amine (0.1 mL, 0.40 mmol). Then (dimethylamino)pyridine (0.036 g, 0.30 mmol) and di(1H-imidazol-1-yl) methanone (0.048 g, 0.30 mmol) was added and the mixture was stirred at 90° C. for 12 h. The mixture was concentrated. The residue was purified by semi-preparative reverse phase HPLC (42-72% acetonitrile in water+0.225% formic acid, over 7 min). Then the collected fraction was concentrated to remove most of the acetonitrile, then lyophilized to afford the product 4-(1-((2-(2,6-dioxopiperidin-3-yl)-1-oxoisoindolin-5-yl)methyl)-5-fluoro-6-(3-fluoroazetidin-1-yl)-1H-pyrazolo[3,4-b]pyridin-4-yl)-3-fluorobenzonitrile (0.028 g, 0.05 mmol, 40.5% yield, 98% purity) as an off-white solid. ¹H NMR (400 MHz, DMSO-d₆) δ 11.01-10.97 (m, 1H), 8.15-8.12 (m, 1H), 7.93-7.86 (m, 2H), 7.76 (s, 1H), 7.70 (d, J=7.6 Hz, 1H), 7.52 (s, 1H), 7.43 (d, J=7.6 Hz, 1H), 5.64-5.44 (m, 3H), 5.09 (dd, J=5.6, 13.2 Hz, 1H), 4.65-4.51 (m, 2H), 4.45-4.25 (m, 4H), 2.95-2.86 (m, 1H), 2.58 (d, J=18.0 Hz, 1H), 2.38-2.35 (m, 1H), 2.00-1.96 (m, 1H). MS (ESI) m/z: 586.2 [M+1]⁺.

### Example 44:3-(5-{[4-(3-chloro-5-ethoxypyridin-2-yl)-6-(3-fluoroazetidin-1-yl)-1H-pyrazolo[3,4-b]pyridin-1-yl]methyl}-1-oxo-2,3-dihydro-1H-isoindol-2-yl) piperidine-2,6-dione

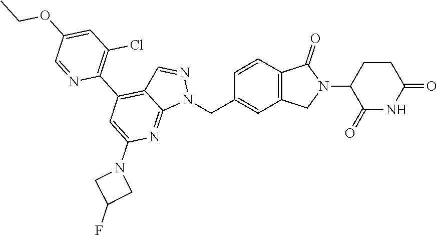 [SMILES: CCOc1cnc(-c2cc(N3CC(F)C3)nc3c2cnn3Cc2ccc3c(c2)CN(C2CCC(=O)NC2=O)C3=O)c(Cl)c1]

A. 3-chloro-5-ethoxy-2-(trimethylstannyl)pyridine. To a solution of 2-bromo-3-chloro-5-ethoxypyridine (250 mg, 1.057 mmol) in Toluene (5 mL) was added hexamethylditin (0.241 mL, 1.163 mmol). The mixture was degassed for 30 seconds. tetrakis(triphenylphosphine) palladium (0)(122 mg, 0.106 mmol) was added and the mixture was heated to 100° C. for 4 h. The reaction mixture was filtered through a 0.45 micron filter, washed with DCM (5 mL), and the filtrate was concentrated under reduced pressure to obtain crude 3-chloro-5-ethoxy-2-(trimethylstannyl)pyridine. MS (ESI) m/z: 320.0 [M]⁺.

B. tert-butyl(S)-5-amino-4-(5-((4-(3-chloro-5-ethoxypyridin-2-yl)-6-(3-fluoroazetidin-1-yl)-1H-pyrazolo[3,4-b]pyridin-1-yl)methyl)-1-oxoisoindolin-2-yl)-5-oxopentanoate. To tert-butyl(S)-5-amino-4-(5-((6-(3-fluoroazetidin-1-yl)-4-iodo-1H-pyrazolo[3,4-b]pyridin-1-yl)methyl)-1-oxoisoindolin-2-yl)-5-oxopentanoate (Intermediate F) (150 mg, 0.227 mmol) in Dioxane (5 mL) was added 3-chloro-5-ethoxy-2-(trimethylstannyl)pyridine (145 mg, 0.453 mmol), copper (I) iodide (2.203 mg, 0.011 mmol), and lithium chloride (19.61 mg, 0.453 mmol). The mixture was degassed for 30 seconds. Tetrakis(triphenylphosphine) palladium (0)(26.7 mg, 0.023 mmol) was added and the mixture was heated to 100° C. overnight. The reaction mixture was filtered through celite, washed with DCM (10 mL), and concentrated under reduced pressure. The crude material was purified by preparative HPLC (Formic acid) and the fractions were concentrated under reduced pressure to get tert-butyl(S)-5-amino-4-(5-((4-(3-chloro-5-ethoxypyridin-2-yl)-6-(3-fluoroazetidin-1-yl)-1H-pyrazolo[3,4-b]pyridin-1-yl)methyl)-1-oxoisoindolin-2-yl)-5-oxopentanoate (58 mg, 0.081 mmol, 36% yield) as an off white solid. MS (ESI) m/z: 678.2 [M]⁺.

C. 3-(5-((4-(3-chloro-5-ethoxypyridin-2-yl)-6-(3-fluoroazetidin-1-yl)-1H-pyrazolo[3,4-b]pyridin-1-yl)methyl)-1-oxoisoindolin-2-yl) piperidine-2,6-dione. tert-butyl (S)-5-amino-4-(5-((4-(3-chloro-5-ethoxypyridin-2-yl)-6-(3-fluoroazetidin-1-yl)-1H-pyrazolo[3,4-b]pyridin-1-yl)methyl)-1-oxoisoindolin-2-yl)-5-oxopentanoate (160 mg, 0.201 mmol) in acetonitrile (5 mL) was added p-Toluenesulfonic acid (156 mg, 0.803 mmol) and the reaction mixture was heated to 70° C. overnight. The reaction mixture was concentrated under reduced pressure. The residue was purified by reverse phase HPLC (0.1% formic acid) and the fractions were concentrated under reduced pressure. The concentrate was lyophilized using (ACN:Water) to afford the 3-(5-((4-(3-chloro-5-ethoxypyridin-2-yl)-6-(3-fluoroazetidin-1-yl)-1H-pyrazolo[3,4-b]pyridin-1-yl)methyl)-1-oxoisoindolin-2-yl) piperidine-2,6-dione (30.1 mg, 0.049 mmol, 25% yield) as a white solid. ¹H NMR (400 MHz, DMSO-d₆) δ:10.99 (s, 1H), 8.44 (d, J=2.0 Hz, 1H), 7.78 (d, J=2.5 Hz, 1H), 7.76 (s, 1H), 7.70 (d, J=7.5 Hz, 1H), 7.49 (s, 1H), 7.41 (d, J=8.0 Hz, 1H), 6.55 (s, 1H), 5.45-5.63 (m, 3H), 5.06-5.14 (m, 1H), 4.40-4.47 (m, 3H), 4.12-4.25 (m, 5H), 2.87-2.93 (m, 1H), 2.61 (br s, 1H), 2.38 (br s, 1H), 1.96-2.01 (m, 1H), 1.39 (t, J=7.0 Hz, 3H). MS (ESI) m/z: 604.2 [M]⁺.

### Example 46:2-(1-((2-(2,6-dioxopiperidin-3-yl)-1-oxoisoindolin-5-yl)methyl)-6-(3-fluoroazetidin-1-yl)-1H-pyrazolo[3,4-b]pyridin-4-yl)-5-ethoxynicotinonitrile

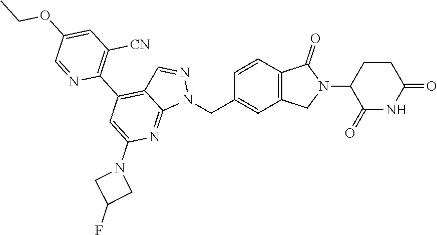 [SMILES: CCOc1cnc(-c2cc(N3CC(F)C3)nc3c2cnn3Cc2ccc3c(c2)CN(C2CCC(=O)NC2=O)C3=O)c(C#N)c1]

A. 2-chloro-5-ethoxynicotinonitrile. To the stirred solution of 2-chloro-5-hydroxynicotinonitrile (1)(500 mg, 3.24 mmol) in DMF (5 ml) was added cesium carbonate (3162 mg, 9.71 mmol) followed by ethyl iodide (0.392 ml, 4.85 mmol) at RT. The resultant reaction mixture was stirred at RT for 2 h. The reaction was monitored by TLC which showed new spot and UPLC-MS showed desired product mass. To the reaction mixture, water (30 ml) was added, extracted with ethyl acetate (40 ml×2). To the collected organic layer brine wash (20 ml×4) has given. The organic layer was dried using anhydrous sodium sulfate, concentrated under reduced pressure to afford 2-chloro-5-ethoxynicotinonitrile (550 mg, 1.837 mmol, 57% yield) as an off-white solid. MS (ESI, +ve) m/z: 183.0 (M+1)⁺.

B. 5-ethoxy-2-(trimethylstannyl)nicotinonitrile. To the stirred solution of 2-chloro-5-ethoxynicotinonitrile (400 mg, 2.190 mmol) in toluene (8 ml) was added 1,1,1,2,2,2-hexamethyldistannane (1077 mg, 3.29 mmol). The reaction mixture was degassed by N2 for 10 min followed by addition of Tetrakis(triphenylphosphine) palladium (0)(506 mg, 0.438 mmol) at rt. The resultant reaction mixture was stirred at 100° C. for 16 h. The reaction was monitored by UPLC-MS, which showed desired product mass. The reaction mass filtered through celite bed and washed with ethyl acetate (30 mL×3). The collected organic layer was concentrated under reduced pressure to afford crude (1.2 gm) 5-ethoxy-2-(trimethylstannyl)nicotinonitrile as a brown liquid, which was used as such without purification. MS (ESI, +ve) m/z: 313.0 (M+1)⁺.

C. tert-butyl(S)-5-amino-4-(5-((4-(3-cyano-5-ethoxypyridin-2-yl)-6-(3-fluoroazetidin-1-yl)-1H-pyrazolo[3,4-b]pyridine-1-yl)methyl)-1-oxoisoindolin-2-yl)-5-oxopentanoate. To the stirred solution of tert-butyl(S)-5-amino-4-(5-((6-(3-fluoroazetidin-1-yl)-4-iodo-1H-pyrazolo[3,4-b]pyridin-1-yl)methyl)-1-oxoisoindolin-2-yl)-5-oxopentanoate (Intermediate F)(200 mg, 0.308 mmol) in dioxane (5 mL) was added 5-ethoxy-2-(trimethylstannyl)nicotinonitrile (432 mg, 1.388 mmol) followed by lithium chloride (26.1 mg, 0.617 mmol). The reaction mixture was degassed by N2 for 10 min. Then Tetrakis(triphenylphosphine) palladium (0)(35.6 mg, 0.031 mmol) and copper (I) iodide (11.75 mg, 0.062 mmol) were added at rt. The resultant reaction mixture was stirred at 100° C. for 19 h. The reaction mixture was treated with water (10 ml) and extracted with ethyl acetate (20 mL×2). The combined organic layers were dried with anhydrous sodium sulfate, filtered, and concentrated under reduced pressure. The crude material was purified by using silica gel chromatography (25 g SiO₂, 30-400 nm mesh) using a gradient of 0-100% ethyl acetate in petroleum ether. The pure fractions were concentrated under reduced pressure. The compound was re-purified by preparative HPLC using the following method. (Column-Xselect CSH C18 (250×19) mm 10.0 μm, Mobile phase A: 0.1% formic acid in water, Mobile phase B: acetonitrile, Flow: 15 mL/min, Time: 0-50 min, Gradient: 10-90% B). The pure fractions were concentrated under reduced pressure to afford tert-butyl(S)-5-amino-4-(5-((4-(3-cyano-5-ethoxypyridin-2-yl)-6-(3-fluoroazetidin-1-yl)-1H-pyrazolo[3,4-b]pyridine-1-yl)methyl)-1-oxoisoindolin-2-yl)-5-oxopentanoate (50 mg, 0.072 mmol, 23% yield) as a yellow solid. MS (ESI, +ve) m/z: 699.2 (M+1)⁺.

D. 2-(1-((2-(2,6-dioxopiperidin-3-yl)-1-oxoisoindolin-5-yl)methyl)-6-(3-fluoroazetidin-1-yl)-1H-pyrazolo[3,4-b]pyridin-4-yl)-5-ethoxynicotinonitrile. To the stirred solution of tert-butyl(S)-5-amino-4-(5-((4-(3-cyano-5-ethoxypyridin-2-yl)-6-(3-fluoroazetidin-1-yl)-1H-pyrazolo[3,4-b]pyridin-1-yl)methyl)-1-oxoisoindolin-2-yl)-5-oxopentanoate (45 mg, 0.067 mmol) in acetonitrile (3 mL) was added p-toluenesulfonic acid (46.4 mg, 0.269 mmol) at room temperature. The resultant reaction mixture was stirred at 65° C. for 2 h. The mixture was concentrated and purified by preparative HPLC using the following method. Column: Atlantis T3 (250×19) mm 10.0 μm, Mobile phase A: 0.1% formic acid in water, Mobile phase B: Acetonitrile, Flow: 15 mL/min, Time: 0-45 min, Gradient: 15-85% B). Pure fractions were concentrated under reduced pressure and lyophilized to afford 2-(1-((2-(2,6-dioxopiperidin-3-yl)-1-oxoisoindolin-5-yl)methyl)-6-(3-fluoroazetidin-1-yl)-1H-pyrazolo[3,4-b]pyridin-4-yl)-5-ethoxynicotinonitrile (15 mg, 0.025 mmol, 37% yield) as a yellow solid. ¹H NMR (400 MHz, DMSO-d6, 2 rotamers) δ:10.98 (s, 1H), 8.75 (d, J=2.5 Hz, 1H), 8.17 (d, J=3.0 Hz, 1H), 7.95 (s, 1H), 7.69 (d, J=8.0 Hz, 1H), 7.48 (s, 1H), 7.41 (d, J=8.0 Hz, 1H), 6.80 (s, 1H), 5.47-5.65 (m, 3H), 5.09 (dd, J=13.3, 5.3 Hz, 1H), 4.39-4.51 (m, 3H), 4.25-4.34 (m, 3H), 4.13-4.24 (m, 2H), 2.85-2.95 (m, 1H), 2.57-2.62 (m, 1H), 2.32-2.35 (m, 1H), 1.94-2.02 (m, 1H), 1.40 (t, J=7.0 Hz, 3H). MS (ESI, +ve) m/z: 595.3 (M+1)⁺.

### Example 49: (3S)-3-[5-({4-[3-chloro-5-(difluoromethoxy)pyridin-2-yl]-6-(3-fluoroazetidin-1-yl)-1H-pyrazolo[3,4-b]pyridin-1-yl}methyl)-1-oxo-2,3-dihydro-1H-isoindol-2-yl]piperidine-2,6-dione

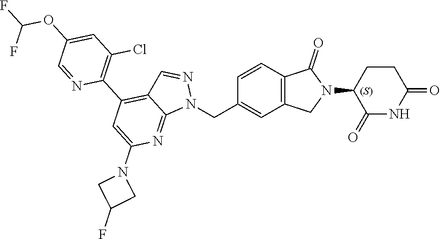

A. 2-bromo-3-chloro-5-(difluoromethoxy)pyridine. To a solution of 6-bromo-5-chloropyridin-3-ol (0.500 g, 2.40 mmol) in N,N-dimethylformamide (6 mL) was added sodium 2-chloro-2,2-difluoroacetate (0.731 g, 4.80 mmol) and potassium carbonate (0.430 g, 3.12 mmol). The mixture was stirred at 80° C. for 2 h. The mixture was diluted with water (20 mL), extracted with ethyl acetate (20 mL×3). The combined organic layer was dried over anhydrous sodium sulfate, filtered and concentrated under reduced pressure. It was purified by silica column chromatography (0˜8% ethyl acetate in petroleum ether) to afford 2-bromo-3-chloro-5-(difluoromethoxy)pyridine (0.350 g, 1.35 mmol, 57% yield) as a yellow oil. ¹H NMR (400 MHz, CDCl₃) δ 8.22 (d, J=2.8 Hz, 1H), 7.62 (d, J=2.8 Hz, 1H), 6.57 (t, J=71.6 Hz, 1H). MS (ESI) m/z: 259.9 [M+1]⁺.

B. tert-butyl(S)-5-amino-4-(5-((4-(3-chloro-5-(difluoromethoxy)pyridin-2-yl)-6-fluoro-1H-pyrazolo[3,4-b]pyridin-1-yl)methyl)-1-oxoisoindolin-2-yl)-5-oxopentanoate. To a solution of 2-bromo-3-chloro-5-(difluoromethoxy)pyridine (0.100 g, 0.39 mmol) and tert-butyl (S)-5-amino-4-(5-((6-fluoro-4-(tributylstannyl)-1H-pyrazolo[3,4-b]pyridin-1-yl)methyl)-1-oxoisoindolin-2-yl)-5-oxopentanoate (Intermediate E)(0.322 g, 0.43 mmol in N,N-dimethylacetamide (2 mL) was added PCy3-Pd-G3 (0.028 g, 0.04 mmol). The mixture was stirred at 90° C. for 12 h under nitrogen. The mixture was quenched with potassium fluoride aqueous solution (8 mL) and diluted with water (8 mL), extracted with ethyl acetate (10 mL×3). The combined organic layer was dried over anhydrous sodium sulfate, filtered and concentrated under reduced pressure. The residue was purified by semi-preparative reverse phase HPLC (43-73% acetonitrile in water+0.225% formic acid, over 10 min). Then the collected fraction was concentrated to remove most of the acetonitrile, then lyophilized to afford the product tert-butyl (S)-5-amino-4-(5-((4-(3-chloro-5-(difluoromethoxy)pyridin-2-yl)-6-fluoro-1H-pyrazolo[3,4-b]pyridin-1-yl)methyl)-1-oxoisoindolin-2-yl)-5-oxopentanoate (0.093 g, 0.14 mmol, 37% yield) as a colorless oil. MS (ESI) m/z: 645.4 [M+1]⁺.

C. tert-butyl(S)-5-amino-4-(5-((4-(3-chloro-5-(difluoromethoxy)pyridin-2-yl)-6-(3-fluoroazetidin-1-yl)-1H-pyrazolo[3,4-b]pyridin-1-yl)methyl)-1-oxoisoindolin-2-yl)-5-oxopentanoate. To a solution of tert-butyl(S)-5-amino-4-(5-((4-(3-chloro-5-(difluoromethoxy)pyridin-2-yl)-6-fluoro-1H-pyrazolo[3,4-b]pyridin-1-yl)methyl)-1-oxoisoindolin-2-yl)-5-oxopentanoate (0.093 g, 0.14 mmol) in acetonitrile (2 mL) was added 3-fluoroazetidine; hydrochloride (0.019 g, 0.17 mmol) and N-ethyl-N-isopropylpropan-2-amine (0.1 mL, 0.58 mmol). The mixture was stirred at 60° C. for 2 h. The mixture was diluted with water (10 mL) and extracted with ethyl acetate (10 mL×3). The combined organic layers were dried over anhydrous sodium sulfate, filtered, and concentrated under reduced pressure to afford tert-butyl(S)-5-amino-4-(5-((4-(3-chloro-5-(difluoromethoxy)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.090 g, 0.13 mmol, 89% yield) as a yellow oil. MS (ESI) m/z: 700.4 [M+1]⁺.

D. (S)-3-(5-((4-(3-chloro-5-(difluoromethoxy)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione. To a solution of tert-butyl(S)-5-amino-4-(5-((4-(3-chloro-5-(difluoromethoxy)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.070 g, 0.10 mmol) in acetonitrile (1 mL) was added 4-methylbenzenesulfonic acid (0.069 g, 0.40 mmol). The mixture was stirred at 60° C. for 2 h. The mixture was added with bicarbonate sodium aqueous solution (10 mL) and extracted with ethyl acetate (10 mL×3). The combined organic layer was dried over anhydrous sodium sulfate, filtered and concentrated under reduced pressure. The residue was purified by semi-preparative reverse phase HPLC (40-70% acetonitrile in water+0.225% formic acid, over 10 min). Then the collected fraction was concentrated to remove most of the acetonitrile, then lyophilized to afford the crude product. The crude product was separated by SFC separation (Column: DAICEL CHIRALPAK OJ (250 mm*30 mm, 10 μm), Mobile phase: Phase A for CO₂, and Phase B for IPA (0.1% NH3H2O); Gradient elution: IPA (0.1% NH₃H₂O) in CO₂ from 40% to 40%, Flow rate: 150 mL/min; 9.8 min) to afford one fraction. The fraction was concentrated under reduced pressure to afford a residue. The residue was diluted with water (10 mL) and extracted with ethyl acetate (10 mL×3). The combined organic layer was dried over anhydrous sodium sulfate, filtered, and concentrated under reduced pressure. The residue was re-dissolved in water (10 mL) and acetonitrile (5 mL), then lyophilized to afford the product(S)-3-(5-((4-(3-chloro-5-(difluoromethoxy)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione (0.008 g, 0.01 mmol, 12% yield, 99.8% purity, 99.1% ee) as a yellow oil. ¹H NMR (400 MHz, DMSO-d6) δ=10.97 (s, 1H), 8.66 (d, J=2.4 Hz, 1H), 8.16 (d, J=2.4 Hz, 1H), 7.75 (s, 1H), 7.70-7.66 (m, 1H), 7.49-7.30 (m, 3H), 6.57 (s, 1H), 5.63-5.46 (m, 3H), 5.09 (dd, J=5.6, 13.6 Hz, 1H), 4.49-4.40 (m, 3H), 4.32-4.27 (m, 1H), 4.22-4.13 (m, 2H), 2.94-2.85 (m, 1H), 2.61-2.60 (m, 1H), 2.42-2.36 (m, 1H), 2.00-1.94 (m, 1H). MS (ESI) m/z: 626.0 [M+1]⁺.

### Example 60:4-(6-(azetidin-1-yl)-1-((2-(2,6-dioxopiperidin-3-yl)-1-oxoisoindolin-5-yl)methyl)-3-methyl-1H-pyrazolo[3,4-b]pyridin-4-yl)-3-chlorobenzonitrile

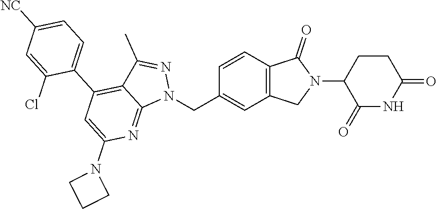 [SMILES: Cc1nn(Cc2ccc3c(c2)CN(C2CCC(=O)NC2=O)C3=O)c2nc(N3CCC3)cc(-c3ccc(C#N)cc3Cl)c12]

A. 3-bromo-6-fluoro-4-iodo-1H-pyrazolo[3,4-b]pyridine. A mixture of 6-fluoro-4-iodo-1H-pyrazolo[3,4-b]pyridine (Intermediate A)(1.000 g, 3.80 mmol) and N-bromosuccinimide (1.353 g, 7.60 mmol) in DMF (10 mL) was stirred at 25° C. for 12 h. The reaction mixture was diluted with water (100 mL) and extracted with ethyl acetate (20 mL×2). The combined organic layers were dried over anhydrous sodium sulfate, filtered, and concentrated under vacuum. The residue was purified by silica gel chromatography (0-18% ethyl acetate in petroleum ether) to give 3-bromo-6-fluoro-4-iodo-1H-pyrazolo[3,4-b]pyridine (1.300 g, 3.80 mmol, 100% yield) as a yellow oil, which was used in the next step directly. MS (ESI) m/z: 341.9 [M+1]⁺.

B. product(S)-tert-butyl 5-amino-4-(5-((3-bromo-6-fluoro-4-iodo-1H-pyrazolo[3,4-b]pyridin-1-yl)methyl)-1-oxoisoindolin-2-yl)-5-oxopentanoate. A mixture of 3-bromo-6-fluoro-4-iodo-1H-pyrazolo[3,4-b]pyridine (1.300 g, 3.80 mmol), (S)-tert-butyl 5-amino-4-(5-(bromomethyl)-1-oxoisoindolin-2-yl)-5-oxopentanoate (Intermediate C)(1.567 g, 3.80 mmol) and cesium carbonate (2.471 g, 7.60 mmol) in DMF (20 mL) was stirred at 50° C. for 2 h. The reaction mixture was diluted with water (200 mL) and adjusted to pH 7 with 1 M phosphoric acid. The mixture was extracted with ethyl acetate (50 mL×2). The combined organic layers were dried over anhydrous sodium sulfate, filtered, and concentrated under vacuum. The residue was purified by silica gel chromatography (0-100% ethyl acetate in petroleum ether) to give(S)-tert-butyl 5-amino-4-(5-((3-bromo-6-fluoro-4-iodo-1H-pyrazolo[3,4-b]pyridin-1-yl)methyl)-1-oxoisoindolin-2-yl)-5-oxopentanoate (0.600 g, 0.89 mmol, 24% yield) as a yellow solid. ¹H NMR (400 MHz, DMSO-d₆) δ 7.75 (s, 1H), 7.66 (d, J=7.6 Hz, 1H), 7.53 (s, 1H), 7.44 (s, 1H), 7.38 (d, J=8.0 Hz, 1H), 7.16 (s, 1H), 5.67 (s, 2H), 4.69 (dd, J=4.0, 10.4 Hz, 1H), 4.58-4.49 (m, 1H), 4.47-4.37 (m, 1H), 2.16-2.10 (m, 3H), 1.98-1.90 (m, 1H), 1.32-1.28 (m, 9H). MS (ESI) m/z: 674.0 [M+1]⁺.

C. (S)-tert-butyl 5-amino-4-(5-((3-bromo-4-(2-chloro-4-cyanophenyl)-6-fluoro-1H-pyrazolo[3,4-b]pyridin-1-yl)methyl)-1-oxoisoindolin-2-yl)-5-oxopentanoate. A mixture of(S)-tert-butyl 5-amino-4-(5-((3-bromo-6-fluoro-4-iodo-1H-pyrazolo[3,4-b]pyridin-1-yl)methyl)-1-oxoisoindolin-2-yl)-5-oxopentanoate (0.600 g, 0.89 mmol), 3-chloro-4-(4,4,5,5-tetramethyl-1,3,2-dioxaborolan-2-yl)benzonitrile (0.353 g, 1.34 mmol), [2-(2-aminophenyl)phenyl]-chloro-palladium; dicyclohexyl-[3-(2,4,6-triisopropylphenyl)phenyl]phosphane (0.076 g, 0.09 mmol) and 2 M potassium carbonate (0.9 mL, 1.78 mmol) in dioxane (10 mL) was stirred at 85° C. for 24 h under nitrogen. The reaction mixture was concentrated under vacuum and then purified by silica gel chromatography (0-70% ethyl acetate in petroleum ether) to give the product(S)-tert-butyl 5-amino-4-(5-((3-bromo-4-(2-chloro-4-cyanophenyl)-6-fluoro-1H-pyrazolo[3,4-b]pyridin-1-yl)methyl)-1-oxoisoindolin-2-yl)-5-oxopentanoate (0.200 g, 0.29 mmol, 33% yield) as a light yellow solid. ¹H NMR (400 MHz, DMSO-d₆) δ 8.33 (s, 1H), 8.05 (d, J=8.0 Hz, 1H), 7.80-7.72 (m, 1H), 7.69 (d, J=7.6 Hz, 1H), 7.59-7.51 (m, 2H), 7.46 (d, J=7.6 Hz, 1H), 7.25 (s, 1H), 7.16 (s, 1H), 5.81-5.66 (m, 2H), 4.70 (dd, J=3.6, 10.4 Hz, 1H), 4.62-4.53 (m, 1H), 4.48-4.40 (m, 1H), 2.13 (s, 3H), 1.98-1.85 (m, 1H), 1.30 (s, 9H). MS (ESI) m/z: 683.0 [M+1]⁺.

D. (S)-tert-butyl 5-amino-4-(5-((4-(2-chloro-4-cyanophenyl)-6-fluoro-3-methyl-1H-pyrazolo[3,4-b]pyridin-1-yl)methyl)-1-oxoisoindolin-2-yl)-5-oxopentanoate. A mixture of(S)-tert-butyl 5-amino-4-(5-((3-bromo-4-(2-chloro-4-cyanophenyl)-6-fluoro-1H-pyrazolo[3,4-b]pyridin-1-yl)methyl)-1-oxoisoindolin-2-yl)-5-oxopentanoate (0.160 g, 0.23 mmol), 2,4,6-trimethyl-1,3,5,2,4,6-trioxatriborinane (0.018 g, 0.14 mmol), tetrakis(triphenylphosphine) palladium (0)(0.027 g, 0.02 mmol) and cesium carbonate (0.153 g, 0.47 mmol) in dioxane (5 mL) and water (0.5 mL) was stirred at 85° C. for 24 h under nitrogen. The reaction mixture was purified by preparative TLC (50% ethyl acetate in petroleum ether, R_{f}=0.3) to give the product (S)-tert-butyl 5-amino-4-(5-((4-(2-chloro-4-cyanophenyl)-6-fluoro-3-methyl-1H-pyrazolo[3,4-b]pyridin-1-yl)methyl)-1-oxoisoindolin-2-yl)-5-oxopentanoate (0.020 g, 0.03 mmol, 14% yield) as a yellow solid. ¹H NMR (400 MHz, DMSO-d₆) δ 8.37-8.30 (m, 1H), 8.07-7.99 (m, 1H), 7.77 (d, J=8.0 Hz, 1H), 7.67 (d, J=7.6 Hz, 1H), 7.52 (s, 2H), 7.42 (d, J=8.0 Hz, 1H), 7.20-7.13 (m, 1H), 7.06 (s, 1H), 5.78-5.60 (m, 2H), 4.69 (dd, J=3.2, 10.0 Hz, 1H), 4.58-4.51 (m, 1H), 4.47-4.39 (m, 1H), 2.13 (s, 3H), 2.02-1.87 (m, 4H), 1.30 (d, J=2.4 Hz, 9H). MS (ESI) m/z: 617.3 [M+1]⁺.

E. (S)-tert-butyl 5-amino-4-(5-((6-(azetidin-1-yl)-4-(2-chloro-4-cyanophenyl)-3-methyl-1H-pyrazolo[3,4-b]pyridin-1-yl)methyl)-1-oxoisoindolin-2-yl)-5-oxopentanoate. The mixture of(S)-tert-butyl 5-amino-4-(5-((4-(2-chloro-4-cyanophenyl)-6-fluoro-3-methyl-1H-pyrazolo[3,4-b]pyridin-1-yl)methyl)-1-oxoisoindolin-2-yl)-5-oxopentanoate (0.030 g, 0.05 mmol), azetidine hydrochloride (0.007 g, 0.07 mmol) and N-ethyl-N-isopropylpropan-2-amine (0.013 g, 0.10 mmol) in acetonitrile (2 mL) was stirred at 80° C. for 2 h. The reaction mixture was concentrated under reduced pressure to give the crude product(S)-tert-butyl 5-amino-4-(5-((6-(azetidin-1-yl)-4-(2-chloro-4-cyanophenyl)-3-methyl-1H-pyrazolo[3,4-b]pyridin-1-yl)methyl)-1-oxoisoindolin-2-yl)-5-oxopentanoate (0.031 g, 0.05 mmol, 97% yield) as a yellow solid, which was used in the next step directly. MS (ESI) m/z: 645.3 [M+1]⁺.

F. (S)-5-amino-4-(5-((6-(azetidin-1-yl)-4-(2-chloro-4-cyanophenyl)-3-methyl-1H-pyrazolo[3,4-b]pyridin-1-yl)methyl)-1-oxoisoindolin-2-yl)-5-oxopentanoic acid. To a solution of(S)-tert-butyl 5-amino-4-(5-((6-(azetidin-1-yl)-4-(2-chloro-4-cyanophenyl)-3-methyl-1H-pyrazolo[3,4-b]pyridin-1-yl)methyl)-1-oxoisoindolin-2-yl)-5-oxopentanoate (0.031 g, 0.05 mmol) in dichloromethane (1 mL) was added TFA (0.3 mL, 3.89 mmol) at 25° C. Then the mixture was stirred at 25° C. for 2 h. The reaction mixture was diluted with water (20 mL) and extracted with dichloromethane (10 mL×2). The combined organic layers were dried over anhydrous sodium sulfate, filtered and concentrated under reduced pressure to give the crude product(S)-5-amino-4-(5-((6-(azetidin-1-yl)-4-(2-chloro-4-cyanophenyl)-3-methyl-1H-pyrazolo[3,4-b]pyridin-1-yl)methyl)-1-oxoisoindolin-2-yl)-5-oxopentanoic acid (0.028 g, 0.05 mmol, 99% yield) as a light yellow solid, which was used in the next step directly. MS (ESI) m/z: 598.2 [M+1]⁺.

G. 4-(6-(Azetidin-1-yl)-1-((2-(2,6-dioxopiperidin-3-yl)-1-oxoisoindolin-5-yl)methyl)-3-methyl-1H-pyrazolo[3,4-b]pyridin-4-yl)-3-chlorobenzonitrile. The mixture of (S)-5-amino-4-(5-((6-(azetidin-1-yl)-4-(2-chloro-4-cyanophenyl)-3-methyl-1H-pyrazolo[3,4-b]pyridin-1-yl)methyl)-1-oxoisoindolin-2-yl)-5-oxopentanoic acid (0.028 g, 0.05 mmol), carbonyl diimidazole (0.015 g, 0.09 mmol), 4-dimethylaminopyridine (0.006 g, 0.05 mmol) and N-ethyl-N-isopropylpropan-2-amine (0.012 g, 0.09 mmol) in acetonitrile (1 mL) was stirred at 80° C. for 12 h. The reaction mixture was purified by semi-preparative reverse phase-HPLC (39-69% acetonitrile in water+0.225% formic acid, over 10 min). The desired fractions were lyophilized to afford the product 4-(6-(Azetidin-1-yl)-1-((2-(2,6-dioxopiperidin-3-yl)-1-oxoisoindolin-5-yl)methyl)-3-methyl-1H-pyrazolo[3,4-b]pyridin-4-yl)-3-chlorobenzonitrile (0.004 g, 0.01 mmol, 13% yield, 98% purity) as an off-white solid. ¹H NMR (400 MHz, DMSO-d₆) δ 11.10-10.81 (m, 1H), 8.32-8.20 (m, 1H), 8.02-7.88 (m, 1H), 7.74-7.60 (m, 2H), 7.51 (s, 1H), 7.42 (d, J=7.6 Hz, 1H), 6.33-6.06 (m, 1H), 5.62-5.43 (m, 2H), 5.09 (dd, J=5.2, 13.2 Hz, 1H), 4.50-4.38 (m, 1H), 4.37-4.25 (m, 1H), 4.16-4.04 (m, 4H), 2.96-2.84 (m, 1H), 2.64-2.56 (m, 1H), 2.44-2.32 (m, 4H), 2.04-1.93 (m, 1H), 1.81 (s, 2H). MS (ESI) m/z: 580.3 [M+1]⁺.

### Example 61: (S)-3-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl) piperidine-2,6-dione

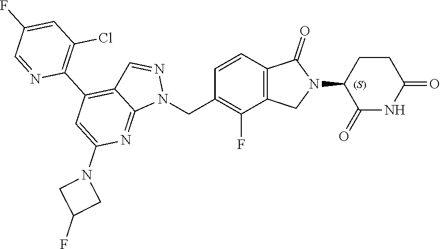

### Example 109: (R)-3-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl) piperidine-2,6-dione

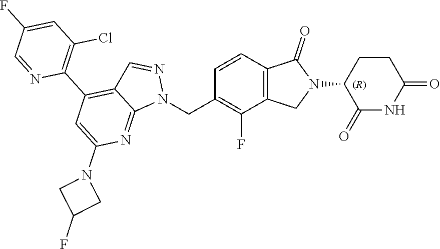

A. tert-butyl(S)-5-amino-4-(4-fluoro-5-((6-fluoro-4-iodo-1H-pyrazolo[3,4-b]pyridin-1-yl)methyl)-1-oxoisoindolin-2-yl)-5-oxopentanoate. A mixture of 6-fluoro-4-iodo-1H-pyrazolo[3,4-b]pyridine (Intermediate A)(1.000 g, 3.80 mmol), tert-butyl(S)-5-amino-4-(4-fluoro-5-(((methylsulfonyl)oxy)methyl)-1-oxoisoindolin-2-yl)-5-oxopentanoate (Intermediate K)(2.535 g, 5.70 mmol) and cesium carbonate (2.471 g, 7.60 mmol) in DMF (20 mL) was stirred at 50° C. for 2 h. The reaction mixture was diluted with water (200 mL) and extracted with ethyl acetate (50 mL×2). The combined organic layers were concentrated under vacuum. The residue was purified by semi-preparative reverse phase-HPLC (50-80% acetonitrile in water+0.225% formic acid, over 17 min). The desired fractions were extracted with ethyl acetate (50 mL×2). The combined organic layers were dried over anhydrous sodium sulfate solid, filtered, and concentrated under vacuum to give tert-butyl(S)-5-amino-4-(4-fluoro-5-((6-fluoro-4-iodo-1H-pyrazolo[3,4-b]pyridin-1-yl)methyl)-1-oxoisoindolin-2-yl)-5-oxopentanoate (1.100 g, 1.80 mmol, 47% yield) as a yellow solid. ¹H NMR (400 MHz, CDCl₃) δ 7.90 (s, 1H), 7.56 (d, J=7.6 Hz, 1H), 7.31 (d, J=6.8 Hz, 1H), 7.28 (s, 1H), 6.31 (s, 1H), 5.72 (s, 2H), 5.55 (s, 1H), 4.93-4.86 (m, 1H), 4.67-4.61 (m, 1H), 4.54-4.46 (m, 1H), 2.41-2.22 (m, 3H), 2.17-2.10 (m, 1H), 1.42 (s, 9H). MS (ESI) m/z: 612.3 [M+1]⁺.

B. tert-butyl(S)-5-amino-4-(5-((4-(3-chloro-5-fluoropyridin-2-yl)-6-fluoro-1H-pyrazolo[3,4-b]pyridin-1-yl)methyl)-4-fluoro-1-oxoisoindolin-2-yl)-5-oxopentanoate. A mixture of tert-butyl(S)-5-amino-4-(4-fluoro-5-((6-fluoro-4-iodo-1H-pyrazolo[3,4-b]pyridin-1-yl)methyl)-1-oxoisoindolin-2-yl)-5-oxopentanoate (0.250 g, 0.41 mmol), tributyl-(3-chloro-5-fluoro-2-pyridyl) stannane (prepared analogously to Intermediate M)(0.206 g, 0.49 mmol), tetrakis(triphenylphosphine) palladium (0)(0.037 g, 0.04 mmol) and copper iodide (0.016 g, 0.08 mmol) in dioxane (5 mL) was stirred at 100° C. for 12 h under nitrogen. The reaction mixture was quenched with aqueous potassium fluoride solution and extracted with ethyl acetate (10 mL×2). The combined organic layers were concentrated under vacuum. The residue was further purified by preparative TLC (60% ethyl acetate in petroleum ether) to give the crude product tert-butyl(S)-5-amino-4-(5-((4-(3-chloro-5-fluoropyridin-2-yl)-6-fluoro-1H-pyrazolo[3,4-b]pyridin-1-yl)methyl)-4-fluoro-1-oxoisoindolin-2-yl)-5-oxopentanoate (0.280 g, 0.46 mmol) as a yellow oil, which was used in the next step directly. MS (ESI) m/z: 615.3 [M+1]⁺.

C. tert-butyl(S)-5-amino-4-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl)-5-oxopentanoate. A mixture of tert-butyl(S)-5-amino-4-(5-((4-(3-chloro-5-fluoropyridin-2-yl)-6-fluoro-1H-pyrazolo[3,4-b]pyridin-1-yl)methyl)-4-fluoro-1-oxoisoindolin-2-yl)-5-oxopentanoate (0.280 g, 0.46 mmol), 3-fluoroazetidine hydrochloride (0.061 g, 0.55 mmol) and N-ethyl-N-isopropylpropan-2-amine (0.118 g, 0.91 mmol) in acetonitrile (5 mL) was stirred at 80° C. for 3 h. The reaction mixture was concentrated under vacuum. The residue was purified by preparative TLC (50% ethyl acetate in petroleum ether) to give the product tert-butyl(S)-5-amino-4-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl)-5-oxopentanoate (0.170 g, 0.25 mmol, 56% yield) as a yellow solid. ¹H NMR (400 MHz, DMSO-d₆) δ 8.78 (d, J=2.4 Hz, 1H), 8.33 (dd, J=2.4, 8.4 Hz, 1H), 7.74 (s, 1H), 7.57 (s, 1H), 7.51 (d, J=7.6 Hz, 1H), 7.36 (t, J=6.8 Hz, 1H), 7.20 (s, 1H), 6.55 (s, 1H), 5.68-5.43 (m, 3H), 4.69 (dd, J=4.0, 10.8 Hz, 1H), 4.64-4.54 (m, 2H), 4.50-4.37 (m, 2H), 4.24-4.10 (m, 2H), 2.21-2.11 (m, 3H), 2.06-1.99 (m, 1H), 1.30 (s, 9H). MS (ESI) m/z: 670.3 [M+1]⁺.

D. (S)-5-amino-4-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl)-5-oxopentanoic acid. To a solution of tert-butyl(S)-5-amino-4-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl)-5-oxopentanoate (0.170 g, 0.25 mmol) in dichloromethane (2 mL) was added TFA (0.02 mL, 0.25 mmol) at 25° C. Then the mixture was stirred at 25° C. for 1 h. The reaction mixture was diluted with water (20 mL) and adjusted to pH 6 with sodium sulfite solution. Then the mixture was extracted with dichloromethane (10 mL×2). The combined organic layers were dried over anhydrous sodium sulfate, filtered, and concentrated under vacuum to give(S)-5-amino-4-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl)-5-oxopentanoic acid (0.155 g, 0.25 mmol, 99.5% yield) as a white solid, which was used in the next step directly.

E. 3-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl) piperidine-2,6-dione. The mixture of(S)-5-amino-4-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl)-5-oxopentanoic acid (0.151 g, 0.25 mmol), N-ethyl-N-isopropylpropan-2-amine (0.1 mL, 0.76 mmol), 4-dimethylaminopyridine (0.031 g, 0.25 mmol) and carbonyl diimidazole (0.082 g, 0.50 mmol) in acetonitrile (3 mL) was stirred at 60° C. for 4 h. The reaction mixture was concentrated under vacuum. The residue was further purified by semi-preparative reverse phase-HPLC (42-62% acetonitrile in water+0.225% formic acid, over 7 min). The desired fractions were lyophilized to afford the product 3-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl) piperidine-2,6-dione (0.050 g, 0.08 mmol, 33% yield) as a white solid. MS (ESI) m/z: 596.4 [M+1]⁺.

F. (R)-3-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl) piperidine-2,6-dione. 3-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl) piperidine-2,6-dione (0.050 g, 0.08 mmol) was separated by SFC separation (Column: DAICEL CHIRALPAK IC (250 mm*30 mm, 10 μm), Mobile phase: Phase A for CO₂, and Phase B for IPA+ACN(NEU); Gradient elution: IPA+ACN(NEU in CO₂ from 70% to 70%, Flow rate: 80 mL/min; 3.5 min, 43 min) to afford two fractions. The fractions of the earlier eluting peak were concentrated under reduced pressure to afford a residue. The residue was re-dissolved in water (20 mL) and acetonitrile (5 mL), then lyophilized to afford Example 61 (S)-3-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl) piperidine-2,6-dione (0.017 g, 0.03 mmol, 33% yield, 99.5% purity, 100% ee %) as an off-white solid. ¹H NMR (400 MHz, DMSO-d₆) δ 11.14-10.91 (m, 1H), 8.78 (d, J=2.4 Hz, 1H), 8.39-8.30 (m, 1H), 7.74 (s, 1H), 7.56 (d, J=7.6 Hz, 1H), 7.38 (t, J=6.8 Hz, 1H), 6.56 (s, 1H), 5.67 (s, 2H), 5.64-5.44 (m, 1H), 5.11 (dd, J=4.8, 13.2 Hz, 1H), 4.58 (d, J=17.6 Hz, 1H), 4.51-4.38 (m, 3H), 4.24-4.10 (m, 2H), 2.97-2.85 (m, 1H), 2.63-2.56 (m, 1H), 2.40 (d, J=4.4 Hz, 1H), 2.03-1.95 (m, 1H). MS (ESI) m/z: 596.3 [M+1]⁺.

The fractions of the later eluting peak were concentrated under vacuum. The residue was re-dissolved in water (20 mL) and acetonitrile (5 mL), then lyophilized to afford Example 109 (R)-3-(5-((4-(3-chloro-5-fluoropyridin-2-yl)-6-(3-fluoroazetidin-1-yl)-1H-pyrazolo[3,4-b]pyridin-1-yl)methyl)-4-fluoro-1-oxoisoindolin-2-yl) piperidine-2,6-dione (0.022 g, 0.04 mmol, 43% yield, 98.5% purity, 98.3% ee %) as an off-white solid. ¹H NMR (400 MHz, DMSO-d₆) δ 11.11-10.91 (m, 1H), 8.78 (d, J=2.4 Hz, 1H), 8.34 (dd, J=2.4, 8.8 Hz, 1H), 7.74 (s, 1H), 7.56 (d, J=7.6 Hz, 1H), 7.38 (t, J=7.2 Hz, 1H), 6.56 (s, 1H), 5.71-5.46 (m, 3H), 5.11 (dd, J=4.8, 12.8 Hz, 1H), 4.62-4.54 (m, 1H), 4.49-4.38 (m, 3H), 4.23-4.11 (m, 2H), 2.95-2.86 (m, 1H), 2.60 (s, 1H), 2.42-2.39 (m, 1H), 2.03-1.95 (m, 1H). MS (ESI) m/z: 596.3 [M+1]⁺.

### Example 78: (3R)-3-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl) piperidine-2,6-dione

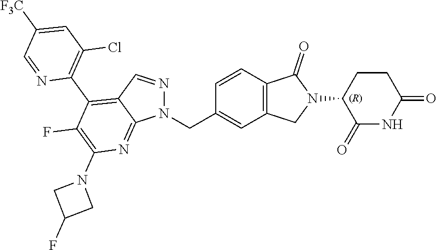

A. tert-butyl (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoate. To a solution of tert-butyl(S)-5-amino-4-(5-((5-fluoro-6-(3-fluoroazetidin-1-yl)-4-iodo-1H-pyrazolo[3,4-b]pyridin-1-yl)methyl)-1-oxoisoindolin-2-yl)-5-oxopentanoate (Intermediate H)(0.200 g, 0.30 mmol) and 3-chloro-2-(tributylstannyl)-5-(trifluoromethyl)pyridine (Intermediate M)(0.282 g, 0.60 mmol) in dioxane (2 mL) was added tetrakis(triphenylphosphine) palladium (0)(0.027 g, 0.03 mmol) and cuprous iodide (0.006 g, 0.03 mmol). The mixture was stirred at 90° C. for 12 h under nitrogen. The mixture was quenched with saturated potassium fluoride aqueous solution (10 mL) and the mixture was stirred for 20 min. Then the mixture was filtered and the filtrate was diluted with water (20 mL), extracted with ethyl acetate (10 mL×3). The combined organic layers were dried over anhydrous sodium sulfate, filtered and concentrated under reduced pressure. Then the residue was purified by preparative TLC (75% ethyl acetate in petroleum ether) to afford the product tert-butyl (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoate (0.140 g, 0.19 mmol, 65% yield) as a yellow oil. ¹H NMR (400 MHz, CDCl₃) δ 8.94 (s, 1H), 8.15 (d, J=2.0 Hz, 1H), 7.80 (d, J=8.0 Hz, 1H), 7.59 (s, 1H), 7.48 (d, J=7.6 Hz, 1H), 7.39 (s, 1H), 6.35 (s, 1H), 5.65 (s, 2H), 541-5.39 (m, 2H), 4.89 (dd, J=6.4, 8.8 Hz, 1H), 4.63-4.51 (m, 2H), 4.47-4.38 (m, 4H), 2.39-2.07 (m, 4H), 1.41 (s, 9H). MS (ESI) m/z: 720.2 [M+1]⁺.

B. (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoic acid. To a solution of tert-butyl (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoate (0.500 g, 0.69 mmol) in dichloromethane (6 mL) was added TFA (2 mL, 26.12 mmol). The mixture was stirred at 25° C. for 2 h. The mixture was adjusted to pH 6 with aqueous sodium bicarbonate solution, diluted with water (10 mL), and extracted with ethyl acetate (15 mL×3). The combined organic layers were dried over anhydrous sodium sulfate, filtered, and concentrated en vacuo to afford (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoic acid (0.450 g, 0.68 mmol, 98% yield) as a yellow oil. The material was used directly in the next step.

C. (3R)-3-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl) piperidine-2,6-dione. To a solution of (4S)-5-amino-4-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl)-5-oxopentanoic acid (0.070 g, 0.11 mmol) in acetonitrile (2 mL) was added N-ethyl-N-isopropylpropan-2-amine (0.1 mL, 0.32 mmol). Then di(1H-imidazol-1-yl) methanone (0.034 g, 0.21 mmol) and N,N-dimethylpyridin-2-amine (0.026 g, 0.21 mmol) was added and the mixture was stirred at 60° C. for 12 h under nitrogen. The residue was purified by semi-preparative reverse phase HPLC (47-77% acetonitrile in water+0.225% formic acid, over 7 min). The desired fractions were concentrated en vacuo and then lyophilized to afford the product. The product was separated by SFC separation (Column: DAICEL CHIRALCEL OX (250 mm×30 mm, 10 μm), Mobile phase: Phase A for CO₂, and Phase B for IPA (0.05% DEA); Gradient elution: IPA (0.05% DEA) in CO₂ from 60% to 60%, Flow rate: 80 mL/min; 5 min, 40 min) to afford two fractions. Fraction 2 was concentrated under vacuum to afford a residue. The residue was diluted with water (10 mL) and extracted with ethyl acetate (10 mL×3). The combined organic layers were dried over anhydrous sodium sulfate, filtered and concentrated under reduced pressure. The residue was re-dissolved in water (5 mL) and acetonitrile (5 mL), then lyophilized to afford the product (3R)-3-(5-((4-(3-chloro-5-(trifluoromethyl)pyridin-2-yl)-5-fluoro-6-(3-fluoroazetidin-1-yl)-1H-pyrazolo[3,4-b]pyridin-1-yl)methyl)-1-oxoisoindolin-2-yl) piperidine-2,6-dione (0.026 g, 0.04 mmol, 39% yield, 99.6% purity, 100% ee %) as white solid. ¹H NMR (400 MHz, DMSO-d₆) δ 10.98 (s, 1H), 9.16 (d, J=1.2 Hz, 1H), 8.77 (d, J=1.2 Hz, 1H), 7.75-7.67 (m, 2H), 7.52 (s, 1H), 7.44 (d, J=8.0 Hz, 1H), 5.67-5.42 (m, 3H), 5.09 (dd, J=4.8, 13.2 Hz, 1H), 4.68-4.52 (m, 2H), 4.49-4.38 (m, 1H), 4.37-4.23 (m, 3H), 2.98-2.82 (m, 1H), 2.63-2.55 (m, 1H), 2.42-2.29 (m, 1H), 2.03-1.94 (m, 1H). MS (ESI) m/z: 646.3 [M+1]⁺.

### Example 87: (R)-3-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione

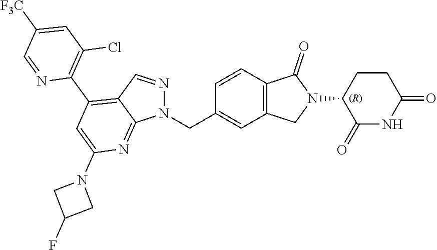

A. tert-butyl(S)-5-amino-4-(5-((4-(3-chloro-5-(trifluoromethyl)pyridin-2-yl)-6-fluoro-1H-pyrazolo[3,4-b]pyridin-1-yl)methyl)-1-oxoisoindolin-2-yl)-5-oxopentanoate. To a solution of tert-butyl(S)-5-amino-4-(5-((6-fluoro-4-iodo-1H-pyrazolo[3,4-b]pyridin-1-yl)methyl)-1-oxoisoindolin-2-yl)-5-oxopentanoate (Intermediate D)(0.250 g, 0.42 mmol) and 3-chloro-2-(tributylstannyl)-5-(trifluoromethyl)pyridine (Intermediate M)(0.238 g, 0.51 mmol) in 1,4-dioxane (10 mL) was added tetrakis(triphenylphosphine) palladium (0.039 g, 0.04 mmol) and cuprous iodide (0.016 g, 0.08 mmol). The mixture was stirred for 12 hours at 100° C. under nitrogen. The reaction mixture was quenched by aqueous potassium fluoride and filtered. The filtrate was concentrated to give a residue. The residue was purified by preparative TLC (50% ethyl acetate in petroleum ether) to afford tert-butyl(S)-5-amino-4-(5-((4-(3-chloro-5-(trifluoromethyl)pyridin-2-yl)-6-fluoro-1H-pyrazolo[3,4-b]pyridin-1-yl)methyl)-1-oxoisoindolin-2-yl)-5-oxopentanoate (0.180 g, 0.28 mmol, 66% yield) as a yellow oil. ¹H NMR (400 MHz, DMSO-d₆) δ 9.18 (s, 1H), 8.80 (d, J=1.2 Hz, 1H), 8.20 (s, 1H), 7.67 (d, J=8.0 Hz, 1H), 7.56-7.48 (m, 2H), 7.44-7.38 (m, 2H), 7.16 (s, 1H), 5.77 (s, 2H), 4.69 (dd, J=4.0, 10.4 Hz, 1H), 4.59-4.36 (m, 2H), 2.17-2.07 (m, 3H), 1.97-1.88 (m, 1H), 1.29 (s, 9H). MS (ESI) m/z: 647.3 [M+1]⁺.

B. tert-butyl(S)-5-amino-4-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate. To a solution of tert-butyl(S)-5-amino-4-(5-((4-(3-chloro-5-(trifluoromethyl)pyridin-2-yl)-6-fluoro-1H-pyrazolo[3,4-b]pyridin-1-yl)methyl)-1-oxoisoindolin-2-yl)-5-oxopentanoate (0.180 g, 0.28 mmol) and 3-fluoroazetidine; hydrochloride (0.037 g, 0.33 mmol) in acetonitrile (10 mL) was added N-ethyl-N-isopropylpropan-2-amine (0.2 mL, 0.83 mmol). The reaction was stirred for 12 hours at 80° C. The reaction was adjusted to pH 6 with 1 M phosphoric acid solution and extracted with ethyl acetate (50 mL×3). Then organic layers were dried over anhydrous sodium sulfate, filtered and concentrated to give tert-butyl(S)-5-amino-4-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.170 g, 0.24 mmol, 87% yield) as yellow solid and the crude product was used in next step directly. MS (ESI) m/z: 702.3 [M+1]⁺.

C. (S)-5-amino-4-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoic acid. To a solution of tert-butyl(S)-5-amino-4-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.170 g, 0.24 mmol) in dichloromethane (6 mL) was added 2,2,2-TFA (2.0 mL, 26.12 mmol) and stirred for 2 hours at 25° C. The reaction was adjusted to pH 6 with aqueous sodium bicarbonate solution and extracted with ethyl acetate (50 mL×3). The combined organic layers were dried over anhydrous sodium sulfate, filtered, and concentrated to give(S)-5-amino-4-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoic acid (0.140 g, 0.22 mmol, 90% yield) as a yellow solid.

D. (R)-3-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione. To a solution of(S)-5-amino-4-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl)-5-oxopentanoic acid (0.140 g, 0.22 mmol) and N-ethyl-N-isopropylpropan-2-amine (0.1 mL, 0.65 mmol) in acetonitrile (15 mL) was added N,N′-carbonyl diimidazole (0.088 g, 0.54 mmol) and N,N-dimethylpyridin-4-amine (0.029 mg, 0.24 mmol). The reaction was stirred for 12 hours at 80° C. The mixture was purified by semi-preparative reverse phase HPLC (45-75% acetonitrile in water+0.225% formic acid, 7 min). The desired fractions were combined and extracted with ethyl acetate (50 mL×2). The combined organic layers were dried over anhydrous sodium sulfate, filtered, and concentrated under reduced pressure. The residue was separated by SFC separation (Column: DAICEL CHIRALPAK IC (250 mm×30 mm, 10 μm)), Mobile phase: Phase A: carbon dioxide, Phase B: isophthalic+acetonitrile (0.05% DEA); Gradient elution: isophthalic+acetonitrile (0.05% DEA) in carbon dioxide from 60% to 60%, Flow rate: 80 mL/min; 5.7 min, 50 min) to afford two fractions. Fraction 2 was concentrated under vacuum to afford a residue. The residue was re-dissolved in water (10 mL) and acetonitrile (20 mL), then lyophilized to afford the product (R)-3-(5-((4-(3-chloro-5-(trifluoromethyl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione (0.029 g, 0.05 mmol, 21% yield, 99.4% purity, 100% ee %). ¹H NMR (400 MHz, DMSO-d₆) δ 10.98 (s, 1H), 9.12 (s, 1H), 8.71 (d, J=1.2 Hz, 1H), 7.79 (s, 1H), 7.70 (d, J=7.6 Hz, 1H), 7.50 (s, 1H), 7.42 (d, J=8.0 Hz, 1H), 6.62 (s, 1H), 5.68-5.44 (m, 3H), 5.09 (dd, J=5.2, 13.1 Hz, 1H), 4.55-4.37 (m, 3H), 4.33-4.26 (m, 1H), 4.24-4.10 (m, 2H), 2.96-2.82 (m, 1H), 2.65-2.54 (m, 1H), 2.39-2.30 (m, 1H), 2.03-1.91 (m, 1H). MS (ESI) m/z: 628.2 [M+1]⁺.

### Example 108: (3S)-3-[5-({4-[3-chloro-5-(oxetan-3-yl)pyridin-2-yl]-6-(3-fluoroazetidin-1-yl)-1H-pyrazolo[3,4-b]pyridin-1-yl}methyl)-1-oxo-2,3-dihydro-1H-isoindol-2-yl]piperidine-2,6-dione

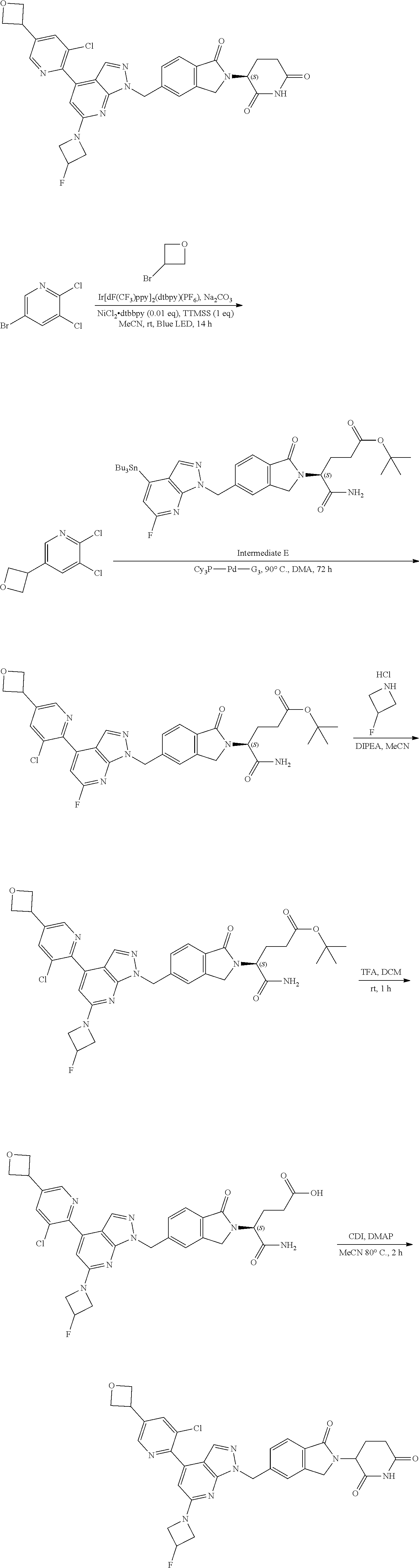

A. 2,3-dichloro-5-(oxetan-3-yl)pyridine. To a 40 mL vial equipped with a stir bar was added 5-bromo-2,3-dichloro-pyridine (2.000 g, 8.81 mmol), 3-bromooxetane (1570 g, 11.46 mmol), Ir[dF(CF₃)ppy]₂ (dtbpy)(PF₆)(0.099 g, 0.09 mmol), NiCl₂·dtbbpy (0.011 g, 0.09 mmol), Tris(trimethylsilyl) silane (TTMSS 2.192 g, 8.81 mmol), sodium carbonate (1869 g, 17.63 mmol) in acetonitrile (20 mL). The vial was placed under nitrogen and sealed. The reaction was stirred and irradiated with a 34 W blue LED lamp (7 cm away), with cooling fan to keep the reaction temperature at 25° C. for 14 h. The reaction mixture was concentrated under reduced pressure. The residue was purified by silica gel chromatography (0-70% ethyl acetate in petroleum ether). The residue was further purified by semi-preparative reverse phase-HPLC (25-55% acetonitrile in water+0.225% formic acid, over 10 min). The desired fractions were combined and extracted with ethyl acetate (50 mL×2). The combined organic layers were dried over anhydrous sodium sulfate solid, filtered, and concentrated under reduced pressure to give the product 2,3-dichloro-5-(oxetan-3-yl)pyridine (0.400 g, 1.96 mmol, 22% yield) as a yellow solid. ¹H NMR (400 MHz, CDCl₃) δ 8.27 (d, J=2.0 Hz, 1H), 7.98 (d, J=2.0 Hz, 1H), 5.14 (dd, J=6.4, 8.0 Hz, 2H), 4.69 (t, J=6.4 Hz, 2H), 4.26-4.17 (m, 1H). MS (ESI) m/z: 203.9 [M+1]⁺.

B. tert-butyl(S)-5-amino-4-(5-((4-(3-chloro-5-(oxetan-3-yl)pyridin-2-yl)-6-fluoro-1H-pyrazolo[3,4-b]pyridin-1-yl)methyl)-1-oxoisoindolin-2-yl)-5-oxopentanoate. A mixture of 2,3-dichloro-5-(oxetan-3-yl)pyridine (0.200 g, 0.98 mmol), tert-butyl(S)-5-amino-4-(5-((6-fluoro-4-(tributylstannyl)-1H-pyrazolo[3,4-b]pyridin-1-yl)methyl)-1-oxoisoindolin-2-yl)-5-oxopentanoate (Intermediate E)(0.742 g, 0.98 mmol) and Cy₃P—Pd-G₃ (0.072 g, 0.10 mmol) in N,N-dimethylacetamide (5 mL) was stirred at 90° C. for 72 h under nitrogen. The reaction mixture was diluted with water (50 mL) and extracted with ethyl acetate (20 mL×2). The combined organic layers were concentrated under reduced pressure. The residue was purified by silica gel chromatography (0-16% methyl alcohol in dichloromethane) to give the crude product. The crude product was further purified by semi-preparative reverse phase-HPLC (51-71% acetonitrile in water+0.225% formic acid, over 8 min). The desired fractions were combined and extracted with ethyl acetate (20 mL×2). The combined organic layers were dried over anhydrous sodium sulfate solid, filtered, and concentrated under reduced pressure to give tert-butyl(S)-5-amino-4-(5-((4-(3-chloro-5-(oxetan-3-yl)pyridin-2-yl)-6-fluoro-1H-pyrazolo[3,4-b]pyridin-1-yl)methyl)-1-oxoisoindolin-2-yl)-5-oxopentanoate (0.110 g, 0.17 mmol, 18% yield) as a light yellow solid, which was used in the next step directly. MS (ESI), m/z: 635.3 [M+1]⁺.

C. tert-butyl(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate. A mixture of tert-butyl(S)-5-amino-4-(5-((4-(3-chloro-5-(oxetan-3-yl)pyridin-2-yl)-6-fluoro-1H-pyrazolo[3,4-b]pyridin-1-yl)methyl)-1-oxoisoindolin-2-yl)-5-oxopentanoate (0.110 g, 0.17 mmol), 3-fluoroazetidine hydrochloride (0.023 g, 0.21 mmol) and N-ethyl-N-isopropylpropan-2-amine (0.1 mL, 0.35 mmol) in acetonitrile (3 mL) was stirred at 80° C. for 2 h. The reaction mixture was concentrated under reduced pressure to give the crude product tert-butyl(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.119 g, 0.17 mmol, 99% yield) as a light yellow solid, which was used in the next step directly. MS (ESI) m/z: 690.1 [M+1]⁺.

D. (S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoic acid. A mixture of tert-butyl(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.109 g, 0.16 mmol) and trifluoroacetic acid (0.4 mL, 5.35 mmol) in dichloromethane (2 mL) was stirred at 25° C. for 1 h. The reaction mixture was adjusted pH to 7 with sodium carbonate solution and extracted with ethyl acetate (20 mL×2). The combined organic layers were dried over anhydrous sodium sulfate solid, filtered, and concentrated under reduced pressure to give the crude product(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoic acid (0.100 g, 0.16 mmol, 99.9% yield) as a yellow solid, which was used in the next step directly.

E. (3S)-3-[5-({4-[3-chloro-5-(oxetan-3-yl)pyridin-2-yl]-6-(3-fluoroazetidin-1-yl)-1H-pyrazolo[3,4-b]pyridin-1-yl}methyl)-1-oxo-2,3-dihydro-1H-isoindol-2-yl]piperidine-2,6-dione. The mixture of(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoic acid (0.100 g, 0.16 mmol), 4-dimethylaminopyridine (0.019 g, 0.16 mmol), carbonyl diimidazole (0.051 g, 0.32 mmol) and N-ethyl-N-isopropylpropan-2-amine (0.061 g, 0.47 mmol) in acetonitrile (3 mL) was stirred at 80° C. for 2 h. The reaction mixture was concentrated under reduced pressure. The residue was purified by semi-preparative reverse phase-HPLC (28-58% acetonitrile in water+0.225% formic acid, over 10 min). The desired fractions were lyophilized to afford the crude product. The crude product was further purified by semi-preparative reverse phase-HPLC (28-58% acetonitrile in water+10 mM ammonium bicarbonate, over 10 min). The desired fractions were lyophilized to afford 3-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione (0.010 g, 0.02 mmol, 10% yield, 96.7% purity) as a white solid. MS (ESI) m/z: 616.3 [M+1]⁺.

3-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione (0.010 g, 0.0200 mmol) was separated by SFC separation (Column: REGIS(S,S)WHELK-O1 (250 mm×25 mm, 10 μm), Mobile phase: Phase A for CO₂, and Phase B for MeOH (0.1% NH₃H₂O); Gradient elution: IPA/ACN (0.1% NH₃H₂O) in CO₂ from 60% to 60%, Flow rate: 80 mL/min; 8.8 min, 70 min) to afford two fractions. Fraction 1 was concentrated under reduced pressure. The residue was re-dissolved in water (20 mL) and acetonitrile (5 mL), then lyophilized to afford the product (3S)-3-[5-({4-[3-chloro-5-(oxetan-3-yl)pyridin-2-yl]-6-(3-fluoroazetidin-1-yl)-1H-pyrazolo[3,4-b]pyridin-1-yl}methyl)-1-oxo-2,3-dihydro-1H-isoindol-2-yl]piperidine-2,6-dione (0.004 g, 0.01 mmol, 39% yield, 99.2% purity, 92.3% ee %) as an off-white solid. ¹H NMR (400 MHz, DMSO-d₆) δ 11.09-10.86 (m, 1H), 8.75-8.66 (m, 1H), 8.25 (d, J=1.6 Hz, 1H), 7.76 (s, 1H), 7.69 (d, J=7.6 Hz, 1H), 7.52-7.47 (m, 1H), 7.44-7.38 (m, 1H), 6.57 (s, 1H), 5.66-5.44 (m, 3H), 5.13 (d, J=4.4 Hz, 1H), 4.98 (dd, J=6.4, 8.4 Hz, 2H), 4.73 (t, J=6.4 Hz, 2H), 4.46-4.41 (m, 3H), 4.33-4.25 (m, 1H), 4.23-4.12 (m, 2H), 2.96-2.83 (m, 1H), 2.62-2.59 (m, 1H), 2.38-2.35 (m, 1H), 2.01-1.94 (m, 1H). MS (ESI) m/z: 616.3 [M+1]⁺.

### Example 127: (R)-3-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione

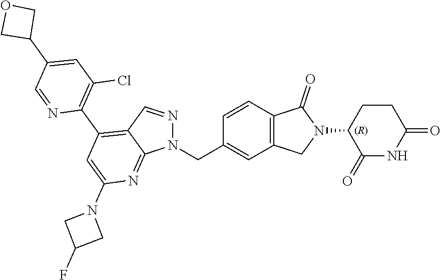

A. tert-butyl(S)-5-amino-4-(5-((4-(3-chloro-5-(oxetan-3-yl)pyridin-2-yl)-6-fluoro-1H-pyrazolo[3,4-b]pyridin-1-yl)methyl)-1-oxoisoindolin-2-yl)-5-oxopentanoate. A mixture of 2,3-dichloro-5-(oxetan-3-yl)pyridine (synthesis described for Example 108)(0.200 g, 0.98 mmol), tert-butyl(S)-5-amino-4-(5-((6-fluoro-4-(tributylstannyl)-1H-pyrazolo[3,4-b]pyridin-1-yl)methyl)-1-oxoisoindolin-2-yl)-5-oxopentanoate (Intermediate E)(0.742 g, 0.98 mmol) and Cy3P-Pd-G3 (0.072 g, 0.10 mmol) in N,N-dimethylacetamide (10 mL) was stirred at 90° C. for 72 h under nitrogen. The reaction mixture was diluted with water (50 mL) and extracted with ethyl acetate (20 mL×2). The combined organic layers were concentrated under vacuum. The residue was purified by silica gel chromatography (0-16% methanol in dichloromethane) to give the crude product. The crude product was further purified by semi-preparative reverse phase-HPLC (51-71% acetonitrile in water+0.225% formic acid, over 8 min). The desired fractions were extracted with ethyl acetate (20 mL×2). The combined organic layers were dried over anhydrous sodium sulfate, filtered, and concentrated under vacuum to give the product tert-butyl(S)-5-amino-4-(5-((4-(3-chloro-5-(oxetan-3-yl)pyridin-2-yl)-6-fluoro-1H-pyrazolo[3,4-b]pyridin-1-yl)methyl)-1-oxoisoindolin-2-yl)-5-oxopentanoate (0.110 g, 0.17 mmol, 18% yield) as a light yellow solid, which was used in the next step directly. MS (ESI) m/z: 635.3 [M+1]⁺.

B. tert-butyl(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate. A mixture of tert-butyl(S)-5-amino-4-(5-((4-(3-chloro-5-(oxetan-3-yl)pyridin-2-yl)-6-fluoro-1H-pyrazolo[3,4-b]pyridin-1-yl)methyl)-1-oxoisoindolin-2-yl)-5-oxopentanoate (0.110 g, 0.17 mmol), 3-fluoroazetidine hydrochloride (0.023 g, 0.21 mmol) and N-ethyl-N-isopropylpropan-2-amine (0.1 mL, 0.35 mmol) in acetonitrile (2 mL) was stirred at 80° C. for 2 h. The reaction mixture was concentrated under vacuum. The residue was purified by semi-preparative reverse phase-HPLC (36-66% acetonitrile in water+10 mM ammonium bicarbonate, over 8 min). The desired fractions were lyophilized to afford the product tert-butyl(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.090 g, 0.13 mmol, 75% yield) as a white solid, which was used in the next step directly. MS (ESI) m/z: 690.3 [M+1]⁺.

C. (S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoic acid. A mixture of tert-butyl(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoate (0.09 g, 0.13 mmol) and TFA (0.5 mL, 6.53 mmol) in dichloromethane (2 mL) was stirred at 25° C. for 1 h. The reaction mixture was adjusted to pH 7 with sodium carbonate solution and extracted with ethyl acetate (20 mL×2). The combined organic layers were dried over anhydrous sodium sulfate, filtered, and concentrated under reduced pressure to give the crude product(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoic acid (0.082 g, 0.13 mmol, 99% yield) as a yellow solid, which was used in the next step directly.

D. (R)-3-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione. A mixture of(S)-5-amino-4-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl)-5-oxopentanoic acid (0.082 g, 0.13 mmol), carbonyl diimidazole (0.042 g, 0.26 mmol), 4-dimethylaminopyridine (0.016 g, 0.13 mmol) and N-ethyl-N-isopropylpropan-2-amine (0.050 g, 0.39 mmol) in acetonitrile (3 mL) was stirred at 60° C. for 2 h. The reaction mixture was concentrated under vacuum. The residue was purified by semi-preparative reverse phase-HPLC (28-58% acetonitrile in water+0.225% formic acid, over 7 min). The desired fractions were lyophilized to afford the product. The product was further separated by SFC separation (Column: REGIS(S,S)WHELK-O1 (250 mm×25 mm, 10 μm), Mobile phase: Phase A for CO₂, and Phase B for isopropanol+acetonitrile (Neu); Gradient elution: isopropanol+acetonitrile (Neu) in CO₂ from 60% to 60%, Flow rate: 80 mL/min; 10.8 min, 70 min) to afford two fractions. Fraction 1 was concentrated under vacuum to afford a residue. The residue was re-dissolved in water (20 mL) and acetonitrile (5 mL), then lyophilized to afford the product 1 (R)-3-(5-((4-(3-chloro-5-(oxetan-3-yl)pyridin-2-yl)-6-(3-fluoroazetidin-1-yl)-1H-pyrazolo[3,4-b]pyridin-1-yl)methyl)-1-oxoisoindolin-2-yl) piperidine-2,6-dione (0.006 g, 0.01 mmol, 8% yield, 99.7% purity, 100% ee %) as an off-white solid. ¹H NMR (400 MHz, DMSO-d₆) δ 10.97 (s, 1H), 8.71 (d, J=1.6 Hz, 1H), 8.25 (d, J=1.6 Hz, 1H), 7.74 (s, 1H), 7.69 (d, J=8.0 Hz, 1H), 7.49 (s, 1H), 7.41 (d, J=8.0 Hz, 1H), 6.56 (s, 1H), 5.69-5.44 (m, 3H), 5.09 (dd, J=5.2, 13.2 Hz, 1H), 4.98 (dd, J=6.0, 8.4 Hz, 2H), 4.73 (t, J=6.4 Hz, 2H), 4.52-4.37 (m, 4H), 4.34-4.26 (m, 1H), 4.24-4.10 (m, 2H), 2.98-2.82 (m, 1H), 2.61-2.56 (m, 1H), 2.42-2.32 (m, 1H), 2.02-1.93 (m, 1H). MS (ESI) m/z: 616.3 [M+1]⁺.

### Example 137:6-(1-{[2-(2,6-dioxopiperidin-3-yl)-1-oxo-2,3-dihydro-1H-isoindol-5-yl]methyl}-6-[(propan-2-yl)amino]-1H-pyrazolo[3,4-b]pyridin-4-yl)-5-fluoropyridine-3-carbonitrile

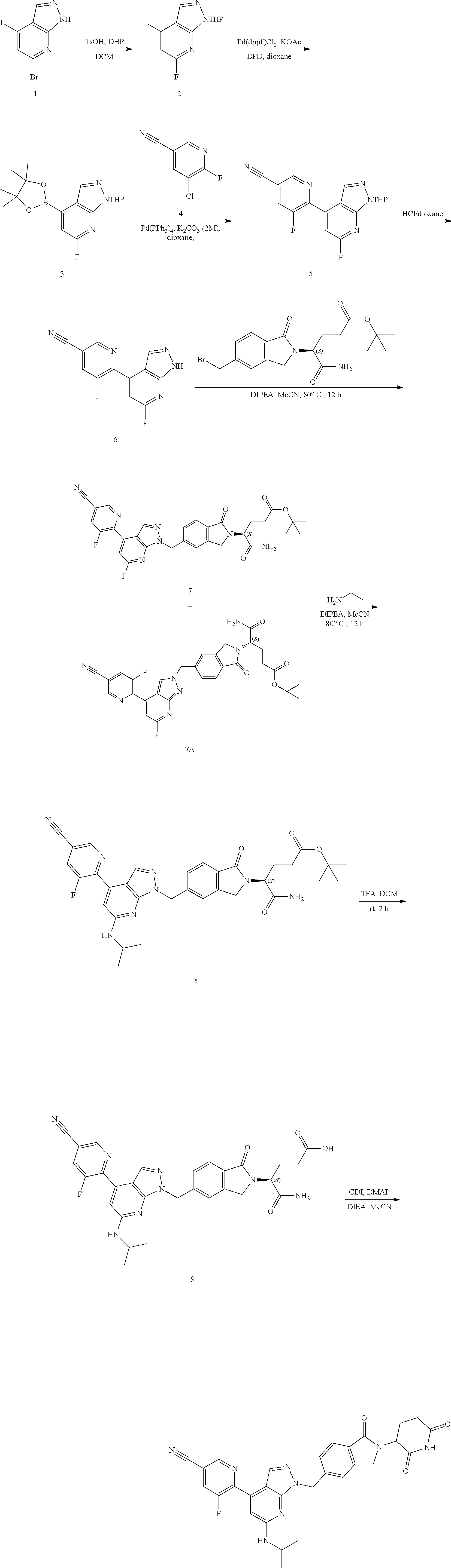

A. 6-fluoro-4-iodo-1-(tetrahydro-2H-pyran-2-yl)-1H-pyrazolo[3,4-b]pyridine. A mixture of 6-fluoro-4-iodo-1H-pyrazolo[3,4-b]pyridine (1.000 g, 3.80 mmol, Intermediate A), 3,4-dihydro-2H-pyran (0.640 g, 7.60 mmol) and 4-methylbenzenesulfonic acid (0.065 g, 0.38 mmol) in dichloromethane (10 mL) was stirred at 25° C. for 2 h. The reaction mixture was diluted with water (20 mL) and adjusted pH to 7 with sodium carbonate solid. Then the mixture was extracted with dichloromethane (10 mL×2). The combined organic layers were dried over anhydrous sodium sulfate solid, filtered, and concentrated under reduced pressure. The residue was purified by silica gel chromatography (0-17% ethyl acetate in petroleum ether) to give 6-fluoro-4-iodo-1-(tetrahydro-2H-pyran-2-yl)-1H-pyrazolo[3,4-b]pyridine (1.040 g, 3.00 mmol, 79% yield. ¹H NMR (400 MHz, CDCl₃) δ 7.92 (s, 1H), 7.26 (d, J=1.2 Hz, 1H), 5.93 (dd, J=2.4, 10.6 Hz, 1H), 4.18-4.03 (m, 1H), 3.85-3.75 (m, 1H), 2.67-2.53 (m, 1H), 2.23-2.09 (m, 1H), 2.02-1.89 (m, 1H), 1.83-1.73 (m, 2H), 1.62-1.47 (m, 1H).

B. 6-fluoro-1-(tetrahydro-2H-pyran-2-yl)-4-(4,4,5,5-tetramethyl-1,3,2-dioxaborolan-2-yl)-1H-pyrazolo[3,4-b]pyridine. A mixture of 6-fluoro-4-iodo-1-(tetrahydro-2H-pyran-2-yl)-1H-pyrazolo[3,4-b]pyridine (1.040 g, 3.00 mmol), bis(pinacolato)diboron (1.141 g, 4.49 mmol), (1,1′-bis(diphenylphosphino) ferrocene) palladium (II) dichloride (0.245 g, 0.30 mmol) and potassium acetate (0.588 g, 5.99 mmol) in dioxane (2 mL) was stirred at 110° C. for 12 h under nitrogen. The reaction mixture was purified by prep-TLC (0-30% ethyl acetate in petroleum ether) to give the product 6-fluoro-1-(tetrahydro-2H-pyran-2-yl)-4-(4,4,5,5-tetramethyl-1,3,2-dioxaborolan-2-yl)-1H-pyrazolo[3,4-b]pyridine (0.560 g, 1.61 mmol, 54% yield) as a brown oil. ¹H NMR (400 MHz, CDCl₃) δ 8.33 (s, 1H), 7.19 (d, J=1.2 Hz, 1H), 5.98 (dd, J=2.4, 10.6 Hz, 1H), 4.14-4.11 (m, 1H), 3.84-3.79 (m, 1H), 2.63 (ddt, J=4.4, 10.8, 12.8 Hz, 1H), 1.96-1.92 (m, 1H), 1.85-1.75 (m, 3H), 1.64-1.61 (m, 1H), 1.40 (s, 12H).

C. 5-chloro-6-(6-fluoro-1-(tetrahydro-2H-pyran-2-yl)-1H-pyrazolo[3,4-b]pyridin-4-yl)nicotinonitrile. A mixture of 6-fluoro-1-(tetrahydro-2H-pyran-2-yl)-4-(4,4,5,5-tetramethyl-1,3,2-dioxaborolan-2-yl)-1H-pyrazolo[3,4-b]pyridine (0.560 g, 1.61 mmol), 6-chloro-5-fluoro-pyridine-3-carbonitrile (0.202 g, 1.29 mmol), tetrakis(triphenylphosphine) palladium (0)(0.137 g, 0.16 mmol) and 2 M potassium carbonate (1.6 mL, 3.23 mmol) in dioxane (20 mL) was stirred at 85° C. for 12 h under nitrogen. The reaction mixture was diluted with water (20 mL) and extracted with ethyl acetate (10 mL×2). The combined organic layers were dried over anhydrous sodium sulfate solid, filtered, and concentrated under reduced pressure. The residue was purified by prep-TLC (30% ethyl acetate in petroleum ether) to give 5-chloro-6-(6-fluoro-1-(tetrahydro-2H-pyran-2-yl)-1H-pyrazolo[3,4-b]pyridin-4-yl)nicotinonitrile (0.175 g, 0.51 mmol, 31.8% yield) as a yellow solid. ¹H NMR (400 MHz, CDCl₃) δ 8.99-8.92 (m, 1H), 8.49 (s, 1H), 7.93 (dd, J=1.6, 10.0 Hz, 1H), 7.43 (d, J=1.2 Hz, 1H), 6.06 (dd, J=2.4, 10.8 Hz, 1H), 4.15 (td, J=2.0, 11.6 Hz, 1H), 3.84 (dt, J=2.4, 11.6 Hz, 1H), 2.72-2.58 (m, 1H), 2.25-2.12 (m, 1H), 2.05-1.94 (m, 1H), 1.85-1.78 (m, 2H), 1.69-1.64 (m, 1H). MS (ESI) m/z: 258.1 [M−84]⁺.

D. 5-fluoro-6-(6-fluoro-1H-pyrazolo[3,4-b]pyridin-4-yl)nicotinonitrile. A mixture of 5-chloro-6-(6-fluoro-1-(tetrahydro-2H-pyran-2-yl)-1H-pyrazolo[3,4-b]pyridin-4-yl)nicotinonitrile (0.175 g, 0.51 mmol) in 4 M hydrogen chloride/dioxane (3.0 mL, 12.00 mmol) was stirred at 25° C. for 2 h. The reaction mixture was diluted with water (20 mL) and extracted with ethyl acetate (10 mL×2). The combined organic layers were dried over anhydrous sodium sulfate solid, filtered, and concentrated under reduced pressure. The residue was purified by prep-TLC (30% ethyl acetate in petroleum ether) to give 5-fluoro-6-(6-fluoro-1H-pyrazolo[3,4-b]pyridin-4-yl)nicotinonitrile (0.080 g, 0.31 mmol, 61% yield) as a light yellow solid. MS (ESI) m/z: 258.1 [M+1]⁺.

E. tert-butyl(S)-5-amino-4-(5-((4-(5-cyano-3-fluoropyridin-2-yl)-6-fluoro-1H-pyrazolo[3,4-b]pyridin-1-yl)methyl)-1-oxoisoindolin-2-yl)-5-oxopentanoate. The mixture of 5-fluoro-6-(6-fluoro-1H-pyrazolo[3,4-b]pyridin-4-yl)nicotinonitrile (0.080 g, 0.31 mmol), tert-butyl(S)-5-amino-4-(5-(bromomethyl)-1-oxoisoindolin-2-yl)-5-oxopentanoate (0.154 g, 0.37 mmol, Intermediate C) and N-ethyl-N-isopropylpropan-2-amine (0.080 g, 0.62 mmol) in acetonitrile (2 mL) was stirred at 80° C. for 12 h. The reaction mixture was purified by semi-preparative reverse phase-HPLC (43-73% acetonitrile in water+0.225% formic acid, over 10 min). Then the collection fractions were extracted with ethyl acetate (20 mL×2). The combined organic layers were dried over anhydrous sodium sulfate solid, filtered, and concentrated under reduced pressure to give the products: product 1 tert-butyl(S)-5-amino-4-(5-((4-(5-cyano-3-fluoropyridin-2-yl)-6-fluoro-1H-pyrazolo[3,4-b]pyridin-1-yl)methyl)-1-oxoisoindolin-2-yl)-5-oxopentanoate (0.071 g, 0.12 mmol, 38.8% yield) as a light yellow solid. ¹H NMR (400 MHz, DMSO-d₆) δ 9.18 (s, 1H), 8.76 (dd, J=1.6, 10.8 Hz, 1H), 8.47 (s, 1H), 7.66 (d, J=8.0 Hz, 1H), 7.52 (s, 2H), 7.47 (s, 1H), 7.38 (d, J=8.0 Hz, 1H), 7.16 (s, 1H), 5.78 (s, 2H), 4.69 (dd, J=4.0, 10.4 Hz, 1H), 4.60-4.48 (m, 1H), 4.46-4.35 (m, 1H), 2.18-2.08 (m, 3H), 1.97-1.89 (m, 1H), 1.29 (s, 9H). MS (ESI) m/z: 588.2 [M+1]. Product 2 tert-butyl(S)-5-amino-4-(5-((4-(5-cyano-3-fluoropyridin-2-yl)-6-fluoro-2H-pyrazolo[3,4-b]pyridin-2-yl)methyl)-1-oxoisoindolin-2-yl)-5-oxopentanoate (0.052 g, 0.09 mmol, 28.5% yield) as a light yellow solid. ¹H NMR (400 MHz, DMSO-d₆) δ 9.15 (s, 1H), 8.99 (s, 1H), 8.75 (dd, J=1.6, 11.2 Hz, 1H), 7.69 (d, J=7.6 Hz, 1H), 7.58 (s, 1H), 7.54 (s, 1H), 7.51-7.43 (m, 2H), 7.16 (s, 1H), 5.84 (s, 2H), 4.69 (dd, J=4.0, 10.4 Hz, 1H), 4.60-4.52 (m, 1H), 4.48-4.39 (m, 1H), 2.15-2.10 (m, 3H), 1.98-1.90 (m, 1H), 1.30 (s, 9H). MS (ESI) m/z: 588.2 [M+1]⁺.

F. tert-butyl(S)-5-amino-4-(5-((4-(5-cyano-3-fluoropyridin-2-yl)-6-(isopropylamino)-1H-pyrazolo[3,4-b]pyridin-1-yl)methyl)-1-oxoisoindolin-2-yl)-5-oxopentanoate. A mixture of tert-butyl(S)-5-amino-4-(5-((4-(5-cyano-3-fluoropyridin-2-yl)-6-fluoro-1H-pyrazolo[3,4-b]pyridin-1-yl)methyl)-1-oxoisoindolin-2-yl)-5-oxopentanoate (0.071 g, 0.12 mmol), propan-2-amine (0.014 g, 0.24 mmol) and N-ethyl-N-isopropylpropan-2-amine (0.1 mL, 0.24 mmol) in acetonitrile (2 mL) was stirred at 80° C. for 12 h. The reaction mixture was diluted with water (20 mL) and extracted with ethyl acetate (5 mL×2). The combined organic layers were dried over anhydrous sodium sulfate solid, filtered, and concentrated under reduced pressure to give the crude product tert-butyl(S)-5-amino-4-(5-((4-(5-cyano-3-fluoropyridin-2-yl)-6-(isopropylamino)-1H-pyrazolo[3,4-b]pyridin-1-yl)methyl)-1-oxoisoindolin-2-yl)-5-oxopentanoate (0.075 g, 0.12 mmol, 99% yield) as a yellow solid. MS (ESI) m/z: 627.4 [M+1]⁺.

G. (S)-5-amino-4-(5-((4-(5-cyano-3-fluoropyridin-2-yl)-6-(isopropylamino)-1H-pyrazolo[3,4-b]pyridin-1-yl)methyl)-1-oxoisoindolin-2-yl)-5-oxopentanoic acid. To a solution of tert-butyl(S)-5-amino-4-(5-((4-(5-cyano-3-fluoropyridin-2-yl)-6-(isopropylamino)-1H-pyrazolo[3,4-b]pyridin-1-yl)methyl)-1-oxoisoindolin-2-yl)-5-oxopentanoate (0.075 g, 0.12 mmol) in dichloromethane (2 mL) was added trifluoroacetic acid (0.5 mL, 6.73 mmol) at 25° C. Then the mixture was stirred at 25° C. for 2 h. The reaction mixture was diluted with water (20 mL) and extracted with ethyl acetate (10 mL×2). The combined organic extracts were dried over anhydrous sodium sulfate solid, filtered, and concentrated under reduced pressure to give the crude product(S)-5-amino-4-(5-((4-(5-cyano-3-fluoropyridin-2-yl)-6-(isopropylamino)-1H-pyrazolo[3,4-b]pyridin-1-yl)methyl)-1-oxoisoindolin-2-yl)-5-oxopentanoic acid (0.068 g, 0.12 mmol, 99.6% yield) as a yellow solid. MS (ESI) m/z: 571.3 [M+1]⁺.

H. 6-(1-{[2-(2,6-dioxopiperidin-3-yl)-1-oxo-2,3-dihydro-1H-isoindol-5-yl]methyl}-6-[(propan-2-yl)amino]-1H-pyrazolo[3,4-b]pyridin-4-yl)-5-fluoropyridine-3-carbonitrile. A mixture of(S)-5-amino-4-(5-((4-(5-cyano-3-fluoropyridin-2-yl)-6-(isopropylamino)-1H-pyrazolo[3,4-b]pyridin-1-yl)methyl)-1-oxoisoindolin-2-yl)-5-oxopentanoic acid (0.068 g, 0.12 mmol), 4-dimethylaminopyridine (0.015 g, 0.12 mmol), carbonyl diimidazole (0.039 g, 0.24 mmol), and N-ethyl-N-isopropylpropan-2-amine (0.031 g, 0.24 mmol) in acetonitrile (2 mL) was stirred at 80° C. for 2 h. The reaction mixture was filtered and the filtrate was purified semi-preparative reverse phase-HPLC (36-66% acetonitrile in water+0.225% formic acid, over 10 min). The desired fractions were lyophilized to afford 6-(1-{[2-(2,6-dioxopiperidin-3-yl)-1-oxo-2,3-dihydro-1H-isoindol-5-yl]methyl}-6-[(propan-2-yl)amino]-1H-pyrazolo[3,4-b]pyridin-4-yl)-5-fluoropyridine-3-carbonitrile (0.016 g, 0.03 mmol, 23.9% yield, 96% purity) as a yellow solid. ¹H NMR (400 MHz, DMSO-d₆) δ 11.09-10.80 (m, 1H), 9.09 (s, 1H), 8.63 (dd, J=1.6, 11.2 Hz, 1H), 8.46 (s, 1H), 7.98 (s, 1H), 7.67 (d, J=7.6 Hz, 1H), 7.50 (s, 1H), 7.43-7.31 (m, 2H), 6.97 (s, 1H), 5.60 (s, 2H), 5.08 (dd, J=5.2, 13.2 Hz, 1H), 4.44-4.38 (m, 1H), 4.33-4.24 (m, 1H), 4.23-4.13 (m, 1H), 2.94-2.84 (m, 1H), 2.59 (d, J=2.8 Hz, 1H), 2.44-2.35 (m, 1H), 2.02-1.92 (m, 1H), 1.21 (dd, J=2.0, 6.4 Hz, 6H). MS (ESI) m/z: 553.2 [M+1]⁺.

### Example 140: (3S)-3-(5-((4-(3-chloro-5-(trifluoromethyl)pyridin-2-yl)-5-fluoro-6-((S)-3-hydroxy-3-methylpyrrolidin-1-yl)-1H-pyrazolo[3,4-b]pyridin-1-yl)methyl)-1-oxoisoindolin-2-yl) piperidine-2,6-dione

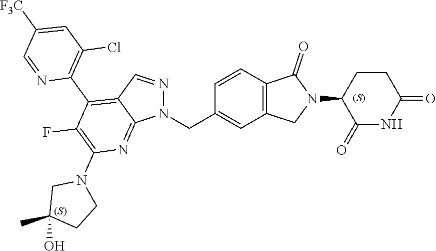

A. tert-butyl(S)-5-amino-4-(5-((6-((S)-3-hydroxy-3-methylpyrrolidin-1-yl)-4-iodo-1H-pyrazolo[3,4-b]pyridin-1-yl)methyl)-1-oxoisoindolin-2-yl)-5-oxopentanoate. The mixture of tert-butyl(S)-5-amino-4-(5-((6-fluoro-4-iodo-1H-pyrazolo[3,4-b]pyridin-1-yl)methyl)-1-oxoisoindolin-2-yl)-5-oxopentanoate (Intermediate C)(0.200 g, 0.34 mmol), (S)-3-methylpyrrolidin-3-ol (0.038 g, 0.37 mmol) and N-ethyl-N-isopropylpropan-2-amine (0.2 mL, 1.01 mmol) in acetonitrile (10 mL) was stirred for 12 hours at 50° C. The reaction mixture was purified by semi-preparative reverse phase-HPLC (40-70% acetonitrile in water+0.225% formic acid, over 10 min). The desired fractions were combined and extracted with ethyl acetate (30 mL×2). The combined organic layers were dried over anhydrous sodium sulfate, filtered, and concentrated under reduced pressure to give the product tert-butyl(S)-5-amino-4-(5-((6-((S)-3-hydroxy-3-methylpyrrolidin-1-yl)-4-iodo-1H-pyrazolo[3,4-b]pyridin-1-yl)methyl)-1-oxoisoindolin-2-yl)-5-oxopentanoate (0.190 g, 0.28 mmol, 84% yield) as a yellow solid. MS (ESI) m/z: 675.2 [M+1]⁺.

B. tert-butyl(S)-5-amino-4-(5-((5-fluoro-6-((S)-3-hydroxy-3-methylpyrrolidin-1-yl)-4-iodo-1H-pyrazolo[3,4-b]pyridin-1-yl)methyl)-1-oxoisoindolin-2-yl)-5-oxopentanoate. To a solution of tert-butyl(S)-5-amino-4-(5-((6-((S)-3-hydroxy-3-methylpyrrolidin-1-yl)-4-iodo-1H-pyrazolo[3,4-b]pyridin-1-yl)methyl)-1-oxoisoindolin-2-yl)-5-oxopentanoate (0.190 g, 0.28 mmol) and silver carbonate (0.172 g, 0.62 mmol) in acetonitrile (2 mL) was added a solution of 1-chloromethyl-4-fluoro-1,4-diazoniabicyclo[2.2.2]octane bis(Tetrafluoroborate)(0.110 g, 0.28 mmol) in water (1 mL) The reaction mixture was divided into seven batches and stirred for 2 hours at 0° C. The reaction mixture was filtered and concentrated to remove acetonitrile. The mixture was diluted with water and extracted with ethyl acetate (10 mL×2). The combined organic layers were concentrated under vacuum. The residue was purified by semi-preparative reverse phase-HPLC (50-80% acetonitrile in water+0.225% formic acid, over 7 min). The desired fractions were combined and extracted with ethyl acetate (30 mL×2). The combined organic layers were dried over anhydrous sodium sulfate, filtered, and concentrated under vacuum to give tert-butyl(S)-5-amino-4-(5-((5-fluoro-6-((S)-3-hydroxy-3-methylpyrrolidin-1-yl)-4-iodo-1H-pyrazolo[3,4-b]pyridin-1-yl)methyl)-1-oxoisoindolin-2-yl)-5-oxopentanoate (0.120 g, 0.17 mmol, 56% yield) as a white solid. ¹H NMR (400 MHz, CDCl₃) δ 7.77 (d, J=8.0 Hz, 1H), 7.58 (s, 1H), 7.45 (d, J=8.0 Hz, 1H), 7.38 (s, 1H), 6.35 (s, 1H), 5.55 (s, 2H), 5.46 (s, 1H), 4.87 (dd, J=6.0, 8.8 Hz, 1H), 4.43 (q, J=17.2 Hz, 2H), 3.98-3.83 (m, 2H), 3.77 (dd, J=2.0, 12.0 Hz, 1H), 3.67-3.60 (m, 1H), 2.35-2.21 (m, 3H), 2.14-2.03 (m, 3H), 1.53 (s, 3H), 1.40 (s, 9H). MS (ESI) m/z: 693.1 [M+1]⁺.

C. tert-butyl(S)-5-amino-4-(5-((5-fluoro-6-((S)-3-hydroxy-3-methylpyrrolidin-1-yl)-4-(tributylstannyl)-1H-pyrazolo[3,4-b]pyridin-1-yl)methyl)-1-oxoisoindolin-2-yl)-5-oxopentanoate. A mixture of tert-butyl(S)-5-amino-4-(5-((5-fluoro-6-((S)-3-hydroxy-3-methylpyrrolidin-1-yl)-4-iodo-1H-pyrazolo[3,4-b]pyridin-1-yl)methyl)-1-oxoisoindolin-2-yl)-5-oxopentanoate (0.080 g, 0.12 mmol), 1,1,1,2,2,2-hexabutyldistannane (0.500 g, 0.86 mmol), tri-o-tolylphosphane (0.007 g, 0.02 mmol), triethylamine (0.1 mL, 0.29 mmol) and palladium diacetate (0.003 g, 0.01 mmol) in acetonitrile (2 mL) was stirred at 85° C. for 4 h under nitrogen. The mixture was diluted with water (10 mL), extracted with ethyl acetate (10 mL×3). The combined organic layers were dried over anhydrous sodium sulfate, filtered, and concentrated under vacuum. It was purified by aluminium oxide chromatography (neutral aluminium oxide, 100% ethyl acetate) to afford tert-butyl(S)-5-amino-4-(5-((5-fluoro-6-((S)-3-hydroxy-3-methylpyrrolidin-1-yl)-4-(tributylstannyl)-1H-pyrazolo[3,4-b]pyridin-1-yl)methyl)-1-oxoisoindolin-2-yl)-5-oxopentanoate (0.070 g, 0.08 mmol, 71% yield) as a yellow oil. MS (ESI) m/z: 857.3 [M+1]⁺.

D. tert-butyl (4S)-5-amino-4-(5-((4-(3-chloro-5-(trifluoromethyl)pyridin-2-yl)-5-fluoro-6-((S)-3-hydroxy-3-methylpyrrolidin-1-yl)-1H-pyrazolo[3,4-b]pyridin-1-yl)methyl)-1-oxoisoindolin-2-yl)-5-oxopentanoate. A mixture of tert-butyl(S)-5-amino-4-(5-((5-fluoro-6-((S)-3-hydroxy-3-methylpyrrolidin-1-yl)-4-(tributylstannyl)-1H-pyrazolo[3,4-b]pyridin-1-yl)methyl)-1-oxoisoindolin-2-yl)-5-oxopentanoate (0.070 g, 0.08 mmol), 2-bromo-3-chloro-5-(trifluoromethyl)pyridine (0.043 g, 0.16 mmol) and PCy3-Pd-G3 (0.006 g, 0.01 mmol) in N, N-dimethylacetamide (1 mL) was stirred for 30 hours at 90° C. The reaction mixture was quenched with an aqueous potassium fluoride solution and extracted with ethyl acetate (20 mL×2). The combined organic layers concentrated under reduced pressure. The residue was purified by semi-preparative reverse phase-HPLC (25-55% acetonitrile in water+0.225% formic acid, over 10 min). The desired fractions were lyophilized to afford the product tert-butyl (4S)-5-amino-4-(5-((4-(3-chloro-5-(trifluoromethyl)pyridin-2-yl)-5-fluoro-6-((S)-3-hydroxy-3-methylpyrrolidin-1-yl)-1H-pyrazolo[3,4-b]pyridin-1-yl)methyl)-1-oxoisoindolin-2-yl)-5-oxopentanoate (0.020 g, 0.03 mmol, 33% yield) as a yellow solid. MS (ESI) m/z: 746.2 [M+1]⁺.

E. (3S)-3-(5-((4-(3-chloro-5-(trifluoromethyl)pyridin-2-yl)-5-fluoro-6-((S)-3-hydroxy-3-methylpyrrolidin-1-yl)-1H-pyrazolo[3,4-b]pyridin-1-yl)methyl)-1-oxoisoindolin-2-yl) piperidine-2,6-dione. To a solution of tert-butyl (4S)-5-amino-4-(5-((4-(3-chloro-5-(trifluoromethyl)pyridin-2-yl)-5-fluoro-6-((S)-3-hydroxy-3-methylpyrrolidin-1-yl)-1H-pyrazolo[3,4-b]pyridin-1-yl)methyl)-1-oxoisoindolin-2-yl)-5-oxopentanoate (0.018 g, 0.02 mmol) and 4-methylbenzenesulfonic acid (0.021 g, 0.12 mmol) in acetonitrile (2 mL) was stirred for 20 hours at 60° C. The reaction mixture was diluted with water (10 mL) and adjusted pH to 7 with sodium carbonate solid. The mixture was extracted with ethyl acetate (10 mL×2). The combined organic layers were concentrated under reduced pressure. The residue was purified by semi-preparative reverse phase-HPLC (45-75% acetonitrile in water+0.225% formic acid, over 7 min). The desired fractions were lyophilized to afford (35)-3-(5-((4-(3-chloro-5-(trifluoromethyl)pyridin-2-yl)-5-fluoro-6-((S)-3-hydroxy-3-methylpyrrolidin-1-yl)-1H-pyrazolo[3,4-b]pyridin-1-yl)methyl)-1-oxoisoindolin-2-yl) piperidine-2,6-dione (0.006 g, 0.01 mmol, 34% yield, 96.9% purity, 99.9% ee %) as a yellow solid. ¹H NMR (400 MHz, DMSO-d₆) δ 11.06-10.90 (m, 1H), 9.15 (s, 1H), 8.75 (s, 1H), 7.70 (d, J=8.0 Hz, 1H), 7.61 (s, 1H), 7.50 (s, 1H), 7.44 (d, J=8.4 Hz, 1H), 5.61 (s, 2H), 5.08 (dd, J=5.2, 13.2 Hz, 1H), 4.87 (s, 1H), 4.48-4.25 (m, 2H), 3.86-3.75 (m, 2H), 3.67-3.55 (m, 2H), 2.92-2.84 (m, 1H), 2.61 (dd, J=2.4, 4.0 Hz, 1H), 2.40-2.35 (m, 1H), 2.02-1.94 (m, 1H), 1.93-1.85 (m, 2H), 1.36 (s, 3H). MS (ESI) m/z: 672.2 [M+1]⁺.

Each compound in the Table 1 (e.g., Ex #) below can be prepared according to the cited examples in the column “Procedures” (e.g., follow Procedure for Example 3 to synthesize Example 1). One of ordinary skill in the art may appreciate the modifications necessary to synthesize the following compounds using the novel synthetic schemes described above.

TABLE 1

| Ex # | Procedures | Structure | Name | LCMS | NMR |
|---|---|---|---|---|---|
| 1 | 3 | 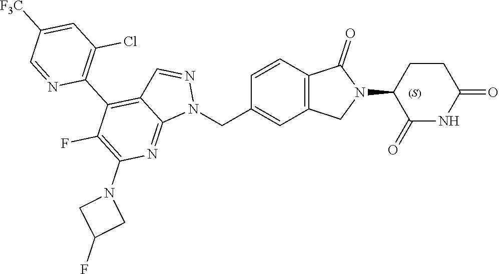 | (3S)-3-[5-({4- [3-chloro-5- (trifluoromethyl) pyridin-2-yl]- 5-fluoro-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 646.3 | ¹H NMR (400 MHz, DMSO-d6) 10.98 (s, 1H), 9.16 (d, J = 1.2 Hz, 1H), 8.77 (d, J = 1.2 Hz, 1H), 7.75-7.67 (m, 2H), 7.52 (s, 1H), 7.44 (d, J = 8.0 Hz, 1H), 5.67-5.42 (m, 3H), 5.09 (dd, J = 4.8, 13.2 Hz, 1H), 4.68-4.52 (m, 2H), 4.49-4.38 (m, 1H), 4.37-4.23 (m, 3H), 2.98-2.82 (m, 1H), 2.63- 2.55 (m, 1H), 2.42- 2.29 (m, 1H), 2.03-1.94 (m, 1H) |
| 2 | 2 | 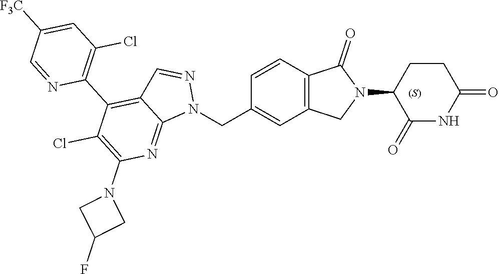 | (3S)-3-[5-({5- chloro-4-[3- chloro-5- (trifluoromethyl) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 662.1 | ¹H NMR (400 MHz, DMSO-d6) 10.98 (br s, 1H), 9.16 (d, J = 1.0 Hz, 1H), 8.79 (d, J = 1.5 Hz, 1H), 7.72 (d, J = 8.0 Hz, 1H), 7.67 (s, 1H), 7.55 (s, 1H), 7.48 (d, J = 7.5 Hz, 1H), 5.73-5.61 (m, 2H), 5.59-5.38 (m, 1H), 5.10 (dd, J = 5.3, 13.3 Hz, 1H), 4.77-4.61 (m, 2H), 4.50-4.26 (m, 4H), 2.98- 2.85 (m, 1H), 2.65- 2.56 (m, 1H), 2.43-2.34 (m, 1H), 2.06-1.94 (m, 1H |
| 3 | 3 | 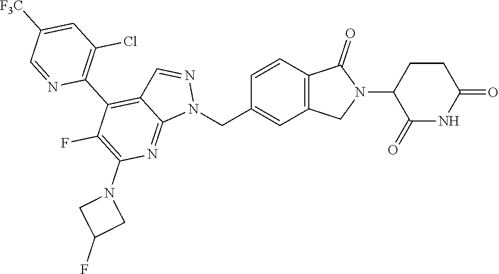 [SMILES: O=C1CCC(N2Cc3cc(Cn4ncc5c(-c6ncc(C(F)(F)F)cc6Cl)c(F)c(N6CC(F)C6)nc54)ccc3C2=O)C(=O)N1] | 3-[5-({4-[3- chloro-5- (trifluoromethyl) pyridin-2-yl]- 5-fluoro-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 646.2 | ¹H NMR (400 MHz, DMSO) 11.17-10.76 (m, 1H), 9.16 (d, J = 1.2 Hz, 1H), 8.78 (d, J = 1.6 Hz, 1H), 7.74-7.68 (m, 2H), 7.53-7.49 (m, 1H), 7.43 (d, J = 7.6 Hz, 1H), 5.64 (s, 2H), 5.62-5.43 (m, 1H), 5.10 (dd, J = 5.2, 12.8 Hz, 1H), 4.65- 4.53 (m, 2H), 4.47-4.41 (m, 1H), 4.37-4.24 (m, 3H), 2.96-2.84 (m, 1H), 2.58-2.56 (m, 1H), 2.43- 2.36 (m, 1H), 2.03- 1.94 (m, 1H) |
| 4 | 2, 32 | 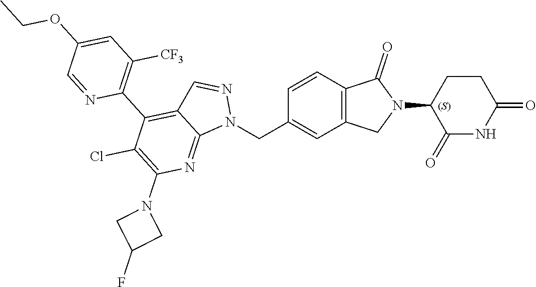 | (3S)-3-[5-({5- chloro-4-[5- ethoxy-3- (trifluoromethyl) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 672.0 | ¹H NMR (400 MHz, DMSO-d6) 10.97 (br s, 1H), 8.70 (d, J = 2.5 Hz, 1H), 7.90 (d, J = 2.5 Hz, 1H), 7.71 (d, J = 7.5 Hz, 1H), 7.55-7.49 (m, 2H), 7.45 (d, J = 8.0 Hz, 1H), 5.72-5.57 (m, 2H), 5.57- 5.34 (m, 1H), 5.10 (dd, J = 5.0, 13.0 Hz, 1H), 4.75-4.57 (m, 2H), 4.49- 4.26 (m, 6H), 2.98- 2.85 (m, 1H), 2.64-2.56 (m, 1H), 2.43-2.35 (m, 1H), 2.02-1.95 (m, 1H), 1.42 (t, J = 6.8 Hz, 3H) |
| 5 | 6, 32, 44, 46, 49 | 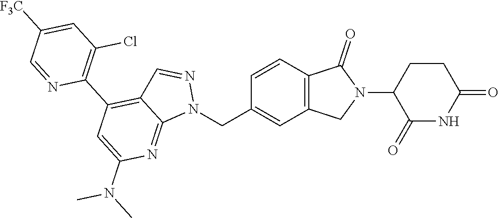 [SMILES: CN(C)c1cc(-c2ncc(C(F)(F)F)cc2Cl)c2cnn(Cc3ccc4c(c3)CN(C3CCC(=O)NC3=O)C4=O)c2n1] | 3-[5-({4-[3- chloro-5- (trifluoromethyl) pyridin-2-yl]- 6- (dimethylamino)- 1H- pyrazolo[3,4- b]pyridin-1- yl}methyl)-1- oxo-2,3- dihydro-1H- isoindol-2- yl]piperidine- 2,6-dione | 598.2 | ¹H NMR (400 MHz, DMSO-d6): 10.99 (s, 1H), 9.06-9.19 (m, 1H), 8.66-8.76 (m, 1H), 7.73 (s, 1H), 7.70 (d, J = 8.0 Hz, 1H), 7.52 (s, 1H), 7.44 (d, J = 8.0 Hz, 1H), 6.89 (s, 1H), 5.65 (s, 2H), 5.10 (dd, J = 13.3, 5.3 Hz, 1H), 4.20-4.49 (m, 2H), 3.18 (s, 6H), 2.82-2.96 (m, 1H), 2.59 (br d, J = 16.0 Hz, 1H), 2.27- 2.43 (m, 1H), 1.90-2.05 (m, 1H) |
| 6 | 6 | 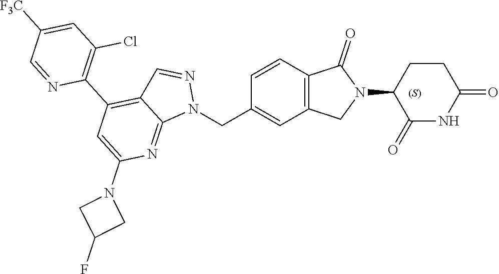 | (3S)-3-[5-({4- [3-chloro-5- (trifluoromethyl) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 628 | ¹H NMR (400 MHz, DMSO-d6) 10.97 (s, 1H), 9.13 (dd, J = 1.0, 2.0 Hz, 1H), 8.78-8.67 (m, 1H), 7.80 (s, 1H), 7.70 (d, J = 7.5 Hz, 1H), 7.51 (s, 1H), 7.43 (d, J = 8.0 Hz, 1H), 6.63 (s, 1H), 5.65 (s, 2H), 5.69-5.45 (m, 1H), 5.10 (dd, J = 5.0, 13.5 Hz, 1H), 4.53-4.39 (m, 3H), 4.35-4.27 (m, 1H), 4.25-4.14 (m, 2H), 2.98- 2.85 (m, 1H), 2.64- 2.56 (m, 1H), 2.43-2.30 (m, 1H), 2.04-1.94 (m, 1H) |
| 7 | 6, 32, 44, 46, 49 | 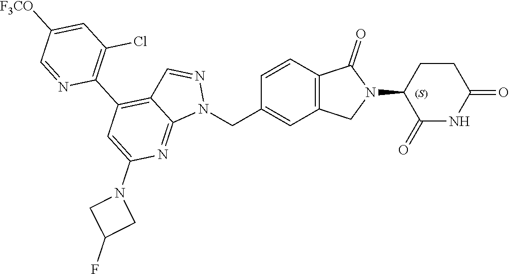 | (S)-3-(5-((4-(3- chloro-5- (trifluoromethoxy) pyridin-2- yl)-6-(3- fluoroazetidin- 1-yl)-1H- pyrazolo[3,4- b]pyridin-1- yl)methyl)-1- oxoisoindolin- 2- yl)piperidine- 2,6-dione | 644.0 | ¹H NMR (DMSO-d6) δ: 10.96 (s, 1H), 8.86 (s, 1H), 8.46 (s, 1H), 7.78 (s, 1H), 7.67-7.72 (m, J = 7.9 Hz, 1H), 7.49 (s, 1H), 7.39-7.44 (m, J = 8.0 Hz, 1H), 6.60 (s, 1H), 5.44- 5.66 (m, 3H), 5.09 (dd, J = 13.1, 5.0 Hz, 1H), 4.38-4.50 (m, 3H), 4.26- 4.34 (m, 1H), 4.12-4.24 (m, 2H), 2.84-2.95 (m, 1H), 2.59 (br d, J = 17.6 Hz, 1H), 2.32-2.39 (m, 1H), 1.93-2.03 (m, 1H) |
| 8 | 3, 9 | 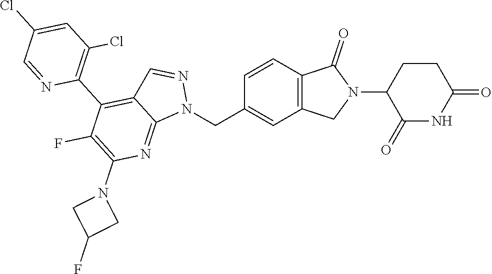 [SMILES: O=C1CCC(N2Cc3cc(Cn4ncc5c(-c6ncc(Cl)cc6Cl)c(F)c(N6CC(F)C6)nc54)ccc3C2=O)C(=O)N1] | 3-(5-{[4-(3,5- dichloropyridin- 2-yl)-5-fluoro- 6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 612.1 | ¹H NMR (400 MHz, DMSO-d6) 10.98 (s, 1H), 8.83 (d, J = 2.4 Hz, 1H), 8.53 (d, J = 2.0 Hz, 1H), 7.71-7.67 (m, 2H), 7.50 (s, 1H), 7.43 (d, J = 8.0 Hz, 1H), 5.63-5.44 (m, 3H), 5.09 (dd, J = 4.8, 13.2 Hz, 1H), 4.63- 4.53 (m, 2H), 4.46-4.42 (m, 1H), 4.33-4.25 (m, 3H), 2.95-2.85 (m, 1H), 2.60 (d, J = 2.4 Hz, 1H), 2.41-2.34 (m, 1H), 2.00- 1.95 (m, 1H) |
| 9 | 9 | 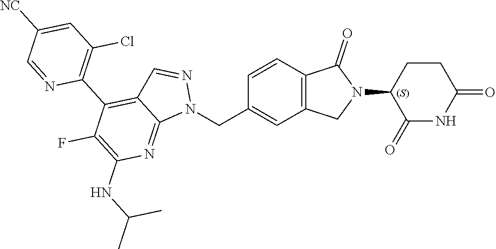 | (3S)-3-(5-{[4- (3-chloro-5- fluoropyridin- 2-yl)-5-fluoro- 6-[(propan-2- yl)amino]-1H- pyrazolo[3,4- b]pyridin-1- yl]methyl}-1- oxo-2,3- dihydro-1H- isoindol-2- yl)piperidine- 2,6-dione | 580.2 | ¹H NMR (400 MHz, DMSO-d6) 10.97 (d, J = 2.0 Hz, 1H), 8.81 (d, J = 2.4 Hz, 1H), 8.37 (dd, J = 2.8, 8.0 Hz, 1H), 7.74- 7.68 (m, 1H), 7.52 (s, 2H), 7.44 (d, J = 8.0 Hz, 1H), 7.18 (d, J = 7.6 Hz, 1H), 5.60 (s, 2H), 5.09 (dd, J = 4.8, 13.2 Hz, 1H), 4.45-4.27 (m, 3H), 2.88 (d, J = 13.6 Hz, 1H), 2.59 (s, 1H), 2.40-2.37 (m, 1H), 1.99-1.95 (m, 1H), 1.25 (dd, J = 2.8, 6.8 Hz, 6H) |
| 10 | 2 | 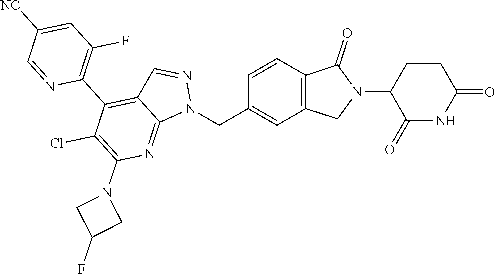 [SMILES: N#Cc1cnc(-c2c(Cl)c(N3CC(F)C3)nc3c2cnn3Cc2ccc3c(c2)CN(C2CCC(=O)NC2=O)C3=O)c(F)c1] | 4-(5-chloro-1- {[2-(2,6- dioxopiperidin- 3-yl)-1-oxo- 2,3-dihydro- 1H-isoindol-5- yl]methyl}-6- (3- fluoroazetidin- 1-yl)-1H- pyrazolo[3,4- b]pyridin-4- yl)-3- fluorobenzonitrile | 602.2 | ¹H NMR (400 MHz, DMSO-d6) 10.97 (s, 1H), 8.14-8.11 (m, 1H), 7.92-7.90 (m, 1H), 7.77- 7.69 (m, 2H), 7.64 (s, 1H), 7.55 (s, 1H), 7.46 (d, J = 7.6 Hz, 1H), 5.64 (s, 2H), 5.56-5.38 (m, 1H), 5.09 (dd, J = 5.6, 13.0 Hz, 1H), 4.73-4.61 (m, 2H), 4.46-4.40 (m, 2H), 4.34-4.28 (m, 2H), 2.94- 2.85 (m, 1H), 2.61- 2.57 (m, 1H), 2.38 (d, J = 6.0 Hz, 1H), 2.01-1.95 (m, 1H) |
| 11 | 6, 32, 44, 46, 49 | 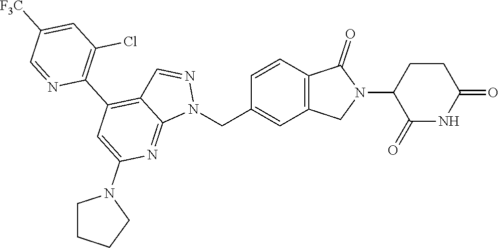 [SMILES: O=C1CCC(N2Cc3cc(Cn4ncc5c(-c6ncc(C(F)(F)F)cc6Cl)cc(N6CCCC6)nc54)ccc3C2=O)C(=O)N1] | 3-[5-({4-[3- chloro-5- (trifluoromethyl) pyridin-2-yl]- 6-(pyrrolidin- 1-yl)-1H- pyrazolo[3,4- b]pyridin-1- yl}methyl)-1- oxo-2,3- dihydro-1H- isoindol-2- yl]piperidine- 2,6-dione | 624.1 | ¹H NMR ( 400 MHz, DMSO-d6): 10.98 (s, 1H), 9.12 (s, 1H), 8.71 (s, 1H), 7.72 (s, 1H), 7.70 (d, J = 7.8 Hz, 1H), 7.52 (s, 1H), 7.45 (d, J = 7.9 Hz, 1H), 6.70 (s, 1H), 5.63 (s, 2H), 5.10 (dd, J = 13.3, 5.0 Hz, 1H), 4.23-4.50 (m, 2H), 3.55 (br s, 4H), 2.83-2.97 (m, 1H), 2.54- 2.70 (m, 2H), 2.32-2.39 (m, 1H), 1.97-2.01 (m, 4H) |
| 12 | 6, 32, 44, 46, 49 | 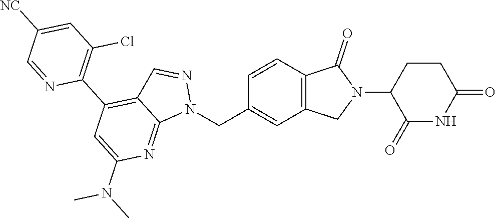 [SMILES: CN(C)c1cc(-c2ncc(C#N)cc2Cl)c2cnn(Cc3ccc4c(c3)CN(C3CCC(=O)NC3=O)C4=O)c2n1] | 3-chloro-4-[6- (dimethylamino)- 1-{[2-(2,6- dioxopiperidin- 3-yl)-1-oxo- 2,3-dihydro- 1H-isoindol-5- yl]methyl}-1H- pyrazolo[3,4- b]pyridin-4- yl]benzonitrile | 554.1 | ¹H NMR (400 MHz, DMSO-d6) 10.98 (s, 1H), 8.49 (s, 1H), 8.27 (d, J = 1.2 Hz, 1H), 7.98 (dd, J = 1.2, 8.0 Hz, 1H), 7.74 (d, J = 8.0 Hz, 1H), 7.69 (d, J = 7.6 Hz, 1H), 7.60 (s, 1H), 7.53 (s, 1H), 7.45 (d, J = 8.0 Hz, 1H), 6.64 (s, 1H), 5.62 (s, 2H), 5.09 (dd, J = 5.2, 13.2 Hz, 1H), 4.45-4.26 (m, 2H), 3.17 (s, 6H), 2.94-2.85 (m, 1H), 2.61-2.56 (m, 1H), 2.37 (d, J = 4.0 Hz, 1H), 2.00-1.96 (m, 1H) |
| 13 | 13 | 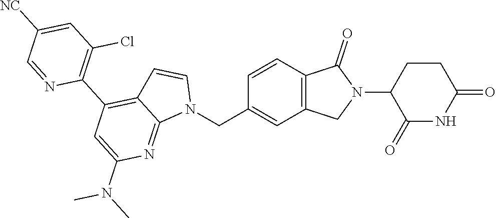 [SMILES: CN(C)c1cc(-c2ncc(C#N)cc2Cl)c2ccn(Cc3ccc4c(c3)CN(C3CCC(=O)NC3=O)C4=O)c2n1] | 5-chloro-6-(6- (dimethylamino)- 1-((2-(2,6- dioxopiperidin- 3-yl)-1- oxoisoindolin- 5-yl)methyl)- 1H- pyrrolo[2,3- b]pyridin-4- yl)nicotinonitrile | 553.2 | ¹H NMR (400 MHz, DMSO-d6) δ 10.98 (s, 1H), 8.22 (d, J = 1.6 Hz, 1H), 7.96-7.91 (m, 1H), 7.71-7.64 (m, 2H), 7.58 (s, 1H), 7.49 (d, J = 8.0 Hz, 1H), 7.27 (d, J = 3.6 Hz, 1H), 6.46 (s, 1H), 5.99 (d, J = 3.6 Hz, 1H), 5.46 (s, 2H), 5.13-5.05 (m, 1H), 4.45-4.41 (m, 1H), 4.32-4.25 (m, 1H), 3.09 (s, 6H), 2.95-2.85 (m, 1H), 2.61-2.55 (m, 1H), 2.42-2.31 (m, 1H), 2.03-1.90 (m, 1H) |
| 14 | 14 | 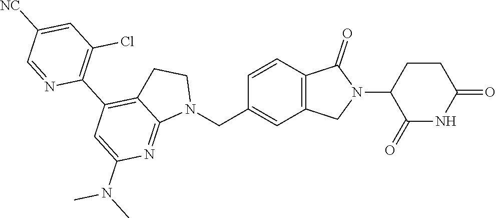 [SMILES: CN(C)c1cc(-c2ncc(C#N)cc2Cl)c2c(n1)N(Cc1ccc3c(c1)CN(C1CCC(=O)NC1=O)C3=O)CC2] | 5-chloro-6-(6- (dimethylamino)- 1-((2-(2,6- dioxopiperidin- 3-yl)-1- oxoisoindolin- 5-yl)methyl)- 2,3-dihydro- 1H- pyrrolo[2,3- b]pyridin-4- yl)nicotinonitrile | 555.2 | ¹H NMR (400MHz, DMSO-d₆) δ 10.98 (s, 1H), 8.16 (d, J = 1.6 Hz, 1H), 7.90-7.85 (m, 1H), 7.70 (d, J = 8.0 Hz, 1H), 7.61-7.54 (m, 2H), 7.49 (d, J = 8.0 Hz, 1H), 5.57 (s, 1H), 5.14-5.07 (m, 1H), 4.62 (s, 2H), 4.43 (s, 1H), 4.33-4.29 (m, 1H), 3.37 (t, J = 8.0 Hz, 2H), 2.97 (s, 6H), 2.92-2.87 (m, 1H), 2.65-2.61 (m, 3H), 2.44-2.34 (m, 1H), 2.04-1.95 (m, 1H) |
| 15 | 15 | 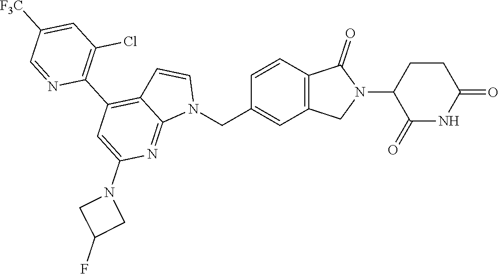 [SMILES: O=C1CCC(N2Cc3cc(Cn4ccc5c(-c6ncc(C(F)(F)F)cc6Cl)cc(N6CC(F)C6)nc54)ccc3C2=O)C(=O)N1] | 3-(5-((4-(3- chloro-5- (trifluoromethyl) pyridin-2-yl)- 6-(3- fluoroazetidin- 1-yl)-1H- pyrrolo[2,3- b]pyridin-1- yl)methyl)-1- oxoisoindolin- 2- yl)piperidine- 2,6-dione | 627.0 | ¹H NMR (400 MHz, DMSO-d6) δ 10.98 (s, 1H), 9.11-9.07 (m, 1H), 8.66 (d, J = 1.3 Hz, 1H), 7.71 (d, J = 7.8 Hz, 1H), 7.56 (s, 1H), 7.49 (s, 1H), 7.35 (d, J = 3.5 Hz, 1H), 6.44 (s, 1H), 6.16 (d, J = 3.4 Hz, 1H), 5.64-5.42 (m, 3H), 5.10 (dd, J = 13.2, 5.0 Hz, 1H), 4.49-4.40 (m, 1H), 4.40-4.25 (m, 3H), 4.15-3.98 (m, 2H), 2.97- 2.85 (m, 1H), 2.62- 2.57 (m, 1H), 2.40-2.33 (m, 1H), 2.03-1.93 (m, 1H) |
| 16 | 16 | 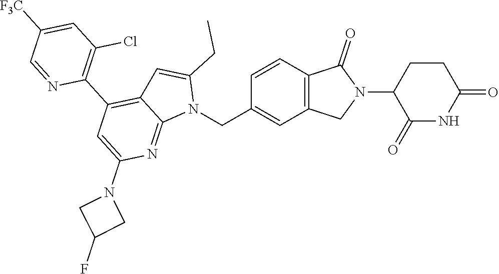 [SMILES: CCc1cc2c(-c3ncc(C(F)(F)F)cc3Cl)cc(N3CC(F)C3)nc2n1Cc1ccc2c(c1)CN(C1CCC(=O)NC1=O)C2=O] | 3-(5-((4-(3- chloro-5- (trifluoromethyl) pyridin-2-yl)- 2-ethyl-6-(3- fluoroazetidin- 1-yl)-1H- pyrrolo[2,3- b]pyridin-1- yl)methyl)-1- oxoisoindolin- 2- yl)piperidine- 2,6-dione | 655.1 | ¹H NMR (400 MHz, DMSO-d₆) δ 10.97 (s, 1H), 9.04 (s, 1H), 8.58 (s, 1H), 8.22 (s, 1H), 7.71 (d, J = 8.0 Hz, 1H), 7.55 (s, 1H), 7.47 (d, J = 7.6 Hz, 1H), 6.43-6.29 (m, 1H), 5.89-5.80 (m, 1H), 5.80- 5.65 (m, 1H), 5.36- 5.22 (m, 1H), 5.16-5.06 (m, 1H), 5.05-4.95 (m, 1H), 4.93-4.84 (m, 1H), 4.58-4.45 (m, 1H), 4.41 (d, J = 17.6 Hz, 1H), 4.34- 4.23 (m, 1H), 4.12- 3.77 (m, 2H), 2.98-2.82 (m, 1H), 2.70-2.64 (m, 2H), 2.62-2.55 (m, 1H), 2.40-2.34 (m, 1H), 2.04- 1.94 (m, 1H), 1.19 (t, J = 7.6 Hz, 3H) |
| 17 | 17 | 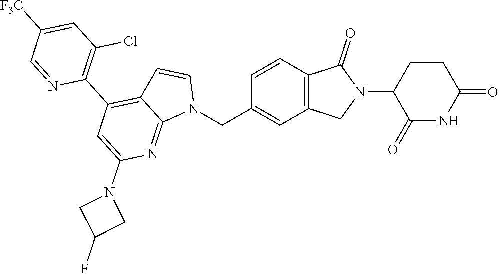 [SMILES: O=C1CCC(N2Cc3cc(Cn4ccc5c(-c6ncc(C(F)(F)F)cc6Cl)cc(N6CC(F)C6)nc54)ccc3C2=O)C(=O)N1] | 3-(5-((4-(3- chloro-5- fluoropyridin- 2-yl)-6-(3- fluoroazetidin- 1-yl)-1H- pyrrolo[2,3- b]pyridin-1- yl)methyl)-1- oxoisoindolin- 2- yl)piperidine- 2,6-dione | 576.8 | ¹H NMR (500 MHz, DMSO-d6) δ ppm 1.91- 2.03 (m, 1 H) 2.32-2.41 (m, 2 H) 2.57-2.62 (m, 1 H) 2.83-2.96 (m, 1 H) 4.00-4.12 (m, 2 H) 4.25- 4.47 (m, 4 H) 5.04- 5.14 (m, 1 H) 5.42-5.61 (m, 3 H) 6.12 (d, J = 3.46 Hz, 1 H) 6.38 (s, 1 H) 7.29-7.35 (m, 1 H) 7.47 (d, J = 7.87 Hz, 1 H) 7.55 (s, 1 H) 7.67-7.73 (m, 1 H) 8.26-8.32 (m, 1 H) 8.73-8.76 (m, 1 H) 10.97 (s, 1 H) |
| 18 | 19 | 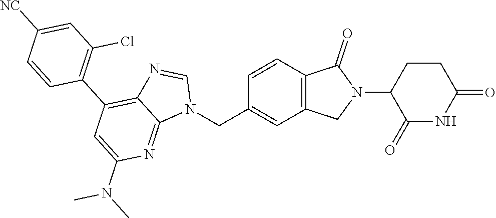 [SMILES: CN(C)c1cc(-c2ccc(C#N)cc2Cl)c2ncn(Cc3ccc4c(c3)CN(C3CCC(=O)NC3=O)C4=O)c2n1] | 5-chloro-6-(5- (dimethylamino)- 3-((2-(2,6- dioxopiperidin- 3-yl)-1- oxoisoindolin- 5-yl)methyl)- 3H- imidazo[4,5- b]pyridin-7- yl)nicotinonitrile | 554.2 | ¹H NMR (400MHz, DMSO-d₆) δ 11.02- 10.77 (m, 1H), 8.25- 8.16 (m, 2H), 7.97-7.89 (m, 1H), 7.74-7.69 (m, 2H), 7.67 (s, 1H), 7.59 (d, J = 8.0 Hz, 1H), 6.57 (s, 1H), 5.49 (s, 2H), 5.14- 5.04 (m, 1H), 4.49-4.38 (m, 1H), 4.35-4.25 (m, 1H), 3.10 (s, 6H), 2.96- 2.83 (m, 1H), 2.58 (d, J = 17.2 Hz, 1H), 2.41-2.29 (m, 1H), 2.03-1.92 (m, 1H) |
| 19 | 19 | 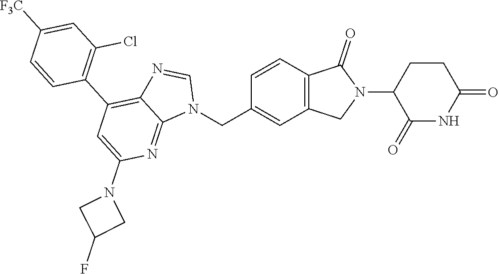 [SMILES: O=C1CCC(N2Cc3cc(Cn4cnc5c(-c6ccc(C(F)(F)F)cc6Cl)cc(N6CC(F)C6)nc54)ccc3C2=O)C(=O)N1] | 3-(5-((7-(2- chloro-4- (trifluoromethyl) phenyl)-5-(3- fluoroazetidin- 1-yl)-3H- imidazo[4,5- b]pyridin-3- yl)methyl)-1- oxoisoindolin- 2- yl)piperidine- 2,6-dione | 627.2 | ¹H NMR (400 MHz, DMSO-d₆) δ 10.98 (s, 1H), 8.27 (s, 1H), 8.03 (s, 1H), 7.89-7.81 (m, 1H), 7.79-7.69 (m, 2H), 7.68- 7.64 (m, 1H), 7.62- 7.54 (m, 1H), 6.42 (s, 1H), 5.64-5.42 (m, 3H), 5.13-5.06 (m, 1H), 4.49- 4.42 (m, 1H), 4.39- 4.29 (m, 3H), 4.15-4.03 (m, 3H), 2.89 (dd, J = 2.4, 5.6 Hz, 1H), 2.58 (s, 1H), 2.40 (d, J = 1.6 Hz, 1H), 2.03-1.93 (m, 1H) |
| 20 | 21 | 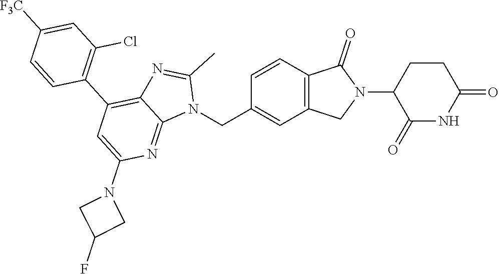 [SMILES: Cc1nc2c(-c3ccc(C(F)(F)F)cc3Cl)cc(N3CC(F)C3)nc2n1Cc1ccc2c(c1)CN(C1CCC(=O)NC1=O)C2=O] | 3-(5-((7-(2- chloro-4- (trifluoromethyl) phenyl)-5-(3- fluoroazetidin- 1-yl)-2-methyl- 3H- imidazo[4,5- b]pyridin-3- yl)methyl)-1- oxoisoindolin- 2- yl)piperidine- 2,6-dione | 641.3 | ¹H NMR (400 MHz, DMSO-d₆) δ 11.04- 10.89 (m, 1H), 8.03 (s, 1H), 7.87-7.81 (m, 1H), 7.74 (t, J = 8.0 Hz, 2H), 7.50 (s, 1H), 7.43 (d, J = 8.0 Hz, 1H), 6.37 (s, 1H), 5.62-5.41 (m, 3H), 5.09 (dd, J = 5.2, 13.2 Hz, 1H), 4.49-4.41 (m, 1H), 4.39-4.27 (m, 3H), 4.13- 3.99 (m, 2H), 2.95- 2.84 (m, 1H), 2.62-2.60 (m, 1H), 2.41 (s, 3H), 2.37 (br d, J = 4.0 Hz, 1H), 2.02-1.94 (m, 1H) |
| 21 | 21 | 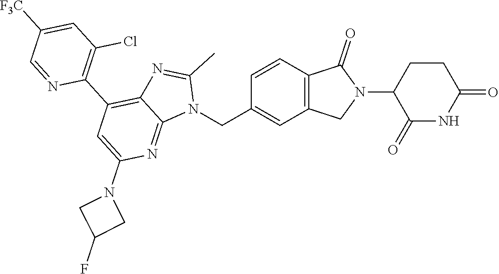 [SMILES: Cc1nc2c(-c3ncc(C(F)(F)F)cc3Cl)cc(N3CC(F)C3)nc2n1Cc1ccc2c(c1)CN(C1CCC(=O)NC1=O)C2=O] | 3-(5-((7-(3- chloro-5- (trifluoromethyl) pyridin-2-yl)- 5-(3- fluoroazetidin- 1-yl)-2-methyl- 3H- imidazo[4,5- b]pyridin-3- yl)methyl)-1- oxoisoindolin- 2- yl)piperidine- 2,6-dione | 642.3 | ¹H NMR (400 MHz, DMSO-d₆) δ 10.97 (s, 1H), 9.07 (d, J = 0.8 Hz, 1H), 8.64 (d, J = 1.2 Hz, 1H), 7.73 (d, J = 7.6 Hz, 1H), 7.48 (s, 1H), 7.43 (d, J = 8.0 Hz, 1H), 6.42 (s, 1H), 5.59-5.44 (m, 3H), 5.09 (dd, J = 5.2, 13.2 Hz, 1H), 4.47-4.29 (m, 4H), 4.11-4.03 (m, 2H), 2.94-2.85 (m, 1H), 2.61 (s, 1H), 2.41 (s, 3H), 2.35 (dd, J = 4.8, 13.6 Hz, 1H), 2.02-1.94 (m, 1H) |
| 22 | 19 | 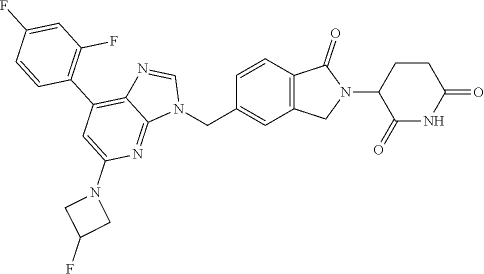 [SMILES: O=C1CCC(N2Cc3cc(Cn4cnc5c(-c6ccc(F)cc6F)cc(N6CC(F)C6)nc54)ccc3C2=O)C(=O)N1] | 3-(5-((7-(2,4- difluorophenyl)- 5-(3- fluoroazetidin- 1-yl)-3H- imidazo[4,5- b]pyridin-3- yl)methyl)-1- oxoisoindolin- 2- yl)piperidine- 2,6-dione | 561.3 | ¹H NMR (400 MHz, DMSO-d₆) δ 10.97 (s, 1H), 8.27 (s, 1H), 8.00- 7.85 (m, 1H), 7.72 (d, J = 7.6 Hz, 1H), 7.63 (s, 1H), 7.55 (d, J = 7.6 Hz, 1H), 7.45-7.37 (m, 1H), 7.24 (dt, J = 2.4, 8.4 Hz, 1H), 6.45 (s, 1H), 5.63-5.41 (m, 3H), 5.09 (dd, J = 5.2, 13.2 Hz, 1H), 4.48- 4.27 (m, 4H), 4.14-4.01 (m, 2H), 2.96-2.84 (m, 1H), 2.60 (d, J = 2.4 Hz, 1H), 2.43-2.34 (m, 1H), 2.02-1.93 (m, 1H) |
| 23 | 21 | 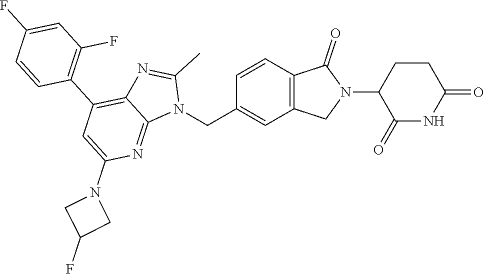 [SMILES: Cc1nc2c(-c3ccc(F)cc3F)cc(N3CC(F)C3)nc2n1Cc1ccc2c(c1)CN(C1CCC(=O)NC1=O)C2=O] | 3-(5-((7-(2,4- difluorophenyl)- 5-(3- fluoroazetidin- 1-yl)-2-methyl- 3H- imidazo[4,5- b]pyridin-3- yl)methyl)-1- oxoisoindolin- 2- yl)piperidine- 2,6-dione | 575.2 | ¹H NMR (400 MHz, DMSO-d₆) δ 10.97 (t, J = 6.4 Hz, 1H), 7.97-7.86 (m, 1H), 7.72 (d, J = 7.6 Hz, 1H), 7.48 (s, 1H), 7.45-7.37 (m, 2H), 7.25 (dt, J = 2.4, 8.4 Hz, 1H), 6.42 (s, 1H), 5.62-5.40 (m, 3H), 5.09 (dd, J = 5.2, 13.2 Hz, 1H), 4.49- 4.25 (m, 4H), 4.14-3.98 (m, 2H), 2.97-2.83 (m, 1H), 2.61 (dd, J = 2.0, 4.0 Hz, 1H), 2.43 (s, 3H), 2.35 (dd, J = 4.4, 13.2 Hz, 1H), 2.02-1.91 (m, 1H) |
| 24 | 21 | 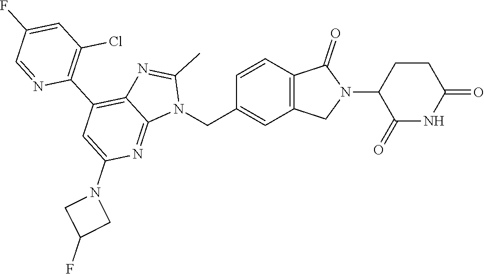 [SMILES: Cc1nc2c(-c3ncc(F)cc3Cl)cc(N3CC(F)C3)nc2n1Cc1ccc2c(c1)CN(C1CCC(=O)NC1=O)C2=O] | 3-(5-((7-(3- chloro-5- fluoropyridin- 2-yl)-5-(3- fluoroazetidin- 1-yl)-2-methyl- 3H- imidazo[4,5- b]pyridin-3- yl)methyl)-1- oxoisoindolin- 2- yl)piperidine- 2,6-dione | 592.3 | ¹H NMR (400 MHz, DMSO-d₆) δ 10.97 (s, 1H), 8.72 (d, J = 2.4 Hz, 1H), 8.26 (dd, J = 2.4, 8.4 Hz, 1H), 7.76-7.69 (m, 1H), 7.48 (s, 1H), 7.42 (d, J = 7.6 Hz, 1H), 6.34 (s, 1H), 5.62-5.41 (m, 3H), 5.09 (dd, J = 5.2, 13.2 Hz, 1H), 4.50-4.40 (m, 1H), 4.39-4.27 (m, 3H), 4.13-4.01 (m, 2H), 2.94- 2.85 (m, 1H), 2.60 (d, J = 1.6 Hz, 1H), 2.45 (s, 1H), 2.41 (s, 3H), 2.03- 1.94 (m, 1H) |
| 25 | 19 | 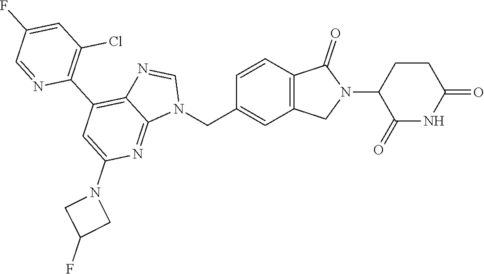 [SMILES: O=C1CCC(N2Cc3cc(Cn4cnc5c(-c6ncc(F)cc6Cl)cc(N6CC(F)C6)nc54)ccc3C2=O)C(=O)N1] | 3-(5-((7-(3- chloro-5- fluoropyridin- 2-yl)-5-(3- fluoroazetidin- 1-yl)-3H- imidazo[4,5- b]pyridin-3- yl)methyl)-1- oxoisoindolin- 2- yl)piperidine- 2,6-dione | 578.2 | ¹H NMR (400 MHz, DMSO-d₆) δ 11.04- 10.94 (m, 1H), 8.73 (d, J = 2.4 Hz, 1H), 8.30-8.23 (m, 2H), 7.73 (d, J = 7.6 Hz, 1H), 7.64 (s, 1H), 7.59-7.55 (m, 1H), 6.39 (s, 1H), 5.63-5.41 (m, 3H), 5.14-5.05 (m, 1H), 4.49-4.41 (m, 1H), 4.39- 4.27 (m, 3H), 4.16- 4.02 (m, 2H), 2.95-2.85 (m, 1H), 2.62-2.58 (m, 1H), 2.41-2.37 (m, 1H), 2.03-1.93 (m, 1H) |
| 26 | 26 | 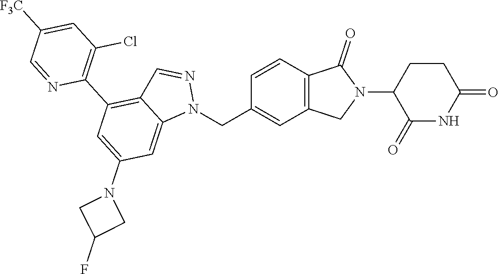 [SMILES: O=C1CCC(N2Cc3cc(Cn4ncc5c(-c6ncc(C(F)(F)F)cc6Cl)cc(N6CC(F)C6)cc54)ccc3C2=O)C(=O)N1] | 3-(5-((4-(3- chloro-5- (trifluoromethyl) pyridin-2-yl)- 6-(3- fluoroazetidin- 1-yl)-1H- indazol-1- yl)methyl)-1- oxoisoindolin- 2- yl)piperidine- 2,6-dione | 627.2 | 1H NMR (400 MHz, DMSO-d6) δ 10.90 (s, 1H), 9.01 (s, 1H), 8.58 (d, J = 1.8 Hz, 1H), 7.72 (s, 1H), 7.63 (d, J = 7.8 Hz, 1H), 7.37 (s, 1H), 7.31 (d, J = 7.7 Hz, 1H), 6.73 (s, 1H), 6.62 (d, J = 1.5 Hz, 1H), 5.66 (s, 2H), 5.57- 5.34 (m, 1H), 5.07-4.96 (m, 1H), 4.41-4.31 (m, 1H), 4.25-4.13 (m, 3H), 3.98-3.86 (m, 2H), 2.81 (br dd, J = 13.1, 4.8 Hz, 1H), 2.53 (br d, J = 2.0 Hz, 1H), 2.35-2.26 (m, 1H), 1.96-1.86 (m, 1H) |
| 27 | 27 | 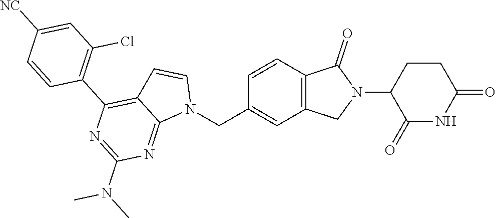 [SMILES: CN(C)c1nc(-c2ccc(C#N)cc2Cl)c2ccn(Cc3ccc4c(c3)CN(C3CCC(=O)NC3=O)C4=O)c2n1] | 3-chloro-4-(2- (dimethylamino)- 7-((2-(2,6- dioxopiperidin- 3-yl)-1- oxoisoindolin- 5-yl)methyl)- 7H- pyrrolo[2,3- d]pyrimidin-4- yl)benzonitrile | 554.2 | ¹H NMR (400 MHz, DMSO-d₆) δ 10.98 (s, 1H), 8.23 (s, 1H), 8.55 (s, 1H), 7.96 (d, 1H), 7.77 (dd, J = 8.0, 16.0, Hz, 2H), 7.60 (s, 1H), 7.52 (d, J = 7.6, Hz, 1H), 7.33 (d, J = 3.2, Hz, 1H), 6.16 (d, J = 3.6 Hz, 1H), 5.45 (s, 2H), 5.12-5.08 (m, 1H), 4.47-4.29 (m, 2H), 3.18 (s, 3H), 2.94-2.90 (m, 1H), 2.61-2.59 (m, 1H), 2.36-2.34 (m, 1H), 2.00- 1.97 (m, 1H) |
| 28 | 27 | 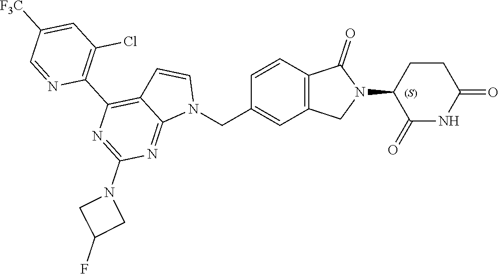 | (S)-3-(5-((4-(3- chloro-5- (trifluoromethyl) pyridin-2-yl)- 2-(3- fluoroazetidin- 1-yl)-7H- pyrrolo[2,3- d]pyrimidin-7- yl)methyl)-1- oxoisoindolin- 2- yl)piperidine- 2,6-dione | 628.1 | ¹H NMR (400 MHz, DMSO-d6) δ: 10.98 (s, 1H), 9.09-9.12 (m, 1H), 8.67 (d, J = 1.3 Hz, 1H), 7.73 (d, J = 7.8 Hz, 1H), 7.55 (s, 1H), 7.48 (d, J = 7.6 Hz, 1H), 7.41 (d, J = 3.6 Hz, 1H), 6.35 (d, J = 3.6 Hz, 1H), 5.39-5.67 (m, 3H), 5.10 (dd, J = 13.3, 5.1 Hz, 1H), 4.36-4.49 (m, 3H), 4.27- 4.35 (m, 1H), 4.06-4.20 (m, 2H), 2.84-2.97 (m, 1H), 2.66-2.71 (m, 1H), 2.32-2.36 (m, 1H), 1.93- 2.03 (m, 1H) |
| 29 | 6, 32, 44, 46, 49 | 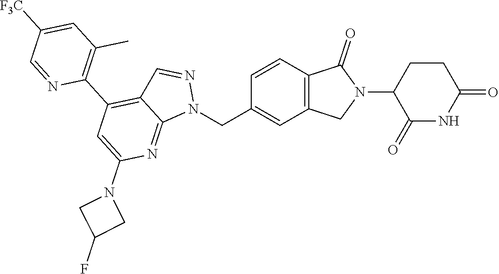 [SMILES: Cc1cc(C(F)(F)F)cnc1-c1cc(N2CC(F)C2)nc2c1cnn2Cc1ccc2c(c1)CN(C1CCC(=O)NC1=O)C2=O] | 3-(5-{[6-(3- fluoroazetidin- 1-yl)-4-[3- methyl-5- (trifluoromethyl) pyridin-2-yl]- 1H- pyrazolo[3,4- b]pyridin-1- yl]methyl}-1- oxo-2,3- dihydro-1H- isoindol-2- yl)piperidine- 2,6-dione | 608.2 | ¹H NMR (400 MHz, DMSO-d6): 10.98 (s, 1H), 8.95 (s, 1H), 8.31 (d, J = 1.8 Hz, 1H), 7.74 (s, 1H), 7.70 (d, J = 7.8 Hz, 1H), 7.50 (s, 1H), 7.42 (d, J = 7.4 Hz, 1H), 6.50 (s, 1H), 5.32-5.73 (m, 3H), 5.10 (dd, J = 13.2, 5.1 Hz, 1H), 4.36-4.55 (m, 3H), 4.25-4.36 (m, 1H), 4.11- 4.25 (m, 2H), 2.83-2.99 (m, 1H), 2.61 (br d, J = 2.3 Hz, 1H), 2.39 (s, 4H), 1.93-2.03 (m, 1H) |
| 30 | 30 | 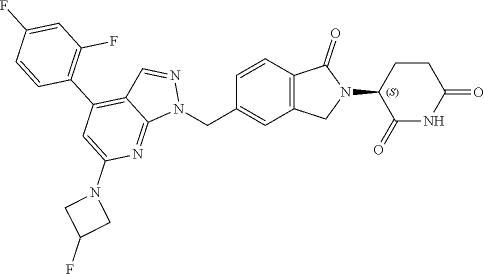 | (3S)-3-(5-{[4- (2,4- difluorophenyl)- 6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 561.0 | ¹H NMR (400 MHz, DMSO-d6, 2 rotamers): 10.98 (s, 1H), 7.73-7.82 (m, 2H), 7.70 (d, J = 7.8 Hz, 1H), 7.45-7.54 (m, 2H), 7.42 (d, J = 7.9 Hz, 1H), 7.29 (t, J = 8.3 Hz, 1H), 6.45 (s, 1H), 5.44- 5.68 (m, 3H), 5.10 (dd, J = 13.3, 5.1 Hz, 1H), 4.39-4.50 (m, 3H), 4.12- 4.33 (m, 3H), 2.85-2.95 (m, 1H), 2.55-2.63 (m, 1H), 2.32-2.39 (m, 1H), 1.93-2.03 (m, 1H) |
| 31 | 3, 9 | 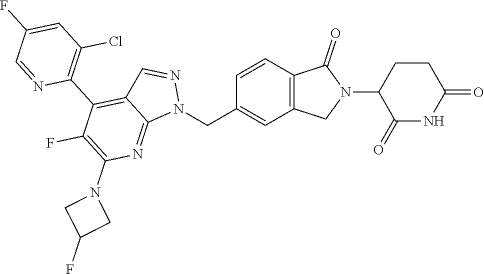 [SMILES: O=C1CCC(N2Cc3cc(Cn4ncc5c(-c6ncc(F)cc6Cl)c(F)c(N6CC(F)C6)nc54)ccc3C2=O)C(=O)N1] | 3-(5-{[4-(3- chloro-5- fluoropyridin- 2-yl)-5-fluoro- 6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 596.2 | ¹H NMR (400 MHz, DMSO-d6) 10.99 (s, 1H), 8.82 (d, J = 2.4 Hz, 1H), 8.39 (dd, J = 2.4, 8.4 Hz, 1H), 7.70 (d, J = 8.0 Hz, 1H), 7.66 (s, 1H), 7.51 (s, 1H), 7.43 (d, J = 7.6 Hz, 1H), 5.66-5.43 (m, 3H), 5.09 (dd, J = 5.2, 13.6 Hz, 1H), 4.62- 4.57 (m, 2H), 4.54-4.25 (m, 4H), 2.95-2.85 (m, 1H), 2.61 (d, J = 1.6 Hz, 1H), 2.40-2.34 (m, 1H), 1.99-1.96 (m, 1H) |
| 32 | 32 | 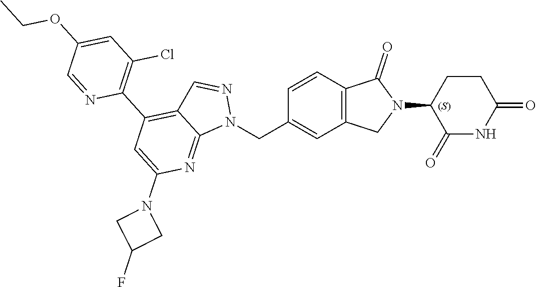 | (3S)-3-[5-({4- [5-ethoxy-3- (trifluoromethyl) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 638.2 | ¹H NMR (400 MHz, DMSO-d6) 10.97 (br s, 1H), 8.68 (d, J = 3.0 Hz, 1H), 7.87 (d, J = 3.0 Hz, 1H), 7.70 (d, J = 7.5 Hz, 1H), 7.59 (s, 1H), 7.49 (s, 1H), 7.45-7.38 (m, 1H), 6.32 (s, 1H), 5.66-5.43 (m, 3H), 5.09 (dd, J = 5.0, 13.0 Hz, 1H), 4.49- 4.36 (m, 3H), 4.35-4.27 (m, 3H), 4.23-4.09 (m, 2H), 2.97-2.85 (m, 1H), 2.63-2.60 (m, 1H), 2.44- 2.36 (m, 1H), 2.03- 1.94 (m, 1H), 1.41 (t, J = 7.0 Hz, 3H) |
| 33 | 6 using Intermediate L | 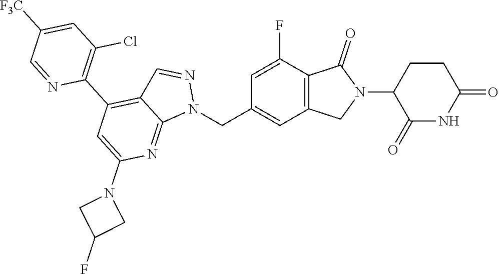 [SMILES: O=C1CCC(N2Cc3cc(Cn4ncc5c(-c6ncc(C(F)(F)F)cc6Cl)cc(N6CC(F)C6)nc54)cc(F)c3C2=O)C(=O)N1] | 3-[5-({4-[3- chloro-5- (trifluoromethyl) pyridin-2-yl]- 6-(3- fluoroazetidin- 1-yl)-1H- pyrazolo[3,4- b]pyridin-1- yl}methyl)-7- fluoro-1-oxo- 2,3-dihydro- 1H-isoindol-2- yl]piperidine- 2,6-dione | 646.3 | ¹H NMR (400 MHz, DMSO-d6) 10.99 (s, 1H), 9.12 (d, J = 1.2 Hz, 1H), 8.72 (d, J = 1.6 Hz, 1H), 7.82 (s, 1H), 7.27 (s, 1H), 7.20 (d, J = 10.4 Hz, 1H), 6.63 (s, 1H), 5.68- 5.43 (m, 3H), 5.05 (dd, J = 5.2, 13.2 Hz, 1H), 4.52- 4.39 (m, 3H), 4.35- 4.27 (m, 1H), 4.26-4.08 (m, 2H), 2.97-2.81 (m, 1H), 2.60 (dd, J = 1.6, 4.0 Hz, 1H), 2.47-2.41 (m, 1H), 2.03-1.91 (m, 1H) |
| 34 | 6, 32, 44, 46, 49 | 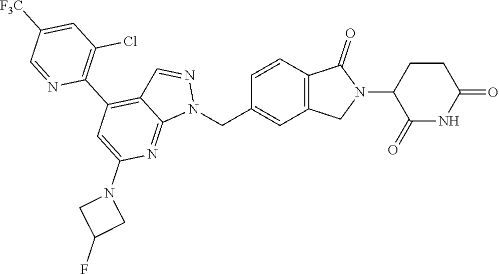 [SMILES: O=C1CCC(N2Cc3cc(Cn4ncc5c(-c6ncc(C(F)(F)F)cc6Cl)cc(N6CC(F)C6)nc54)ccc3C2=O)C(=O)N1] | 3-[5-({4-[3- chloro-5- (trifluoromethyl) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 628.2 | ¹H NMR (400 MHz, DMSO) 11.01-10.90 (m, 1H), 11.05-10.88 (m, 1H), 9.12 (s, 1H), 8.71 (s, 1H), 7.79 (s, 1H), 7.74-7.65 (m, 1H), 7.51- 7.47 (m, 1H), 7.42 (d, J = 7.2 Hz, 1H), 6.62 (s, 1H), 5.64 (s, 2H), 5.58- 5.38 (m, 1H), 5.09 (dd, J = 5.2, 13.2 Hz, 1H), 4.53- 4.39 (m, 3H), 4.33- 4.27 (m, 1H), 4.24-4.13 (m, 2H), 2.95-2.84 (m, 1H), 2.60 (d, J = 1.6 Hz, 1H), 2.36 (s, 1H), 2.02- 1.93 (m, 1H) |
| 35 | 6, 32, 44, 46, 49 | 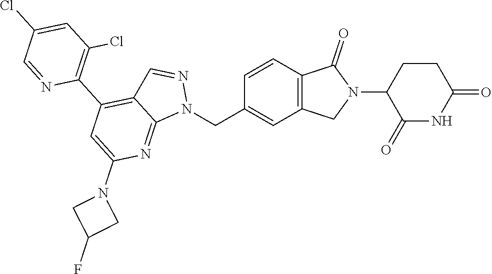 [SMILES: O=C1CCC(N2Cc3cc(Cn4ncc5c(-c6ncc(Cl)cc6Cl)cc(N6CC(F)C6)nc54)ccc3C2=O)C(=O)N1] | 3-(5-{[4-(3,5- dichloropyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 594.2 | ¹H NMR (400 MHz, DMSO-d6) 10.98 (s, 1H), 8.80 (d, J = 2.0 Hz, 1H), 8.48 (d, J = 2.0 Hz, 1H), 7.77 (s, 1H), 7.69 (d, J = 7.6 Hz, 1H), 7.48 (s, 1H), 7.40 (d, J = 8.0 Hz, 1H), 6.58 (s, 1H), 5.63- 5.46 (m, 3H), 5.09 (dd, J = 4.8, 13.2 Hz, 1H), 4.49- 4.39 (m, 3H), 4.31- 4.27 (m, 1H), 4.22-4.13 (m, 2H), 2.94-2.85 (m, 1H), 2.60 (d, J = 2.4 Hz, 1H), 2.36 (d, J = 4.8 Hz, 1H), 1.98-1.94 (m, 1H) |
| 36 | 3, 9, 41, 140 | 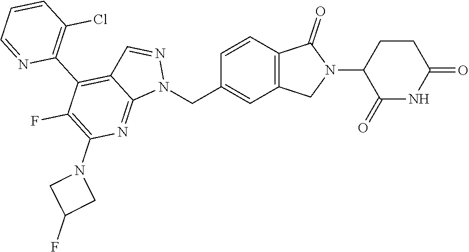 [SMILES: O=C1CCC(N2Cc3cc(Cn4ncc5c(-c6ncccc6Cl)c(F)c(N6CC(F)C6)nc54)ccc3C2=O)C(=O)N1] | 3-(5-{[4-(3- chloropyridin- 2-yl)-5-fluoro- 6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 578.2 | ¹H NMR (400 MHz, DMSO-d6) 10.98 (s, 1H), 8.73 (d, J = 4.4 Hz, 1H), 8.19 (d, J = 1.6 Hz, 1H), 7.71-7.69 (m, 1H), 7.64-7.63 (m, 2H), 7.51 (s, 1H), 7.44 7.44 (s, 1H), 5.64-5.60 (m, 3H), 5.09 (dd, J = 5.2, 13.6 Hz, 1H), 4.60- 4.42 (m, 2H), 4.32-4.25 (m, 4H), 4.33- 4.25 (m, 3H), 2.94- 2.90 (m, 1H), 2.60 (d, J = 2.8 Hz, 1H), 2.38-2.37 (m, 1H), 2.01-1.97 (m, 1H) |
| 37 | 3, 9, 41, 140 | 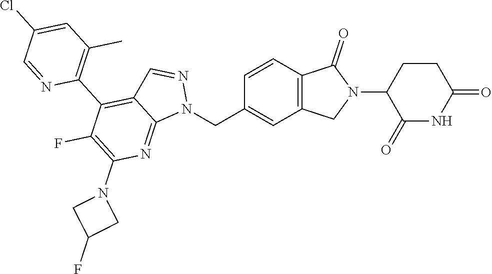 [SMILES: Cc1cc(Cl)cnc1-c1c(F)c(N2CC(F)C2)nc2c1cnn2Cc1ccc2c(c1)CN(C1CCC(=O)NC1=O)C2=O] | 3-(5-{[4-(5- chloro-3- methylpyridin- 2-yl)-5-fluoro- 6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 592.3 | ¹H NMR (400 MHz, DMSO-d6) 10.98 (s, 1H), 8.63 (d, J = 2.0 Hz, 1H), 8.06 (d, J = 1.6 Hz, 1H), 7.70 (d, J = 8.0 Hz, 1H), 7.62 (s, 1H), 7.50 (s, 1H), 7.43 (d, J = 7.6 Hz, 1H), 5.72-5.40 (m, 3H), 5.09 (dd, J = 5.2, 13.2 Hz, 1H), 4.67-4.48 (m, 2H), 4.47-4.22 (m, 4H), 2.97-2.82 (m, 1H), 2.59 (d, J = 17.2 Hz, 1H), 2.38- 2.31 (m, 1H), 2.21 (s, 3H), 2.02-1.92 (m, 1H) |
| 38 | 2 | 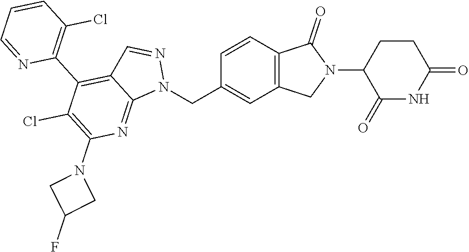 [SMILES: O=C1CCC(N2Cc3cc(Cn4ncc5c(-c6ncccc6Cl)c(Cl)c(N6CC(F)C6)nc54)ccc3C2=O)C(=O)N1] | 3-(5-{[5- chloro-4-(3- chloropyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 594.0 | ¹H NMR (400 MHz, DMSO-d6) 10.99 (s, 1H), 8.70 (d, J = 4.4 Hz, 1H), 8.18 (d, J = 7.6 Hz, 1H), 7.71 (d, J = 8.0 Hz, 1H), 7.62 (dd, J = 4.8, 8.4 Hz, 1H), 7.54 (s, 2H), 7.46 (d, J = 8.0 Hz, 1H), 5.70-5.39 (m, 3H), 5.10 (dd, J = 5.2, 13.2 Hz, 1H), 4.70-4.62 (m, 2H), 4.47-4.28 (m, 4H), 2.94- 2.85 (m, 1H), 2.58 (d, J = 18.0 Hz, 1H), 2.41- 2.37 (m, 1H), 1.99-1.96 (m, 1H) |
| 39 | 6, 32, 44, 46, 49 | 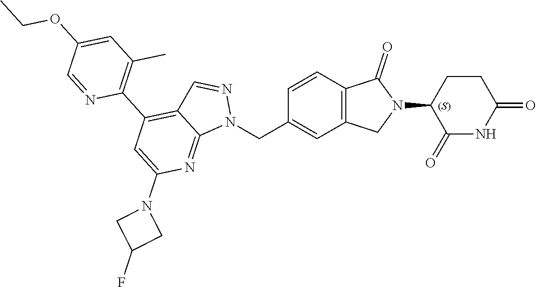 | (3S)-3-(5-{[4- (4-ethoxy-2- methylphenyl)- 6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 583.4 | ¹H NMR (400 MHz, DMSO) 10.98 (s, 1H), 7.6 (d, J = 8.0 Hz, 1H), 7.60 (s, 1H), 7.6 (s, 1H), 7.6 (d, J = 8.4 Hz, 1H), 7.2 (d, J = 8.4 Hz, 1H), 6.96 (d, J = 2.4 Hz, 1H), 6.8 (dd, J = 2.4, 8.4 Hz, 1H), 6.4 (s, 1H), 5.6-5.2 (m, 3H), 5.2 (dd, J = 4.8, 13.2 Hz, 1H), 4.48-4.36 (m, 3H), 4.4-4.24 (m, 1H), 4.20-4.04 (m, 4H), 2.96-2.84 (m, 1H), 2.62 (s, 1H), 2.42-2.4 (m, 1H), 2.2 (s, 3H), 2.04- 1.94 (m, 1H), 1.6 (t, J = 6.8 Hz, 3H) |
| 40 | 61 | 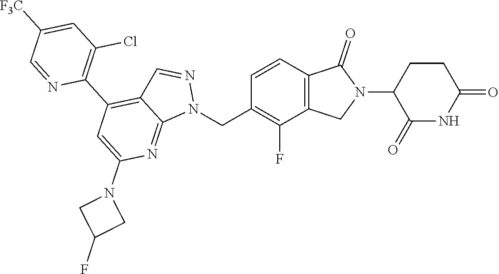 [SMILES: O=C1CCC(N2Cc3c(ccc(Cn4ncc5c(-c6ncc(C(F)(F)F)cc6Cl)cc(N6CC(F)C6)nc54)c3F)C2=O)C(=O)N1] | 3-[5-({4-[3- chloro-5- (trifluoromethyl) pyridin-2-yl]- 6-(3- fluoroazetidin- 1-yl)-1H- pyrazolo[3,4- b]pyridin-1- yl}methyl)-4- fluoro-1-oxo- 2,3-dihydro- 1H-isoindol-2- yl]piperidine- 2,6-dione | 646.3 | ¹H NMR (400 MHz, DMSO-d6) 11.02(s, 1H), 9.16 (s, 1H), 8.72 (s, 1H), 7.80 (s, 1H), 7.56 (d, J = 7.6 Hz, 1H), 7.45-7.36 (m, 1H), 6.64 (s, 1H), 5.72-5.46 (m, 3H), 5.12 (dd, J = 4.8, 13.2 Hz, 1H), 4.60 (d, J = 17.6 Hz, 1H), 4.52- 4.38 (m, 3H), 4.24-4.12(m, 2H), 2.96- 2.86 (m, 1H), 2.62 (d, J = 2.4 Hz, 1H), 2.45-2.40 (m, 1H), 2.04-1.96 (m, 1H) |
| 41 | 41 | 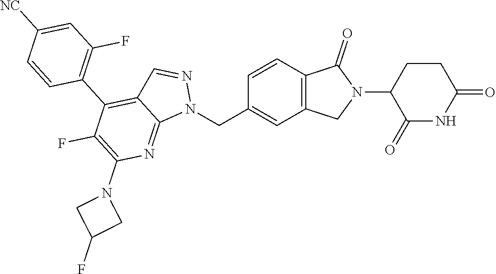 [SMILES: N#Cc1ccc(-c2c(F)c(N3CC(F)C3)nc3c2cnn3Cc2ccc3c(c2)CN(C2CCC(=O)NC2=O)C3=O)c(F)c1] | 4-(1-{[2-(2,6- dioxopiperidin- 3-yl)-1-oxo- 2,3-dihydro- 1H-isoindol-5- yl]methyl}-5- fluoro-6-(3- fluoroazetidin- 1-yl)-1H- pyrazolo[3,4- b]pyridin-4- yl)-3- fluorobenzonitrile | 586.2 | ¹H NMR (400 MHz, DMSO-d6) 11.01-10.97 (m, 1H), 8.15-8.12 (m, 1H), 7.93-7.86 (m, 2H), 7.76 (s, 1H), 7.70 (d, J = 7.6 Hz, 1H), 7.52 (s, 1H), 7.43 (d, J = 7.6 Hz, 1H), 5.64-5.44 (m, 3H), 5.09 (dd, J = 5.6, 13.2 Hz, 1H), 4.65-4.51 (m, 2H), 4.45-4.25 (m, 4H), 2.95- 2.86 (m, 1H), 2.58 (d, J = 18.0 Hz, 1H), 2.38- 2.35 (m, 1H), 2.00-1.96 (m, 1H) |
| 42 | 6, 32, 44, 46, 49 using Intermediate L | 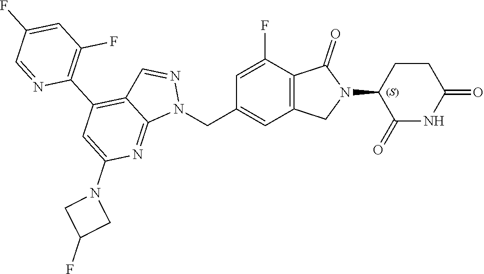 | (3S)-3-(5-{[4- (3-chloro-5- fluoropyridin- 2-yl)-6-(3- fluoroazetidin- 1-yl)-1H- pyrazolo[3,4- b]pyridin-1- yl]methyl}-7- fluoro-1-oxo- 2,3-dihydro- 1H-isoindol-2- yl)piperidine- 2,6-dione | 596.3 | ¹H NMR (400 MHz, DMSO-d6) 10.98 (s, 1H), 8.79-8.77 (m, 1H), 8.34 (dd, J = 2.4, 8.4 Hz, 1H), 7.77 (s, 1H), 7.26 (s, 1H), 7.19 (d, J = 10.4 Hz, 1H), 6.57 (s, 1H), 5.63- 5.46 (m, 3H), 5.05 (dd, J = 5.2, 13.6 Hz, 1H), 4.49- 4.39 (m, 3H), 4.33- 4.28 (m, 1H), 4.22-4.13 (m, 2H), 2.93-2.84 (m, 1H), 2.60-2.55 (m, 1H), 2.32 (dd, J = 4.4, 13.2 Hz, 1H), 2.00-1.96 (m, 1H) |
| 43 | 32 using Cas # 71690- 06-7 | 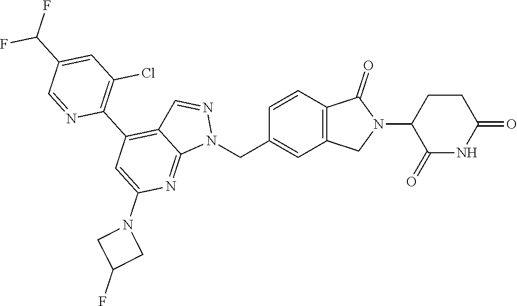 [SMILES: O=C1CCC(N2Cc3cc(Cn4ncc5c(-c6ncc(C(F)F)cc6Cl)cc(N6CC(F)C6)nc54)ccc3C2=O)C(=O)N1] | 3-[5-({4-[3- chloro-5- (difluoromethyl) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 610.2 | ¹H NMR (400 MHz, DMSO-d6) = 10.97 (s, 1H), 8.93 (d, J = 1.5 Hz, 1H), 8.41 (d, J = 1.0 Hz, 1H), 7.78 (s, 1H), 7.70 (d, J = 8.0 Hz, 1H), 7.50 (s, 1H), 7.43 (d, J = 7.5 Hz, 1H), 7.40-7.12 (m, 1H), 6.62 (s, 1H), 5.72-5.42 (m, 3H), 5.09 (dd, J = 5.0, 13.0 Hz, 1H), 4.52- 4.39 (m, 3H), 4.36-4.27 (m, 1H), 4.26-4.12 (m, 2H), 2.93-2.87 (m, 1H), 2.65-2.57 (m, 1H), 2.43- 2.32 (m, 1H), 2.01-1.95 (m, 1H) |
| 44 | 44 | 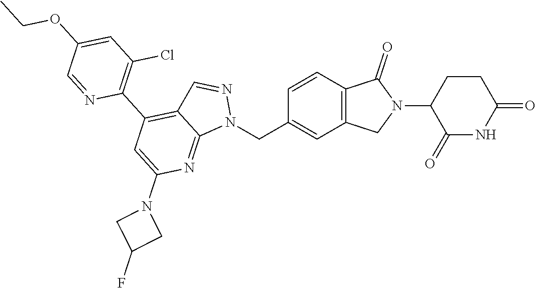 [SMILES: CCOc1cnc(-c2cc(N3CC(F)C3)nc3c2cnn3Cc2ccc3c(c2)CN(C2CCC(=O)NC2=O)C3=O)c(Cl)c1] | 3-(5-{[4-(3- chloro-5- ethoxypyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 604.2 | ¹H NMR (400 MHz, DMSO-d6, 2 rotamers): 10.99 (s, 1H), 8.44 (d, J = 2.0 Hz, 1H), 7.78 (d, J = 2.5 Hz, 1H), 7.76 (s, 1H), 7.70 (d, J = 7.5 Hz, 1H), 7.49 (s, 1H), 7.41 (d, J = 8.0 Hz, 1H), 6.55 (s, 1H), 5.45-5.63 (m, 3H), 5.06-5.14 (m, 1H), 4.40- 4.47 (m, 3H), 4.12-4.25 (m, 5H), 2.87-2.93 (m, 1H), 2.61 (br s, 1H), 2.38 (br s, 1H), 1.96-2.01 (m, 1H), 1.39 (t, J = 7.0 Hz, 3H) |
| 45 | 6, 32, 44, 46, 49 | 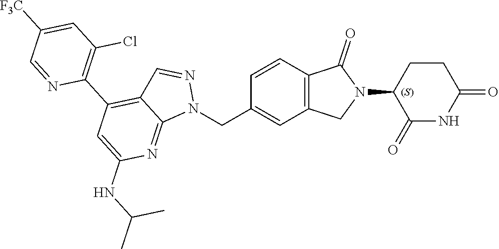 | (3S)-3-[5-({4- [3-chloro-5- (trifluoromethyl) pyridin-2-yl]- 6-[(propan-2- yl)amino]-1H- pyrazolo[3,4- b]pyridin-1- yl}methyl)-1- oxo-2,3- dihydro-1H- isoindol-2- yl]piperidine- 2,6-dione | 612.3 | ¹H NMR (400 MHz, DMSO-d6): 10.98 (s, 1H), 9.09-9.12 (m, 1H), 8.67-8.69 (m, 1H), 7.69 (d, J = 7.8 Hz, 1H), 7.65 (s, 1H), 7.53 (s, 1H), 7.44 (d, J = 8.5 Hz, 1H), 7.29 (d, J = 7.1 Hz, 1H), 6.67 (s, 1H), 5.60 (s, 2H), 5.10 (dd, J = 13.2, 5.1 Hz, 1H), 4.26-4.48 (m, 2H), 4.19 (dq, J = 13.2, 6.5 Hz, 1H), 2.85-2.96 (m, 1H), 2.59 (br dd, J = 15.2, 2.2 Hz, 1H), 2.30-2.39 (m, 1H), 1.93-2.02 (m, 1H), 1.22 (dd, J = 6.4, 2.6 Hz, 6H) |
| 46 | 46 | 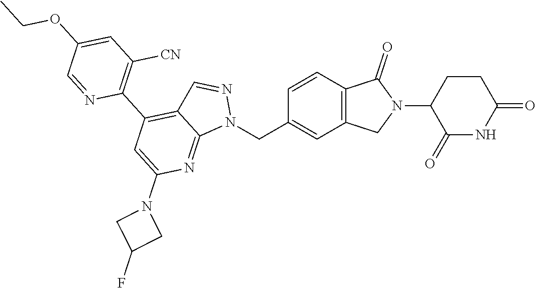 [SMILES: CCOc1cnc(-c2cc(N3CC(F)C3)nc3c2cnn3Cc2ccc3c(c2)CN(C2CCC(=O)NC2=O)C3=O)c(C#N)c1] | 2-(1-{[2-(2,6- dioxopiperidin- 3-yl)-1-oxo- 2,3-dihydro- 1H-isoindol-5- yl]methyl}-6- (3- fluoroazetidin- 1-yl)-1H- pyrazolo[3,4- b]pyridin-4- yl)-5- ethoxypyridine- 3-carbonitrile | 595.2 | ¹H NMR (400 MHz, DMSO-d6, 2 rotamers): 10.98 (s, 1H), 8.75 (d, J = 2.5 Hz, 1H), 8.17 (d, J = 3.0 Hz, 1H), 7.95 (s, 1H), 7.69 (d, J = 8.0 Hz, 1H), 7.48 (s, 1H), 7.41 (d, J = 8.0 Hz, 1H), 6.80 (s, 1H), 5.47-5.65 (m, 3H), 5.09 (dd, J = 13.3, 5.3 Hz, 1H), 4.39-4.51 (m, 3H), 4.25-4.34 (m, 3H), 4.13- 4.24 (m, 2H), 2.85-2.95 (m, 1H), 2.57-2.62 (m, 1H), 2.32-2.35 (m, 1H), 1.94-2.02 (m, 1H), 1.40 (t, J = 7.0 Hz, 3H) |
| 47 | 6, 32, 44, 46, 49 | 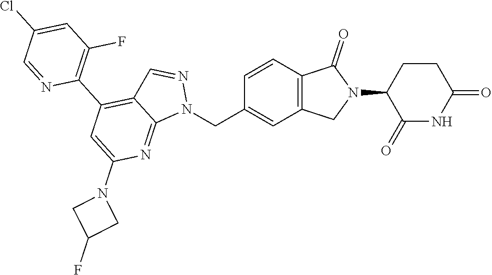 [SMILES: O=C1CC[C@H](N2Cc3cc(Cn4ncc5c(-c6ncc(Cl)cc6F)cc(N6CC(F)C6)nc54)ccc3C2=O)C(=O)N1] | (3S)-3-(5-{[4- (5-chloro-3- fluoropyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 578.2 | ¹H NMR (400 MHz, DMSO-d6, 2 rotamers): 10.98 (s, 1H), 8.75-8.79 (m, 1H), 8.36 (dd, J = 11.0, 2.0 Hz, 1H), 8.07 (d, J = 1.5 Hz, 1H), 7.66- 7.73 (m, J = 8.0 Hz, 1H), 7.47 (s, 1H), 7.37-7.43 (m, J = 8.0 Hz, 1H), 6.73- 6.76 (m, 1H), 5.45-5.65 (m, 3H), 5.09 (dd, J = 13.3, 5.3 Hz, 1H), 4.39-4.51 (m, 3H), 4.13- 4.33 (m, 3H), 2.84-2.95 (m, 1H), 2.66-2.69 (m, 1H), 2.32-2.39 (m, 1H), 1.92-2.04 (m, 1H) |
| 48 | 30 | 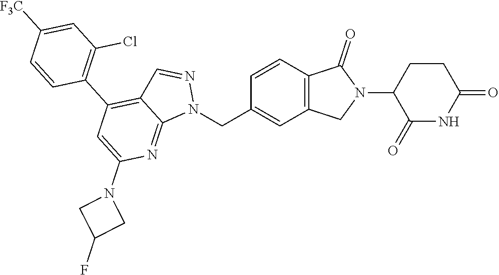 [SMILES: O=C1CCC(N2Cc3cc(Cn4ncc5c(-c6ccc(C(F)(F)F)cc6Cl)cc(N6CC(F)C6)nc54)ccc3C2=O)C(=O)N1] | 3-[5-({4-[2- chloro-4- (trifluoromethyl) phenyl]-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 627.3 | ¹H NMR (400 MHz, DMSO-d6) 10.97 (s, 1H), 8.08 (s, 1H), 7.88 (dd, J = 1.2, 8.4 Hz, 1H), 7.77 (d, J = 8.0 Hz, 1H), 7.71-7.69 (m, 2H), 7.52 (s, 1H), 7.44 (d, J = 8.4 Hz, 1H), 6.43 (s, 1H), 5.67-5.45 (m, 3H), 5.09 (dd, J = 4.8, 13.2 Hz, 1H), 4.49-4.40 (m, 3H), 4.32-4.13 (m, 3H), 2.94- 2.85 (m, 1H), 2.60 (d, J = 1.6 Hz, 1H), 2.37 (d, J = 4.4 Hz, 1H), 2.01-1.96 (m, 1H) |
| 49 | 49 | 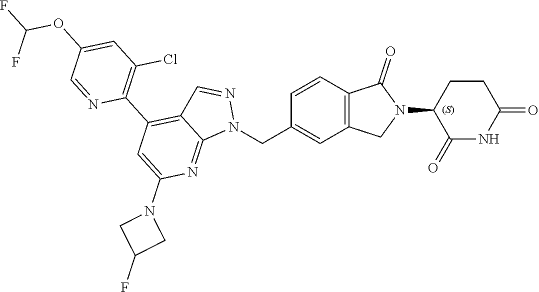 | (3S)-3-[5-({4- [3-chloro-5- (difluoromethoxy) pyridin-2- yl]-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 626.0 | ¹H NMR (400 MHz, DMSO-d6) = 10.97 (s, 1H), 8.66 (d, J = 2.4 Hz, 1H), 8.16 (d, J = 2.4 Hz, 1H), 7.75 (s, 1H), 7.70- 7.66 (m, 1H), 7.49-7.30 (m, 3H), 6.57 (s, 1H), 5.63-5.46 (m, 3H), 5.09 (dd, J = 5.6, 13.6 Hz, 1H), 4.49-4.40 (m, 3H), 4.32-4.27 (m, 1H), 4.22- 4.13 (m, 2H), 2.94- 2.85 (m, 1H), 2.61-2.60 (m, 1H), 2.42-2.36 (m, 1H), 2.00-1.94 (m, 1H) |
| 50 | 6, 32, 44, 46, 49 | 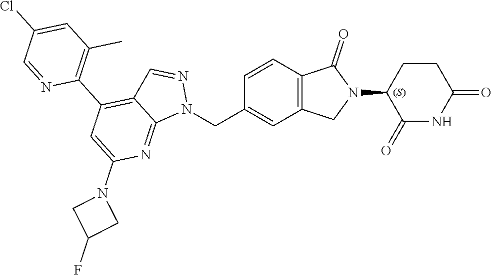 | (3S)-3-(5-{[4- (5-chloro-3- methylpyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 574.2 | ¹H NMR (400 MHz, DMSO-d6) 10.97 (s, 1H), 8.61 (d, J = 2.0 Hz, 1H), 8.06-8.00 (m, 1H), 7.72 (s, 1H), 7.70 (d, J = 7.5 Hz, 1H), 7.49 (s, 1H), 7.42 (d, J = 7.5 Hz, 1H), 6.44 (s, 1H), 5.64 (s, 3H), 5.09 (dd, J = 5.0, 13.5 Hz, 1H), 4.53-4.36 (m, 3H), 4.34-4.26 (m, 1H), 4.23-4.11 (m, 2H), 2.99- 2.84 (m, 1H), 2.63- 2.56 (m, 1H), 2.42-2.29 (m, 4H), 2.03-1.94 (m, 1H) |
| 51 | 6, 32, 44, 46, 49 | 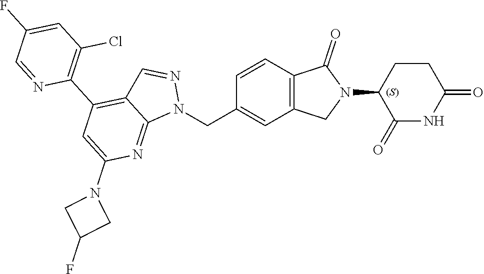 | (3S)-3-(5-{[4- (3-chloro-5- fluoropyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 578.2 | ¹H NMR (400 MHz, DMSO-d6) 10.97 (s, 1H), 8.78 (d, J = 2.4 Hz, 1H), 8.34 (dd, J = 2.4, 8.4 Hz, 1H), 7.74 (s, 1H), 7.69 (d, J = 7.6 Hz, 1H), 7.49 (s, 1H), 7.41 (d, J = 8.4 Hz, 1H), 6.56 (s, 1H), 5.63-5.47 (m, 3H), 5.09 (dd, J = 5.2, 13.2 Hz, 1H), 4.49-4.39 (m, 3H), 4.31-4.27 (m, 1H), 4.22- 4.13 (m, 2H), 2.94- 2.85 (m, 1H), 2.60 (dd, J = 1.6, 3.6 Hz, 1H), 2.39- 2.36 (m, 1H), 1.99-1.96 (m, 1H) |
| 52 | 30 using Intermediate N | 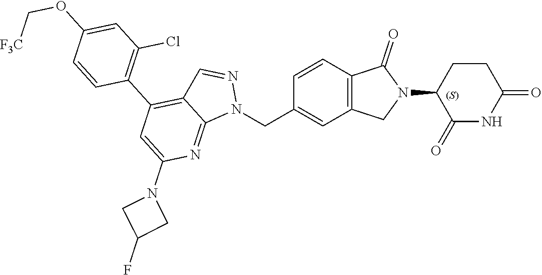 | (3S)-3-[5-({4- [2-chloro-4- (2,2,2- trifluoroethoxy) phenyl]-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 657.0 | ¹H NMR (400 MHz, DMSO-d6) 10.93 (s, 1H), 7.69 (d, J = 8.0 Hz, 1H), 7.63 (s, 1H), 7.52 (s, 2H), 7.41 (d, J = 2.8 Hz, 2H), 7.21 (s, 1H), 6.34 (s, 1H), 5.61 (s, 3H), 5.09 (dd, J = 4.8, 13.2 Hz, 1H), 4.91 (q, J = 8.8 Hz, 2H), 4.48-4.39 (m, 3H), 4.32-4.27 (m, 1H), 4.21- 4.12 (m, 2H), 2.94- 2.84 (m, 1H), 2.61 (dd, J = 2.0, 4.4 Hz, 1H), 2.42- 2.37 (m, 1H), 2.01-1.91 (m, 1H) |
| 53 | 6, 32, 44, 46, 49 | 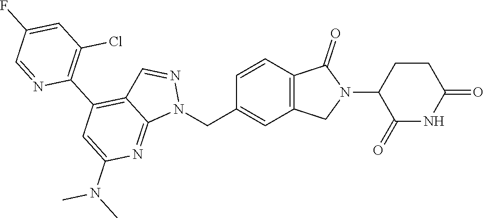 [SMILES: CN(C)c1cc(-c2ncc(F)cc2Cl)c2cnn(Cc3ccc4c(c3)CN(C3CCC(=O)NC3=O)C4=O)c2n1] | 3-(5-{[4-(3- chloro-5- fluoropyridin- 2-yl)-6- [methyl (trifluoromethyl) amino]-1H- pyrazolo[3,4- b]pyridin-1- yl]methyl }-1- oxo-2,3- dihydro-1H- isoindol-2- yl)piperidine- 2,6-dione | 602.3 | ¹H NMR (400 MHz, DMSO-d6) 11.01-10.92 (m, 1H), 8.83 (d, J = 2.4 Hz, 1H), 8.38 (dd, J = 2.4, 8.8 Hz, 1H), 8.02 (s, 1H), 7.70 (d, J = 8.0 Hz, 1H), 7.55 (s, 1H), 7.47 (d, J = 7.6 Hz, 1H), 7.26 (d, J = 1.2 Hz, 1H), 5.76 (s, 2H), 5.09 (dd, J = 5.2, 13.2 Hz, 1H), 4.47-4.39 (m, 1H), 4.34-4.26 (m, 1H), 3.39 (d, J = 2.0 Hz, 3H), 2.96-2.83 (m, 1H), 2.61-2.55 (m, 1H), 2.41- 2.33 (m, 1H), 2.02- 1.92 (m, 1H) |
| 54 | 6, 32, 44, 46, 49 | 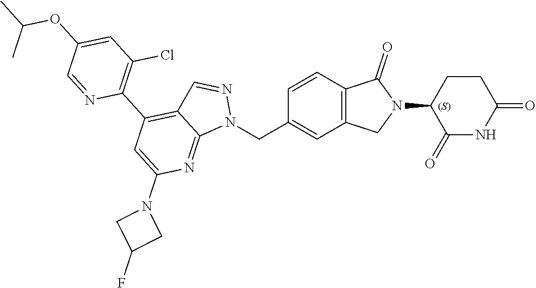 | (3S)-3-[5-({4- [3-chloro-5- (propan-2- yloxy)pyridin- 2-yl]-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 618.3 | ¹H NMR (400 MHz, DMSO-d6): 10.98 (s, 1H), 8.41 (d, J = 2.5 Hz, 1H), 7.74-7.81 (m, 2H), 7.70 (d, J = 8.0 Hz, 1H), 7.49 (s, 1H), 7.41 (d, J = 7.5 Hz, 1H), 6.55 (s, 1H), 5.41-5.70 (m, 3H), 5.10 (dd, J = 13.5, 5.0 Hz, 1H), 4.87 (quin, J = 6.1 Hz, 1H), 4.37-4.52 (m, 3H), 4.25-4.34 (m, 1H), 4.11-4.23 (m, 2H), 2.90 (ddd, J = 18.0, 13.3, 5.3 Hz, 1H), 2.55-2.64 (m, 1H), 2.34-2.42 (m, 1H), 1.93-2.02 (m, 1H), 1.34 (d, J = 6.0 Hz, 6H) |
| 55 | 6, 32, 44, 46, 49 | 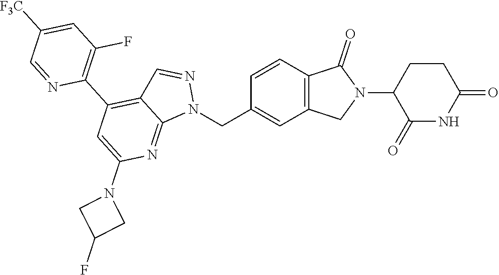 [SMILES: O=C1CCC(N2Cc3cc(Cn4ncc5c(-c6ncc(C(F)(F)F)cc6F)cc(N6CC(F)C6)nc54)ccc3C2=O)C(=O)N1] | 3-[5-({4-[3- fluoro-5- (trifluoromethyl) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 612.2 | ¹H NMR (400 MHz, DMSO-d6, 2 rotamers): 10.98 (s, 1H), 9.09 (s, 1H), 8.58 (d, J = 9.5 Hz, 1H), 8.09 (d, J = 1.6 Hz, 1H), 7.70 (d, J = 7.6 Hz, 1H), 7.49 (s, 1H), 7.41 (d, J = 7.6 Hz, 1H), 6.81 (d, J = 1.1 Hz, 1H), 5.47-5.68 (m, 3H), 5.09 (dd, J = 13.2, 5.1 Hz, 1H), 4.40-4.50 (m, 3H), 4.17- 4.32 (m, 3H), 2.86-2.96 (m, 1H), 2.59-2.63 (m, 1H), 2.36-2.42 (m, 1H), 1.95-2.02 (m, 1H) |
| 56 | 6, 32, 44, 46, 49 | 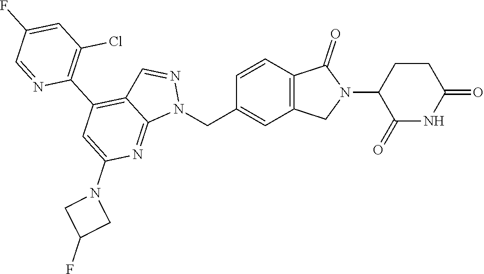 [SMILES: O=C1CCC(N2Cc3cc(Cn4ncc5c(-c6ncc(F)cc6Cl)cc(N6CC(F)C6)nc54)ccc3C2=O)C(=O)N1] | 3-(5-{[4-(3- chloro-5- fluoropyridin- 2-yl)-6-(3- fluoroazetidin- 1-yl)-1H- pyrazolo[3,4- b]pyridin-1- yl]methyl }-1- oxo-2,3- dihydro-1H- isoindol-2- yl)piperidine- 2,6-dione | 578.3 | ¹H NMR (400 MHz, DMSO-d6) 10.98 (s, 1H), 8.78 (d, J = 2.4 Hz, 1H), 8.34 (dd, J = 2.4, 8.4 Hz, 1H), 7.75 (s, 1H), 7.69 (d, J = 8.0 Hz, 1H), 7.49 (s, 1H), 7.41 (d, J = 8.0 Hz, 1H), 6.56 (s, 1H), 5.63- 5.47 (m, 3H), 5.09 (dd, J = 5.2, 13.2 Hz, 1H), 4.49- 4.39 (m, 3H), 4.31- 4.27 (m, 1H), 4.22-4.13 (m, 2H), 2.93-2.85 (m, 1H), 2.58 (dd, J = 2.0, 15.6 Hz, 1H), 2.37 (d, J = 4.4 Hz, 1H), 1.99-1.96 (m, 1H) |
| 57 | 6, 32, 44, 46, 49 | 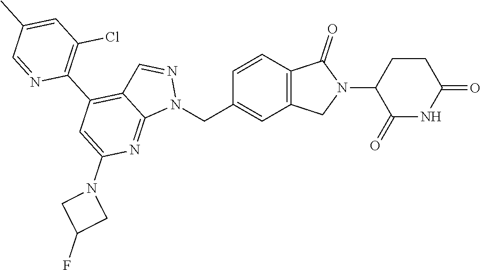 [SMILES: Cc1cnc(-c2cc(N3CC(F)C3)nc3c2cnn3Cc2ccc3c(c2)CN(C2CCC(=O)NC2=O)C3=O)c(Cl)c1] | 3-(5-{[4-(3- chloro-5- methylpyridin- 2-yl)-6-(3- fluoroazetidin- 1-yl)-1H- pyrazolo[3,4- b]pyridin-1- yl]methyl }-1- oxo-2,3- dihydro-1H- isoindol-2- yl)piperidine- 2,6-dione | 574.2 | ¹H NMR (400 MHz, DMSO-d6) 10.98 (s, 1H), 8.55 (d, J = 0.8 Hz, 1H), 8.00 (d, J = 1.2 Hz, 1H), 7.73 (s, 1H), 7.69 (d, J = 8.0 Hz, 1H), 7.48 (s, 1H), 7.40 (d, J = 7.6 Hz, 1H), 6.55 (s, 1H), 5.63 (s, 2H), 5.61-5.46 (m, 1H), 5.09 (dd, J = 5.2, 13.2 Hz, 1H), 4.48-4.39 (m, 3H), 4.31-4.27 (m, 1H), 4.21-4.13 (m, 2H), 2.94- 2.85 (m, 1H), 2.60- 2.56 (m, 1H), 2.40 (s, 3H), 2.37-2.33 (m, 1H), 1.99-1.95 (m, 1H) |
| 58 | 30 | 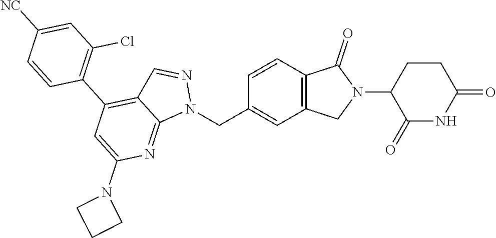 [SMILES: N#Cc1ccc(-c2cc(N3CCC3)nc3c2cnn3Cc2ccc3c(c2)CN(C2CCC(=O)NC2=O)C3=O)c(Cl)c1] | 4-[6-(azetidin- 1-yl)-1-{[2- (2,6- dioxopiperidin- 3-yl)-1-oxo- 2,3-dihydro- 1H-isoindol-5- yl]methyl}-1H- pyrazolo[3,4- b]pyridin-4- yl]-3- chlorobenzonitrile | 566.1 | ¹H NMR (400 MHz, DMSO-d6) 10.97 (d, J = 0.8 Hz, 1H), 8.48 (s, 1H), 8.26 (d, J = 1.2 Hz, 1H), 7.97 (dd, J = 1.2, 8.0 Hz, 1H), 7.74-7.66 (m, 2H), 7.61 (s, 1H), 7.51 (s, 1H), 7.42 (d, J = 7.6 Hz, 1H), 6.29 (s, 1H), 5.60 (s, 2H), 5.09 (dd, J = 4.8, 13.2 Hz, 1H), 4.47-4.40 (m, 1H), 4.33-4.25 (m, 1H), 4.10 (t, J = 7.2 Hz, 4H), 2.96-2.83 (m, 1H), 2.58 (dd, J = 2.4, 15.2 Hz, 1H), 2.42-2.32 (m, 3H), 2.04-1.93 (m, 1H) |
| 59 | 6, 32, 44, 46, 49 | 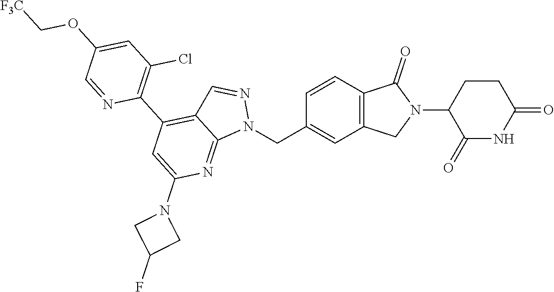 [SMILES: O=C1CCC(N2Cc3cc(Cn4ncc5c(-c6ncc(OCC(F)(F)F)cc6Cl)cc(N6CC(F)C6)nc54)ccc3C2=O)C(=O)N1] | 3-[5-({4-[3- chloro-5- (2,2,2- trifluoroethoxy) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 658.2 | ¹H NMR (400 MHz, DMSO-d₆) δ = 10.98 (s, 1H), 8.57 (d, J = 2.5 Hz, 1H), 8.02 (d, J = 2.5 Hz, 1H), 7.78-7.65 (m, 2H), 7.49 (s, 1H), 7.41 (d, J = 8.0 Hz, 1H), 6.56 (s, 1H), 5.68-5.43 (m, 3H), 5.13- 4.99 (m, 3H), 4.58- 4.38 (m, 3H), 4.36-4.26 (m, 1H), 4.26-4.12 (m, 2H), 2.98-2.77 (m, 1H), 2.68-2.59 (m, 1H), 2.37- 2.31 (m, 1H), 2.04- 1.92 (m, 1H) |
| 60 | 60 | 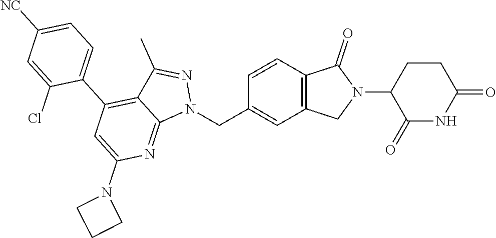 [SMILES: Cc1nn(Cc2ccc3c(c2)CN(C2CCC(=O)NC2=O)C3=O)c2nc(N3CCC3)cc(-c3ccc(C#N)cc3Cl)c12] | 4-[6-(azetidin- 1-yl)-1-{[2- (2,6- dioxopiperidin- 3-yl)-1-oxo- 2,3-dihydro- 1H-isoindol-5- yl]methyl}-3- methyl-1H- pyrazolo[3,4- b]pyridin-4- yl]-3- chlorobenzonitrile | 580.3 | ¹H NMR (400 MHz, DMSO-d6) 11.10-10.81 (m, 1H), 8.32-8.20 (m, 1H), 8.02-7.88 (m, 1H), 7.74-7.60 (m, 2H), 7.51 (s, 1H), 7.42 (d, J = 7.6 Hz, 1H), 6.33-6.06 (m, 1H), 5.62-5.43 (m, 2H), 5.09 (dd, J = 5.2, 13.2 Hz, 1H), 4.50-4.38 (m, 1H), 4.37-4.25 (m, 1H), 4.16-4.04 (m, 4H), 2.96- 2.84 (m, 1H), 2.64- 2.56 (m, 1H), 2.44-2.32 (m, 4H), 2.04-1.93 (m, 1H), 1.81 (s, 2H) |
| 61 | 61 | 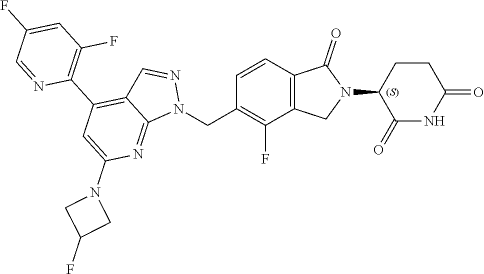 | (3S)-3-(5-{[4- (3-chloro-5- fluoropyridin- 2-yl)-6-(3- fluoroazetidin- 1-yl)-1H- pyrazolo[3,4- b]pyridin-1- yl]methyl}-4- fluoro-1-oxo- 2,3-dihydro- 1H-isoindol-2- yl)piperidine- 2,6-dione | 596.3 | ¹H NMR (400 MHz, DMSO-d6) 11.14-10.91 (m, 1H), 8.78 (d, J = 2.4 Hz, 1H), 8.39-8.30 (m, 1H), 7.74 (s, 1H), 7.56 (d, J = 7.6 Hz, 1H), 7.38 (t, J = 6.8 Hz, 1H), 6.56 (s, 1H), 5.67 (s, 2H), 5.64- 5.44 (m, 1H), 5.11 (dd, J = 4.8, 13.2 Hz, 1H), 4.58 (d, J = 17.6 Hz, 1H), 4.51- 4.38 (m, 3H), 4.24- 4.10 (m, 2H), 2.97-2.85 (m, 1H), 2.63-2.56 (m, 1H), 2.40 (d, J = 4.4 Hz, 1H), 2.03-1.95 (m, 1H) |
| 62 | 30 | 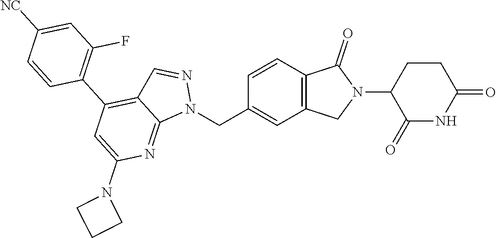 [SMILES: N#Cc1ccc(-c2cc(N3CCC3)nc3c2cnn3Cc2ccc3c(c2)CN(C2CCC(=O)NC2=O)C3=O)c(F)c1] | 4-[6-(azetidin- 1-yl)-1-{[2- (2,6- dioxopiperidin- 3-yl)-1-oxo- 2,3-dihydro- 1H-isoindol-5- yl]methyl}-1H- pyrazolo[3,4- b]pyridin-4- yl]-3- fluorobenzonitrile | 550.0 | ¹H NMR (400 MHz, DMSO-d6) 11.05-10.89 (m, 1H), 8.47 (s, 1H), 8.08 (d, J = 10.4 Hz, 1H), 7.93-7.83 (m, 2H), 7.76 (d, J = 2.4 Hz, 1H), 7.68 (d, J = 7.6 Hz, 1H), 7.53- 7.45 (m, 1H), 7.44-7.35 (m, 1H), 6.40 (s, 1H), 5.61 (s, 2H), 5.08 (dd, J = 4.8, 13.2 Hz, 1H), 4.47- 4.39 (m, 1H), 4.34-4.24 (m, 1H), 4.11 (t, J = 7.2 Hz, 4H), 2.89 (ddd, J = 5.2, 13.2, 17.6 Hz, 1H), 2.63-2.58 (m, 1H), 2.37 (t, J = 7.6 Hz, 3H), 2.03- 1.93 (m, 1H) |
| 63 | 6, 32, 44, 46, 49 | 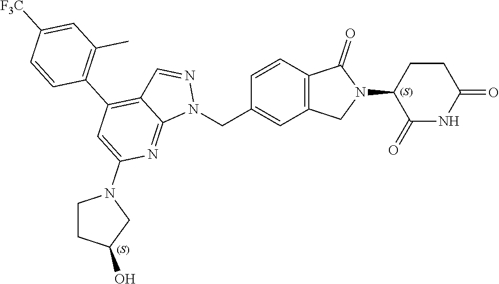 | (3S)-3-[5-({6- [(3S)-3- hydroxypyrrolidin- 1-yl]-4-[2- methyl-4- (trifluoromethyl) phenyl]-1H- pyrazolo[3,4- b]pyridin-1- yl}methyl)-1- oxo-2,3- dihydro-1H- isoindol-2- yl]piperidine- 2,6-dione | 619.3 | ¹H NMR (400 MHz, DMSO-d6) δ 10.90 (s, 1H), 7.70 (s, 1H), 7.66- 7.58 (m, 2H), 7.53-7.44 (m, 3H), 7.39 (d, J = 7.8 Hz, 1H), 6.29 (s, 1H), 5.54 (s, 2H), 5.02 (dd, J = 13.2, 5.1 Hz, 1H), 4.93 (d, J = 3.7 Hz, 1H), 4.41- 4.19 (m, 3H), 3.54 (br dd, J = 11.4, 4.5 Hz, 4H), 2.83 (ddd, J = 17.6, 13.7, 5.4 Hz, 1H), 2.55-2.52 (m, 1H), 2.32-2.25 (m, 1H), 2.23 (s, 3H), 2.04-1.82 (m, 3H) |
| 64 | 6, 32, 44, 46, 49 | 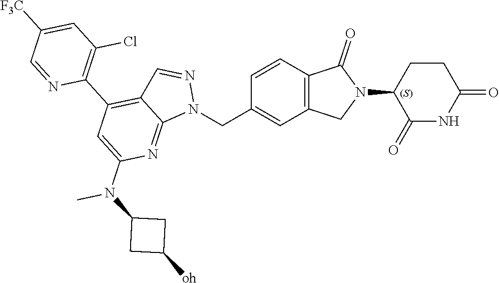 | (3S)-3-[5-({4- [3-chloro-5- (trifluoromethyl) pyridin-2-yl]- 6- {methyl[(1s,3s)- 3- hydroxycyclobutyl] amino}- 1H- pyrazolo[3,4- b]pyridin-1- yl}methyl)-1- oxo-2,3- dihydro-1H- isoindol-2- yl]piperidine- 2,6-dione | 654.0 | ¹H NMR (400 MHz, DMSO-d6): 10.96 (s, 1H), 9.11 (d, J = 1.2 Hz, 1H), 8.69 (d, J = 1.6 Hz, 1H), 7.72-7.68 (m, 2H), 7.50 (s, 1H), 7.44 (d, J = 8 Hz, 1H), 6.87 (s, 1H), 5.64 (s, 2H), 5.10-5.06 (m, 2H), 4.45 (brd, J = 17.6 Hz, 1H), 4.31-4.27 (m, 2H), 3.89 (d, J = 6.8 Hz, 1H), 4.32 (brd, J = 17.6 Hz, 1H), 3.1 (s, 3H), 2.94-2.86 (m, 1H), 2.65- 2.58 (m, 3H), 2.38-2.32 (m, 1H), 2.04-1.96 (m, 3H) |
| 65 | 6, 32, 44, 46, 49 | 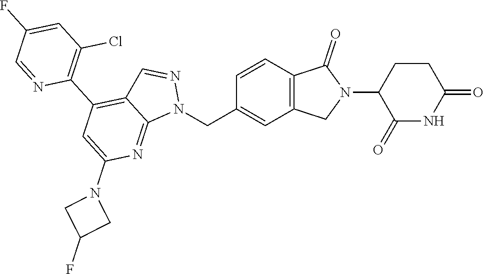 [SMILES: O=C1CCC(N2Cc3cc(Cn4ncc5c(-c6ncc(F)cc6Cl)cc(N6CC(F)C6)nc54)ccc3C2=O)C(=O)N1] | 3-(5-{[4-(3,5- difluoropyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 562.1 | ¹H NMR (400 MHz, DMSO-d₆) δ = 10.98 (s, 1H), 8.77 (d, J = 2.4 Hz, 1H), 8.21 (ddd, J = 2.4, 8.8, 11.1 Hz, 1H), 8.06 (d, J = 1.6 Hz, 1H), 7.69 (d, J = 7.8 Hz, 1H), 7.51- 7.37 (m, 2H), 6.73 (d, J = 1.3 Hz, 1H), 5.70-5.59 (m, 3H), 5.59-5.46 (m, 1H), 5.09 (dd, J = 5.1, 13.3 Hz, 1H), 4.54-4.36 (m, 3H), 4.34-4.13 (m, 3H), 2.90 (ddd, J = 5.4, 13.6, 17.4 Hz, 1H), 2.38- 2.33 (m, 1H), 2.15-1.90 (m, 1H) |
| 66 | 6, 32, 44, 46, 49 | 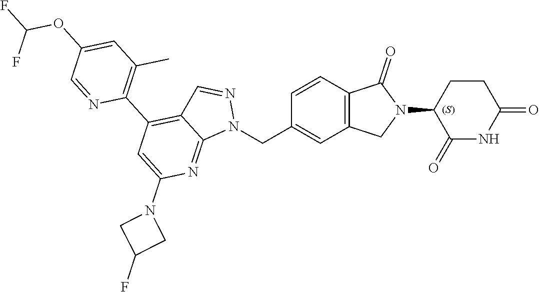 | (3S)-3-[5-({4- [5- (difluoromethoxy)- 3- methylpyridin- 2-yl]-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 606.2 | ¹H NMR (400 MHz, DMSO-d6): 10.98 (s, 1H), 8.48 (d, J = 2.5 Hz, 1H), 7.75 (d, J = 2.5 Hz, 1H), 7.67-7.73 (m, 2H), 7.20-7.62 (m, 3H), 6.43 (s, 1H), 5.42-5.70 (m, 3H), 5.10 (dd, J = 13.3, 5.3 Hz, 1H), 4.36-4.50 (m, 3H), 4.26-4.35 (m, 1H), 4.10-4.23 (m, 2H), 2.83- 2.97 (m, 1H), 2.59 (br d, J = 18.0 Hz, 1H), 2.31- 2.38 (m, 4H), 1.93-2.03 (m, 1H) |
| 67 | 3, 9 | 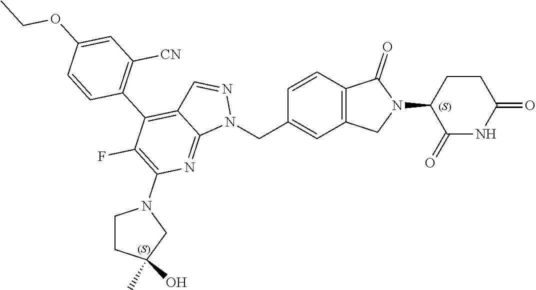 | (3S)-3-(5-{[4- (2-chloro-4- ethoxyphenyl)- 5-fluoro-6- [(3S)-3- hydroxy-3- methylpyrrolidin- 1-yl]-1H- pyrazolo[3,4- b]pyridin-1- yl]methyl}-1- oxo-2,3- dihydro-1H- isoindol-2- yl)piperidine- 2,6-dione | 647.2 | ¹H NMR (400 MHz, DMSO-d₆) δ = 10.99 (s, 1H), 7.70 (d, J = 8.0 Hz, 1H), 7.53-7.42 (m, 4H), 7.23 (t, J = 2.5 Hz, 1H), 7.07 (dd, J = 2.3, 8.8 Hz, 1H), 5.65-5.52 (m, 2H), 5.10 (dd, J = 5.3, 13.3 Hz, 1H), 4.84 (d, J = 2.5 Hz, 1H), 4.49-4.24 (m, 2H), 4.14 (q, J = 7.0 Hz, 2H), 3.89-3.75 (m, 2H), 3.66-3.52 (m, 2H), 3.00- 2.82 (m, 1H), 2.69- 2.59 (m, 1H), 2.39-2.32 (m, 1H), 2.03-1.83 (m, 3H), 1.40-1.35 (m, 6H) |
| 68 | 30 | 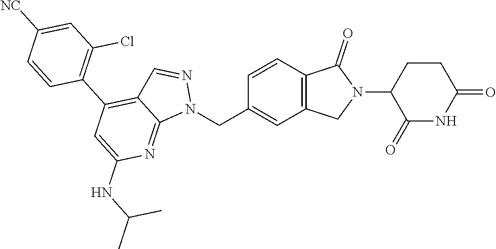 [SMILES: CC(C)Nc1cc(-c2ccc(C#N)cc2Cl)c2cnn(Cc3ccc4c(c3)CN(C3CCC(=O)NC3=O)C4=O)c2n1] | 3-chloro-4-(1- {[2-(2,6- dioxopiperidin- 3-yl)-1-oxo- 2,3-dihydro- 1H-isoindol-5- yl]methyl}-6- [(propan-2- yl)amino]-1H- pyrazolo[3,4- b]pyridin-4- yl)benzonitrile | 568.3 | ¹H NMR (400 MHz, DMSO-d6) 11.07-10.85 (m, 1H), 8.25 (d, J = 1.6 Hz, 1H), 7.95 (dd, J = 1.6, 8.0 Hz, 1H), 7.69 (t, J = 7.6 Hz, 2H), 7.57- 7.48 (m, 2H), 7.45 (d, J = 8.0 Hz, 1H), 7.27-7.17 (m, 1H), 6.37 (s, 1H), 5.57 (s, 2H), 5.08 (dd, J = 5.2, 13.2 Hz, 1H), 4.47- 4.38 (m, 1H), 4.35-4.25 (m, 1H), 4.23-4.13 (m, 1H), 2.96-2.81 (m, 1H), 2.60 (d, J = 0.8 Hz, 1H), 2.44-2.34 (m, 1H), 2.02- 1.91 (m, 1H), 1.21 (dd, J = 1.6, 6.4 Hz, 6H) |
| 69 | 6, 32, 44, 46, 49 | 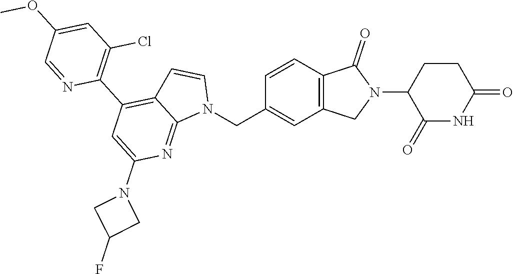 [SMILES: COc1cnc(-c2cc(N3CC(F)C3)nc3c2ccn3Cc2ccc3c(c2)CN(C2CCC(=O)NC2=O)C3=O)c(Cl)c1] | 3-(5-{[4-(3- chloro-5- methoxypyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 590.3 | ¹H NMR (400 MHz, DMSO) 11.08-10.81 (m, 1H), 8.45 (d, J = 2.4 Hz, 1H), 7.79 (d, J = 2.4 Hz, 1H), 7.75 (s, 1H), 7.69 (d, J = 7.6 Hz, 1H), 7.48 (s, 1H), 7.41 (d, J = 8.0 Hz, 1H), 6.54 (s, 1H), 5.63 (s, 2H), 5.57-5.40 (m, 1H), 5.09 (dd, J = 5.2, 13.2 Hz, 1H), 4.49- 4.38 (m, 3H), 4.34-4.26 (m, 1H), 4.23-4.10 (m, 2H), 3.94 (s, 3H), 2.96- 2.83 (m, 1H), 2.60 (d, J = 2.4 Hz, 1H), 2.37 (s, 1H), 2.02-1.92 (m, 1H) |
| 70 | 30 using Intermediate B | 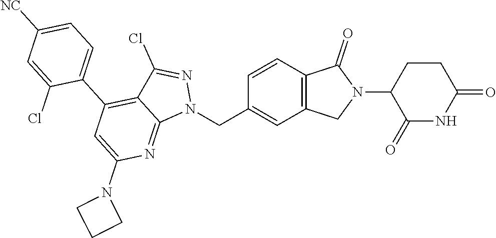 [SMILES: N#Cc1ccc(-c2cc(N3CCC3)nc3c2c(Cl)nn3Cc2ccc3c(c2)CN(C2CCC(=O)NC2=O)C3=O)c(Cl)c1] | 4-[6-(azetidin- 1-yl)-3-chloro- 1-{[2-(2,6- dioxopiperidin- 3-yl)-1-oxo- 2,3-dihydro- 1H-isoindol-5- yl]methyl}-1H- pyrazolo[3,4- b]pyridin-4- yl]-3- chlorobenzonitrile | 600.0 | ¹H NMR (400 MHz, DMSO-d6) 11.11-10.85 (m, 1H), 8.23 (d, J = 1.6 Hz, 1H), 7.96 (dd, J = 1.6, 8.0 Hz, 1H), 7.71 (d, J = 8.0 Hz, 1H), 7.66 (d, J = 8.0 Hz, 1H), 7.54 (d, J = 2.0 Hz, 1H), 7.44 (d, J = 7.6 Hz, 1H), 6.25 (s, 1H), 5.61-5.48 (m, 2H), 5.09 (dd, J = 5.2, 13.6 Hz, 1H), 4.48-4.42 (m, 1H), 4.34-4.28 (m, 1H), 4.12 (t, J = 7.6 Hz, 4H), 2.93-2.86 (m, 1H), 2.61 (d, J = 2.4 Hz, 1H), 2.42- 2.34 (m, 3H), 2.02-1.94 (m, 1H) |
| 71 | 30 | 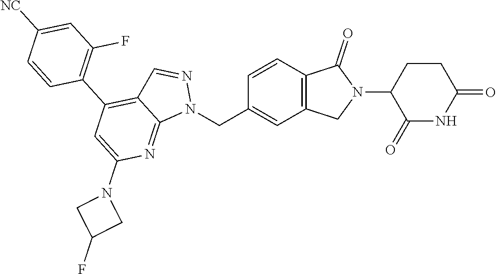 [SMILES: N#Cc1ccc(-c2cc(N3CC(F)C3)nc3c2cnn3Cc2ccc3c(c2)CN(C2CCC(=O)NC2=O)C3=O)c(F)c1] | 4-(1-{[2-(2,6- dioxopiperidin- 3-yl)-1-oxo- 2,3-dihydro- 1H-isoindol-5- yl]methyl}-6- (3- fluoroazetidin- 1-yl)-1H- pyrazolo[3,4- b]pyridin-4- yl)-3- fluorobenzonitrile | 568.1 | ¹H NMR (400 MHz, DMSO-d6) 10.98-10.90 (m, 1H), 8.08 (d, J = 9.6 Hz, 1H), 7.90-7.88 (m, 2H), 7.81 (d, J = 2.4 Hz, 1H), 7.69 (d, J = 7.6 Hz, 1H), 7.50 (s, 1H), 7.41 (d, J = 7.6 Hz, 1H), 6.51 (s, 1H), 5.63 (s, 2H), 5.60- 5.47 (m, 1H), 5.08 (dd, J = 5.2, 13.2 Hz, 1H), 4.49- 4.40 (m, 3H), 4.31- 4.26 (m, 1H), 4.22-4.14 (m, 2H), 2.92 (s, 1H), 2.60 (d, J = 2.4 Hz, 1H), 2.37-2.35 (m, 1H), 2.00- 1.95 (m, 1H) |
| 72 | 6, 32, 44, 46, 49 | 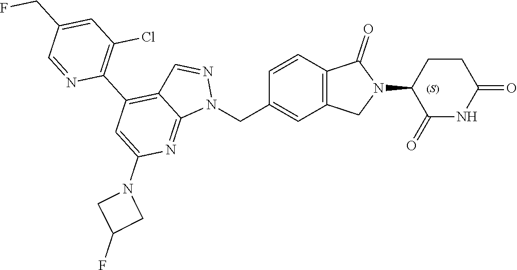 | (3S)-3-[5-({4- [3-chloro-5- (fluoromethyl) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 592.1 | ¹H NMR (400 MHz, DMSO-d6) 10.96 (br s, 1H), 8.77 (t, J = 1.8 Hz, 1H), 8.24 (t, J = 1.8 Hz, 1H), 7.75 (s, 1H), 7.69 (d, J = 8.0 Hz, 1H), 7.49 (s, 1H), 7.42 (d, J = 8.0 Hz, 1H), 6.59 (s, 1H), 5.66 (s, 1H), 5.65-5.44 (m, 4H), 5.08 (dd, J = 5.0, 13.0 Hz, 1H), 4.54-4.38 (m, 3H), 4.35-4.26 (m, 1H), 4.24-4.10 (m, 2H), 2.89 (ddd, J = 5.3, 13.6, 17.6 Hz, 1H), 2.62-2.55 (m, 1H), 2.42-2.33 (m, 1H), 2.02-1.92 (m, 1H) |
| 73 | 6, 32, 44, 46, 49 | 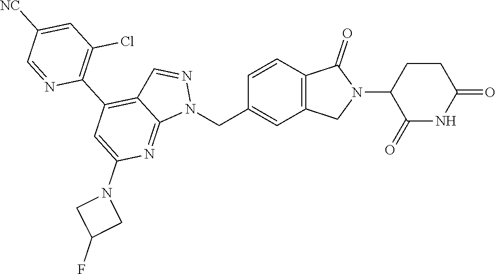 [SMILES: N#Cc1cnc(-c2cc(N3CC(F)C3)nc3c2cnn3Cc2ccc3c(c2)CN(C2CCC(=O)NC2=O)C3=O)c(Cl)c1] | 5-chloro-6-(1- {[2-(2,6- dioxopiperidin- 3-yl)-1-oxo- 2,3-dihydro- 1H-isoindol-5- yl]methyl}-6- (3- fluoroazetidin- 1-yl)-1H- pyrazolo[3,4- b]pyridin-4- yl)pyridine-3- carbonitrile | 585.2 | ¹H NMR (400 MHz, DMSO-d6) 10.97 (s, 1H), 9.16 (d, J = 1.6 Hz, 1H), 8.84 (d, J = 1.6 Hz, 1H), 7.77 (s, 1H), 7.69 (d, J = 8.0 Hz, 1H), 7.49 (s, 1H), 7.41 (d, J = 8.0 Hz, 1H), 6.62 (s, 1H), 5.64- 5.45 (m, 3H), 5.09 (dd, J = 5.2, 13.2 Hz, 1H), 4.50- 4.41 (m, 3H), 4.33- 4.26 (m, 1H), 4.23-4.14 (m, 2H), 2.94-2.86 (m, 1H), 2.61-2.56 (m, 1H), 2.41-2.33 (m, 1H), 2.03- 1.93 (m, 1H) |
| 74 | 6, 32, 44, 46, 49 | 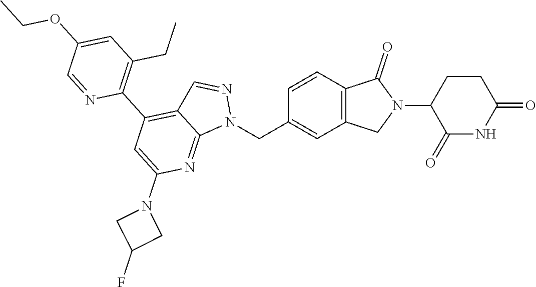 [SMILES: CCOc1cnc(-c2cc(N3CC(F)C3)nc3c2cnn3Cc2ccc3c(c2)CN(C2CCC(=O)NC2=O)C3=O)c(CC)c1] | 3-(5-{[4-(5- ethoxy-3- ethylpyridin-2- yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 598.2 | ¹H NMR (400 MHz, DMSO-d6): 10.98 (s, 1H), 8.25 (d, J = 2.8 Hz, 1H), 7.70 (d, J = 7.8 Hz, 1H), 7.63 (s, 1H), 7.49 (s, 1H), 7.39-7.44 (m, 2H), 6.32 (s, 1H), 5.37-5.73 (m, 3H), 5.10 (dd, J = 13.3, 5.1 Hz, 1H), 4.35-4.54 (m, 3H), 4.26- 4.35 (m, 1H), 4.08-4.26 (m, 4H), 2.82-3.00 (m, 1H), 2.55-2.66 (m, 3H), 2.27-2.43 (m, 1H), 1.93- 2.04 (m, 1H), 1.39 (t, J = 6.9 Hz, 3H), 1.09 (t, J = 7.5 Hz, 3H) |
| 75 | 3, 9, 41, 140 | 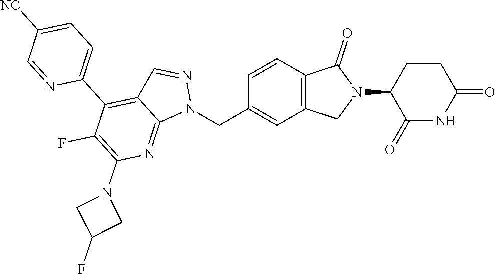 [SMILES: N#Cc1ccc(-c2c(F)c(N3CC(F)C3)nc3c2cnn3Cc2ccc3c(c2)CN([C@H]2CCC(=O)NC2=O)C3=O)nc1] | 6-[1-({2-[(3S)- 2,6- dioxopiperidin- 3-yl]-1-oxo- 2,3-dihydro- 1H-isoindol-5- yl}methyl)-5- fluoro-6-(3- fluoroazetidin- 1-yl)-1H- pyrazolo[3,4- b]pyridin-4- yl]pyridine-3- carbonitrile | 567.2 | ¹H NMR (400 MHz, DMSO-d6) ppm 10.98 (s, 1H), 9.28 (dd, J = 2.1, 0.9 Hz, 1H), 8.53 (dd, J = 8.3, 2.2 Hz, 1H), 8.08- 8.16 (m, 2H), 7.70 (d, J = 7.9 Hz, 1H), 7.48 (s, 1H), 7.41 (d, J = 8.9 Hz, 1H), 5.44-5.69 (m, 3H), 5.09 (dd, J = 13.4, 5.1 Hz, 1H), 4.53-4.71 (m, 2H), 4.25-4.46 (m, 4H), 2.85- 2.96 (m, 1H), 2.30-2.41 (m, 1H), 1.94-2.03 (m, 1H), 1.20-1.32 (m, 1H) |
| 76 | 6, 32, 44, 46, 49 using Intermediate I | 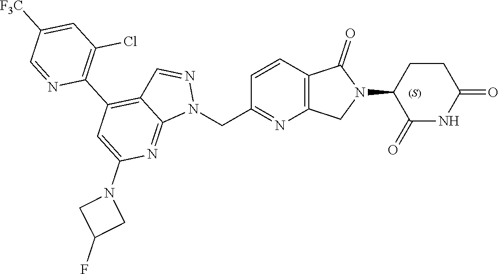 | (3S)-3-[2-({4- [3-chloro-5- (trifluoromethyl) pyridin-2-yl]- 6-(3- fluoroazetidin- 1-yl)-1H- pyrazolo[3,4- b]pyridin-1- yl}methyl)-5- oxo- 5H,6H,7H- pyrrolo[3,4- b]pyridin-6- yl]piperidine- 2,6-dione | 629.2 | ¹H NMR (400 MHz, DMSO-d₆) δ 11.11- 10.86 (m, 1H), 9.13 (d, J = 1.2 Hz, 1H), 8.73 (d, J = 1.6 Hz, 1H), 8.11 (d, J = 8.0 Hz, 1H), 7.84 (s, 1H), 7.15 (d, J = 8.0 Hz, 1H), 6.64 (s, 1H), 5.75 (s, 2H), 5.64-5.42 (m, 1H), 5.22- 5.10 (m, 1H), 4.58-4.29 (m, 4H), 4.21-4.09 (m, 2H), 2.97-2.84 (m, 1H), 2.63-2.55 (m, 1H), 2.44- 2.38 (m, 1H), 2.05-1.97 (m, 1H) |
| 77 | 6, 32, 44, 46, 49 using Intermediate J | 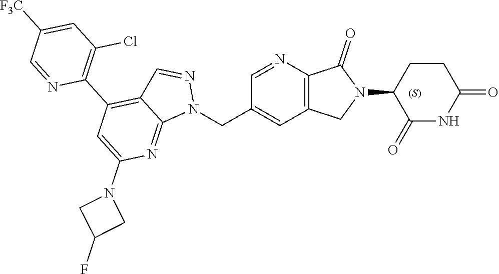 | (3S)-3-[3-({4- [3-chloro-5- (trifluoromethyl) pyridin-2-yl]- 6-(3- fluoroazetidin- 1-yl)-1H- pyrazolo[3,4- b]pyridin-1- yl}methyl)-7- oxo- 5H,6H,7H- pyrrolo[3,4- b]pyridin-6- yl]piperidine- 2,6-dione | 629.2 | ¹H NMR (400 MHz, DMSO-d6) 11.08-10.93 (m, 1H), 9.12 (s, 1H), 8.81-8.69 (m, 2H), 7.94 (s, 1H), 7.80 (s, 1H), 6.63 (s, 1H), 5.73-5.66 (m, 2H), 5.65-5.43 (m, 1H), 5.13 (dd, J = 5.4, 13.2 Hz, 1H), 4.52-4.40 (m, 3H), 4.38-4.30 (m, 1H), 4.25-4.13 (m, 2H), 2.96- 2.82 (m, 1H), 2.64- 2.56 (m, 1H), 2.41-2.34 (m, 1H), 2.02-1.95 (m, 1H) |
| 78 | 78 | 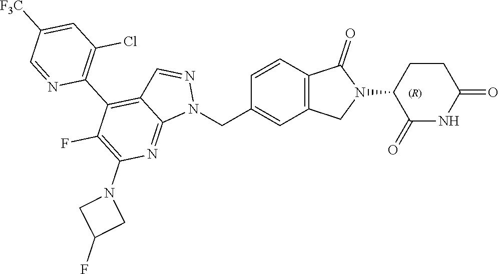 | (3R)-3-[5-({4- [3-chloro-5- (trifluoromethyl) pyridin-2-yl]- 5-fluoro-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 646.3 | ¹H NMR (400 MHz, DMSO-d6) 10.98 (s, 1H), 9.16 (d, J = 1.2 Hz, 1H), 8.77 (d, J = 1.2 Hz, 1H), 7.75-7.67 (m, 2H), 7.52 (s, 1H), 7.44 (d, J = 8.0 Hz, 1H), 5.67-5.42 (m, 3H), 5.09 (dd, J = 4.8, 13.2 Hz, 1H), 4.68-4.52 (m, 2H), 4.49-4.38 (m, 1H), 4.37-4.23 (m, 3H), 2.98-2.82 (m, 1H), 2.63- 2.55 (m, 1H), 2.42- 2.29 (m, 1H), 2.03-1.94 (m, 1H) |
| 79 | 6, 32, 44, 46, 49 | 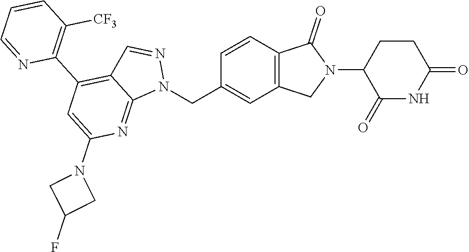 [SMILES: O=C1CCC(N2Cc3cc(Cn4ncc5c(-c6ncccc6C(F)(F)F)cc(N6CC(F)C6)nc54)ccc3C2=O)C(=O)N1] | 3-(5-{[6-(3- fluoroazetidin- 1-yl)-4-[3- (trifluoromethyl) pyridin-2-yl]- 1H- pyrazolo[3,4- b]pyridin-1- yl]methyl}-1- oxo-2,3- dihydro-1H- isoindol-2- yl)piperidine- 2,6-dione | 594.3 | ¹H NMR (400 MHz, DMSO) 10.97 (s, 1H), 8.97 (d, J = 4.0 Hz, 1H), 8.41 (d, J = 8.0 Hz, 1H), 7.77 (dd, J = 4.4, 8.0 Hz, 1H), 7.70 (d, J = 7.6 Hz, 1H), 7.58 (s, 1H), 7.49 (s, 1H), 7.41 (d, J = 8.4 Hz, 1H), 6.36 (s, 1H), 5.63 (s, 2H), 5.62-5.44 (m, 1H), 5.16-5.04 (m, 1H), 4.47- 4.38 (m, 3H), 4.34- 4.26 (m, 1H), 4.21-4.08 (m, 2H), 2.94-2.85 (m, 1H), 2.60 (d, J = 2.4 Hz, 1H), 2.37 (d, J = 4.4 Hz, 1H), 2.02-1.94 (m, 1H) |
| 80 | 6, 32, 44, 46, 49 | 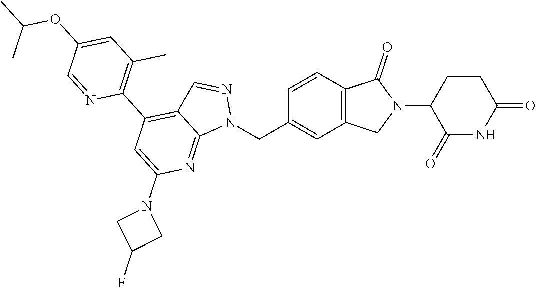 [SMILES: Cc1cc(OC(C)C)cnc1-c1cc(N2CC(F)C2)nc2c1cnn2Cc1ccc2c(c1)CN(C1CCC(=O)NC1=O)C2=O] | 3-(5-{[6-(3- fluoroazetidin- 1-yl)-4-[3- methyl-5- (propan-2- yloxy)pyridin- 2-yl]-1H- pyrazolo[3,4- b]pyridin-1- yl]methyl}-1- oxo-2,3- dihydro-1H- isoindol-2- yl)piperidine- 2,6-dione | 598.3 | ¹H NMR (400 MHz, DMSO-d6): 10.98 (s, 1H), 8.23 (d, J = 2.6 Hz, 1H), 7.63-7.76 (m, 2H), 7.48 (s, 1H), 7.37-7.44 (m, 2H), 6.39 (s, 1H), 5.42-5.69 (m, 3H), 5.10 (dd, J = 13.3, 5.1 Hz, 1H), 4.71-4.84 (m, 1H), 4.35- 4.51 (m, 3H), 4.25-4.33 (m, 1H), 4.08-4.23 (m, 2H), 2.82-2.98 (m, 1H), 2.55-2.64 (m, 1H), 2.28- 2.42 (m, 4H), 1.92-2.05 (m, 1H), 1.33 (d, J = 6.0 Hz, 6H) |
| 81 | 6, 32, 44, 46, 49 | 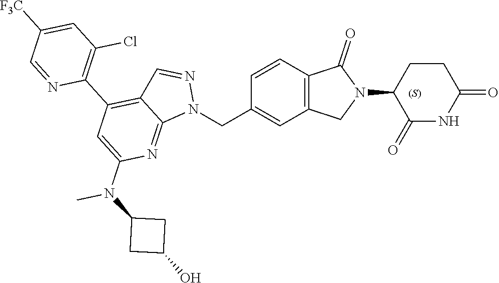 | (3S)-3-[5-({4- [3-chloro-5- (trifluoromethyl) pyridin-2-yl]- 6- {methyl[(1r,3r)- 3- hydroxycyclobutyl] amino}-1H- pyrazolo[3,4- b]pyridin-1- yl}methyl)-1- oxo-2,3- dihydro-1H- isoindol-2- yl]piperidine- 2,6-dione | 654.0 | ¹H NMR (400 MHz, DMSO-d6): 10.96 (s, 1H), 9.11 (d, J = 0.8 Hz, 1H), 8.69 (d, J = 1.2 Hz, 1H), 7.73-7.68 (m, 2H), 7.52 (s, 1H), 7.45 (d, J = 7.6 Hz, 1H), 6.84 (s, 1H), 5.64 (s, 2H), 5.10-5.06 (m, 3H), 4.46 (brd, J = 17.6 Hz, 1H), 4.32-4.27 (m, 2H), 3.1 (s, 3H), 2.94-2.86 (m, 1H), 2.65- 2.58 (m, 1H), 2.38-2.32 (m, 1H), 2.04-1.96 (m, 3H) |
| 82 | 6, 32, 44, 46, 49 | 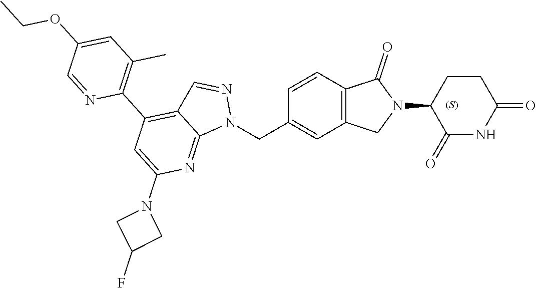 | (3S)-3-(5-{[4- (5-ethoxy-3- methylpyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 584.2 | ¹H NMR (400 MHz, DMSO-d6): 10.98 (s, 1H), 7.65-7.76 (m, 2H), 7.48 (s, 1H), 7.37-7.44 (m, 2H), 6.39 (s, 1H), 5.40-5.70 (m, 2H), 5.10 (dd, J = 13.0, 5.0 Hz, 1H), 4.35-4.52 (m, 2H), 4.25- 4.33 (m, 1H), 4.08-4.22 (m, 3H), 3.40-3.55 (m, 2H), 2.83-2.97 (m, 1H), 2.61 (br s, 2H), 2.33 (s, 4H), 1.87-2.07 (m, 1H), 1.84-2.06 (m, 1H), 1.38 (t, J = 7.0 Hz, 3H) |
| 83 | 6, 32, 44, 46, 49 | 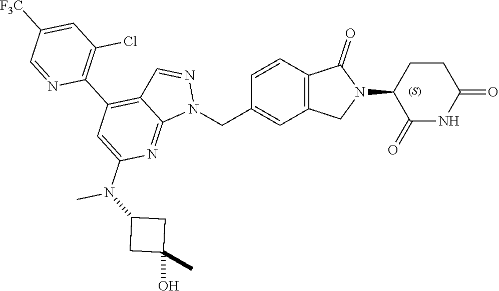 | (3S)-3-[5-({4- [3-chloro-5- (trifluoromethyl) pyridin-2-yl]- 6- {methyl[(1r,3s)- 3-hydroxy-3- methylcyclobutyl] amino}-1H- pyrazolo[3,4- b]pyridin-1- yl}methyl)-1- oxo-2,3- dihydro-1H- isoindol-2- yl]piperidine- 2,6-dione | 668.07 | ¹H NMR (400 MHz, DMSO-d6): 10.98 (s, 1H), 9.12 (dd, J = 1.9, 0.8 Hz, 1H), 8.70 (dd, J = 2.0, 0.6 Hz, 1H), 7.73 (s, 1H), 7.71 (d, J = 8.1 Hz, 1H), 7.52 (s, 1H), 7.43-7.49 (m, 1H), 6.90 (s, 1H), 5.65 (s, 2H), 4.85-5.18 (m, 2H), 4.27-4.47 (m, 3H), 3.10 (s, 3H), 2.84- 2.96 (m, 1H), 2.61 (br d, J = 2.9 Hz, 1H), 2.32-2.38 (m, 3H), 2.19-2.27 (m, 2H), 1.91-2.04 (m, 1H), 1.32 (s, 3H) |
| 84 | 6, 32, 44, 46, 49 | 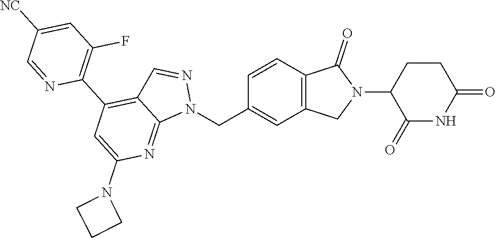 [SMILES: N#Cc1cnc(-c2cc(N3CCC3)nc3c2cnn3Cc2ccc3c(c2)CN(C2CCC(=O)NC2=O)C3=O)c(F)c1] | 6-[6-(azetidin- 1-yl)-1-{[2- (2,6- dioxopiperidin- 3-yl)-1-oxo- 2,3-dihydro- 1H-isoindol-5- yl]methyl}-1H- pyrazolo[3,4- b]pyridin-4- yl]-5- fluoropyridine- 3-carbonitrile | 551.2 | ¹H NMR (400 MHz, DMSO-d6) 10.97 (s, 1H), 9.10 (d, J = 1.2 Hz, 1H), 8.66 (dd, J = 1.6, 10.8 Hz, 1H), 8.03 (d, J = 1.6 Hz, 1H), 7.68 (d, J = 7.6 Hz, 1H), 7.49-7.44 (m, 1H), 7.38 (d, J = 8.0 Hz, 1H), 6.71 (d, J = 1.2 Hz, 1H), 5.63 (s, 2H), 5.08 (dd, J = 5.2, 13.2 Hz, 1H), 4.46-4.39 (m, 1H), 4.33-4.25 (m, 1H), 4.12 (t, J = 7.6 Hz, 4H), 2.95-2.83 (m, 1H), 2.58 (dd, J = 2.0, 17.2 Hz, 1H), 2.43-2.34 (m, 3H), 2.03-1.92 (m, 1H) |
| 85 | 6, 32, 44, 46, 49 | 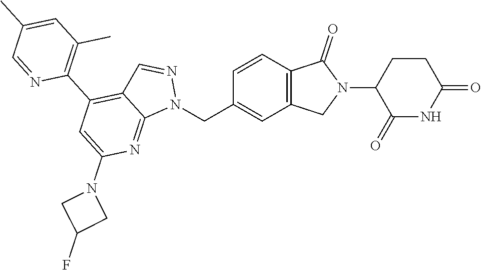 [SMILES: Cc1cnc(-c2cc(N3CC(F)C3)nc3c2cnn3Cc2ccc3c(c2)CN(C2CCC(=O)NC2=O)C3=O)c(C)c1] | 3-(5-{[4-(3,5- dimethylpyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 554.2 | ¹H NMR (400 MHz, DMSO-d6): 10.97 (s, 1H), 8.39 (s, 1H), 7.68- 7.71 (m, 2H), 7.64 (s, 1H), 7.49 (s, 1H), 7.41 (d, J = 7.5 Hz, 1H), 6.40 (s, 1H), 5.63 (s, 1H), 5.39- 5.69 (m, 1H), 5.09 (dd, J = 13.3, 5.1 Hz, 1H), 4.37-4.49 (m, 3H), 4.27- 4.35 (m, 1H), 4.11-4.23 (m, 3H), 2.85-2.95 (m, 1H), 2.56-2.62 (m, 1H), 2.33-2.39 (m, 4H), 2.28 (s, 3H), 1.94-2.03 (m, 1H) |
| 86 | 6, 32, 44, 46, 49 | 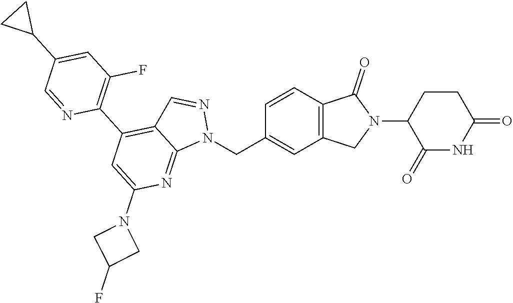 [SMILES: O=C1CCC(N2Cc3cc(Cn4ncc5c(-c6ncc(C7CC7)cc6F)cc(N6CC(F)C6)nc54)ccc3C2=O)C(=O)N1] | 3-(5-{[4-(5- cyclopropyl-3- fluoropyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 584.2 | ¹H NMR (400 MHz, DMSO-d₆) δ = 10.98 (s, 1H), 8.53 (t, J = 1.7 Hz, 1H), 8.08 (d, J = 1.5 Hz, 1H), 7.69 (d, J = 7.8 Hz, 1H), 7.59 (dd, J = 1.8, 12.6 Hz, 1H), 7.47 (s, 1H), 7.40 (d, J = 8.0 Hz, 1H), 6.71 (d, J = 1.0 Hz, 1H), 5.69-5.59 (m, 2H), 5.48 (qd, J = 2.9, 5.8 Hz, 1H), 5.09 (dd, J = 5.0, 13.3 Hz, 1H), 4.51-4.39 (m, 3H), 4.33-4.13 (m, 3H), 2.96-2.84 (m, 1H), 2.48-2.25 (m, 2H), 2.17- 2.07 (m, 1H), 2.03- 1.93 (m, 1H), 1.19-1.06 (m, 2H), 0.98-0.85 (m, 2H) |
| 87 | 87 | 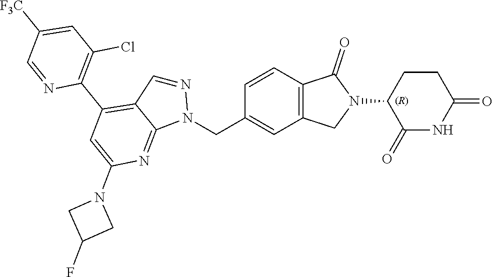 | (3R)-3-[5-({4- [3-chloro-5- (trifluoromethyl) pyridin-2-yl]- 6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 628.2 | ¹H NMR (400 MHz, DMSO-d6) 10.98 (s, 1H), 9.12 (s, 1H), 8.71 (d, J = 1.2 Hz, 1H), 7.79 (s, 1H), 7.70 (d, J = 7.6 Hz, 1H), 7.50 (s, 1H), 7.42 (d, J = 8.0 Hz, 1H), 6.62 (s, 1H), 5.68-5.44 (m, 3H), 5.09 (dd, J = 5.2, 13.1 Hz, 1H), 4.55-4.37 (m, 3H), 4.33-4.26 (m, 1H), 4.24-4.10 (m, 2H), 2.96- 2.82 (m, 1H), 2.65- 2.54 (m, 1H), 2.39-2.30 (m, 1H), 2.03-1.91 (m, 1H) |
| 88 | 6, 32, 44, 46, 49 | 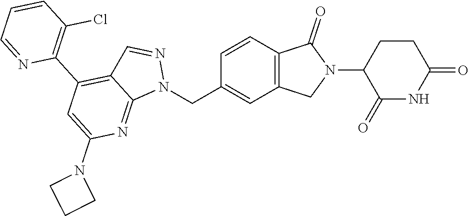 [SMILES: O=C1CCC(N2Cc3cc(Cn4ncc5c(-c6ncccc6Cl)cc(N6CCC6)nc54)ccc3C2=O)C(=O)N1] | 3-(5-{[6- (azetidin-1-yl)- 4-(3- chloropyridin- 2-yl)-1H- pyrazolo[3,4- b]pyridin-1- yl]methyl}-1- oxo-2,3- dihydro-1H- isoindol-2- yl)piperidine- 2,6-dione | 560.1 | ¹H NMR (400 MHz, DMSO-d6) 10.97 (s, 1H), 8.70 (dd, J = 1.2, 4.8 Hz, 1H), 8.15 (dd, J = 1.2, 8.0 Hz, 1H), 7.73 (s, 1H), 7.69 (d, J = 8.0 Hz, 1H), 7.58 (dd, J = 4.8, 8.4 Hz, 1H), 7.49 (s, 1H), 7.41 (d, J = 8.0 Hz, 1H), 6.57 (s, 1H), 5.64 (s, 2H), 5.62-5.46 (m, 1H), 5.09 (dd, J = 5.2, 13.2 Hz, 1H), 4.49-4.40 (m, 3H), 4.32-4.27 (m, 1H), 4.22- 4.13 (m, 2H), 2.94- 2.85 (m, 1H), 2.61-2.55 (m, 1H), 2.40-2.34 (m, 1H), 2.00-1.95 (m, 1H) |
| 89 | 6, 32, 44, 46, 49 | 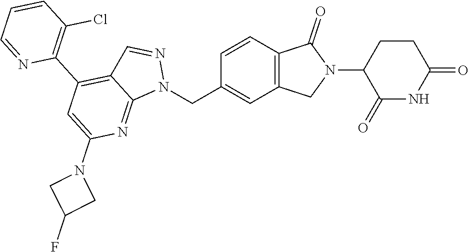 [SMILES: O=C1CCC(N2Cc3cc(Cn4ncc5c(-c6ncccc6Cl)cc(N6CC(F)C6)nc54)ccc3C2=O)C(=O)N1] | 3-(5-{[4-(3- chloropyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 560.1 | ¹H NMR (400 MHz, DMSO-d6) 10.97 (s, 1H), 8.70 (dd, J = 1.2, 4.8 Hz, 1H), 8.15 (dd, J = 1.2, 8.0 Hz, 1H), 7.73 (s, 1H), 7.69 (d, J = 8.0 Hz, 1H), 7.58 (dd, J = 4.8, 8.4 Hz, 1H), 7.49 (s, 1H), 7.41 (d, J = 8.0 Hz, 1H), 6.57 (s, 1H), 5.64 (s, 2H), 5.62-5.46 (m, 1H), 5.09 (dd, J = 5.2, 13.2 Hz, 1H), 4.49-4.40 (m, 3H), 4.32-4.27 (m, 1H), 4.22- 4.13 (m, 2H), 2.94- 2.85 (m, 1H), 2.61-2.55 (m, 1H), 2.40-2.34 (m, 1H), 2.00-1.95 (m, 1H) |
| 90 | 6, 32, 44, 46, 49 | 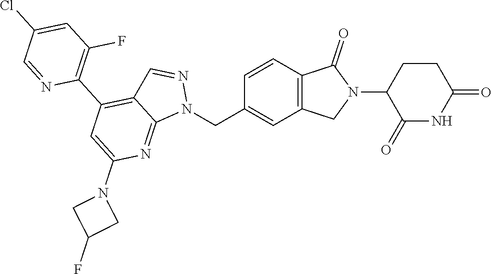 [SMILES: O=C1CCC(N2Cc3cc(Cn4ncc5c(-c6ncc(Cl)cc6F)cc(N6CC(F)C6)nc54)ccc3C2=O)C(=O)N1] | 3-(5-{[4-(5- chloro-3- fluoropyridin- 2-yl)-6-(3- fluoroazetidin- 1-yl)-1H- pyrazolo[3,4- b]pyridin-1- yl]methyl }-1- oxo-2,3- dihydro-1H- isoindol-2- yl)piperidine- 2,6-dione | 578.2 | ¹H NMR (400 MHz, DMSO-d6) 10.97 (s, 1H), 8.76 (s, 1H), 8.35 (dd, J = 1.6, 10.4 Hz, 1H), 8.07 (d, J = 1.6 Hz, 1H), 7.68 (d, J = 8.0 Hz, 1H), 7.47 (s, 1H), 7.39 (d, J = 8.4 Hz, 1H), 6.74 (s, 1H), 5.65 (s, 2H), 5.63- 5.45 (m, 1H), 5.09 (dd, J = 5.2, 13.6 Hz, 1H), 4.50- 4.45 (m, 2H), 4.42- 4.40 (m, 1H), 4.31-4.26 (m, 1H), 4.23-4.15 (m, 2H), 2.94-2.85 (m, 1H), 2.60 (d, J = 2.0 Hz, 1H), 2.36 (d, J = 4.0 Hz, 1H), 2.00-1.93 (m, 1H) |
| 91 | 6, 32, 44, 46, 49 | 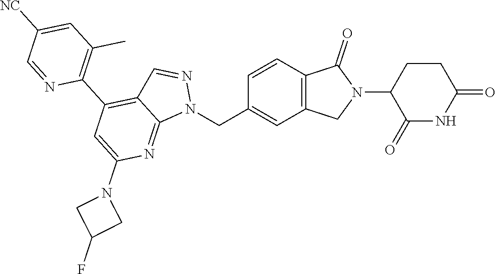 [SMILES: Cc1cc(C#N)cnc1-c1cc(N2CC(F)C2)nc2c1cnn2Cc1ccc2c(c1)CN(C1CCC(=O)NC1=O)C2=O] | 6-(1-{[2-(2,6- dioxopiperidin- 3-yl)-1-oxo- 2,3-dihydro- 1H-isoindol-5- yl]methyl}-6- (3- fluoroazetidin- 1-yl)-1H- pyrazolo[3,4- b]pyridin-4- yl)-5- methylpyridine- 3-carbonitrile | 565.2 | ¹H NMR (400 MHz, DMSO-d6): 10.98 (s, 1H), 9.01 (d, J = 1.4 Hz, 1H), 8.41 (dd, J = 1.9, 0.7 Hz, 1H), 7.72 (s, 1H), 7.70 (d, J = 7.8 Hz, 1H), 7.49 (s, 1H), 7.42 (d, J = 7.8 Hz, 1H), 6.49 (s, 1H), 5.44-5.68 (m, 3H), 5.10 (dd, J = 13.3, 5.1 Hz, 1H), 4.37-4.51 (m, 3H), 4.26-4.34 (m, 1H), 4.11- 4.24 (m, 2H), 2.83-2.98 (m, 1H), 2.55-2.64 (m, 1H), 2.33-2.40 (m, 4H), 1.93-2.03 (m, 1H) |
| 92 | 30 | 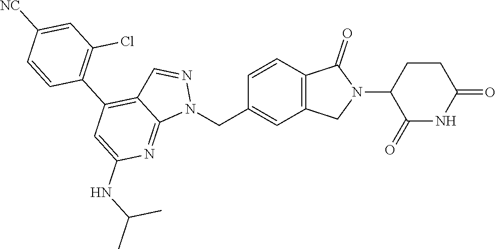 [SMILES: CC(C)Nc1cc(-c2ccc(C#N)cc2Cl)c2cnn(Cc3ccc4c(c3)CN(C3CCC(=O)NC3=O)C4=O)c2n1] | 4-(1-{[2-(2,6- dioxopiperidin- 3-yl)-1-oxo- 2,3-dihydro- 1H-isoindol-5- yl]methyl}-6- [(propan-2- yl)amino]-1H- pyrazolo[3,4- b]pyridin-4- yl)-3- fluorobenzonitrile | 552.1 | ¹H NMR (400 MHz, DMSO-d6) 10.99-10.94 (m, 1H), 8.43 (s, 1H), 8.07-8.05 (m, 1H), 7.87- 7.81 (m, 2H), 7.69- 7.67 (m, 2H), 7.52 (s, 1H), 7.43 (d, J = 7.6 Hz, 1H), 7.22 (d, J = 7.2 Hz, 1H), 6.51 (s, 1H), 5.58 (s, 2H), 5.08 (dd, J = 4.8, 13.2 Hz, 1H), 4.44-4.26 (m, 2H), 4.22-4.14 (m, 1H), 2.94-2.84 (m, 1H), 2.60-2.55 (m, 1H), 2.38 (d, J = 4.4 Hz, 1H), 1.99- 1.94 (m, 1H), 1.20 (dd, J = 2.0, 6.4 Hz, 6H) |
| 93 | 6, 32, 44, 46, 49 | 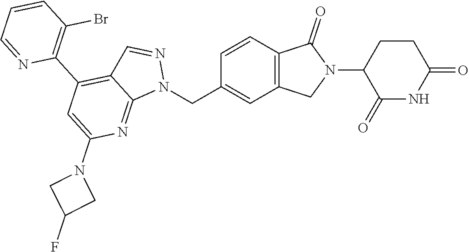 [SMILES: O=C1CCC(N2Cc3cc(Cn4ncc5c(-c6ncccc6Br)cc(N6CC(F)C6)nc54)ccc3C2=O)C(=O)N1] | 3-(5-{[4-(3- bromopyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 604.2 | ¹H NMR (400 MHz, DMSO-d6) 11.10-10.84 (m, 1H), 8.72 (dd, J = 1.2, 4.4 Hz, 1H), 8.29 (dd, J = 1.2, 8.0 Hz, 1H), 7.73-7.63 (m, 2H), 7.53- 7.37 (m, 3H), 6.52 (s, 1H), 5.67-5.43 (m, 3H), 5.09 (dd, J = 5.2, 13.2 Hz, 1H), 4.51-4.36 (m, 3H), 4.34-4.25 (m, 1H), 4.23-4.10 (m, 2H), 2.98- 2.82 (m, 1H), 2.62- 2.55 (m, 1H), 2.42-2.35 (m, 1H), 2.06-1.91 (m, 1H) |
| 94 | 6, 32, 44, 46, 49 | 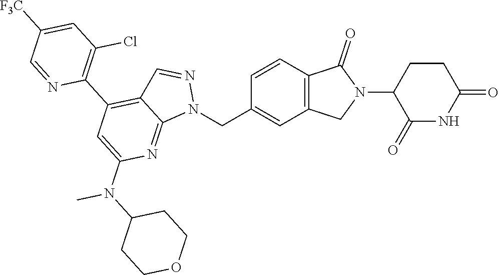 [SMILES: CN(c1cc(-c2ncc(C(F)(F)F)cc2Cl)c2cnn(Cc3ccc4c(c3)CN(C3CCC(=O)NC3=O)C4=O)c2n1)C1CCOCC1] | 3-[5-({4-[3- chloro-5- (trifluoromethyl) pyridin-2-yl]- 6- [methyl(oxan- 4-yl)amino]- 1H- pyrazolo[3,4- b]pyridin-1- yl}methyl)-1- oxo-2,3- dihydro-1H- isoindol-2- yl]piperidine- 2,6-dione | 668.1 | ¹H NMR (400 MHz, DMSO-d6): 10.98 (s, 1H), 9.13 (d, J = 1.0 Hz, 1H), 8.71 (d, J = 1.3 Hz, 1H), 7.73 (s, 1H), 7.69 (d, J = 7.9 Hz, 1H), 7.55 (s, 1H), 7.47 (d, J = 7.6 Hz, 1H), 6.94 (s, 1H), 5.65 (s, 2H), 5.09 (dd, J = 13.2, 5.2 Hz, 1H), 4.72 (br t, J = 8.3 Hz, 1H), 4.26-4.46 (m, 2H), 3.97 (br dd, J = 10.9, 3.8 Hz, 2H), 3.49 (br t, J = 11.5 Hz, 2H), 3.00 (s, 3H), 2.83-2.96 (m, 1H), 2.65-2.70 (m, 1H), 2.32- 2.37 (m, 1H), 1.93-2.02 (m, 1H), 1.83 (qd, J = 12.3, 4.5 Hz, 2H), 1.55-1.65 (m, 2H) |
| 95 | 30 | 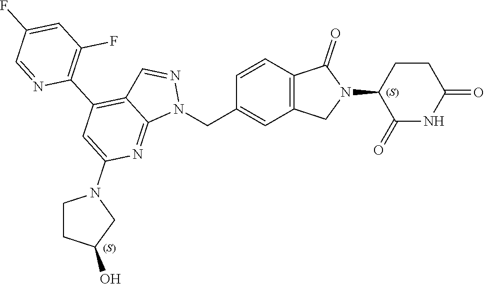 | (3S)-3-(5-{[4- (2,4- difluorophenyl)- 6-[(3S)-3- hydroxypyrrolidin- 1-yl]-1H- pyrazolo[3,4- b]pyridin-1- yl]methyl}-1- oxo-2,3- dihydro-1H- isoindol-2- yl)piperidine- 2,6-dione | 573.2 | ¹H NMR (400 MHz, DMSO-d6) δ 10.90 (s, 1H), 7.69 (td, J = 8.6, 6.5 Hz, 1H), 7.64 (d, J = 2.2 Hz, 1H), 7.62 (d, J = 8.1 Hz, 1H), 7.45-7.35 (m, 3H), 7.24-7.17 (m, 1H), 6.41 (s, 1H), 5.54 (s, 2H), 5.02 (dd, J = 13.2, 4.9 Hz, 1H), 4.93 (d, J = 3.7 Hz, 1H), 4.40-4.18 (m, 3H), 3.63-3.47 (m, 3H), 3.46- 3.36 (m, 1H), 2.83 (ddd, J = 17.4, 13.6, 5.4 Hz, 1H), 2.55-2.48 (m, 2H), 2.35-2.21 (m, 1H), 2.05- 1.81 (m, 3H) |
| 96 | 30 | 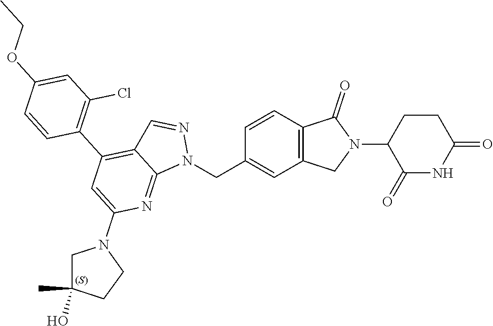 | 3-(5-{[4-(2- chloro-4- ethoxyphenyl)- 6-[(3S)-3- hydroxy-3- methylpyrrolidin- 1-yl]-1H- pyrazolo[3,4- b]pyridin-1- yl]methyl}-1- oxo-2,3- dihydro-1H- isoindol-2- yl)piperidine- 2,6-dione | 629.2 | ¹H NMR (400 MHz, DMSO-d6): 10.98 (s, 1H), 7.70 (d, J = 7.9 Hz, 1H), 7.56 (s, 1H), 7.51 (s, 1H), 7.41-7.48 (m, 2H), 7.20 (d, J = 2.5 Hz, 1H), 7.05 (dd, J = 8.6, 2.5 Hz, 1H), 6.34 (s, 1H), 5.60 (s, 2H), 5.09 (dd, J = 13.1, 4.9 Hz, 1H), 4.84 (s, 1H), 4.44 (d, J = 17.6 Hz, 1H), 4.29 (d, J = 17.6 Hz, 1H), 4.13 (q, J = 7.0 Hz, 2H), 3.47-3.75 (m, 3H), 3.39 (br d, J = 10.9 Hz, 1H), 2.85-2.97 (m, 1H), 2.59- 2.64 (m, 1H), 2.36-2.43 (m, 1H), 1.88-2.05 (m, 3H), 1.34-1.39 (m, 6H) |
| 97 | 6, 32, 44, 46, 49 | 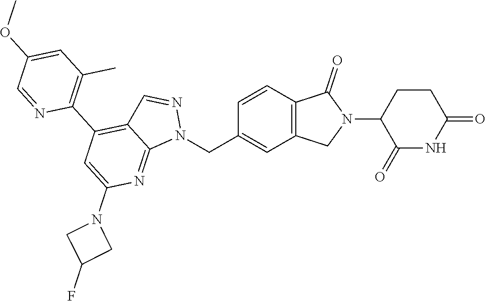 [SMILES: COc1cnc(-c2cc(N3CC(F)C3)nc3c2cnn3Cc2ccc3c(c2)CN(C2CCC(=O)NC2=O)C3=O)c(C)c1] | 3-(5-{[6-(3- fluoroazetidin- 1-yl)-4-(5- methoxy-3- methylpyridin- 2-yl)-1H- pyrazolo[3,4- b]pyridin-1- yl]methyl}-1- oxo-2,3- dihydro-1H- isoindol-2- yl)piperidine- 2,6-dione | 570.3 | ¹H NMR (400 MHz, DMSO-d6): 10.98 (s, 1H), 8.28 (d, J = 2.5 Hz, 1H), 7.63-7.77 (m, 2H), 7.09-7.51 (m, 3H), 6.39 (s, 1H), 5.42-5.68 (m, 3H), 5.10 (dd, J = 13.0, 5.0 Hz, 1H), 4.36-4.51 (m, 3H), 4.26-4.34 (m, 1H), 4.09-4.23 (m, 2H), 3.90 (s, 3H), 2.83-2.96 (m, 1H), 2.56-2.64 (m, 1H), 2.34 (s, 4H), 1.94-2.03 (m, 1H) |
| 98 | 6, 32, 44, 46, 49 | 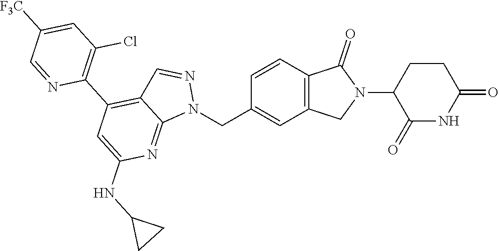 [SMILES: O=C1CCC(N2Cc3cc(Cn4ncc5c(-c6ncc(C(F)(F)F)cc6Cl)cc(NC6CC6)nc54)ccc3C2=O)C(=O)N1] | 3-[5-({4-[3- chloro-5- (trifluoromethyl) pyridin-2-yl]- 6- (cyclopropyl- amino)-1H- pyrazolo[3,4- b]pyridin-1- yl}methyl)-1- oxo-2,3- dihydro-1H- isoindol-2- yl]piperidine- 2,6-dione | 610.2 | ¹H NMR (400 MHz, DMSO-d6) ppm 10.98 (s, 1H), 9.12 (d, J = 1.0 Hz, 1H), 8.69 (d, J = 1.5 Hz, 1H), 7.60-7.74 (m, 3H), 7.55 (s, 1H), 7.45 (d, J = 8.0 Hz, 1H), 6.78 (s, 1H), 5.58-5.68 (m, 2H), 5.10 (dd, J = 13.0, 5.0 Hz, 1H), 4.44 (d, J = 17.5 Hz, 1H), 4.30 (d, J = 17.5 Hz, 1H), 2.81-2.98 (m, 1H), 2.77 (br s, 1H), 2.30-2.49 (m, 2H), 1.93-2.03 (m, 1H), 0.72-0.85 (m, 2H), 0.45-0.59 (m, 2H) |
| 99 | 6, 32, 44, 46, 49 | 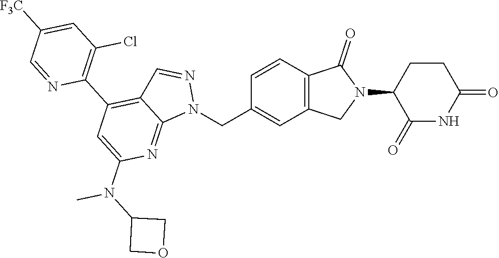 [SMILES: CN(c1cc(-c2ncc(C(F)(F)F)cc2Cl)c2cnn(Cc3ccc4c(c3)CN([C@H]3CCC(=O)NC3=O)C4=O)c2n1)C1COC1] | (3S)-3-[5-({4- [3-chloro-5- (trifluoromethyl) pyridin-2-yl]- 6- [methyl(oxetan- 3-yl)amino]- 1H- pyrazolo[3,4- b]pyridin-1- yl}methyl)-1- oxo-2,3- dihydro-1H- isoindol-2- yl]piperidine- 2,6-dione | 640.2 | ¹H NMR (400 MHz, DMSO-d6): 10.97 (s, 1H), 9.20 (d, J = 1.20 Hz, 1H), 8.71 (d, J = 1.20 Hz, 1H), 7.78 (s, 1H), 7.71 (d, J = 8.00 Hz, 1H), 7.50 (s, 1H), 7.44 (d, J = 9.20 Hz, 1H) 6.95 (s, 1H), 5.65 (s, 2H), 5.24-5.20 (m, 1H), 5.12-5.07 (m, 1H), 4.85- 4.82 (m, 2H), 4.72-4.68 (m, 2H), 4.47-4.28 (m, 2H), 3.10 (s, 3H), 2.90- 2.87 (m, 1H), 2.61-2.57 (m, 1H), 2.50-2.34 (m, 1H), 2.33-1.91 (m, 1H) |
| 100 | 6, 32, 44, 46, 49 | 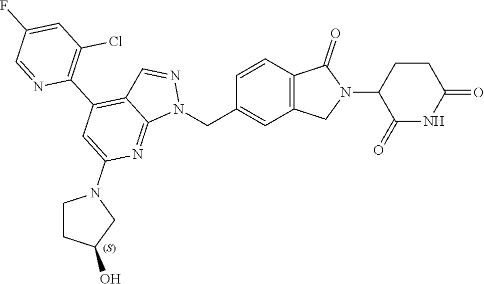 | 3-(5-{[4-(3- chloro-5- fluoropyridin- 2-yl)-6-[(3S)- 3- hydroxypyrrolidin- 1-yl]-1H- pyrazolo[3,4- b]pyridin-1- yl]methyl}-1- oxo-2,3- dihydro-1H- isoindol-2- yl)piperidine- 2,6-dione | 590.1 | ¹H NMR (400 MHz, DMSO-d6): 10.99 (s, 1H), 8.79 (d, J = 2.5 Hz, 1H), 8.34 (dd, J = 8.5, 2.5 Hz, 1H), 7.70 (d, J = 7.8 Hz, 1H), 7.67 (s, 1H), 7.50 (s, 1H), 7.44 (d, J = 7.6 Hz, 1H), 6.63 (s, 1H), 5.63 (s, 2H), 5.09 (dd, J = 13.3, 5.1 Hz, 1H), 4.93-5.07 (m, 1H), 4.38- 4.53 (m, 2H), 4.29 (d, J = 17.4 Hz, 1H), 3.53- 3.74 (m, 3H), 3.40-3.53 (m, 1H), 2.82-3.01 (m, 1H), 2.56-2.62 (m, 1H), 2.31-2.40 (m, 1H), 1.87- 2.13 (m, 3H) |
| 101 | 30 using Intermediate I | 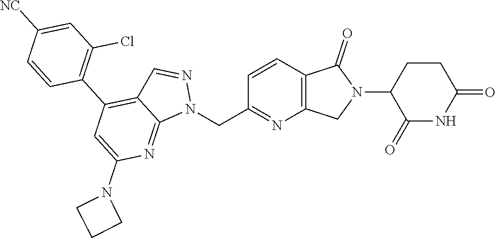 [SMILES: N#Cc1ccc(-c2cc(N3CCC3)nc3c2cnn3Cc2ccc3c(n2)CN(C2CCC(=O)NC2=O)C3=O)c(Cl)c1] | 4-[6-(azetidin- 1-yl)-1-{[6- (2,6- dioxopiperidin- 3-yl)-5-oxo- 5H,6H,7H- pyrrolo[3,4- b]pyridin-2- yl]methyl}-1H- pyrazolo[3,4- b]pyridin-4- yl]-3- chlorobenzonitrile | 567.2 | 1H NMR (400 MHz, DMSO) 11.01 (d, J = 2.4 Hz, 1H), 8.28 (d, J = 1.2 Hz, 1H), 8.11 (d, J = 8.0 Hz, 1H), 7.99 (dd, J = 1.6, 7.9 Hz, 1H), 7.75 (d, J = 8.0 Hz, 1H), 7.66 (s, 1H), 7.15 (d, J = 8.0 Hz, 1H), 6.31 (s, 1H), 5.71 (s, 2H), 5.16 (dd, J = 5.2, 13.2 Hz, 1H), 4.52 (d, J = 18.0 Hz, 1H), 4.38-4.30 (m, 1H), 4.07 (t, J = 7.6 Hz, 4H), 2.97-2.85 (m, 1H), 2.62 (d, J = 1.6 Hz, 1H), 2.44-2.36 (m, 3H), 2.06-1.98 (m, 1H) |
| 102 | 6, 32, 44, 46, 49 | 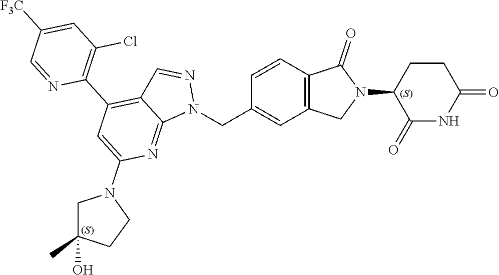 | (3S)-3-[5-({4- [3-chloro-5- (trifluoromethyl) pyridin-2-yl]- 6-[(3S)-3- hydroxy-3- methylpyrrolidin- 1-yl]-1H- pyrazolo[3,4- b]pyridin-1- yl}methyl)-1- oxo-2,3- dihydro-1H- isoindol-2- yl]piperidine- 2,6-dione | 654.2 | ¹H NMR (400 MHz, DMSO-d6): 10.99 (s, 1H), 9.12 (d, J = 0.9 Hz, 1H), 8.71 (d, J = 1.1 Hz, 1H), 7.67-7.79 (m, 2H), 7.39-7.53 (m, 2H), 6.67 (s, 1H), 5.63 (s, 2H), 5.10 (dd, J = 13.3, 5.1 Hz, 1H), 4.87 (s, 1H), 4.24-4.50 (m, 2H), 3.47-3.86 (m, 3H), 3.40 (br d, J = 11.4 Hz, 1H), 2.83-2.97 (m, 1H), 2.61 (br s, 1H), 2.31-2.42 (m, 1H), 1.88- 2.04 (m, 3H), 1.38 (s, 3H) |
| 103 | 6, 32, 44, 46, 49 | 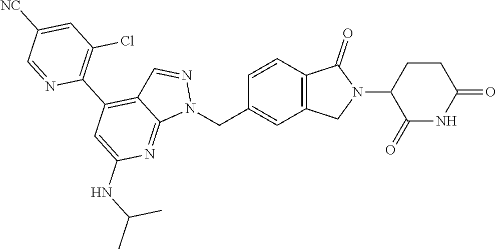 [SMILES: CC(C)Nc1cc(-c2ncc(C#N)cc2Cl)c2cnn(Cc3ccc4c(c3)CN(C3CCC(=O)NC3=O)C4=O)c2n1] | 5-chloro-6-(1- {[2-(2,6- dioxopiperidin- 3-yl)-1-oxo- 2,3-dihydro- 1H-isoindol-5- yl]methyl}-6- [(propan-2- yl)amino]-1H- pyrazolo[3,4- b]pyridin-4- yl)pyridine-3- carbonitrile | 569.2 | ¹H NMR (400 MHz, DMSO-d6) 10.98 (s, 1H), 9.14 (d, J = 1.6 Hz, 1H), 8.81 (d, J = 1.6 Hz, 1H), 7.68 (d, J = 8.0 Hz, 1H), 7.63 (s, 1H), 7.51 (s, 1H), 7.42 (d, J = 7.6 Hz, 1H), 7.35-7.26 (m, 1H), 6.66 (s, 1H), 5.59 (s, 2H), 5.09 (dd, J = 4.8, 13.2 Hz, 1H), 4.48-4.38 (m, 1H), 4.34-4.24 (m, 1H), 4.23-4.10 (m, 1H), 2.95- 2.83 (m, 1H), 2.59 (d, J = 2.4 Hz, 1H), 2.44-2.35 (m, 1H), 2.06-1.90 (m, 1H), 1.21 (dd, J = 2.4, 6.4 Hz, 6H) |
| 104 | 30 using Intermediate I | 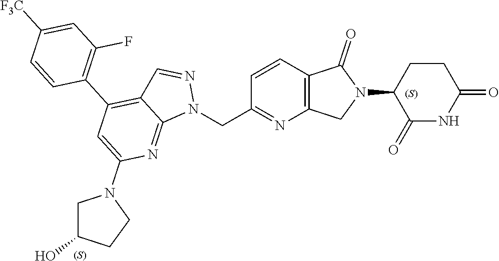 | (3S)-3-[5-({4- [3-fluoro-5- (trifluoromethyl) pyridin-2-yl]- 6-[(3S)-3- hydroxypyrrolidin- 1-yl]-1H- pyrazolo[3,4- b]pyridin-1- yl}methyl)-1- oxo-2,3- dihydro-1H- isoindol-2- yl]piperidine- 2,6-dione | 624.3 | ¹H NMR (DMSO-d6) δ: 10.97 (s, 1H), 9.08 (s, 1H), 8.50-8.62 (m, 1H), 8.02 (d, J = 1.5 Hz, 1H), 7.69 (d, J = 8.1 Hz, 1H), 7.50 (s, 1H), 7.43 (d, J = 8.8 Hz, 1H), 6.89 (s, 1H), 5.65 (s, 2H), 5.01- 5.12 (m, 2H), 4.43 (br d, J = 17.5 Hz, 2H), 4.26- 4.32 (m, 1H), 3.58-3.70 (m, 3H), 3.51 (br s, 1H), 2.85-2.93 (m, 1H), 2.57- 2.63 (m, 1H), 2.32-2.38 (m, 1H), 2.08 (br dd, J = 8.3, 4.4 Hz, 1H), 1.94- 2.01 (m, 2H) |
| 105 | 30 using Intermediate O | 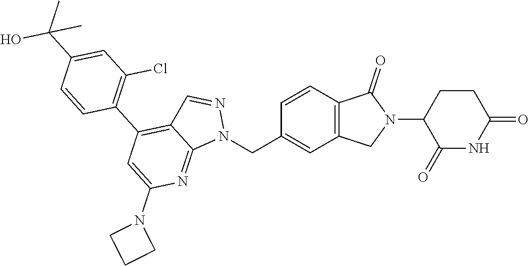 [SMILES: CC(C)(O)c1ccc(-c2cc(N3CCC3)nc3c2cnn3Cc2ccc3c(c2)CN(C2CCC(=O)NC2=O)C3=O)c(Cl)c1] | 3-(5-{[6- (azetidin-1-yl)- 4-[2-chloro-4- (2- hydroxypropan- 2-yl)phenyl]- 1H- pyrazolo[3,4- b]pyridin-1- yl]methyl}-1- oxo-2,3- dihydro-1H- isoindol-2- yl)piperidine- 2,6-dione | 599.3 | ¹H NMR (400 MHz, DMSO-d6) 11.01-10.97 (m, 1H), 7.72-7.69 (m, 2H), 7.60 (s, 1H), 7.55- 7.51 (m, 2H), 7.46-7.41 (m, 2H), 6.25 (s, 1H), 5.59 (s, 2H), 5.28 (s, 1H), 5.09 (dd, J = 5.6, 13.6 Hz, 1H), 4.46-4.41 (m, 1H), 4.31-4.27 (m, 1H), 4.09 (t, J = 7.6 Hz, 4H), 2.94-2.85 (m, 1H), 2.60 (s, 1H), 2.40-2.33 (m, 3H), 1.99-1.94 (m, 1H), 1.47 (s, 6H) |
| 106 | 6, 32, 44, 46, 49 | 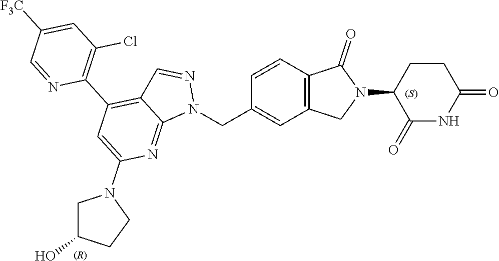 | (3S)-3-[5-({4- [3-chloro-5- (trifluoromethyl) pyridin-2-yl]- 6-[(3R)-3- hydroxypyrrolidin- 1-yl]-1H- pyrazolo[3,4- b]pyridin-1- yl}methyl)-1- oxo-2,3- dihydro-1H- isoindol-2- yl]piperidine- 2,6-dione | 640.2 | ¹H NMR (400 MHz, DMSO-d6): 10.99 (br s, 1H), 9.11-9.14 (m, 1H), 8.71 (d, J = 1.5 Hz, 1H), 7.68-7.73 (m, 2H), 7.51 (s, 1H), 7.45 (d, J = 8.0 Hz, 1H), 6.69 (s, 1H), 5.63 (s, 2H), 5.06-5.13 (m, 1H), 5.00-5.05 (m, 1H), 4.44 (br d, J = 17.0 Hz, 2H), 4.30 (d, J = 17.5 Hz, 1H), 3.55-3.68 (m, 3H), 3.44-3.54 (m, 1H), 2.84-2.95 (m, 1H), 2.55- 2.62 (m, 1H), 2.32-2.38 (m, 1H), 2.02-2.12 (m, 1H), 1.92-2.02 (m, 2H) |
| 107 | 6, 32, 44, 46, 49 | 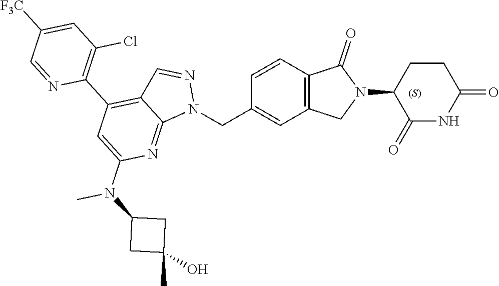 | (3S)-3-[5-({4- [3-chloro-5- (trifluoromethyl) pyridin-2-yl]- 6- {methyl[(1s,3r)- 3-hydroxy-3- methylcyclobutyl] amino}-1H- pyrazolo[3,4- b]pyridin-1- yl}methyl)-1- oxo-2,3- dihydro-1H- isoindol-2- yl]piperidine- 2,6-dione | 668.2 | ¹H NMR (400 MHz, DMSO-d6): 10.97 (br s, 1H), 9.12 (d, J = 1.1 Hz, 1H), 8.70 (d, J = 1.3 Hz, 1H), 7.69-7.75 (m, 2H), 7.53 (s, 1H), 7.45 (d, J = 8.4 Hz, 1H), 6.85 (s, 1H), 5.65 (s, 2H), 5.09 (dd, J = 13.3, 5.1 Hz, 1H), 4.88-5.00 (m, 2H), 4.25- 4.49 (m, 2H), 3.08 (s, 3H), 2.85-2.95 (m, 1H), 2.56-2.65 (m, 1H), 2.27- 2.38 (m, 3H), 2.13-2.22 (m, 2H), 1.92-2.05 (m, 1H), 1.30 (s, 3H) |
| 108 | 108 | 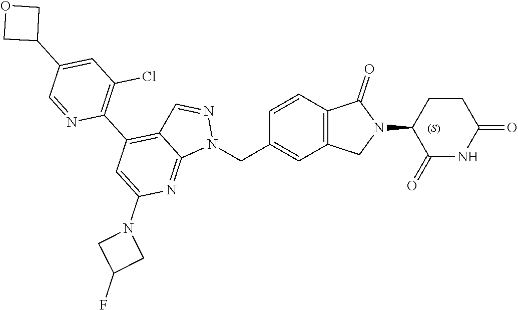 | (3S)-3-[5-({4- [3-chloro-5- (oxetan-3- yl)pyridin-2- yl]-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 616.3 | ¹H NMR (400 MHz, DMSO-d6) 11.09-10.86 (m, 1H), 8.75-8.66 (m, 1H), 8.25 (d, J = 1.6 Hz, 1H), 7.76 (s, 1H), 7.69 (d, J = 7.6 Hz, 1H), 7.52- 7.47 (m, 1H), 7.44-7.38 (m, 1H), 6.57 (s, 1H), 5.66-5.44 (m, 3H), 5.13 (d, J = 4.4 Hz, 1H), 4.98 (dd, J = 6.4, 8.4 Hz, 2H), 4.73 (t, J = 6.4 Hz, 2H), 4.46-4.41 (m, 3H), 4.33- 4.25 (m, 1H), 4.23- 4.12 (m, 2H), 2.96-2.83 (m, 1H), 2.62-2.59 (m, 1H), 2.38-2.35 (m, 1H), 2.01-1.94 (m, 1H) |
| 109 | 6, 32, 44, 46, 49 using Intermediate K | 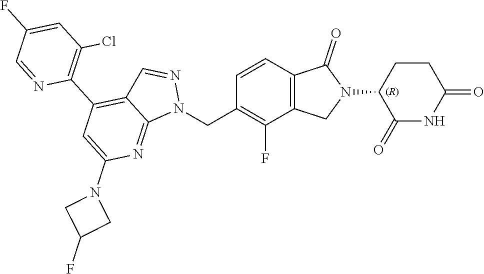 | (3R)-3-(5-{[4- (3-chloro-5- fluoropyridin- 2-yl)-6-(3- fluoroazetidin- 1-yl)-1H- pyrazolo[3,4- b]pyridin-1- yl]methyl}-4- fluoro-1-oxo- 2,3-dihydro- 1H-isoindol-2- yl)piperidine- 2,6-dione | 596.3 | ¹H NMR (400 MHz, DMSO-d6) 11.11-10.91 (m, 1H), 8.78 (d, J = 2.4 Hz, 1H), 8.34 (dd, J = 2.4, 8.8 Hz, 1H), 7.74 (s, 1H), 7.56 (d, J = 7.6 Hz, 1H), 7.38 (t, J = 7.2 Hz, 1H), 6.56 (s, 1H), 5.71- 5.46 (m, 3H), 5.11 (dd, J = 4.8, 12.8 Hz, 1H), 4.62- 4.54 (m, 1H), 4.49- 4.38 (m, 3H), 4.23-4.11 (m, 2H), 2.95-2.86 (m, 1H), 2.60 (s, 1H), 2.42- 2.39 (m, 1H), 2.03-1.95 (m, 1H) |
| 110 | 6, 32, 44, 46, 49 | 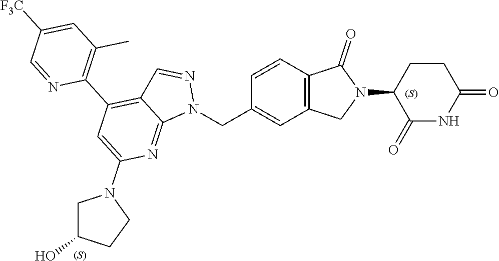 | (3S)-3-[5-({6- [(3S)-3- hydroxypyrrolidin- 1-yl]-4-[3- methyl-5- (trifluoromethyl) pyridin-2-yl]- 1H- pyrazolo[3,4- b]pyridin-1- yl}methyl)-1- oxo-2,3- dihydro-1H- isoindol-2- yl]piperidine- 2,6-dione | 620.2 | ¹H NMR (DMSO-d6) δ: 10.96 (s, 1H), 8.94 (s, 1H), 8.29 (d, J = 2.1 Hz, 1H), 7.70 (d, J = 8.0 Hz, 1H), 7.66 (s, 1H), 7.51 (s, 1H), 7.45 (d, J = 7.9 Hz, 1H), 6.54 (s, 1H), 5.63 (s, 2H), 5.09 (dd, J = 13.2, 5.1 Hz, 1H), 5.01 (d, J = 3.5 Hz, 1H), 4.44 (br d, J = 17.6 Hz, 2H), 4.30 (d, J = 17.5 Hz, 1H), 3.55- 3.69 (m, 3H), 3.49 (br s, 1H), 2.85-2.94 (m, 1H), 2.59 (dt, J = 15.3, 2.2 Hz, 1H), 2.40 (s, 3H), 2.33- 2.38 (m, 1H), 1.93-2.11 (m, 3H) |
| 111 | 6, 32, 44, 46, 49 | 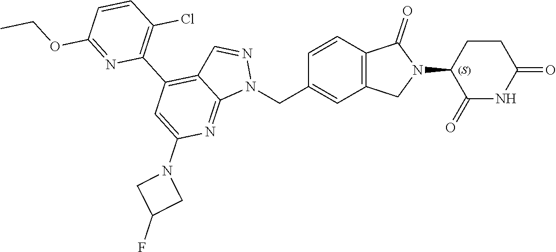 | (3S)-3-(5-{[4- (3-chloro-6- ethoxypyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 604.2 | ¹H NMR (400 MHz, DMSO-d6): 10.98 (br s, 1H), 7.99 (d, J = 8.8 Hz, 1H), 7.84 (s, 1H), 7.70 (d, J = 7.8 Hz, 1H), 7.53 (s, 1H), 7.45 (d, J = 7.9 Hz, 1H), 6.98 (d, J = 8.8 Hz, 1H), 6.62 (s, 1H), 5.42- 5.73 (m, 3H), 5.10 (dd, J = 13.3, 5.0 Hz, 1H), 4.38-4.52 (m, 3H), 4.26- 4.35 (m, 3H), 4.11-4.25 (m, 2H), 2.83-2.99 (m, 1H), 2.35-2.63 (m, 2H), 1.91-2.05 (m, 1H), 1.32 (t, J = 7.0 Hz, 3H) |
| 112 | 6, 32, 44, 46, 49 | 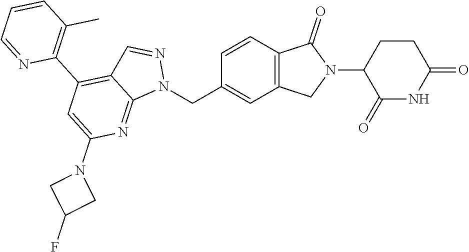 [SMILES: Cc1cccnc1-c1cc(N2CC(F)C2)nc2c1cnn2Cc1ccc2c(c1)CN(C1CCC(=O)NC1=O)C2=O] | 3-(5-{[6-(3- fluoroazetidin- 1-yl)-4-(3- methylpyridin- 2-yl)-1H- pyrazolo[3,4- b]pyridin-1- yl]methyl}-1- oxo-2,3- dihydro-1H- isoindol-2- yl)piperidine- 2,6-dione | 540.3 | ¹H NMR (400 MHz, DMSO) 10.97 (s, 1H), 8.54 (d, J = 4.0 Hz, 1H), 7.86-7.78 (m, 1H), 7.72- 7.66 (m, 2H), 7.48 (s, 1H), 7.41 (dd, J = 4.4, 8.0 Hz, 2H), 6.42 (s, 1H), 5.68-5.45 (m, 3H), 5.09 (dd, J = 5.2, 12.8 Hz, 1H), 4.50-4.39 (m, 3H), 4.34-4.24 (m, 1H), 4.23- 4.10 (m, 2H), 2.96- 2.84 (m, 1H), 2.60 (d, J = 1.2 Hz, 1H), 2.43-2.36 (m, 1H), 2.30 (s, 3H), 2.02-1.93 (m, 1H) |
| 113 | 6, 32, 44, 46, 49 | 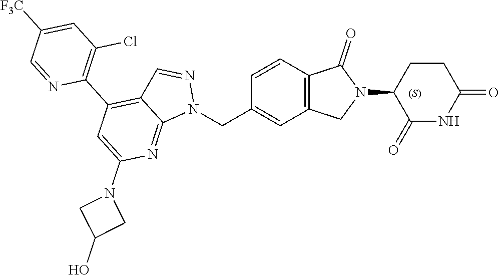 | (3S)-3-[5-({4- [3-chloro-5- (trifluoromethyl) pyridin-2-yl]- 6-(3- hydroxyazetidin- 1-yl)-1H- pyrazolo[3,4- b]pyridin-1- yl}methyl)-1- oxo-2,3- dihydro-1H- isoindol-2- yl]piperidine- 2,6-dione | 626.1 | ¹H NMR (400 MHz, DMSO-d6): 10.98 (br s, 1H), 9.12 (dd, J = 1.9, 0.8 Hz, 1H), 8.69-8.73 (m, 1H), 7.76 (s, 1H), 7.70 (d, J = 7.9 Hz, 1H), 7.50 (s, 1H), 7.42 (d, J = 7.9 Hz, 1H), 6.56 (s, 1H), 5.74 (d, J = 6.8 Hz, 1H), 5.63 (s, 2H), 5.10 (dd, J = 13.3, 5.1 Hz, 1H), 4.59-4.67 (m, 1H), 4.39-4.49 (m, 1H), 4.27-4.36 (m, 3H), 3.85 (dd, J = 9.3, 4.4 Hz, 2H), 2.85-2.96 (m, 1H), 2.56- 2.63 (m, 1H), 2.31-2.39 (m, 1H), 1.93-2.04 (m, 1H |
| 114 | 6, 32, 44, 46, 49 | 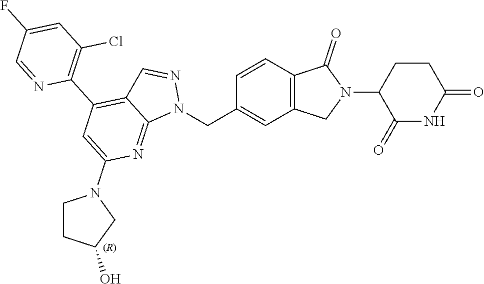 | 3-(5-{[4-(3- chloro-5- fluoropyridin- 2-yl)-6-[(3R)- 3- hydroxypyrrolidin- 1-yl]-1H- pyrazolo[3,4- b]pyridin-1- yl]methyl}-1- oxo-2,3- dihydro-1H- isoindol-2- yl)piperidine- 2,6-dione | 590.1 | ¹H NMR (400 MHz, DMSO-d6): 10.99 (s, 1H), 8.79 (d, J = 2.6 Hz, 1H), 8.34 (dd, J = 8.5, 2.5 Hz, 1H), 7.64-7.73 (m, 2H), 7.50 (s, 1H), 7.44 (d, J = 8.0 Hz, 1H), 6.63 (s, 1H), 5.63 (s, 2H), 5.09 (dd, J = 13.2, 5.1 Hz, 1H), 5.02 (d, J = 3.4 Hz, 1H), 4.39-4.48 (m, 2H), 4.24- 4.34 (m, 1H), 3.54-3.74 (m, 3H), 3.41-3.54 (m, 1H), 2.83-2.98 (m, 1H), 2.56-2.62 (m, 1H), 2.29- 2.41 (m, 1H), 1.91-2.11 (m, 3H) |
| 115 | 30 | 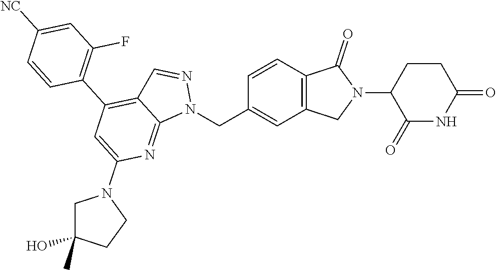 [SMILES: C[C@]1(O)CCN(c2cc(-c3ccc(C#N)cc3F)c3cnn(Cc4ccc5c(c4)CN(C4CCC(=O)NC4=O)C5=O)c3n2)C1] | 4-(1-{[2-(2,6- dioxopiperidin- 3-yl)-1-oxo- 2,3-dihydro- 1H-isoindol-5- yl]methyl}-6- [(3S)-3- hydroxy-3- methylpyrrolidin- 1-yl]-1H- pyrazolo[3,4- b]pyridin-4- yl)-3- fluorobenzonitrile | 594.1 | ¹H NMR (400 MHz, DMSO-d6, 2 rotamers): 10.98 (s, 1H), 8.10 (d, J = 9.9 Hz, 1H), 7.86-7.94 (m, 2H), 7.74 (d, J = 2.4 Hz, 1H), 7.69 (d, J = 7.9 Hz, 1H), 7.50 (s, 1H), 7.43 (d, J = 7.8 Hz, 1H), 6.53 (s, 1H), 5.62 (s, 2H), 5.09 (dd, J = 13.3, 5.1 Hz, 1H), 4.85 (s, 1H), 4.43 (d, J = 17.6 Hz, 1H), 4.29 (d, J = 17.5 Hz, 1H), 3.48- 3.73 (m, 3H), 3.38-3.42 (m, 1H), 2.56-2.64 (m, 1H), 2.32-2.35 (m, 1H), 1.89-2.02 (m, 4H), 1.38 (s, 3H) |
| 116 | 30 with coupling partner cas # 458532-90- 6 | 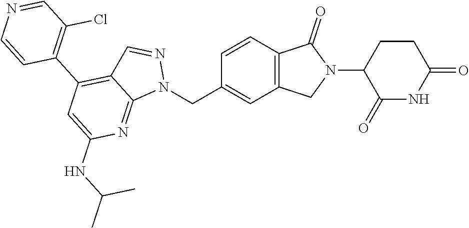 [SMILES: CC(C)Nc1cc(-c2ccncc2Cl)c2cnn(Cc3ccc4c(c3)CN(C3CCC(=O)NC3=O)C4=O)c2n1] | 3-(5-{[4-(3- chloropyridin- 4-yl)-6- [(propan-2- yl)amino]-1H- pyrazolo[3,4- b]pyridin-1- yl]methyl}-1- oxo-2,3- dihydro-1H- isoindol-2- yl)piperidine- 2,6-dione | 544.2 | ¹H NMR (400 MHz, DMSO) 10.97 (s, 1H), 8.81 (s, 1H), 8.64 (d, J = 4.8 Hz, 1H), 7.69 (d, J = 8.0 Hz, 1H), 7.58-7.54 (m, 3H), 7.45 (d, J = 7.6 Hz, 1H), 7.24 (d, J = 6.8 Hz, 1H), 6.42 (s, 1H), 5.58 (s, 2H), 5.09 (dd, J = 5.2, 13.2 Hz, 1H), 4.45- 4.27 (m, 2H), 4.20-4.16 (m, 1H), 2.94-2.90 (m, 1H), 2.61-2.56 (m, 1H), 2.38-2.32 (m, 1H), 2.00- 1.95 (m, 1H), 1.22 (dd, J = 1.8, 6.4 Hz, 6H) |
| 117 | 6, 32, 44, 46, 49 | 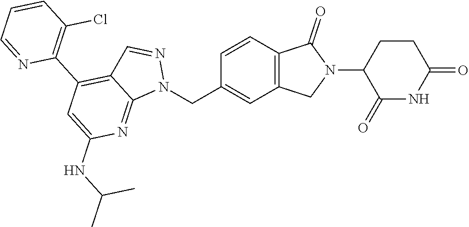 [SMILES: CC(C)Nc1cc(-c2ncccc2Cl)c2cnn(Cc3ccc4c(c3)CN(C3CCC(=O)NC3=O)C4=O)c2n1] | 3-(5-{[4-(3- chloropyridin- 2-yl)-6- [(propan-2- yl)amino]-1H- pyrazolo[3,4- b]pyridin-1- yl]methyl}-1- oxo-2,3- dihydro-1H- isoindol-2- yl)piperidine- 2,6-dione | 544.2 | ¹H NMR (400 MHz, DMSO) 10.97 (s, 1H), 8.68 (dd, J = 1.6, 4.8 Hz, 1H), 8.12 (dd, J = 1.2, 8.0 Hz, 1H), 7.68 (d, J = 7.6 Hz, 1H), 7.59 (s, 1H), 7.54 (dd, J = 4.8, 8.4 Hz, 1H), 7.51 (s, 1H), 7.43 (d, J = 8.0 Hz, 1H), 7.18 (d, J = 6.8 Hz, 1H), 6.61 (s, 1H), 5.59 (s, 2H), 5.09 (dd, J = 5.2, 13.2 Hz, 1H), 4.45-4.27 (m, 2H), 4.21-4.16 (m, 1H), 2.94- 2.85 (m, 1H), 2.60- 2.56 (m, 1H), 2.40-2.34 (m, 1H), 2.00-1.94 (m, 1H), 1.22 (d, J = 2.8 Hz, 3H), 1.20 (d, J = 2.4 Hz, 3H) |
| 118 | 30 using Intermediate J | 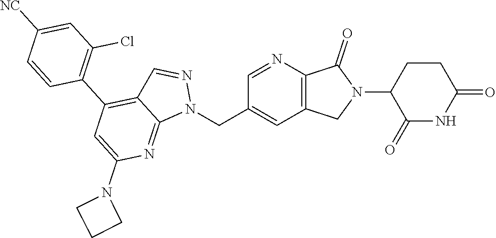 [SMILES: N#Cc1ccc(-c2cc(N3CCC3)nc3c2cnn3Cc2cnc3c(c2)CN(C2CCC(=O)NC2=O)C3=O)c(Cl)c1] | 4-[6-(azetidin- 1-yl)-1-{[6- (2,6- dioxopiperidin- 3-yl)-7-oxo- 5H,6H,7H- pyrrolo[3,4- b]pyridin-3- yl]methyl}-1H- pyrazolo[3,4- b]pyridin-4- yl]-3- chlorobenzonitrile | 567.1 | ¹H NMR (400 MHz, DMSO-d6) 10.67 (s, 1H), 8.77 (d, J = 1.6 Hz, 1H), 8.43 (s, 1H), 8.27 (d, J = 1.6 Hz, 1H), 8.00- 7.94 (m, 2H), 7.71 (d, J = 8.0 Hz, 1H), 7.64-7.62 (m, 1H), 6.30 (s, 1H), 5.65 (s, 2H), 5.13 (dd, J = 5.2, 13.2 Hz, 1H), 4.49- 4.40 (m, 1H), 4.39-4.30 (m, 1H), 4.11 (t, J = 7.6 Hz, 4H), 2.95-2.83 (m, 1H), 2.62-2.57 (m, 1H), 2.42-2.34 (m, 3H), 2.05- 1.93 (m, 1H) |
| 119 | 6, 32, 44, 46, 49 | 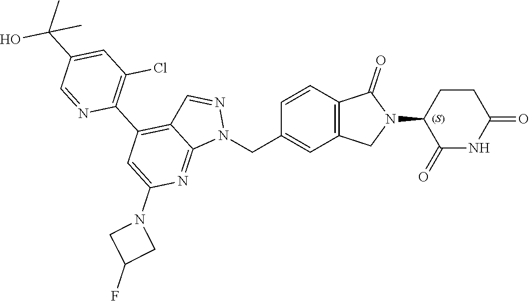 | (3S)-3-[5-({4- [3-chloro-5-(2- hydroxypropan- 2-yl)pyridin- 2-yl]-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 618.2 | ¹H NMR (400 MHz, DMSO-d6): 10.98 (s, 1H), 8.80 (d, J = 1.9 Hz, 1H), 8.13 (d, J = 1.9 Hz, 1H), 7.75 (s, 1H), 7.70 (d, J = 7.9 Hz, 1H), 7.50 (s, 1H), 7.42 (d, J = 8.5 Hz, 1H), 6.57 (s, 1H), 5.64 (s, 3H), 5.48 (s, 2H), 5.10 (dd, J = 13.4, 5.3 Hz, 1H), 4.37-4.51 (m, 4H), 4.26- 4.34 (m, 1H), 4.18-4.24 (m, 1H), 4.13-4.18 (m, 1H), 4.10 (q, J = 5.2 Hz, 1H), 3.17 (d, J = 5.3 Hz, 3H), 2.84-2.97 (m, 1H), 2.61 (br d, J = 1.9 Hz, 2H), 1.94-2.03 (m, 1H) |
| 120 | 6, 32, 44, 46,49 | 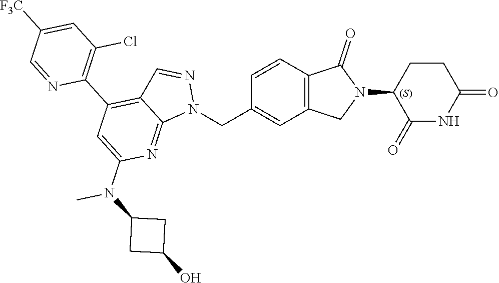 | (3R)-3-[5-({4- [3-chloro-5- (trifluoromethyl) pyridin-2-yl]- 6- {methyl[(1s,3s)- 3- hydroxycyclobutyl] amino}-1H- pyrazolo[3,4- b]pyridin-1- yl}methyl)-1- oxo-2,3- dihydro-1H- isoindol-2- yl]piperidine- 2,6-dione | 654.0 | ¹H NMR (400 MHz, DMSO-d6): 10.96 (s, 1H), 9.11 (d, J = 1.2 Hz, 1H), 8.69 (d, J = 1.6 Hz, 1H), 7.72-7.68 (m, 2H), 7.50 (s, 1H), 7.44 (d, J = 8 Hz, 1H), 6.87 (s, 1H), 5.64 (s, 2H), 5.10-5.06 (m, 2H), 4.45 (brd, J = 17.6 Hz, 1H), 4.31-4.27 (m, 2H), 3.89 (d, J = 6.8 Hz, 1H), 4.32 (brd, J = 17.6 Hz, 1H), 3.1 (s, 3H), 2.94-2.86 (m, 1H), 2.65- 2.58 (m, 3H), 2.38-2.32 (m, 1H), 2.04-1.96 (m, 3H) |
| 121 | 6, 32, 44, 46, 49 | 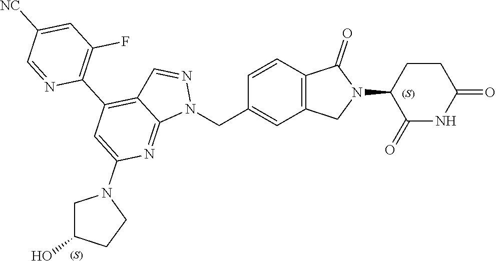 | 6-[1-({2-[(3S)- 2,6- dioxopiperidin- 3-yl]-1-oxo- 2,3-dihydro- 1H-isoindol-5- yl}methyl)-6- [(3S)-3- hydroxypyrrolidin- 1-yl]-1H- pyrazolo[3,4- b]pyridin-4- yl]-5- fluoropyridine- 3-carbonitrile | 581.2 | ¹H NMR (DMSO-d6) δ: 10.97 (s, 1H), 9.12 (t, J = 1.4 Hz, 1H), 8.67 (dd, J = 10.8, 1.7 Hz, 1H), 8.00 (d, J = 1.6 Hz, 1H), 7.69 (d, J = 7.8 Hz, 1H), 7.49 (s, 1H), 7.43 (d, J = 7.5 Hz, 1H), 6.90 (s, 1H), 5.65 (s, 2H), 5.01-5.11 (m, 2H), 4.43 (br d, J = 17.6 Hz, 2H), 4.26- 4.32 (m, 1H), 3.58-3.69 (m, 3H), 2.84-2.96 (m, 1H), 2.56-2.62 (m, 1H), 2.31-2.37 (m, 2H), 2.09 (s, 1H), 1.94-2.01 (m, 2H) |
| 122 | 6, 32, 44, 46, 49 | 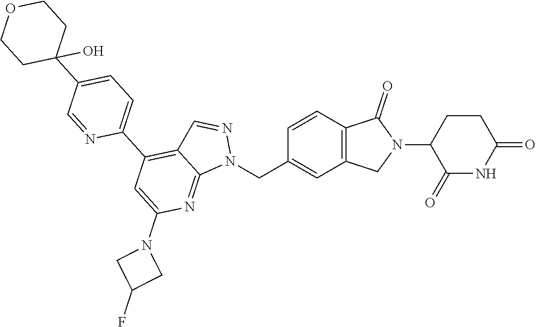 [SMILES: O=C1CCC(N2Cc3cc(Cn4ncc5c(-c6ccc(C7(O)CCOCC7)cn6)cc(N6CC(F)C6)nc54)ccc3C2=O)C(=O)N1] | 3-(5-{[6-(3- fluoroazetidin- 1-yl)-4-[5-(4- hydroxyoxan- 4-yl)pyridin-2- yl]-1H- pyrazolo[3,4- b]pyridin-1- yl]methyl}-1- oxo-2,3- dihydro-1H- isoindol-2- yl)piperidine- 2,6-dione | 626.2 | ¹H NMR (400 MHz, DMSO-d6) ppm 10.98 (br s, 1H), 8.94 (d, J = 2.0 Hz, 1H), 8.31-8.46 (m, 1H), 8.17 (d, J = 8.0 Hz, 1H), 8.06 (dd, J = 8.3, 2.3 Hz, 1H), 7.69 (d, J = 7.5 Hz, 1H), 7.48 (s, 1H), 7.41 (d, J = 8.0 Hz, 1H), 6.95 (s, 1H), 5.46-5.73 (m, 3H), 5.38 (s, 1H), 5.09 (dd, J = 13.0, 5.0 Hz, 1H), 4.37-4.55 (m, 3H), 4.15-4.34 (m, 3H), 3.71- 3.88 (m, 4H), 2.77-3.01 (m, 1H), 2.54-2.62 (m, 1H), 2.29-2.48 (m, 1H), 2.08 (td, J = 12.5, 5.0 Hz, 2H), 1.92-2.02 (m, 1H), 1.63 (br d, J = 12.5 Hz, 2H) |
| 123 | 6, 32, 44, 46, 49 | 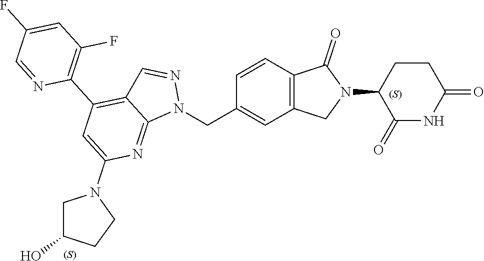 | (3S)-3-(5-{[4- (3,5- difluoropyridin- 2-yl)-6-[(3S)- 3- hydroxypyrrolidin- 1-yl]-1H- pyrazolo[3,4- b]pyridin-1- yl]methyl}-1- oxo-2,3- dihydro-1H- isoindol-2- yl)piperidine- 2,6-dione | 574.2 | ¹H NMR (400 MHz, DMSO-d6): 10.97 (br s, 1H), 8.76 (d, J = 2.4 Hz, 1H), 8.19 (ddd, J = 11.1, 8.8, 2.4 Hz, 1H), 7.98 (d, J = 1.5 Hz, 1H), 7.69 (d, J = 7.8 Hz, 1H), 7.49 (s, 1H), 7.43 (d, J = 7.6 Hz, 1H), 6.81 (s, 1H), 5.64 (s, 2H), 5.06-5.12 (m, 1H), 4.99-5.04 (m, 1H), 4.40- 4.46 (m, 2H), 4.24-4.33 (m, 1H), 3.47-3.68 (m, 4H), 2.83-2.96 (m, 1H), 2.60-2.69 (m, 1H), 2.32- 2.37 (m, 1H), 2.07 (ddt, J = 12.9, 8.5, 4.3 Hz, 1H), 1.92-2.01 (m, 2H) |
| 124 | 30 with Intermediate P | 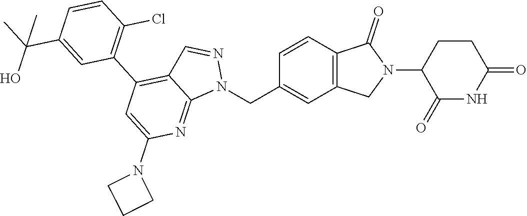 [SMILES: CC(C)(O)c1ccc(Cl)c(-c2cc(N3CCC3)nc3c2cnn3Cc2ccc3c(c2)CN(C2CCC(=O)NC2=O)C3=O)c1] | 3-(5-{[6- (azetidin-1-yl)- 4-[2-chloro-5- (2- hydroxypropan- 2-yl)phenyl]- 1H- pyrazolo[3,4- b]pyridin-1- yl]methyl}-1- oxo-2,3- dihydro-1H- isoindol-2- yl)piperidine- 2,6-dione | 599.3 | ¹H NMR (400 MHz, DMSO-d6) 11.08-10.74 (m, 1H), 7.69 (d, J = 8.0 Hz, 1H), 7.58 (s, 2H), 7.55 (s, 2H), 7.51 (s, 1H), 7.42 (d, J = 8.0 Hz, 1H), 6.25 (s, 1H), 5.60 (s, 2H), 5.18 (s, 1H), 5.11-5.07 (m, 1H), 4.46-4.27 (m, 2H), 4.10 (t, J = 7.6 Hz, 4H), 2.94-2.85 (m, 1H), 2.58 (d, J = 18.0 Hz, 1H), 2.40-2.33 (m, 3H), 2.01- 1.96 (m, 1H), 1.45 (s, 6H) |
| 125 | 6, 32, 44, 46, 49 | 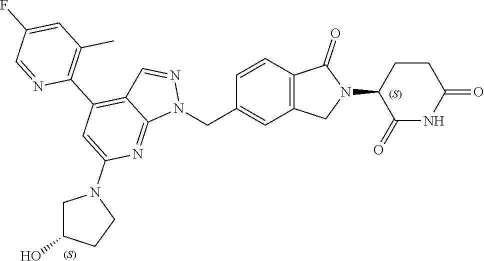 | (3S)-3-(5-{[4- (5-fluoro-3- methylpyridin- 2-yl)-6-[(3S)- 3- hydroxypyrrolidin- 1-yl]-1H- pyrazolo[3,4- b]pyridin-1- yl]methyl}-1- oxo-2,3- dihydro-1H- isoindol-2- yl)piperidine- 2,6-dione | 570.2 | ¹H NMR (DMSO-d6, 400 MHZ) δ: 10.97 (s, 1H), 8.56 (d, J = 2.8 Hz, 1H), 7.82 (dd, J = 10.0, 2.5 Hz, 1H), 7.69 (d, J = 7.8 Hz, 1H), 7.62 (s, 1H), 7.50 (s, 1H), 7.44 (d, J = 7.9 Hz, 1H), 6.46 (s, 1H), 5.62 (s, 2H), 5.09 (dd, J = 13.3, 5.1 Hz, 1H), 5.00 (d, J = 3.5 Hz, 1H), 4.39-4.47 (m, 2H), 4.25-4.35 (m, 1H), 3.41-3.67 (m, 4H), 2.84- 2.96 (m, 1H), 2.53-2.63 (m, 1H), 2.30-2.38 (m, 4H), 1.89-2.11 (m, 3H) |
| 126 | 6, 32, 44, 46, 49 | 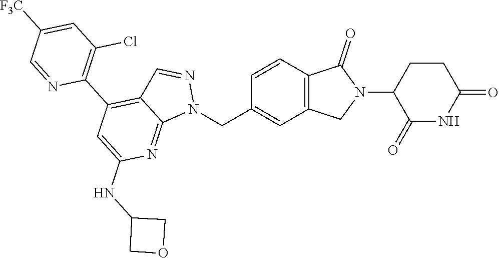 [SMILES: O=C1CCC(N2Cc3cc(Cn4ncc5c(-c6ncc(C(F)(F)F)cc6Cl)cc(NC6COC6)nc54)ccc3C2=O)C(=O)N1] | 3-[5-({4-[3- chloro-5- (trifluoromethyl) pyridin-2-yl]- 6-[(oxetan-3- yl)amino]-1H- pyrazolo[3,4- b]pyridin-1- yl}methyl)-1- oxo-2,3- dihydro-1H- isoindol-2- yl]piperidine- 2,6-dione | 626.1 | ¹H NMR (400 MHz, DMSO-d6): 10.99 (s, 1H), 9.10 (d, J = 1.0 Hz, 1H), 8.69 (d, J = 1.4 Hz, 1H), 7.75 (d, J = 7.9 Hz, 1H), 7.51 (s, 1H), 7.41 (s, 1H), 7.34 (d, J = 7.8 Hz, 1H), 6.40 (s, 1H), 5.71 (s, 2H), 5.11 (dd, J = 13.2, 5.1 Hz, 1H), 4.71-4.92 (m, 1H), 4.30-4.50 (m, 2H), 4.23-4.30 (m, 2H), 3.47- 3.57 (m, 2H), 2.86-2.97 (m, 1H), 2.57-2.70 (m, 2H), 2.31-2.37 (m, 1H), 1.95-2.05 (m, 1H) |
| 127 | 127 | 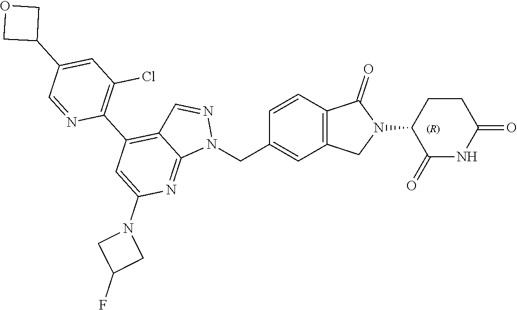 | (3R)-3-[5-({4- [3-chloro-5- (oxetan-3- yl)pyridin-2- yl]-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 616.3 | ¹H NMR (400 MHz, DMSO-d6) 10.97 (s, 1H), 8.71 (d, J = 1.6 Hz, 1H), 8.25 (d, J = 1.6 Hz, 1H), 7.74 (s, 1H), 7.69 (d, J = 8.0 Hz, 1H), 7.49 (s, 1H), 7.41 (d, J = 8.0 Hz, 1H), 6.56 (s, 1H), 5.69- 5.44 (m, 3H), 5.09 (dd, J = 5.2, 13.2 Hz, 1H), 4.98 (dd, J = 6.0, 8.4 Hz, 2H), 4.73 (t, J = 6.4 Hz, 2H), 4.52-4.37 (m, 4H), 4.34- 4.26 (m, 1H), 4.24- 4.10 (m, 2H), 2.98-2.82 (m, 1H), 2.61-2.56 (m, 1H), 2.42-2.32 (m, 1H), 2.02-1.93 (m, 1H) |
| 128 | 6, 32, 44, 46, 49 | 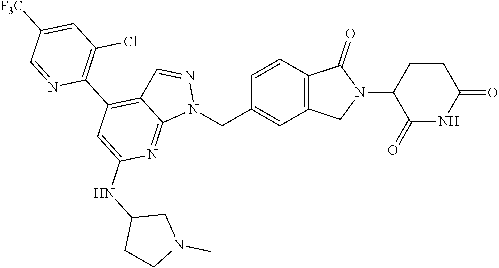 [SMILES: CN1CCC(Nc2cc(-c3ncc(C(F)(F)F)cc3Cl)c3cnn(Cc4ccc5c(c4)CN(C4CCC(=O)NC4=O)C5=O)c3n2)C1] | 3-[5-({4-[3- chloro-5- (trifluoromethyl) pyridin-2-yl]- 6-[(1- methylpyrrolidin- 3-yl)amino]- 1H- pyrazolo[3,4- b]pyridin-1- yl}methyl)-1- oxo-2,3- dihydro-1H- isoindol-2- yl]piperidine- 2,6-dione | 653.3 | ¹H NMR (400 MHz, DMSO-d6): 11.00 (s, 1H), 9.81 (br d, J = 2.5 Hz, 1H), 9.12 (d, J = 1.0 Hz, 1H), 8.72 (d, J = 1.5 Hz, 1H), 7.75 (s, 1H), 7.71- 7.74 (m, 1H), 7.52 (br d, J = 7.5 Hz, 1H), 7.46 (dd, J = 7.8, 3.3 Hz, 1H), 6.75 (d, J = 4.0 Hz, 1H), 5.60- 5.73 (m, 2H), 5.10 (dd, J = 13.5, 5.0 Hz, 1H), 4.52-4.64 (m, 1H), 4.25- 4.49 (m, 2H), 3.62-3.75 (m, 1H), 3.51-3.62 (m, 1H), 3.09-3.21 (m, 1H), 2.88 (br dd, J = 15.0, 4.5 Hz, 4H), 2.55-2.70 (m, 2H), 2.32-2.41 (m, 2H), 1.88-2.07 (m, 2H) |
| 129 | Stille coupling with Intermediate E and cas # 749258-96- 6 | 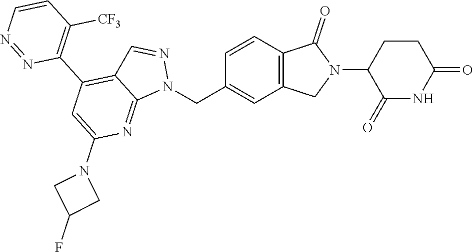 [SMILES: O=C1CCC(N2Cc3cc(Cn4ncc5c(-c6nnccc6C(F)(F)F)cc(N6CC(F)C6)nc54)ccc3C2=O)C(=O)N1] | 3-(5-{[6-(3- fluoroazetidin- 1-yl)-4-[4- (trifluoromethyl) pyridazin-3- yl]-1H- pyrazolo[3,4- b]pyridin-1- yl]methyl}-1- oxo-2,3- dihydro-1H- isoindol-2- yl)piperidine- 2,6-dione | 595.3 | ¹H NMR (400 MHz, DMSO) 10.97 (br, 1H), 9.71 (d, J = 5.2 Hz, 1H), 8.35 (d, J = 5.6 Hz, 1H), 7.70 (d, J = 8.0 Hz, 1H), 7.65 (s, 1H), 7.50 (s, 1H), 7.43 (d, J = 8.0 Hz, 1H), 6.51 (s, 1H), 5.70-5.43 (m, 3H), 5.09 (dd, J = 5.2, 13.2 Hz, 1H), 4.49- 4.38 (m, 3H), 4.34-4.26 (m, 1H), 4.23-4.08 (m, 2H), 2.96-2.85 (m, 1H), 2.64-2.58 (m, 1H), 2.44- 2.35 (m, 1H), 2.02- 1.94 (m, 1H) |
| 130 | 6, 32, 44, 46, 49 | 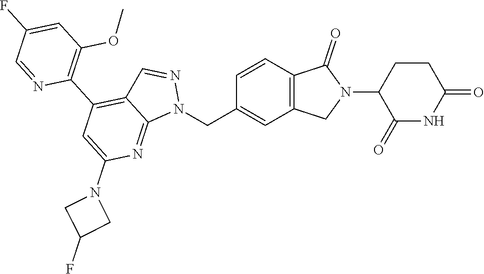 [SMILES: COc1cc(F)cnc1-c1cc(N2CC(F)C2)nc2c1cnn2Cc1ccc2c(c1)CN(C1CCC(=O)NC1=O)C2=O] | 3-(5-{[4-(5- fluoro-3- methoxypyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 574.1 | ¹H NMR (DMSO-d6): 10.98 (s, 1H), 8.38 (d, J = 1.9 Hz, 1H), 7.93 (s, 1H), 7.64-7.80 (m, 2H), 7.47 (s, 1H), 7.40 (d, J = 7.6 Hz, 1H), 6.74 (s, 1H), 5.42-5.72 (m, 3H), 5.09 (dd, J = 13.3, 5.1 Hz, 1H), 4.35-4.55 (m, 3H), 4.23-4.35 (m, 1H), 4.07- 4.23 (m, 2H), 3.91 (s, 3H), 2.82-2.97 (m, 1H), 2.59-2.71 (m, 1H), 2.34- 2.42 (m, 1H), 1.93-2.04 (m, 1H) |
| 131 | 6, 32, 44, 46, 49 | 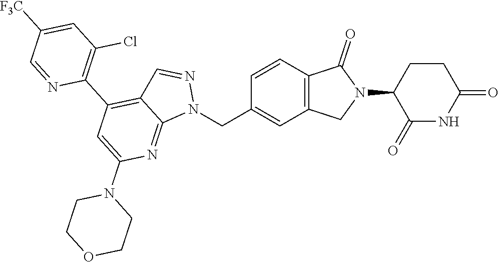 [SMILES: O=C1CC[C@H](N2Cc3cc(Cn4ncc5c(-c6ncc(C(F)(F)F)cc6Cl)cc(N6CCOCC6)nc54)ccc3C2=O)C(=O)N1] | (3S)-3-[5-({4- [3-chloro-5- (trifluoromethyl) pyridin-2-yl]- 6-(morpholin- 4-yl)-1H- pyrazolo[3,4- b]pyridin-1- yl}methyl)-1- oxo-2,3- dihydro-1H- isoindol-2- yl]piperidine- 2,6-dione | 640.0 | ¹H NMR (400 MHz, DMSO-d6): 10.98 (s, 1H), 9.11-9.14 (m, 1H), 8.72 (d, J = 1.4 Hz, 1H), 7.78 (s, 1H), 7.70 (d, J = 7.8 Hz, 1H), 7.52 (s, 1H), 7.44 (d, J = 8.0 Hz, 1H), 7.07 (s, 1H), 5.66 (s, 2H), 5.10 (dd, J = 13.3, 5.1 Hz, 1H), 4.27-4.48 (m, 2H), 3.70-3.77 (m, 4H), 3.68 (br d, J = 4.9 Hz, 4H), 2.83-2.96 (m, 1H), 2.59- 2.70 (m, 1H), 2.32-2.39 (m, 1H), 1.92-2.02 (m, 1H) |
| 132 | 6, 32, 44, 46, 49 | 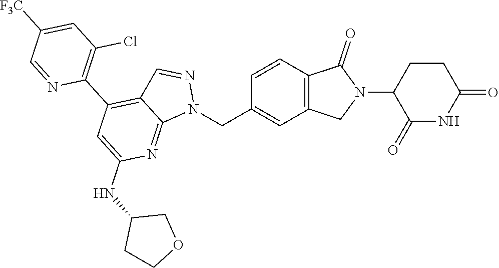 [SMILES: O=C1CCC(N2Cc3cc(Cn4ncc5c(-c6ncc(C(F)(F)F)cc6Cl)cc(N[C@H]6CCOC6)nc54)ccc3C2=O)C(=O)N1] | 3-[5-({4-[3- chloro-5- (trifluoromethyl) pyridin-2-yl]- 6-{[(3S)- oxolan-3- yl]amino}-1H- pyrazolo[3,4- b]pyridin-1- yl}methyl)-1- oxo-2,3- dihydro-1H- isoindol-2- yl]piperidine- 2,6-dione | 640.0 | ¹H NMR (400 MHz, DMSO-d6): 10.98 (s, 1H), 9.11 (dd, J = 1.9, 0.8 Hz, 1H), 8.69 (d, J = 1.3 Hz, 1H), 7.64-7.73 (m, 3H), 7.54 (s, 1H), 7.45 (d, J = 7.8 Hz, 1H), 6.73 (s, 1H), 5.62 (s, 2H), 5.10 (dd, J = 13.3, 5.0 Hz, 1H), 4.48-4.57 (m, 1H), 4.26- 4.48 (m, 2H), 3.99 (dd, J = 8.9, 6.1 Hz, 1H), 3.86 (q, J = 7.4 Hz, 1H), 3.76 (td, J = 8.1, 5.8 Hz, 1H), 3.61 (dd, J = 8.9, 3.9 Hz, 1H), 2.84-2.96 (m, 1H), 2.66-2.69 (m, 1H), 2.33- 2.36 (m, 1H), 2.21-2.31 (m, 1H), 1.94-2.03 (m, 1H), 1.82-1.92 (m, 1H) |
| 133 | 6, 32, 44, 46, 49 | 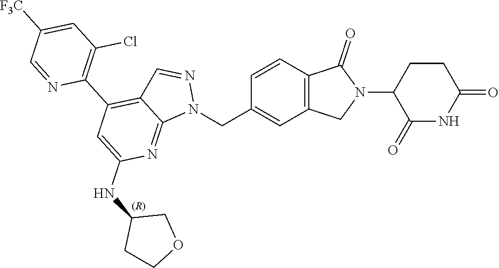 | 3-[5-({4-[3- chloro-5- (trifluoromethyl) pyridin-2-yl]- 6-{[(3R)- oxolan-3- yl]amino}-1H- pyrazolo[3,4- b]pyridin-1- yl}methyl)-1- oxo-2,3- dihydro-1H- isoindol-2- yl]piperidine- 2,6-dione | 640.1 | ¹H NMR (400 MHz, DMSO-d6): 10.98 (s, 1H), 9.11 (dd, J = 1.8, 0.8 Hz, 1H), 8.69 (d, J = 1.4 Hz, 1H), 7.65-7.73 (m, 3H), 7.53 (s, 1H), 7.46 (d, J = 8.5 Hz, 1H), 6.73 (s, 1H), 5.62 (s, 2H), 5.10 (dd, J = 13.2, 5.1 Hz, 1H), 4.48-4.56 (m, 1H), 4.26- 4.47 (m, 2H), 3.99 (dd, J = 8.9, 5.9 Hz, 1H), 3.86 (q, J = 7.3 Hz, 1H), 3.76 (td, J = 8.1, 5.8 Hz, 1H), 3.60 (dd, J = 8.9, 3.9 Hz, 1H), 2.84-2.96 (m, 1H), 2.59-2.69 (m, 1H), 2.32- 2.36 (m, 1H), 2.20-2.30 (m, 1H), 1.94-2.02 (m, 1H), 1.83-1.93 (m, 1H) |
| 134 | 6, 32, 44, 46, 49 | 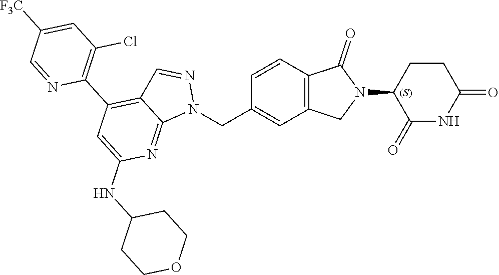 | (3S)-3-[5-({4- [3-chloro-5- (trifluoromethyl) pyridin-2-yl]- 6-[(oxan-4- yl)amino]-1H- pyrazolo[3,4- b]pyridin-1- yl}methyl)-1- oxo-2,3- dihydro-1H- isoindol-2- yl]piperidine- 2,6-dione | 654.0 | ¹H NMR (400 MHz, DMSO-d6): 10.98 (s, 1H), 9.09-9.13 (m, 1H), 8.69 (d, J = 1.5 Hz, 1H), 7.69 (d, J = 8.0 Hz, 1H), 7.66-7.68 (m, 1H), 7.49- 7.54 (m, 1H), 7.41 (dd, J = 13.5, 7.5 Hz, 2H), 6.69 (s, 1H), 5.60 (s, 2H), 5.10 (dd, J = 13.5, 5.0 Hz, 1H), 4.23-4.51 (m, 2H), 4.07 (dt, J = 6.5, 3.3 Hz, 1H), 3.82-3.95 (m, 2H), 3.42- 3.52 (m, 2H), 2.83-2.97 (m, 1H), 2.59-2.70 (m, 1H), 2.32-2.40 (m, 1H), 1.90-2.01 (m, 3H), 1.39- 1.53 (m, 2H) |
| 135 | 6, 32, 44, 46, 49 | 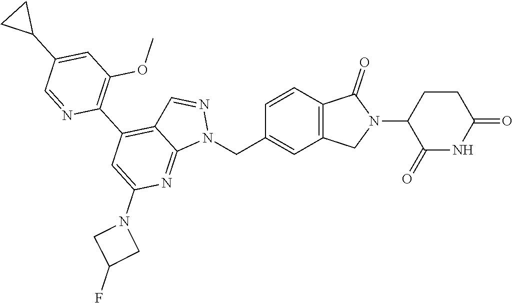 [SMILES: COc1cc(C2CC2)cnc1-c1cc(N2CC(F)C2)nc2c1cnn2Cc1ccc2c(c1)CN(C1CCC(=O)NC1=O)C2=O] | 3-(5-{[4-(5- cyclopropyl-3- methoxypyridin- 2-yl)-6-(3- fluoroazetidin- 1-yl)-1H- pyrazolo[3,4- b]pyridin-1- yl]methyl}-1- oxo-2,3- dihydro-1H- isoindol-2- yl)piperidine- 2,6-dione | 596.1 | ¹H NMR (400 MHz, DMSO-d6): 10.98 (s, 1H), 8.18 (d, J = 1.5 Hz, 1H), 7.96 (s, 1H), 7.69 (d, J = 8.0 Hz, 1H), 7.46 (s, 1H), 7.39 (d, J = 7.5 Hz, 1H), 7.25 (d, J = 2.0 Hz, 1H), 6.76 (s, 1H), 5.39- 5.71 (m, 3H), 5.09 (dd, J = 13.0, 5.0 Hz, 1H), 4.35-4.52 (m, 3H), 4.23- 4.35 (m, 1H), 4.07-4.23 (m, 2H), 3.88 (s, 3H), 2.82-2.97 (m, 1H), 2.60- 2.69 (m, 1H), 2.34-2.42 (m, 1H), 2.02-2.12 (m, 1H), 1.91-2.02 (m, 1H), 1.01-1.14 (m, 2H), 0.84- 0.98 (m, 2H) |
| 136 | 6, 32, 44, 46, 49 | 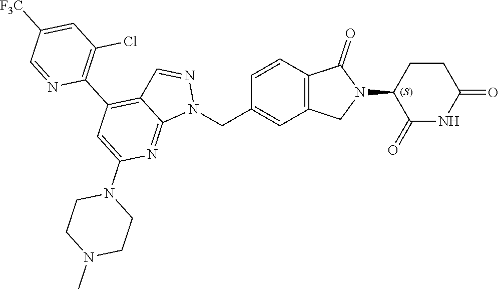 | (3S)-3-[5-({4- [3-chloro-5- (trifluoromethyl) pyridin-2-yl]- 6-(4- methylpiperazin- 1-yl)-1H- pyrazolo[3,4- b]pyridin-1- yl}methyl)-1- oxo-2,3- dihydro-1H- isoindol-2- yl]piperidine- 2,6-dione | 653.2 | ¹H NMR (400 MHz, DMSO-d6): 10.97 (s, 1H), 9.12 (d, J = 1.0 Hz, 1H), 8.71 (d, J = 1.4 Hz, 1H), 7.75 (s, 1H), 7.70 (d, J = 7.9 Hz, 1H), 7.52 (s, 1H), 7.44 (d, J = 7.3 Hz, 1H), 7.07 (s, 1H), 5.66 (s, 2H), 5.10 (dd, J = 13.2, 5.1 Hz, 1H), 4.44 (d, J = 17.4 Hz, 1H), 4.30 (d, J = 17.5 Hz, 1H), 3.70 (br s, 4H), 2.85-2.96 (m, 1H), 2.61 (br s, 1H), 2.42-2.47 (m, 4H), 2.32-2.39 (m, 1H), 2.24 (s, 3H), 1.94-2.02 (m, 1H) |
| 137 | 137 | 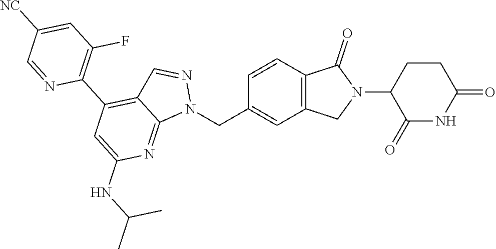 [SMILES: CC(C)Nc1cc(-c2ncc(C#N)cc2F)c2cnn(Cc3ccc4c(c3)CN(C3CCC(=O)NC3=O)C4=O)c2n1] | 6-(1-{[2-(2,6- dioxopiperidin- 3-yl)-1-oxo- 2,3-dihydro- 1H-isoindol-5- yl]methyl}-6- [(propan-2- yl)amino]-1H- pyrazolo[3,4- b]pyridin-4- yl)-5- fluoropyridine- 3-carbonitrile | 553.2 | ¹H NMR (400 MHz, DMSO-d6) 11.09-10.80 (m, 1H), 9.09 (s, 1H), 8.63 (dd, J = 1.6, 11.2 Hz, 1H), 8.46 (s, 1H), 7.98 (s, 1H), 7.67 (d, J = 7.6 Hz, 1H), 7.50 (s, 1H), 7.43-7.31 (m, 2H), 6.97 (s, 1H), 5.60 (s, 2H), 5.08 (dd, J = 5.2, 13.2 Hz, 1H), 4.44-4.38 (m, 1H), 4.33-4.24 (m, 1H), 4.23- 4.13 (m, 1H), 2.94- 2.84 (m, 1H), 2.59 (d, J = 2.8 Hz, 1H), 2.44-2.35 (m, 1H), 2.02-1.92 (m, 1H), 1.21 (dd, J = 2.0, 6.4 Hz, 6H) |
| 138 | 137 using cas # 150010-20- 1 | 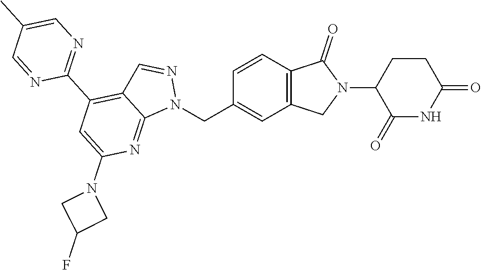 [SMILES: Cc1cnc(-c2cc(N3CC(F)C3)nc3c2cnn3Cc2ccc3c(c2)CN(C2CCC(=O)NC2=O)C3=O)nc1] | 3-(5-{[6-(3- fluoroazetidin- 1-yl)-4-(5- methylpyrimidin- 2-yl)-1H- pyrazolo[3,4- b]pyridin-1- yl]methyl}-1- oxo-2,3- dihydro-1H- isoindol-2- yl)piperidine- 2,6-dione | 541.2 | ¹H NMR (400 MHz, DMSO-d6) 11.04-10.91 (m, 1H), 8.90 (s, 2H), 8.52 (s, 1H), 7.68 (d, J = 7.6 Hz, 1H), 7.46 (s, 1H), 7.40 (d, J = 8.4 Hz, 1H), 7.29 (s, 1H), 5.70-5.46 (m, 3H), 5.08 (dd, J = 5.2, 13.2 Hz, 1H), 4.55- 4.38 (m, 3H), 4.32-4.16 (m, 3H), 2.97-2.80 (m, 1H), 2.62-2.56 (m, 1H), 2.42-2.34 (m, 4H), 2.03- 1.92 (m, 1H) |
| 139 | 30 | 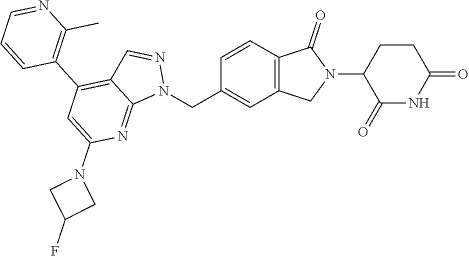 [SMILES: Cc1ncccc1-c1cc(N2CC(F)C2)nc2c1cnn2Cc1ccc2c(c1)CN(C1CCC(=O)NC1=O)C2=O] | 3-(5-{[6-(3- fluoroazetidin- 1-yl)-4-(2- methylpyridin- 3-yl)-1H- pyrazolo[3,4- b]pyridin-1- yl]methyl}-1- oxo-2,3- dihydro-1H- isoindol-2- yl)piperidine- 2,6-dione | 540.3 | ¹H NMR (400 MHz, DMSO-d6) 10.98 (s, 1H), 8.57 (dd, J = 1.6, 4.8 Hz, 1H), 7.76-7.64 (m, 3H), 7.52 (s, 1H), 7.43 (d, J = 7.6 Hz, 1H), 7.37 (dd, J = 4.8, 7.6 Hz, 1H), 6.35 (s, 1H), 5.65-5.44 (m, 3H), 5.09 (dd, J = 5.2, 13.2 Hz, 1H), 4.48-4.39 (m, 3H), 4.33-4.27 (m, 1H), 4.21-4.08 (m, 2H), 2.96-2.83 (m, 1H), 2.63- 2.55 (m, 1H), 2.42- 2.36 (m, 4H), 2.03-1.94 (m, 1H) |
| 140 | 140 | 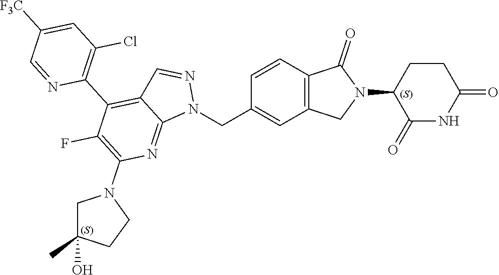 | (3S)-3-[5-({4- [3-chloro-5- (trifluoromethyl) pyridin-2-yl]- 5-fluoro-6- [(3S)-3- hydroxy-3- methylpyrrolidin- 1-yl]-1H- pyrazolo[3,4- b]pyridin-1- yl}methyl)-1- oxo-2,3- dihydro-1H- isoindol-2- yl]piperidine- 2,6-dione | 672.2 | ¹H NMR (400 MHz, DMSO-d₆) δ 11.06- 10.90 (m, 1H), 9.15 (s, 1H), 8.75 (s, 1H), 7.70 (d, J = 8.0 Hz, 1H), 7.61 (s, 1H), 7.50 (s, 1H), 7.44 (d, J = 8.4 Hz, 1H), 5.61 (s, 2H), 5.08 (dd, J = 5.2, 13.2 Hz, 1H), 4.87 (s, 1H), 4.48-4.25 (m, 2H), 3.86-3.75 (m, 2H), 3.67- 3.55 (m, 2H), 2.92- 2.84 (m, 1H), 2.61 (dd, J = 2.4, 4.0 Hz, 1H), 2.40- 2.35 (m, 1H), 2.02-1.94 (m, 1H), 1.93-1.85 (m, 2H), 1.36 (s, 3H) |
| 141 | 6, 32, 44, 46, 49 | 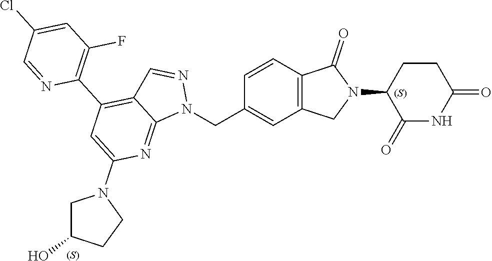 | (3S)-3-(5-{[4- (5-chloro-3- fluoropyridin- 2-yl)-6-[(3S)- 3- hydroxypyrrolidin- 1-yl]-1H- pyrazolo[3,4- b]pyridin-1- yl]methyl}-1- oxo-2,3- dihydro-1H- isoindol-2- yl)piperidine- 2,6-dione | 590.2 | ¹H NMR (DMSO-d6) δ: 10.98 (s, 1H), 8.76 (dd, J = 2.0, 1.0 Hz, 1H), 8.34 (dd, J = 10.5, 2.0 Hz, 1H), 8.00 (d, J = 1.5 Hz, 1H), 7.69 (d, J = 8.0 Hz, 1H), 7.48 (s, 1H), 7.42 (d, J = 8.0 Hz, 1H), 6.83 (s, 1H), 5.64 (s, 2H), 5.09 (dd, J = 13.5, 5.0 Hz, 1H), 5.03 (d, J = 3.5 Hz, 1H), 4.40-4.46 (m, 2H), 4.26- 4.31 (m, 1H), 3.59-3.67 (m, 3H), 3.44-3.53 (m, 1H), 2.85-2.93 (m, 1H), 2.56-2.61 (m, 1H), 2.32- 2.38 (m, 1H), 2.04-2.11 (m, 1H), 1.93-2.01 (m, 2H) |
| 142 | 6, 32, 44, 46, 49 | 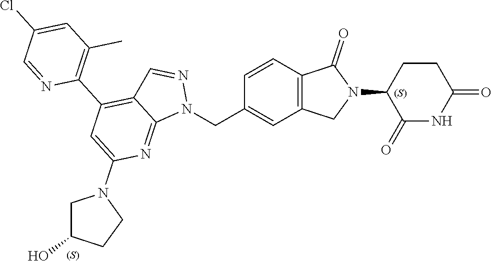 | (3S)-3-(5-{[4- (5-chloro-3- methylpyridin- 2-yl)-6-[(3S)- 3- hydroxypyrrolidin- 1-yl]-1H- pyrazolo[3,4- b]pyridin-1- yl]methyl}-1- oxo-2,3- dihydro-1H- isoindol-2- yl)piperidine- 2,6-dione | 586.2 | ¹H NMR (DMSO-d6, 400 MHz) δ: 10.97 (s, 1H), 8.61 (d, J = 1.9 Hz, 1H), 8.02 (dd, J = 2.4, 0.6 Hz, 1H), 7.69 (d, J = 7.9 Hz, 1H), 7.64 (s, 1H), 7.50 (s, 1H), 7.44 (d, J = 7.8 Hz, 1H), 6.48 (s, 1H), 5.62 (s, 2H), 5.09 (dd, J = 13.4, 5.1 Hz, 1H), 5.00 (d, J = 3.6 Hz, 1H), 4.44 (br d, J = 17.5 Hz, 2H), 4.29 (d, J = 17.5 Hz, 1H), 3.55- 3.68 (m, 3H), 3.48 (br s, 1H), 2.84-2.95 (m, 1H), 2.56-2.63 (m, 1H), 2.30- 2.39 (m, 4H), 1.91-2.11 (m, 3H) |
| 143 | Palau'Chlor treatment of 108 | 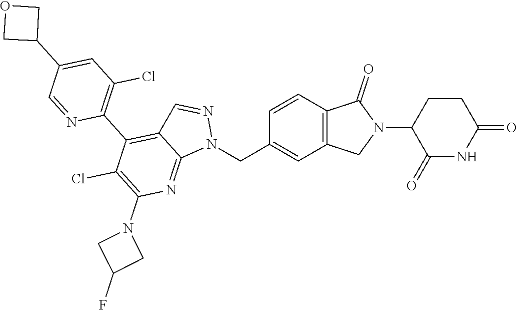 [SMILES: O=C1CCC(N2Cc3cc(Cn4ncc5c(-c6ncc(C7COC7)cc6Cl)c(Cl)c(N6CC(F)C6)nc54)ccc3C2=O)C(=O)N1] | 3-[5-({5- chloro-4-[3- chloro-5- (oxetan-3- yl)pyridin-2- yl]-6-(3- fluoroazetidin- 1-yl)-1H- pyrazolo[3,4- b]pyridin-1- yl}methyl)-1- oxo-2,3- dihydro-1H- isoindol-2- yl]piperidine- 2,6-dione | 561.2 | ¹H NMR (400 MHz, METHANOL-d₄) δ = 8.67 (d, J = 1.9 Hz, 1H), 8.29 (d, J = 2.0 Hz, 1H), 7.79 (d, J = 7.8 Hz, 1H), 7.54-7.49 (m, 3H), 5.78- 5.66 (m, 2H), 5.53- 5.32 (m, 1H), 5.21-5.11 (m, 3H), 4.85-4.81 (m, 2H), 4.77-4.61 (m, 2H), 4.52-4.37 (m, 5H), 2.96- 2.86 (m, 1H), 2.82- 2.73 (m, 1H), 2.55-2.41 (m, 1H), 2.23-2.12 (m, 1H) |
| 144 | 6, 32, 44, 46, 49 | 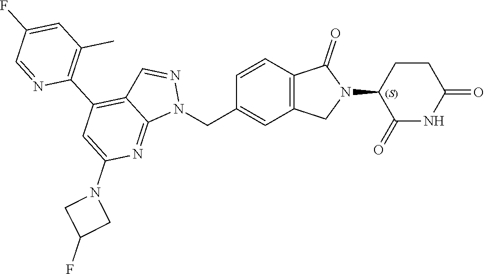 | (S)-3-(5-((4-(5- fluoro-3- methylpyridin- 2-yl)-6-(3- fluoroazetidin- 1-yl)-1H- pyrazolo[3,4- b]pyridin-1- yl)methyl)-1- oxoisoindolin- 2- yl)piperidine- 2,6-dione | 558.4 | ¹H NMR (DMSO-d6) δ: 10.97 (s, 1H), 8.57 (s, 1H), 7.83 (d, J = 9.6 Hz, 1H), 7.70 (s, 1H), 7.70 (d, J = 8.0 Hz, 1H), 7.49 (s, 1H), 7.42 (d, J = 7.9 Hz, 1H), 6.43 (s, 1H), 5.44- 5.66 (m, 3H), 5.09 (dd, J = 13.3, 5.1 Hz, 1H), 4.38-4.49 (m, 3H), 4.27- 4.34 (m, 1H), 4.11-4.23 (m, 2H), 2.85-2.95 (m, 1H), 2.56-2.62 (m, 1H), 2.31-2.40 (m, 4H), 1.95- 2.03 (m, 1H) |
| 145 | 6, 32, 44, 46, 49 | 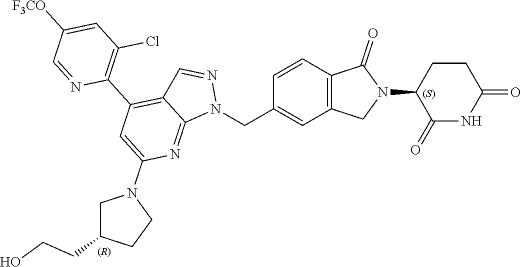 | (S)-3-(5-((4-(3- chloro-5- (trifluoromethyl) pyridin-2-yl)- 6-((R)-3-(2- hydroxyethyl) pyrrolidin-1-yl)- 1H- pyrazolo[3,4- b]pyridin-1- yl)methyl)-1- oxoisoindolin- 2- yl)piperidine- 2,6-dione | 668.2 | ¹H NMR (DMSO-d6) δ: 10.96 (s, 1H), 9.11 (dd, J = 1.9, 0.8 Hz, 1H), 8.68- 8.70 (m, 1H), 7.68-7.72 (m, 2H), 7.51 (s, 1H), 7.43 (d, J = 7.9 Hz, 1H), 6.68 (s, 1H), 5.62 (s, 2H), 5.09 (dd, J = 13.3, 5.1 Hz, 1H), 4.40-4.50 (m, 2H), 4.26-4.34 (m, 1H), 3.61- 3.83 (m, 2H), 3.43-3.56 (m, 3H), 3.12 (br t, J = 9.3 Hz, 1H), 2.84-2.94 (m, 1H), 2.58 (dt, J = 15.4, 2.1 Hz, 1H), 2.29-2.42 (m, 2H), 2.15 (dt, J = 5.7, 2.9 Hz, 1H), 1.93-2.02 (m, 1H), 1.54-1.71 (m, 3H) |
| 146 | 6, 32, 44, 46, 49 | 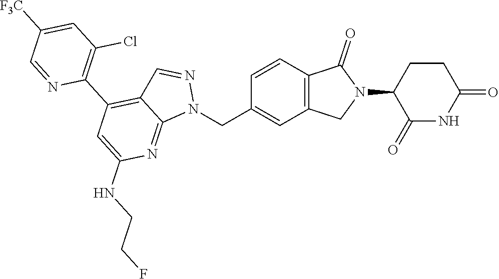 [SMILES: O=C1CC[C@H](N2Cc3cc(Cn4ncc5c(-c6ncc(C(F)(F)F)cc6Cl)cc(NCCF)nc54)ccc3C2=O)C(=O)N1] | (S)-3-(5-((4-(3- chloro-5- (trifluoromethyl) pyridin-2-yl)- 6-((2- fluoroethyl) amino)-1H- pyrazolo[3,4- b]pyridin-1- yl)methyl)-1- oxoisoindolin- 2- yl)piperidine- 2,6-dione | 616.0 | ¹H NMR (DMSO-d6) δ: 10.97 (s, 1H), 9.11 (dd, J = 1.8, 0.8 Hz, 1H), 8.68 (d, J = 1.4 Hz, 1H), 7.66- 7.73 (m, 3H), 7.52 (s, 1H), 7.44 (d, J = 7.8 Hz, 1H), 6.75 (s, 1H), 5.62 (s, 2H), 5.09 (dd, J = 13.3, 5.1 Hz, 1H), 4.70 (t, J = 5.1 Hz, 1H), 4.58 (t, J = 5.0 Hz, 1H), 4.44 (d, J = 17.5 Hz, 1H), 4.30 (d, J = 17.4 Hz, 1H), 3.67-3.80 (m, 2H), 2.83-2.97 (m, 1H), 2.56-2.64 (m, 1H), 2.30- 2.42 (m, 1H), 1.94-2.02 (m, 1H) |

### BIOLOGICAL EXAMPLES

### B1. FAK-HiBiT Degradation Assay

To generate JHH-4-FAK-HiBiT cells for the FAK degradation assay, JHH-4 cells (Japanese Collection of Research Biosources Cell Bank, Japan) were transfected with ribonucleoprotein reagents to introduce, by CRISPR-Cas9 editing, the homozygous knock-in of a HiBiT tag (Promega Corporation, Madison, WI) to the C-terminus of the FAK gene in its natural genomic locus. Clonal cell populations were established by expanding single cells after CRISPR-Cas9 editing, with a clonal cell line named JHH-4-FAK-HiBiT clone d01 confirmed by next generation DNA sequencing to contain the expected HiBiT edited FAK gene and was selected for FAK degradation screening. Cells were selected and maintained in complete DMEM media (DMEM, 10% Heat Inactivated FBS, and Non-Essential Amino Acids at 37° C. and 5% CO₂). Cells were passaged every 3-4 days by reseeding 0.5×10⁶ cells in a T75 flask.

Cells were dispensed into 384-well white plates (Corning #3570, NY) that were pre-spotted with compounds using an acoustic dispenser (Echo acoustic transfer system, Beckman Coulter Life Sciences, Carlsbad, CA) as a 10-point dose response curve with a 3-fold dilution starting at 1 μM and including a DMSO control. Twenty-five microliters of media containing 2000 JHH-4-FAK-HiBiT Clone d01 cells was dispensed per well. Assay plates were incubated at 37° C. with 5% CO2 for two hours. After incubation, 25 μL of Nano-Glo HiBiT Lytic Reagent Working Solution (Promega Corporation, Madison, WI) was added to each well and incubated at room temperature for 10 minutes protected from light. After 10 minutes, luminescence was read on a PHERAstar luminometer (BMG Labtech, Cary, NC).

To determine the half-maximal effective concentration (EC50) values for FAK-HiBIT degradation, a four parameter logistic model was used: (Sigmoidal Dose-Response Model)(FIT=(A+((B−A)/1+((C/x)\hat{ }D)))) where C is the inflection point (EC50), D is the Hill slope, and A and B are the low and high limits of the fit respectively). The lower limit of the fit (value A) is referred to as Ymin. The maximum limit constraint, Ymax, was derived using DMSO control. The curves were processed and evaluated using Dotmatics Atlas (Insightful Science, LLC, Boston, MA). The results are shown in Table 2 below.

### CAL-51 Cell Proliferation Assay

CAL-51 cells were obtained from Bristol Myers Squibb internal cell banks (originally sourced from DSMZ-German Collection of Microorganisms and Cell Cultures GmbH, Germany) and cultured in RPMI complete media (RPMI-1640, 10% Fetal Bovine serum, 1× Antibiotic-Antimycotic, and 1× Non-Essential Amino Acids). Compounds were dispensed into 384-well plates using an acoustic dispenser (Beckman Coulter Life Sciences, Carlsbad, CA) in duplicate with 14 compounds on each plate. For each compound, a 10-point dose response was printed at 4-fold dilutions starting at 10 μM. The DMSO concentration was kept constant for a final assay concentration of 0.1% per well assuming 50 uL final media volume. Compound plates were sealed and frozen at −20° C. until use.

For each assay batch, compound plates were thawed and allowed to reach room temperature, and cells were seeded at 350 cells per well in 50 μl complete RPMI media. After 120 hours of incubation at 37° C. and 5% CO₂, cells were lysed by addition of 25 μL of Cell-Titer-Glo Reagent (Promega Corporation, Madison, WI), as per manufacturer's instructions, protected from light, shaken for 20 minutes, and total luminescence read by a CLARIOstar Plus plate reader (BMG LabTech, Cary, NC). Total luminescence signal is linearly correlated with cell number remaining in the well.

Luminescence data was processed per plate by subtracting reagent-only “blank” well luminescence from all measured values and calculating a percent of DMSO control value for each treatment well. Then, to determine the half-maximal effective concentration (EC50) values for cell growth inhibition, a four parameter logistic model was fit to the data for each DMSO-normalized compound: Sigmoidal Dose-Response Model: (FIT=(A+((B−A)/1+((C/x)\hat{ }D)))) where C is the inflection point (EC50), D is the Hill slope, and A and B are the low and high limits of the fit respectively. The lower limit of the fit (value A) is referred to as Ymin-calculated. The minimal percent of DMSO control that is observed in the concentrations tested for each compound is labeled as “Ymin-obs”, and was recorded and reported in the tables. Sigmoidal fit curves were processed and evaluated using Dotmatics Atlas (Insightful Science, LLC, Boston, MA). The results are shown in Table 2 below.

TABLE 2
FAK HiBiT degradation and CAL-51 cell proliferation.

| | FAK HiBiT EC50 | FAK HiBiT | CAL-51 Prolif EC50 | CAL-51 Prolif |
|---|---|---|---|---|
| Example # | (nM) | Ymin | (nM) | Ymin |
| 1 | 0.14 | 7.57 | 0.17 | 27.59 |
| 2 | 0.24 | 7.25 | 0.43 | 14.10 |
| 3 | 0.29 | 8.97 | 1.42 | 23.98 |
| 4 | 0.42 | 7.37 | 0.52 | 1.90 |
| 5 | 0.42 | 6.93 | 0.36 | 29.57 |
| 6 | 0.50 | 8.05 | 0.40 | 31.07 |
| 7 | 0.57 | 8.83 | 0.36 | 21.87 |
| 8 | 0.55 | 9.05 | 0.85 | 17.44 |
| 9 | 0.55 | 9.43 | 0.46 | 22.89 |
| 10 | 0.56 | 6.93 | 75.43 | 33.21 |
| 11 | 0.64 | 6.28 | 0.34 | 22.89 |
| 12 | 0.67 | 7.50 | 0.99 | 23.97 |
| 13 | 7.86 | 4.03 | NA | NA |
| 14 | 14.59 | 7.20 | 5.06 | 23.56 |
| 15 | 0.95 | 7.50 | 0.89 | 29.00 |
| 16 | 1000.00 | 96.37 | 297.95 | 81.53 |
| 17 | 3.36 | 7.42 | 3.46 | 28.46 |
| 18 | 8.37 | 4.01 | NA | NA |
| 19 | 0.86 | 8.55 | 0.38 | 23.48 |
| 20 | 0.89 | 7.90 | 0.37 | 24.38 |
| 21 | 1.54 | 9.84 | 0.91 | 23.08 |
| 22 | 0.68 | 10.53 | 0.39 | 22.20 |
| 23 | 1.03 | 7.69 | 0.62 | 20.44 |
| 24 | 29.59 | 5.76 | 4.05 | 23.72 |
| 25 | 21.07 | 5.97 | 145.99 | 46.08 |
| 26 | 8.50 | 11.53 | 2.32 | 24.69 |
| 27 | 8.68 | 2.64 | NA | NA |
| 28 | 0.38 | 7.63 | 0.45 | 25.69 |
| 29 | 0.71 | 8.83 | 1.23 | 30.94 |
| 30 | 0.72 | 7.40 | 0.77 | 26.67 |
| 31 | 0.76 | 8.92 | 3.89 | 34.80 |
| 32 | 0.82 | 9.44 | 0.44 | 9.16 |
| 33 | 0.91 | 5.36 | 0.51 | 26.78 |
| 34 | 0.91 | 8.98 | 1.08 | 26.10 |
| 35 | 0.97 | 10.02 | 0.59 | 21.28 |
| 36 | 0.98 | 8.36 | 0.50 | 24.38 |
| 37 | 0.98 | 7.33 | 0.60 | 19.81 |
| 38 | 1.09 | 8.19 | 0.48 | 21.89 |
| 39 | 1.12 | 7.78 | 0.82 | 28.13 |
| 40 | 1.15 | 6.87 | 11.17 | 36.00 |
| 41 | 1.22 | 10.49 | 1.23 | 26.68 |
| 42 | 1.24 | 5.70 | 1.92 | 24.80 |
| 43 | 1.59 | 6.11 | 154.76 | 51.95 |
| 44 | 1.73 | 11.00 | 0.69 | 28.22 |
| 45 | 1.76 | 6.88 | 0.76 | 30.48 |
| 46 | 1.79 | 8.57 | 0.91 | 30.10 |
| 47 | 1.80 | 17.06 | 1.96 | 39.85 |
| 48 | 1.87 | 8.06 | 0.52 | 19.89 |
| 49 | 2.00 | 7.78 | 0.37 | 23.11 |
| 50 | 2.23 | 5.82 | 2.12 | 27.72 |
| 51 | 2.23 | 10.32 | 1.49 | 30.14 |
| 52 | 2.40 | 8.38 | 1.13 | 22.49 |
| 53 | 2.45 | 7.24 | 9.88 | 39.88 |
| 54 | 2.47 | 6.69 | 0.73 | 25.81 |
| 55 | 3.13 | 20.64 | 3.19 | 36.41 |
| 56 | 3.72 | 10.37 | 1.67 | 26.66 |
| 57 | 3.41 | 8.26 | 1.51 | 25.51 |
| 58 | 3.43 | 7.90 | NA | NA |
| 59 | 3.54 | 10.50 | 0.87 | 28.60 |
| 60 | 3.57 | 9.10 | NA | NA |
| 61 | 3.60 | 7.65 | 7.10 | 35.56 |
| 62 | 3.92 | 12.88 | NA | NA |
| 63 | 4.08 | 8.81 | 1.92 | 31.58 |
| 64 | 4.29 | 9.77 | 2.32 | 32.02 |
| 65 | 4.47 | 17.03 | 1.89 | 36.07 |
| 66 | 4.63 | 7.72 | 1.72 | 27.43 |
| 67 | 5.06 | 9.35 | 1.60 | 28.60 |
| 68 | 5.19 | 15.10 | NA | NA |
| 69 | 5.55 | 9.26 | 1.84 | 23.38 |
| 70 | 5.60 | 13.38 | NA | NA |
| 71 | 5.67 | 11.11 | NA | NA |
| 72 | 6.35 | 7.12 | 1.78 | 23.41 |
| 73 | 6.62 | 11.02 | 3.39 | 23.58 |
| 74 | 6.88 | 8.00 | 4.11 | 34.11 |
| 75 | 7.95 | 23.15 | 9.67 | 52.27 |
| 76 | 7.97 | 9.51 | 6.15 | 34.59 |
| 77 | 8.00 | 8.29 | 3.67 | 28.95 |
| 78 | 8.40 | 5.59 | 1.76 | 24.33 |
| 79 | 8.56 | 10.79 | 3.22 | 23.53 |
| 80 | 9.06 | 5.83 | 1.68 | 27.48 |
| 81 | 9.57 | 12.82 | 6.37 | 38.16 |
| 82 | 9.61 | 8.29 | 4.19 | 29.74 |
| 83 | 10.23 | 10.27 | 5.41 | 30.14 |
| 84 | 10.26 | 19.19 | NA | NA |
| 85 | 11.11 | 11.71 | 6.82 | 35.10 |
| 86 | 12.46 | 35.59 | NA | NA |
| 87 | 12.97 | 5.32 | 2.17 | 38.15 |
| 88 | 12.97 | 10.98 | NA | NA |
| 89 | 13.37 | 8.18 | 6.69 | 24.94 |
| 90 | 13.56 | 15.97 | 10.39 | 37.96 |
| 91 | 14.09 | 8.03 | 7.78 | 24.52 |
| 92 | 14.15 | 26.48 | NA | NA |
| 93 | 15.50 | 16.43 | 9.36 | 36.26 |
| 94 | 18.30 | 23.92 | 5.52 | 47.74 |
| 95 | 19.95 | 22.15 | 13.48 | 46.68 |
| 96 | 19.99 | 17.46 | 1.88 | 45.73 |
| 97 | 22.86 | 12.95 | 14.39 | 38.61 |
| 98 | 24.39 | 32.32 | 3.66 | 39.93 |
| 99 | 25.30 | 13.33 | 116.82 | 36.78 |
| 100 | 26.84 | 9.53 | 13.42 | 29.27 |
| 101 | 29.25 | 11.17 | NA | NA |
| 102 | 29.58 | 12.65 | 10.89 | 35.36 |
| 103 | 30.53 | 13.98 | NA | NA |
| 104 | 35.02 | 62.22 | 2036.07 | 79.34 |
| 105 | 35.70 | 27.68 | 4.91 | 55.87 |
| 106 | 37.92 | 17.24 | 54.58 | 53.56 |
| 107 | 38.28 | 18.14 | 8.44 | 44.59 |
| 108 | 40.52 | 13.46 | 18.21 | 28.68 |
| 109 | 42.12 | 11.09 | 10.22 | 35.33 |
| 110 | 45.27 | 14.00 | 185.58 | 10.20 |
| 111 | 45.78 | 73.08 | 551.15 | 60.03 |
| 112 | 46.68 | 18.51 | NA | NA |
| 113 | 51.78 | 21.00 | 9.28 | 38.05 |
| 114 | 55.70 | 14.15 | 34.95 | 40.82 |
| 115 | 65.30 | 53.22 | 117.54 | 59.97 |
| 116 | 67.18 | 34.23 | NA | NA |
| 117 | 73.68 | 25.54 | NA | NA |
| 118 | 79.64 | 12.49 | NA | NA |
| 119 | 84.89 | 40.36 | 27.03 | 59.05 |
| 120 | 87.06 | 12.66 | 3.03 | 28.57 |
| 121 | 95.45 | 40.50 | 1358.88 | 75.47 |
| 122 | 98.07 | 74.73 | NA | NA |
| 123 | 108.94 | 45.54 | 28.34 | 78.23 |
| 124 | 124.48 | 43.76 | NA | NA |
| 125 | 149.23 | 29.00 | 261.64 | 34.48 |
| 126 | 169.09 | 89.33 | 0.47 | 91.32 |
| 127 | 213.26 | 42.87 | 8.94 | 37.97 |
| 128 | 292.21 | 58.00 | 191.42 | 83.49 |
| 129 | 1000.00 | 95.34 | 499.01 | 92.49 |
| 130 | 1000.00 | 84.84 | 3898.06 | 76.29 |
| 131 | 1000.00 | 92.07 | 3.10 | 59.06 |
| 132 | 1000.00 | 89.82 | 0.10 | 89.52 |
| 133 | 1000.00 | 82.45 | 23.29 | 84.63 |
| 134 | 1000.00 | 91.34 | 8.50 | 65.79 |
| 135 | 1000.00 | 87.67 | 10000.00 | 84.14 |
| 136 | 1000.00 | 96.76 | 10000.00 | 95.61 |
| 137 | 1000.00 | 81.91 | NA | NA |
| 138 | 1000.00 | 87.20 | 159.65 | 89.63 |
| 139 | 1000.00 | 93.99 | NA | NA |
| 140 | 3.11 | 9.35 | 2.20 | 29.76 |
| 141 | 9.0 | 53 | NA | NA |
| 142 | NA | NA | NA | NA |
| 143 | NA | NA | NA | NA |
| 144 | 10.14 | 11.17 | 7.14 | 35.43 |
| 145 | 51.91 | 47.34 | 74.75 | 66.44 |
| 146 | 13.05 | 22.43 | 9.40 | 57.23 |

NA: Not available

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

What is claimed is:

1. A compound having a Formula (I), 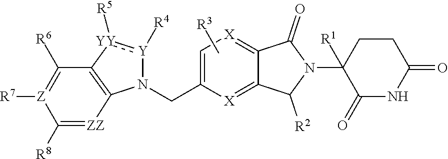
or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein: [symbol]
may be absent or a double bond;
X is independently selected from C and N;
Y is independently selected from C and N;
YY is independently selected from C and N;
Z is independently selected from C and N;
ZZ is independently selected from C and N;
R¹ is independently selected from hydrogen, halogen, and —C₁-C₆ alkyl;
R² is independently selected from hydrogen, halogen, —(═O), —C₁-C₆ alkyl, a 3 to 6 membered cycloalkyl, wherein the alkyl or cycloalkyl may be optionally substituted with —R⁹, —N(R⁹R¹⁰), and —OR⁹;
R³ is independently absent or selected from hydrogen and halogen;
R⁴ is independently absent or selected from hydrogen and —C₁-C₆ alkyl;
R⁵ is independently absent or selected from hydrogen, halogen and —C₁-C₆ alkyl;
R⁶ is independently selected from a 5 to 12 membered aryl and a 5 to 12 membered heteroaryl ring, wherein the aryl and heteroaryl may be optionally substituted with 1, 2, or 3-R⁹, —N(R⁹R¹⁰), and —OR⁹;
R⁷ is hydrogen or halogen;
R⁸ is independently selected from —N(R⁹R¹⁰), and a 4 to 12 membered heterocyclic ring, wherein the heterocyclic ring may be optionally substituted with 1, 2 or 3-R⁹, —N(R⁹R¹⁰), and —OR⁹;
R⁹ is independently selected from hydrogen, halogen, —OR¹⁰, —N(R¹⁰R¹⁰), —C₁-C₆ alkyl, —O—C₁-C₆ alkyl, —CN, a 3 to 12 membered cycloalkyl, and a 4 to 12 membered heterocyclic ring;
wherein the alkyl, cycloalkyl, or heterocyclic ring in R⁹ are each independently unsubstituted or substituted with 1, 2, or 3 R¹⁰ substituents;
R¹⁰ in each instance is independently selected from hydrogen, —OH, —C₁-C₆ alkyl, —C₂-C₆ alkenyl, halogen, —O—(C₁-C₆ alkyl)-, —N(R¹¹R¹¹), a 3 to 12 membered cycloalkyl, a 4 to 12 membered heterocyclic, a 5 to 12 membered aryl and a 5 to 12 membered heteroaryl ring;
wherein the alkyl, alkenyl, cycloalkyl, heterocyclic, aryl or heteroaryl ring in R¹⁰ are each independently unsubstituted or substituted with 1, 2, or 3 R¹¹ substituents; and
R¹¹ is independently selected from hydrogen, halogen, —OH, and —C₁-C₆ alkyl, and further wherein the alkyl, heterocyclic and heteroaryl ring in each R⁶, R⁸, R⁹, and R¹⁰ may include 1, 2 or 3 heteroatoms independently selected from O, N or S.

2. A compound of claim 1, having a Formula (Ia), 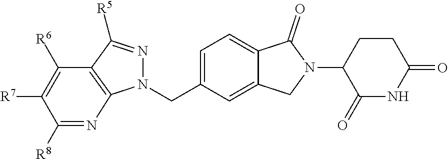
or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
R⁵ is independently absent or selected from hydrogen, halogen or —C₁-C₆ alkyl;
R⁶ is independently selected from a 5 to 12 membered aryl or a 5 to 12 membered heteroaryl ring, wherein the aryl, and heteroaryl may be optionally substituted with 1, 2, or 3-R⁹, —N(R⁹R¹⁰), or —OR⁹;
R⁷ is hydrogen or halogen;
R⁸ is independently selected from —N(R⁹R¹⁰), a 4 to 12 membered heterocyclic, ring, wherein the heterocyclic ring may be optionally substituted with 1, 2 or 3-R⁹, —N(R⁹R¹⁰), or —OR⁹;
R⁹ is independently selected from hydrogen, halogen, —C₁-C₆ alkyl, —OR¹⁰, —N(R¹⁰R¹⁰), —CN, a 3 to 12 membered cycloalkyl, or a 4 to 12 membered heterocyclic ring;
wherein the alkyl, cycloalkyl, or heterocyclic ring in R⁹ are each independently unsubstituted or substituted with 1, 2, or 3 R¹⁰ substituents;
R¹⁰ in each instance is independently selected from hydrogen, halogen, —OH, —C₁-C₆ alkyl, 3 to 12 membered cycloalkyl, and 4 to 12 membered heterocyclic ring; wherein the alkyl, cycloalkyl, and heterocyclic ring in R¹⁰ are each independently unsubstituted or substituted with 1, 2, or 3 R¹¹ substituents;
R¹¹ is independently hydrogen, halogen, —OH, and —C₁-C₆ alkyl, further wherein the cycloalkyl, heterocyclic and heteroaryl cyclic ring in each R⁶, R⁸, R⁹, and R¹⁰ may include 1, 2 or 3 heteroatoms independently selected from O, N or S.

3. The compound of claim 1, wherein the halogen is F or C₁.

4. The compound of claim 1, wherein R⁸ is independently selected from —N(R⁹R¹⁰), and a 4 to 12 membered heterocyclic ring.

5. The compound of claim 1, wherein R⁶ is independently selected from:

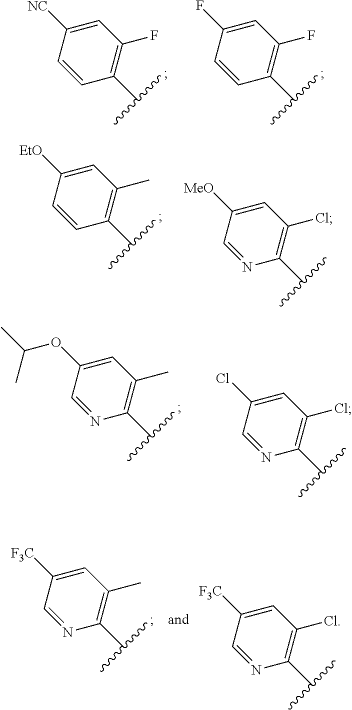

6. The compound of claim 1, wherein R⁸ is independently selected from:

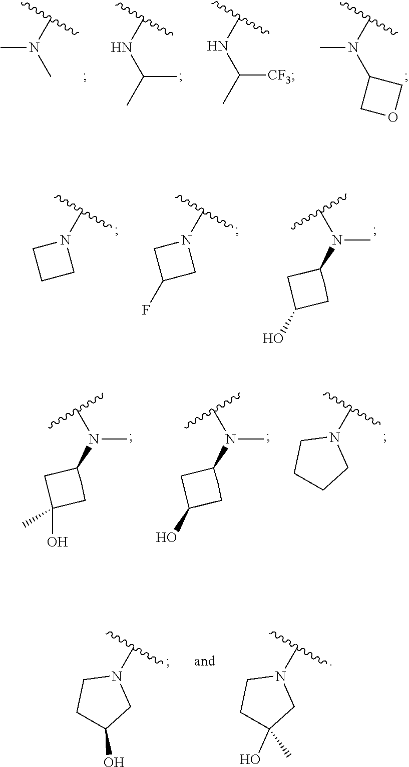

7. A compound of claim 1, having a Formula (Ib), 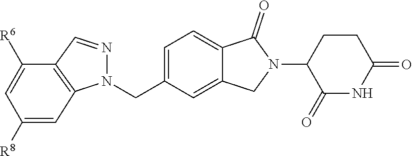
or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
R⁶ is 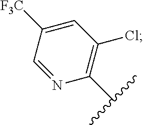 [SMILES: *c1ncc(C(F)(F)F)cc1Cl]
and
R⁸ is

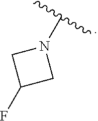 [SMILES: *N1CC(F)C1]

8. A compound of claim 1, having a Formula (Ic), 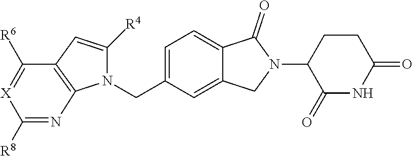
or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
X is C or N;
R⁴ is independently selected from hydrogen or —C₁-C₆ alkyl;
R⁶ is independently selected from 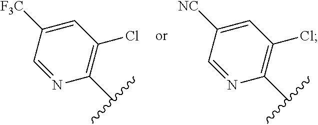 [SMILES: *c1ncc(C(F)(F)F)cc1Cl ; *c1ncc(C#N)cc1Cl]
and
R⁸ is independently selected from 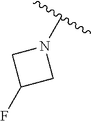 [SMILES: *N1CC(F)C1]
or —N(CH₃CH₃).

9. A compound of claim 1, having a Formula (Id), 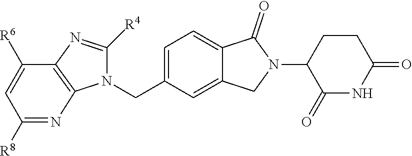
or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
R⁴ is independently selected from hydrogen or —C₁-C₆ alkyl;
R⁶ is independently selected from 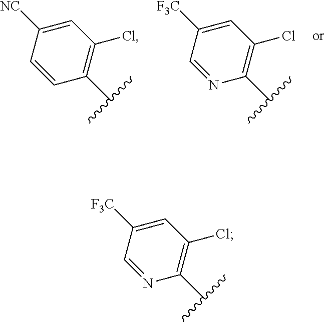 [SMILES: *c1ccc(C#N)cc1Cl ; *c1ncc(C(F)(F)F)cc1Cl ; *c1ncc(C(F)(F)F)cc1Cl]
and
R⁸ is independently selected from

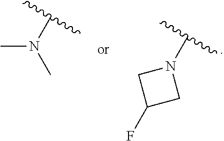

10. A compound of claim 1, having a Formula (Ie), 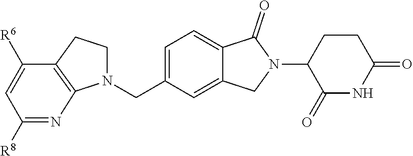
or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof,
wherein:
R⁶ is 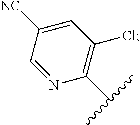 [SMILES: *c1ncc(C#N)cc1Cl]
and
R⁸ is —N(CH₃CH₃).

11. A compound, selected from 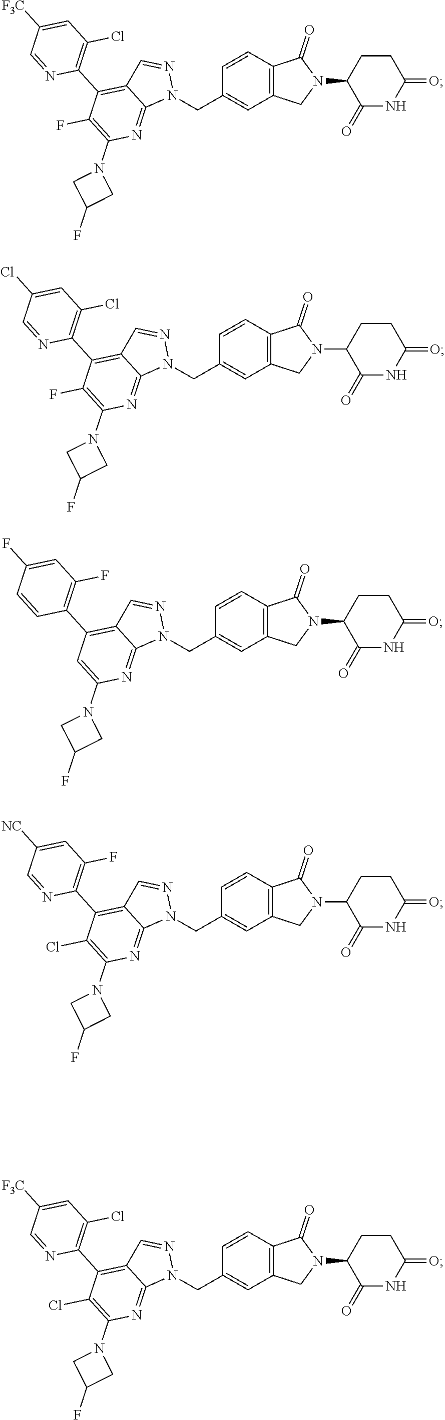 [SMILES: O=C1CC[C@H](N2Cc3cc(Cn4ncc5c(-c6ncc(C(F)(F)F)cc6Cl)c(F)c(N6CC(F)C6)nc54)ccc3C2=O)C(=O)N1 ; O=C1CCC(N2Cc3cc(Cn4ncc5c(-c6ncc(Cl)cc6Cl)c(F)c(N6CC(F)C6)nc54)ccc3C2=O)C(=O)N1 ; O=C1CC[C@H](N2Cc3cc(Cn4ncc5c(-c6ccc(F)cc6F)cc(N6CC(F)C6)nc54)ccc3C2=O)C(=O)N1 ; N#Cc1cnc(-c2c(Cl)c(N3CC(F)C3)nc3c2cnn3Cc2ccc3c(c2)CN(C2CCC(=O)NC2=O)C3=O)c(F)c1 ; O=C1CC[C@H](N2Cc3cc(Cn4ncc5c(-c6ncc(C(F)(F)F)cc6Cl)c(Cl)c(N6CC(F)C6)nc54)ccc3C2=O)C(=O)N1] 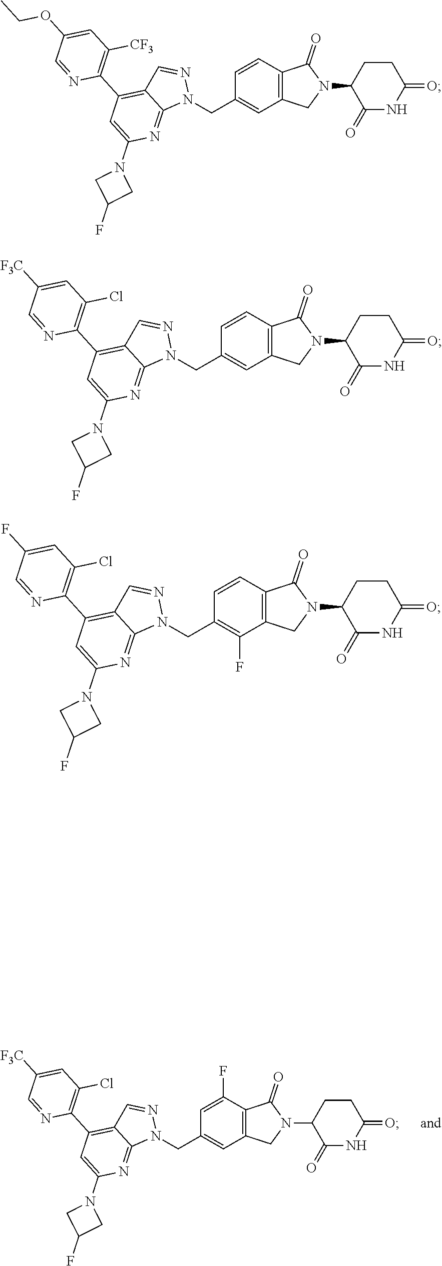 [SMILES: CCOc1cnc(-c2cc(N3CC(F)C3)nc3c2cnn3Cc2ccc3c(c2)CN([C@H]2CCC(=O)NC2=O)C3=O)c(C(F)(F)F)c1 ; O=C1CC[C@H](N2Cc3cc(Cn4ncc5c(-c6ncc(C(F)(F)F)cc6Cl)cc(N6CC(F)C6)nc54)ccc3C2=O)C(=O)N1 ; O=C1CC[C@H](N2Cc3c(ccc(Cn4ncc5c(-c6ncc(F)cc6Cl)cc(N6CC(F)C6)nc54)c3F)C2=O)C(=O)N1 ; O=C1CCC(N2Cc3cc(Cn4ncc5c(-c6ncc(C(F)(F)F)cc6Cl)cc(N6CC(F)C6)nc54)cc(F)c3C2=O)C(=O)N1] 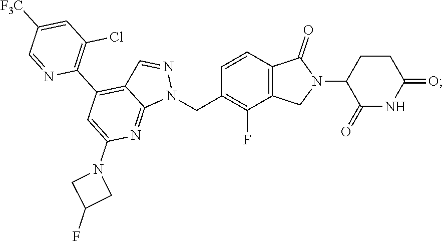 [SMILES: O=C1CCC(N2Cc3c(ccc(Cn4ncc5c(-c6ncc(C(F)(F)F)cc6Cl)cc(N6CC(F)C6)nc54)c3F)C2=O)C(=O)N1]
or a pharmaceutically acceptable salt, solvate, hydrate, co-crystal, clathrate, polymorph or tautomer thereof, a pharmaceutically acceptable salt of the polymorph or tautomer, a stereoisomer of any of the foregoing, or a mixture thereof.

12. A method for reducing FAK protein levels, the method comprising contacting a cell with an effective amount of a compound of claim 1 or a pharmaceutically acceptable salt, tautomer, isotopologue, or stereoisomer thereof.

13. The method of claim 12, wherein the cell is in a subject.

14. A method of preventing or treating cancer in a subject comprising administering to a subject in need thereof an effective amount of a compound of claim 1 or a pharmaceutically acceptable salt, tautomer, isotopologue, or stereoisomer thereof.

15. The method according to claim 14, where the cancer is selected from gastric, lung, pancreatic, ovarian, breast, skin, colon, neuroblastoma, osteosarcoma, uterine, rectal, and kidney cancer.

16. A compound of claim 1 for use as a medicament.

17. The use of a compound of claim 1 or a pharmaceutically acceptable salt, tautomer, isotopologue, or stereoisomer thereof, in the manufacture of a medicament for reducing FAK protein levels.

18. The use of a compound of claim 1 or a pharmaceutically acceptable salt, tautomer, isotopologue, or stereoisomer thereof, in the manufacture of a medicament for the prevention or treatment of cancer.

19. The compound for use according to claim 18, wherein the use further comprises administering a therapeutically effective amount of another second active agent or a support care therapy, wherein the other second active agent is a therapeutic antibody that specifically binds to a cancer antigen, hematopoietic growth factor, cytokine, anti-cancer agent, antibiotic, cox-2 inhibitor, immunomodulatory agent, immunosuppressive agent, corticosteroid or a pharmacologically active mutant or derivative thereof.
